(19) United States
(12) Patent Application Publication
(10) Pub. No.: US 2024/0000859 A1
(43) Pub. Date: Jan. 4, 2024

(54) DESIGNED BACTERIAL COMPOSITIONS FOR TREATING GRAFT-VERSUS-HOST-DISEASE

(71) Applicant: Seres Therapeutics, Inc., Cambridge, MA (US)

(72) Inventors: Matthew R. HENN, Cambridge, MA (US); Edward J. O'BRIEN, Cambridge, MA (US); Ambar PIÑA, Cambridge, MA (US); Marin VULIC, Cambridge, MA (US); Christopher B. FORD, Cambridge, MA (US); Asuncion MARTINEZ, Cambridge, MA (US); Divya BALASUBRAMANIAN, Cambridge, MA (US); Elizabeth HALVORSEN, Cambridge, MA (US); Karen KIESER, Cambridge, MA (US); Mahmoud SALEH, Cambridge, MA (US); Mary-Jane LOMBARDO, Cambridge, MA (US); Sumon DATTA, Cambridge, MA (US); Madhumitha NANDAKUMAR, Cambridge, MA (US); Priyanka NARENDAR, Cambridge, MA (US); Kankana BARDHAN, Cambridge, MA (US)

(73) Assignee: Seres Therapeutics, Inc., Cambridge, MA (US)

(21) Appl. No.: 18/254,449

(22) PCT Filed: Nov. 24, 2021
(86) PCT No.: PCT/US2021/060878

§ 371 Date: May 25, 2023

Related U.S. Application Data
(60) Provisional application No. 63/118,639, filed on Nov. 25, 2020.

(51) Int. Cl. A61K 35/74; A61P 31/04; A61P 29/00

(52) U.S. Cl. CPC A61K 35/74; A61P 31/04; A61P 29/00

(57) ABSTRACT

Provided herein are bacterial compositions that are useful for treating and preventing complications and side effects associated with a disease or disorder, such as those associated with immune suppression, including infections and/or GvHD, for example, in HSCT subjects. The bacterial compositions disclosed herein are designed to exhibit one or more functional features that are useful for the treatment of such diseases and disorders.

### CROSS-REFERENCE TO RELATED APPLICATIONS

This application claims the priority benefit of U.S. Provisional Application No. 63/118,639, filed Nov. 25, 2020, which is herein incorporated by reference in its entirety.

### REFERENCE TO SEQUENCE LISTING SUBMITTED ELECTRONICALLY

The content of the electronically submitted sequence listing in ASCII text file (Name: 4268_020PC02_Seqlisting_ST25.txt; Size: 731,860 bytes; and Date of Creation: Nov. 24, 2021) filed with the application is herein incorporated by reference in its entirety.

### FIELD

The present disclosure relates to bacterial compositions designed to have certain functional features that are useful for treating and/or preventing a range of diseases and disorders related to immune suppression, such as an infection (including a viral infection or reactivation, invasive infection, blood stream infection), graft-versus-host-disease (GvHD) (for example, in subjects undergoing hematopoietic stem cell transplantation (HSCT) or having an allogeneic or autologous immune response), a cancer, or in subjects undergoing chemotherapy or undergoing or having undergone transplantation.

### BACKGROUND

Allogeneic hematopoietic stem cell transplantation (HSCT) is an important treatment with curative intent for hematological malignancies, such as leukemia and lymphoma. As the safety of HSCT has improved over recent decades, it has become an important cancer therapy and is performed more than 25,000 times annually worldwide (Gratwohl A., et al., JAMA 2010 Apr. 28; 303 (16):1617-24, Gooley T. A., et al., N Engl J Med. 2010 Nov. 25; 363(22): 2091-2101). The objective of HSCT is to replace diseased cells in the recipient bone marrow with healthy donor stem cells. Recognition and eradication of residual diseased host cells takes place through cellular immunotherapy provided by the transplant (Juric 2016).

Despite its success, HSCT is not without risks. For instance, chemotherapy is commonly used to condition a patient for transplantation, so that the patient's immune system does not reject the transplantation. However, chemotherapy suppresses the patient's immune system (e.g., decreased circulation of granulocytes and monocytes) and can thereby, increase the patient's susceptibility to infections (Junghanss C., et al., Biol Blood Marrow Transplant 2002; 8(9): 512-20, Blijlevens N. M., et al., Bone Marrow Transplantation 2005; 35: 707-711). Additionally, because of the difficulties in finding a syngenic donor (i.e., genetically identical or sufficiently identical, so that the donor's cells are immunologically compatible with the recipient's immune system), many HSCTs involve allogenic donors. Even with the use of conditioning regimens (e.g., chemotherapy and antibiotics), the donated immune cells can recognize the host cells as foreign and mount an immune response, which can lead to life-threatening inflammation, such as that observed in graft-versus-host disease (GvHD). Accordingly, infection and GvHD account for approximately 40% of deaths in the first 100 days after transplant (D'Souza, A, Fretham C, Lee S J, et al. Current Use of and Trends in Hematopoietic Cell Transplantation in the United States. Biol Blood Marrow Transplant. 2020 May 11:S1083-8791(20)30225-1, D'Souza A., et al. Biol Blood Marrow Transplant 2017 September; 23(9):1417-1421).

Studies conducted at transplant centers have shown that dysbiosis contributes to both infectious and acute GvHD outcomes with significant reductions in overall survival (OS) and increased mortality (Peled NEJM 2020). The combination of conditioning regimen and antibiotic treatment leads to injury of the GI microbiome (Jenq 2015; Baumgartner 2017; Montassier 2016). A state of dysbiosis develops, characterized by expansion of potentially pathogenic bacteria and loss of species diversity (Peled 2020) in some subjects, a condition of species domination arises in which pathogens constitute over 30% of the organisms observed in stool. These organisms may translocate across an impaired GI tract barrier into the blood stream and cause infections. Bloodstream infections caused by the same organisms that are found in the GI tract are found in 25-50% of HSCT recipients (Taur 2012; Tamburini 2018). Moreover, translocation of pathogens may kindle an inflammatory response, influencing the pathophysiology of acute Graft versus Host Disease (aGvHD) (Peled 2020), a complication that affects the skin, upper and lower GI tract, and the liver.

Immune suppression, infections, alterations in gut microbe ecology, inflammation, organ damage, risk of death, and other risks associated with GvHD are not limited to subjects who have or are undergoing therapy for GvHD. Immunes suppression and these additional manifestations of diseases and disorders also present risks for subjects who have or are at risk of developing cancer; subjects undergoing chemotherapy; subjects having an allogenic or an autologous immune response; subjects undergoing or having undergone transplantation; subjects infected with bacterial, viral, or other pathogens (including a viral infection or reactivation, invasive infection, blood stream infection); and other circumstances which cause dysbiosis or immune challenges in a subject.

Accordingly, there remains a need for new and alternative approaches to treating the different diseases and disorders that can occur in HSCT patients, such as infections and GvHD, as well as other diseases and disorders affecting immune suppression, including subjects having cancer, having an allogeneic or an autologous immune response, undergoing chemotherapy, or undergoing or having undergone transplantation.

### BRIEF SUMMARY

Provided herein are bacterial compositions designed to have certain bacterial species and/or strains and/or functional features that are useful for treating and/or preventing a range of diseases and disorders related to immune suppression and associated with, for example, infection, GvHD, an allogeneic or an autologous immune response, chemotherapy, transplantation, as well as related conditions. Also provided are methods of treating diseases and disorders as described herein.

In aspects provided herein the compositions comprise a purified population of bacteria, wherein the purified population of bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1-352. In some aspects, the compositions comprise a purified population of bacteria, wherein the purified population of bacteria comprises Eubacterium maltosivorans, Clostridium aldenense, Clostridium bolteae, Clostridium glycyrrhizinilyticum, Clostridium hylemonae, Clostridium innocuum, Clostridium lavalense, Clostridium scindens, Clostridium spiroforme, Clostridium symbiosum, Eubacterium rectale, Ruminococcus gnavus, Ruminococcus torques, Absiella dolichum, Agathobaculum desmolans, Akkermansia muciniphila, Alistipes finegoldii, Alistipes shahii, Anaerofustis stercorihominis, Anaeromassilibacillus senegalensis, Anaerostipes caccae, Anaerotruncus colihominis, Bacteroides caccae, Faecalibacterium prausnitzii, Faecalicatena contorta, Faecalicatena orotica, Flavonifractor plautii, Gemmiger formicilis, Harryflintia acetispora, Holdemania filiformis, Holdemania massiliensis, Intestinimonas butyriciproducens, Lachnospira pectinoschiza, Lachnospiraceae bacterium 5 1 57FAA, Lactobacillus fermentum, Lactonifactor longoviformis, Longibaculum muris, Longicatena caecimuris, Murimonas intestina, Oscillibacter ruminantium, Bacteroides eggerthii, Bacteroides faecis, Bacteroides intestinalis, Bacteroides koreensis, Bacteroides kribbi, Bacteroides salyersiae, Bacteroides uniformis, Bacteroides vulgatus, Bacteroides xylanisolvens, Barnesiella intestinihominis, Bifidobacterium dentium, Bifidobacterium longum, Bifidobacterium stercoris, Blautia coccoides, Blautia hominis, Blautia hydrogenotrophica, Blautia luti, Blautia obeum, Blautia producta, Blautia wexlerae, Butyricimonas faecihominis, Cellulosilyticum lentocellum, Clostridium butyricum, Ruthenibacterium lactatiformans, Sellimonas intestinalis, Shigella flexneri, Terrisporobacter mayombei, Terrisporobacter petrolearius, Turicibacter sanguinis, Tyzzerella nexilis, Clostridium disporicum, Clostridium subterminale, Clostridium tertium, Collinsella aerofaciens, Coprococcus comes, Coprococcus eutactus, Dorea longicatena, Drancourtella massiliensis, Eggerthella lenta, Eisenbergiella tayi, Emergencia timonensis, Erysipelatoclostridium ramosum, Eubacterium callanderi, Paeniclostridium sordellii, Parabacteroides distasonis, Parabacteroides merdae, Paraclostridium bifermentans, Peptostreptococcus stomatis, Robinsoniella peoriensis, Romboutsia timonensis, Roseburia intestinalis, Roseburia inulinivorans, Ruminococcus albus, Ruminococcus bromii, Ruminococcus faecis, Ruminococcus lactaris, or combinations thereof. In some aspects, the compositions comprise a purified population of bacteria, wherein the purified population of bacteria comprises a species selected from FIG. 1 or combinations thereof. In some aspects, the compositions comprise a purified population of bacteria, wherein the purified population of bacteria comprises species of DE122435.3 (DE10 in FIG. 1), DE122435.1 (DE8 in FIG. 1), or DE122435.4 (DE11 in FIG. 1) or bacteria having a 16S rDNA sequence that is at least 97%, at least 98%, at least 99%, or 100% identical to a 16S rDNA sequence of bacteria in the recited designed compositions. In some aspects, the compositions comprise a purified population of bacteria, wherein the purified population of bacteria comprises species of DE486373.1 (DE23 in FIG. 1) or bacteria having a 16S rDNA sequence that is at least 97%, at least 98%, at least 99%, or 100% identical to a 16S rDNA sequence of bacteria in the recited designed composition.

In aspects described herein, the bacterial compositions described herein are used in methods of treating and/or reducing the risk of an infection in a subject in need thereof, comprising administering to the subject an effective amount of such bacterial compositions or pharmaceutical formulations thereof, wherein the infection includes a blood stream infection, sepsis, tissue infection, invasive infection, a gastrointestinal infection, a viral infection or reactivation, or a combination thereof. In some aspects, the bacterial compositions described herein are used in methods of treating and/or reducing the risk of graft-versus-host-disease (GvHD) in a subject in need thereof, comprising administering to the subject an effective amount of such bacterial compositions or pharmaceutical formulations thereof. In some aspects, the bacterial compositions described herein are used in methods of treating and/or reducing the risk of mucositis in a subject in need thereof, comprising administering to the subject an effective amount of such bacterial compositions or pharmaceutical formulations thereof.

In aspects described herein, the bacterial compositions described herein are used in methods of treating a disease or disorder associated with an allogeneic or an autologous immune response in a subject in need thereof, comprising administering to the subject an effective amount of such bacterial compositions or pharmaceutical formulations thereof. In some aspects, the bacterial compositions described herein are used in methods of treating, reducing, or alleviating a symptom associated with chemotherapy in a subject in need thereof, comprising administering to the subject an effective amount of such bacterial compositions or pharmaceutical formulations thereof. In some aspects, the bacterial compositions described herein are used in methods of preventing, reducing, or treating rejection in a subject undergoing transplantation (e.g., either HSCT or organ), comprising administering to the subject an effective amount of such bacterial compositions or pharmaceutical formulations thereof.

In aspects described herein, the bacterial compositions described herein are used in methods of modulating a biological activity in a subject in need thereof, comprising administering to the subject an effective amount of such bacterial compositions or pharmaceutical formulations thereof, where such biological activity includes short-chain fatty acid production, medium-chain fatty acid production, tryptophan metabolite production, fucosidase activity, Wnt activation, anti-IL-8 activity, or combinations thereof. In some aspects, the bacterial compositions described herein are used in methods of decreasing the number and/or relative abundance of antibiotic resistant bacteria in a gastrointestinal tract of a subject in need thereof, comprising administering to the subject an effective amount of such bacterial compositions or pharmaceutical formulations thereof. In some aspects, the bacterial compositions described herein are used in methods of improving epithelial barrier status, reducing inflammation, and/or reducing mucositis in a gastrointestinal tract of a subject in need thereof, comprising administering to the subject an effective amount of such bacterial compositions or pharmaceutical formulations thereof.

In aspects described herein, the bacterial compositions described herein are used in methods of decreasing mortality due to an invasive infection in a subject in need thereof, comprising administering to the subject an effective amount of such bacterial compositions or pharmaceutical formulations thereof, where the subject is undergoing or has undergone transplantation. In some aspects, the bacterial compositions described herein are used in methods of reducing transplantation-related complications in a subject in need thereof, comprising administering to the subject an effective amount of such bacterial compositions or pharmaceutical formulations thereof, where the subject is undergoing or has undergone transplantation. In some aspects, the bacterial compositions described herein are used in methods of increasing the overall survival and/or progression-free survival of a subject in need thereof, comprising administering to the subject an effective amount of such bacterial compositions or pharmaceutical formulations thereof, where the subject is undergoing or has undergone transplantation.

In aspects described herein, the subjects treated or administered the bacterial compositions or pharmaceutical formulations thereof according to the methods described herein have undergone or are undergoing transplantation. In some aspects, the transplantation is an allogeneic hematopoietic stem cell transplantation (allo-HSCT) or an allogeneic organ transplantation. In some aspects, the transplantation is an autologous hematopoietic stem cell transplantation (allo-HSCT) or an autologous organ transplantation. In some aspects, subjects that have undergone or are undergoing transplantation and administered the bacterial compositions or pharmaceutical formulations thereof have i) an increased prevalence in their stool of one or more strains in the bacterial composition, ii) a decreased abundance in their stool of Enterococcus spp., Enterobacteriaceae spp., or both, iii) a decreased incidence of bloodstream infections including but not limited to bacterial infections (VRE, CRE, or ESBL), fungal infections, or combinations thereof, iv) a decreased incidence of gastrointestinal infections including but not limited to Clostridiodes difficile, viral infections (including but not limited to norovirus, adenovirus, or rotavirus), parasitic infections (including but not limited to Cryptosporidia), or combinations thereof, v) a decreased incidence of acute GvHD including but not limited to acute GvHD Grades II, III, and IV, vi) a decreased incidence of febrile neutropenia, vii) reduced frequency, length, or both frequency and length of hospitalization stay, or vii) any combination thereof, relative to a reference (e.g., a corresponding reference in a subject that did not receive the composition disclosed herein or corresponding reference in the subject prior to the administration of the composition).

In aspects described herein, the subjects treated or administered the bacterial compositions or pharmaceutical formulations thereof according to the methods described herein suffers from a cancer. In some aspects, the cancer comprises acute myeloid leukemia (AML), acute lymphoblastic leukemia (ALL), myelodysplastic syndromes (MDS), myeloproliferative neoplasms (MPN), or combinations thereof.

### BRIEF DESCRIPTION OF THE DRAWINGS/FIGURES

FIG. 1 shows the DEs tested in the VRE and/or CRE decolonization animal models and their strain makeup. The VRE and CRE log reduction values are averaged across all time points and repeated runs.

FIGS. 2A, 2B, and 2C show the diversity of the DEs tested in the VRE and/or CRE decolonization animal models. FIG. 2A shows the STR strain makeups in the DEs tested. FIG. 2B shows the histogram of the numbers of strains in the DEs tested. It shows most DEs tested have 13, 14, 15, 16, or 17 strains. FIG. 2C shows the histogram of the frequencies of strains being included in the DE tested.

FIGS. 3A, 3B, 3C, 3D, 3E, 3F, and 3G summarize log titer reduction of DEs tested in the VRE or CRE decolonization animal models. FIGS. 3A and 3B show the histogram of average VRE or CRE log titer reduction across all time points in the animal models of the DEs tested. FIG. 3C shows that the average log reduction in the VRE titer in the DE treated animals at days 7, 9, 11, 13, 15, 18, and 21 post VRE challenge. FIG. 3D shows that the average log reduction in the CRE titer in the DE treated animals at days 7, 9, 11, 13, 15, 18, and 21 post CRE challenge. FIGS. 3E and 3F demonstrate that the correlations between the number of strains in the DEs and the average log reduction across all time points. The Spearman's correlation coefficient r=0.028, p-value=0.84 and r=−0.025, p-value=0.90 indicate that the number of strains in a DE is not correlated with VRE and CRE decolonization, respectively. FIG. 3G demonstrates the correlation between VRE and CRE titer reduction by a DE. The Spearman's correlation coefficient r=0.14 and p-value=0.49 indicate that the VRE and CRE titer reduction by a DE is not correlated, i.e., VRE and CRE decolonization in the animal models are driven by distinct factors. The log reduction in the titer was determined from the difference between the median of the vehicle only group and DE treatment group at a given time-point.

FIGS. 4A, 4B, 4C, 4D, 4E, and 4F show the effect of strains and strain combinations from the DEs tested on VRE or CRE decolonization estimated by the single strain additive model, which estimates the additive effect of each strain on VRE/CRE decolonization, after accounting for the additive effects of other strains. Each circle indicates a strain. Strains that are perfectly collinear are combined into one circle and these combined strains are connected by “_” in the strain list in FIGS. 4E and 4F. FIG. 4A shows a volcano plot illustrating the estimated strain effect on log VRE titer reduction and the significance of the effect. The dotted line indicates p value of 0.05. The estimated effects of the strains above the dotted line are significant at a p value lower than 0.05. FIG. 4B shows the estimated effect on log VRE titer reduction of each strain. The vertical line indicates 90% confidence interval and the open circle indicates strain or strain combinations with significant estimated effect. FIG. 4C shows a volcano plot illustrating the estimated strain effect on log CRE titer reduction and the significance of the effect. The dotted line indicates p value of 0.05. The estimated effects of the strains above the dotted line are significant at a p value lower than 0.05. FIG. 4D shows the estimated effect on log CRE titer reduction of each strain. The vertical line indicates 90% confidence interval and the open circle indicates strain or strain combinations with significant estimated effect. FIG. 4E records all the results for each strain from VRE single strain additive model including t-statistic, p-value, linear coefficient, confidence interval for linear coefficient. FIG. 4F records all the results for each strain from CRE single strain additive model including t-statistic, p-value, linear coefficient, confidence interval for linear coefficient. A negative value in estimated strain effect on log VRE or CRE suggests that the strain reduces VRE or CRE colonization while a positive value suggests that the strain increases VRE or CRE colonization.

FIGS. 5A, 5B, 5C, and 5D illustrate the analysis results from the strain interaction model, which estimate pairwise (synergistic and antagonistic) interactions between strains on VRE/CRE decolonization, accounting for differences from the expected effects based on single strain additive effects. Each circle indicates a pair of strains. Strain interactions that are perfectly collinear are combined into one circle and these combined strains are connected by “_or_” in the strain list in FIGS. 5C and 5D. FIG. 5A shows the estimated pair interaction effect on log VRE titer reduction of each strain pair. Only strains with estimated effects at p<0.1 from the single strain additive model were included (n=28 for VRE). All pairwise interactions between these strains included in FIG. 5C. The vertical line indicate 90% confidence interval and the open circle indicates strain combinations with significant estimated effect. FIG. 5B shows the estimated pair interaction effect on log CRE titer reduction of each strain pair. Only strains with estimated effects at p<0.1 from the single strain additive model were included (n=6 for CRE). All pairwise interactions between these strains included in FIG. 5D. The vertical line indicate 90% confidence interval and the open circle indicates strain combinations with significant estimated effect. FIG. 5C records all the results for each strain interaction from VRE strain interaction model including t-statistic, p-value, linear coefficient, confidence interval for linear coefficient. FIG. 5D records all the results for each strain from CRE strain interaction model including t-statistic, p-value, linear coefficient, confidence interval for linear coefficient. A negative value in estimated pair interaction effect on log VRE or CRE suggests that the strain pair synergistically reduces VRE or CRE colonization while a positive value suggests that the strain pair antagonistically affect VRE or CRE colonization.

FIGS. 6A, 6B, and 6C show the ability of the DE122435.3 composition to reduce VRE colonization in vivo. FIG. 6A provides a schematic of the experimental design. FIG. 6B provides a comparison of VRE titer in animals treated with a vehicle control (square) or the DE122435.3 composition (circle) over a course of 21 days post VRE challenge. The median VRE CFU per gram of feces was calculated for each group and plotted (n=6 per group). “L.O.D.” refers to the limit of detection. Data were analyzed using the Mann-Whitney t-test and significance was determined as a p-value of p<0.05=*; p<0.01=**. FIG. 6C provides a table showing the log reduction in VRE titer in the DE122435.3 treated animals compared to animals treated with the vehicle control at days 11, 13, 15, 18, and 21 post VRE challenge. The log reduction in VRE titer was determined from the difference between the median of the vehicle only group and DE122435.3 treatment group at a given time-point.

FIGS. 7A, 7B, and 7C show the ability of the DE122435.3 composition to reduce CRE colonization in vivo. FIG. 7A provides a schematic of the experimental design. FIG. 7B provides a comparison of CRE titer in animals treated with a vehicle control (square) or the DE122435.3 composition (circle) over a course of 21 days post CRE challenge. The median CRE CFU per gram of feces was calculated for each group and plotted (n=10 per group). “L.O.D.” refers to the limit of detection. Data were analyzed using the Mann-Whitney t-test and significance was determined as a p-value of p<0.05=*; p<0.01=**; p<0.001=***; and p<0.0001=****. FIG. 7C provides a table showing the log reduction in CRE titer in the DE122435.3 treated animals compared to animals treated with the vehicle control at days 11, 13, 15, 18, and 21 post CRE challenge. The log reduction in CRE titer was determined from the difference between the median of the vehicle only group and DE122435.3 treatment group at a given time-point.

FIG. 8A provides a schematic diagram of the epithelial barrier integrity assay described in Example 4. FIG. 8B provides a comparison of the ability of DE122435.3, SER-287 (Pilot Lot 20, 21, 22), and negative control DE821956.1 to promote barrier integrity in the presence of IFN-γ stimulation. The ‘Reps’ indicate the HDACi values of the four independent experiments for each bacterial composition. Dots represent biological replicates per experiment. Mean and standard deviations are shown for each test article.

FIG. 9A provides a comparison of the ability of supernatants from (i) DE122435.3, (ii) DE122435.1, (iii) DE122435.4, (iv) DE673670.1, (v) pilot lot 20, and (vi) negative control DE821956.1 composition cultures to inhibit IL-8 secretion by HT29 epithelial cells (IECs) after stimulation with TNF-α. FIG. 9B provides a comparison of the pro-inflammatory nature of the following bacterial compositions: (i) DE122435.3, (ii) DE122435.1, (iii) DE122435.4, (iv) DE673670.1, (v) pilot lot 20, and (vi) negative control DE821956.1. In particular, the pro-inflammatory nature of the compositions is assessed by measuring the ability of the supernatant from the different bacterial composition cultures to induce IL-8 secretion in IECs in the absence of TNF-α stimulation. In both FIGS. 9A and 9B, IECs that were either not stimulated with TNF-α or TNF-α alone were used as controls (negative and positive controls, respectively).

FIG. 10 provides a comparison of the ability of different bacterial compositions to inhibit HDAC activity in vitro. The bacterial compositions shown are as follows: (i) DE122435.1, (ii) DE122435.3, (iii) DE122435.4, (iv) DE673670.1, (v) pilot lot 20 (spore-prep composition), and (vi) negative control DE821956.1. HDAC inhibition activity was quantified in vitro using a chemiluminescent assay. Results are expressed as the % HDAC inhibition relative to uninoculated growth media. The ‘Reps’ indicate the HDACi values of the four independent experiments for each bacterial composition. Dots represent biological replicates per experiment (8-12 replicates). Mean and standard deviations are shown for each test article. For each bacterial composition,

FIGS. 11A, 11B, 11C, 11D, and 11E provide a comparison of various functional attributes of five different DEs (i.e., DE122435.1, DE122435.4, DE122435.3, and DE673670.1) and three spore preps disclosed herein as measured in vitro: (i) pilot lot 20, (ii) pilot lot 21, (iii) pilot plot 22, and negative control DE821956.1. The following functional attributes are shown: (i) ability to produce butyrate (FIG. 11A); (ii) ability to produce propionate (FIG. 11B); (iii) ability to produce acetate (FIG. 11C); (iv) ability to produce valerate (FIG. 11D); (v) ability to produce indole (FIG. 11E).

FIG. 12 provides a comparison of the bile acid metabolic activity of the following bacterial compositions: (i) DE122435.3, (ii) DE821956.1, and pilot natural product (PNP) 167020 (i.e., spore-prep composition). The bile acid metabolic activity is shown as with the 7aD derivatives plotted (FIG. 12).

FIG. 13 provides a schematic diagram of the method used to measure the ability of the bacterial compositions disclosed herein to modulate certain gene expression in primary human colonic organoids. Further description can be found in Example 7.

FIGS. 14A, 14B, 14C, 14D, 14E, 14F, 14G, 14H, 14I, 14J, 14K, 14L, 14M, 14N, 14O, and 14P provide a comparison of the ability of different bacterial compositions to prevent IFN-γ-mediated induction of selected gene expression pathways as measured in the primary human colonic organoid assay described in FIG. 13. The bacterial compositions tested include: (i) DE821956.1, an inflammatory bacterial composition, (ii) Pilot lot 20 (i.e., spore-prep composition), (iii) Pilot lot 21 (i.e., spore-prep composition), and (iv) DE122435.3. Medium alone and IFN-γ alone were used as controls. After treatment, colonic organoid lysates were evaluated for gene expression pathways using the Human Autoimmune Panel (NanoString Technologies). Pathway scores represent the high level expression change for all the genes in the panel assigned to a particular pathway. Pathway scores are Z-transform for ease of representation. The score for the following pathways are shown: (1) NF-κB signaling (FIG. 14A), (2) TNF family signaling (FIG. 14B), (3) inflammasomes (FIG. 14C), (4) oxidative stress (FIG. 14D), (5) apoptosis (FIG. 14E), (6) Th2 differentiation (FIG. 14F), (7) Th17 mediated biology (FIG. 14G), (8) complement system (FIG. 14H), (9) type I interferon signaling (FIG. 14I), (10) type II interferon signaling (FIG. 14J), (11) lymphocyte trafficking (FIG. 14K), (12) Toll Like Receptor signaling (FIG. 14L), (13) NLR signaling (FIG. 14M), (14) mTOR (FIG. 14N), (15) MHC Class I antigen presentation (FIG. 14O), and (16) MHC Class II antigen presentation (FIG. 14P).

FIG. 15 provides an analysis of the effects of treatment with IFN-γ relative to media treatment, or DE122435.3 and IFN-γ relative to treatment with IFN-γ alone, on gene expression for individual chemokines, cytokines, and cytokine receptors of relevance for GvHD regulated by the bacterial compositions disclosed. Genes are indicated as decreasing, not changing, or increasing in expression in either IFN-γ treatment relative to media only, or treatment with DE122435.3 and IFN-γ relative to IFN-γ treatment only.

FIGS. 16A and 16B provide a barplot comparison at the individual gene level of the ability of different bacterial compositions to downmodulate the transcription of various chemokines, cytokines, and interleukins in primary human colonic organoids. The bacterial compositions tested include the following: (i) DE122435.3, (ii) Pilot lot 20 (i.e., spore-prep composition), (iii) Pilot lot 21 (i.e., spore-prep composition), and (iv) DE821956.1. The colonic organoids treated with one of the above bacterial compositions were all stimulated with IFN-γ to induce the inflammatory gene expression. Colonic organoids treated with media alone and IFN-γ alone were used as controls. After treatment, colonic organoid lysates were evaluated for gene expression pathways using the Human Autoimmune Panel (NanoString Technologies). Individual gene counts are normalized using a set of house keeping genes using NSolver software Advanced analysis. FIG. 16A shows the normalized gene expression of various chemokines and cytokines. FIG. 16B shows the normalized gene expression of various interleukins.

FIG. 17 provides a barplot comparison of the ability of different bacterial compositions to induce the transcription in primary human colonic organoids treated with (right side) or without (left side) IFN-γ. The bacterial compositions tested include the following: (i) DE122435.3, (ii) Pilot lot 20 (i.e., spore-prep composition), (iii) Pilot lot 21 (i.e., spore-prep composition), and (iv) DE821956.1. As above, colonic organoids treated with one of the above bacterial compositions were all stimulated with IFN-γ to induce the inflammatory gene expression. Colonic organoids treated with media alone and IFN-γ alone were used as controls. After treatment, colonic organoid lysates were evaluated for gene expression pathways using the Human Autoimmune Panel (NanoString String). Individual gene counts are normalized using a set of house keeping genes using NSolver software Advanced analysis.

FIGS. 18A and 18B show the effect of different bacterial compositions on the viability and anti-inflammatory phenotype (skew towards anti-inflammatory IL-10 production compared to pro-inflammatory IL-6 production) of macrophages differentiated from human monocytic THP-1 cells after PMA stimulation. The bacterial compositions tested include: (i) DE122435.3, (ii) DE821956.1 and (iii) three complex bacterial communities derived from healthy humans (pilot natural products, PNP). Bacterial treatments consist of 1% bacterial culture supernatant, 1% supernatant plus multiplicity of infection (MOI) 20 bacterial cells, or MOI20 bacterial cells alone. Macrophages alone (i.e., not treated with a supernatant of a bacterial composition) and macrophages treated with bacterial broth (the FCM4 growth medium) were used as controls.

FIGS. 19A, 19B, 19C, 19D, 19E, 19F, 19G, 19H, and 19I show the effect of 1% bacterial culture supernatant derived from different bacterial compositions on the transcriptional profile of macrophages treated with different bacterial compositions. The bacterial compositions tested include: (i) DE122435.3, (ii) DE821956.1 and (iii) three complex bacterial communities derived from healthy humans (pilot natural products [PNP], PNP167020, PNP167021, PNP167022). Macrophages alone (i.e., not treated with a supernatant of a bacterial composition) and macrophages treated with bacterial broth were used as controls. Scores for Th1 activation (FIG. 19A), Th2 activation (FIG. 19B), lymphocyte activation (FIG. 19C), cell cycle and apoptosis (FIG. 19D), antigen presentation (FIG. 19E), TLR signaling (FIG. 19F), chemokine signaling (FIG. 19G), cytokine signaling (FIG. 19H), and interferon signaling (FIG. 19I) are derived from individual gene expression values within the respective pathways and are derived from a principal component analysis of these gene expression values across samples (scores are computed in the nSolver software).

FIG. 19J provides a dotplot comparison of cytokine production (e.g. IFNγ, IL-13, IL-4, IL-23, IL-6, IL-17, TNFα, IL-1β, CCL2, and CXCL10) in macrophages treated with 1% bacterial supernatant of bacterial compositions (i) DE122435.3, (ii) DE821956.1 and (iii) PNP167020, PNP167021, PNP167022). Dotplots represent the mean±SD with statistical significance analyzed via one-way ANOVA with Tukey's multiple comparisons correction. Significance was determined as a p-value of p<0.05=*, p<0.01=**, p<0.001=***, p<0.0001=****.

FIGS. 20A and 20B show the effect of 1% supernatant of different bacterial compositions on transcriptional upregulation of two innate immune defense pathways in human macrophages. The bacterial compositions tested include: (i) DE122435.3, and (ii) PNP167020, (iii) PNP167021, and (iv) PNP167022. Macrophages alone (i.e., not treated with a supernatant of a bacterial composition) and macrophages treated with bacterial broth (the FCM4 growth medium) were used as controls. FIG. 20A provides a comparison of the upregulation of the C3 gene in the complement pathway from the different treatment groups. FIG. 20B provides a comparison of the expression of the S100A8 and S100A9 genes, which form an antimicrobial chelation complex upon protein synthesis, for the different groups.

FIGS. 21A, 21B, 21C, and 21D provide comparison of CD4+ T cell immune response in germ-free mice (“GF”) colonized with either DE821956.1, DE122435.1, DE122453.3, DE916091.1, or a positive control composition for four weeks. Germ-free mice that did not receive any bacterial composition (“GF”) were used as a negative control. Lymphocytes were isolated from the colonic lamina propria and the frequency of regulatory and effector CD4+ T cell populations were measured by flow cytometry. FIG. 21A shows the frequency of Foxp3+RORγT+ CD4+ Treg cells. FIG. 21B shows the ratio of the frequency of percent FoxP3+RORγT+ CD4+ T cells to percent IFNγ+ CD4+ T cells. FIG. 21C shows the ratio of the frequency of percent FoxP3+RORγT+ CD4+ T cells to percent IL-17A+RORγT+CD4+ T cells. Bar graphs represent the mean±SEM and points represent individual mice. Data were analyzed using one-way ANOVA with a post-hoc Fisher's LSD test. Significance was determined as a p-value of p<0.05=*, p<0.01=**, p<0.001=***, p<0.0001=****. FIG. 21D shows the treatment design.

FIGS. 22A, 22B, and 22C show the efficacy of the combination of DE122435.3 and the combo ICI antibodies (i.e., anti-PD-L1 and anti-CTLA-4) in treating CT26 tumor in an animal model. FIG. 22A shows the treatment schedule. Germ-free mice were treated with the DE122435.3 composition or negative control DE821956.1. Some of the animals additionally received the combo ICI antibodies, while the control animals received an isotype control antibody. FIG. 22B shows a comparison of tumor volume in the animals from the different treatment groups at day 10 post tumor inoculation (i.e., when the antibody administration started). FIG. 22C shows a comparison of tumor volume in the animals from the different treatment groups at day 21 post tumor inoculation (i.e., when the animals received their final antibody administration). In both FIGS. 22B and 22C, the treatment groups shown include: (i) negative control DE821956.1+ isotype control antibody (first bar); (ii) negative control DE821956.1+ combo ICI antibodies (second bar); (iii) DE122435.3+ isotype control antibody (third bar); and (iv) DE122435.3+ combo ICI antibodies (fourth bar).

FIGS. 23A, 23B, and 23C show the tumor growth dynamics in the CT26 tumor animals treated with one of the following: (i) negative control DE821956.1+ isotype control antibody (first bar); (ii) negative control DE821956.1+ combo ICI antibodies (i.e., anti-PD-L1 and anti-CTLA-4) (second bar); (iii) DE122435.3+ isotype control antibody (third bar); and (iv) DE122435.3+ combo ICI antibodies (fourth bar). FIG. 23A shows tumor growth over a period of 21 days post tumor inoculation in animals from all the treatment groups. FIG. 23B shows a comparison of tumor growth in the animals treated with the negative control DE821956.1 in combination with either the combo ICI antibodies or the isotype control. FIG. 23C shows a comparison of tumor growth in the animals treated with the DE122435.3 in combination with either the combo ICI antibodies or the isotype control.

FIGS. 24A and 24B show the percentages of total CD45⁺ T cells and CD8⁺ T cells, respectively, in the tumor tissues of animals treated with one of the following: (i) negative control DE821956.1+ isotype control antibody (first bar); (ii) negative control DE821956.1+ combo ICI antibodies (i.e., anti-PD-L1 and anti-CTLA-4) (second bar); (iii) DE122435.3+ isotype control antibody (third bar); and (iv) DE122435.3+ combo ICI antibodies (fourth bar). In FIG. 24A, the population of CD45⁺ T cells are shown as a percentage of total viable cells. In FIG. 24B, the population of CD8⁺ T cells are shown as a percentage of total CD45+ T cells.

FIGS. 25A, 25B, and 25C show the percentage of effector CD8⁺ T cells in the tumor tissues of animals treated with one of the following: (i) negative control DE821956.1+ isotype control antibody (first bar); (ii) negative control DE821956.1+ combo ICI antibodies (i.e., anti-PD-L1 and anti-CTLA-4) (second bar); (iii) DE122435.3+ isotype control antibody (third bar); and (iv) DE122435.3+ combo ICI antibodies (fourth bar). FIG. 25A shows the percentage of CD8+ T cells that are CD25+CD69+ in the tumor tissues. FIG. 25B shows the percentage of CD8+ T cells in the tumor tissues that express intermediate PD-1 expression. FIG. 25C shows the frequency of CD8+ T cells that express GranzymeB (CD103+) in the tumor tissues.

FIGS. 26A, 26B, and 26C show the percentage of migratory CD8+ T cells in the tumor tissues of animals treated with one of the following: (i) negative control DE821956.1+isotype control antibody (first bar); (ii) negative control DE821956.1+ combo ICI antibodies (i.e., anti-PD-L1 and anti-CTLA-4) (second bar); (iii) DE122435.3+isotype control antibody (third bar); and (iv) DE122435.3+combo ICI antibodies (fourth bar). FIG. 26A shows the percentage of CD8+ T cells that are CD103+ in the tumor tissues. FIG. 26B shows the percentage of CD8+ T cells that are CD103+Granzyme B+ in the tumor tissues. FIG. 26C shows the percentage of CD8+ T cells that are CD103+CD25+CD69+ in the tumor tissues.

FIGS. 27A, 27B, 27C, and 27D show the percentage of exhausted CD8+ T cells in the tumor tissues of animals treated with one of the following: (i) negative control DE821956.1+isotype control antibody (first bar); (ii) negative control DE821956.1+ combo ICI antibodies (i.e., anti-PD-L1 and anti-CTLA-4) (second bar); (iii) DE122435.3+ isotype control antibody (third bar); and (iv) DE122435.3+ combo ICI antibodies (fourth bar). FIG. 27A shows the percentage of CD8+ T cells that are PD-1+ in the tumor tissues. FIG. 27B shows the percentage of CD8+ T cells that are TIM3+LAG3+PD-1+ high in the tumor tissues. FIG. 27C shows the percentage of CD8+ T cells with intermediate PD-1+ expression as a percentage of total CD8+ T cells in the tumor tissues. FIG. 27D shows the percentage of CD8+ T cells with high PD-1+ expression as a percentage of total CD8+ T cells in the tumor tissues.

FIGS. 28A, 28B, 28C, 28D, and 28E show the percentage of the dendritic cells or macrophage populations in the tumor tissues of animals treated with one of the following: (i) negative control DE821956.1+ isotype control antibody (first bar); (ii) negative control DE821956.1+ combo ICI antibodies (i.e., anti-PD-L1 and anti-CTLA-4) (second bar); (iii) DE122435.3+ isotype control antibody (third bar); and (iv) DE122435.3+ combo ICI antibodies (fourth bar). FIG. 28A shows the frequency of CD11c+CD11b-mature dendritic cells as a percentage of total CD45+ cells in the tumor tissues. FIG. 28B shows the frequency of CD11b+ immature dendritic cells as a percentage of total CD45+ cells in the tumor tissues. FIG. 28C shows the percentage of CD11b-CD11c+ mature dendritic cells that are CD103+ in the tumor tissues. FIG. 28D shows the percentage of CD11b+ immature dendritic cells that are CD103+ in the tumor tissues. FIG. 28E shows the frequency of CD11b+F4/80+ macrophage cells as a percentage of total CD45+ cells in the tumor tissues.

FIGS. 29A, 29B, 29C, 29D, 29E, and 29F show the percentages of different T cell populations in the tumor-draining lymph nodes (“TDLNs”) of animals treated with one of the following: (i) negative control DE821956.1+ isotype control antibody (first bar); (ii) negative control DE821956.1+ combo ICI antibodies (i.e., anti-PD-L1 and anti-CTLA-4) (second bar); (iii) DE122435.3+ isotype control antibody (third bar); and (iv) DE122435.3+ combo ICI antibodies (fourth bar). FIG. 29A shows the percentage of CD45+ T cells of total viable cells in the TDLNs. FIG. 29B shows the frequency of CD8+ T cells as a percentage of total CD45+ T cells in the TDLNs. FIG. 29C shows the percentage of CD8+ T cells that are IFNγ+ in the TDLNs. FIG. 29D shows the percentage of CD8+ T cells that are CD69+ in the TDLNs. FIG. 29E shows the percentage of CD69+CD8+ T cells that are CD103+ in the TDLNs. FIG. 29F shows the percentage of CD8+ T cells that are PD-1+ in the TDLNs.

FIGS. 30A and 30B show the percentages of different dendritic cell populations in the tumor-draining lymph nodes (“TDLNs”) of animals treated with one of the following: (i) negative control DE821956.1+ isotype control antibody (first bar); (ii) negative control DE821956.1+ combo ICI antibodies (i.e., anti-PD-L1 and anti-CTLA-4) (second bar); (iii) DE122435.3+ isotype control antibody (third bar); and (iv) DE122435.3+ combo ICI antibodies (fourth bar). FIG. 30A shows the frequency of CD11c+ dendritic cells as a percentage of total CD45+ cells in the TDLNs. FIG. 30B shows the percentage of CD11+ dendritic cells that are MHCI+ in the TDLNs.

FIGS. 31A, 31B, 31C, 31D, 31E, 31F, 31G, 31H, and 31I show the effects of DE122435.3 composition on human T cells in an in vitro assay. The assays use CD3/CD28 activation that does not require feeder cells (antigen-presenting cells) or antigen. The primary human T cells were treated with bacterial supernatants from compositions including DE122435.3, and DE821956.1, DE916091.1, PNP 16720. PNP 16721, PNP 16722 or the negative control bacterial media. FIGS. 31A, 31B, and 31C show the expression of CD45RA (a gene downregulated with T cell activation), CD45RO and CD69 (two activation markers) in CD8 T cells treated with DE122435.3 compared to other treatment groups. FIGS. 31D, 31E, and 31F show the comparison of all treatment groups in normalized gene counts of the expression of IL-24, TNF, and perforin, respectively. FIGS. 31G, 31H, and 31I show the quantification of IFNγ gene expression by multiplexed molecular barcode (e.g., available from NanoString Technologies), IFNγ secreted protein by multiplexed bead-based (e.g., available from Luminex), and intracellular IFNγ protein by flow cytometry, respectively.

FIGS. 32A, 32B, 32C, 32D, and 32E show the effects of DE122435.3 composition on human T cells in an in vitro assay. FIGS. 32A, 32B, 32C, 32D and 32E show the normalized gene counts for T cell inhibitory receptors TIGIT, TIM-3, LAG-3, PD-1 and CTLA-4, respectively, across all treatment groups.

FIG. 33 shows the effect of bacterial media and bacterial compositions DE916091.1, DE821956.1, and DE122435.3 on tumor cells in an in vitro CD8 cytotoxicity assay.

FIGS. 34A, 34B, 34C, 34D, and 34E show the effects of single bacterial strains on human T cells in an in vitro assay. FIGS. 34A, 34B, 34C, 34D, and 34E show the comparison of all treatment groups in normalized gene counts of the expression of IFN γ, TNFα, perforin, GzmB and CD69, respectively.

FIGS. 35A and 35B show the effect of single bacterial strains tumor cells in an in vitro CD8 cytotoxicity assay. FIGS. 35A and 35B compare survival of HT29 target cells when the CD8 T cells were treated with supernatants from single bacterial strains.

FIG. 36 is a graph showing body weight change (%) days after 5-FU (fluorouracil) treatment.

FIGS. 37A, 37B, 37C, 37D, 37E, 37F, 37G, 37H, 37I, 37J, 37K, 37L, 37M, 37N, 37O, 37P, 37Q, and 37R are graphs showing cytokine concentration in serum at 5 and 8 days after 5-FU (fluorouracil) treatment. FIGS. 37A, 37C, 37E, 37G, 37I, 37K, 37M, 37O, and 37Q show the average serum concentrations of duplicates, while FIGS. 37B, 37D, 37F, 37H, 37J, 37L, 37N, 37P, and 37R show serum concentrations of duplicates.

FIG. 38 is a heat map showing cytokine concentration in serum at 5 and 8 days after 5-FU (fluorouracil) treatment across the panel of cytokines.

### DETAILED DESCRIPTION

Applicant has discovered that bacterial compositions comprising certain species of commensal bacteria exhibit certain functional features (e.g., those disclosed herein), and that such compositions can be used to treat and/or reduce the risk of one or more of an infection (including, but not limited to, blood stream infection, sepsis, tissue infection, invasive infection, viral infection or reactivation, and gastrointestinal infection), graft-versus-host-disease (GvHD), or mucositis, e.g., in a subject undergoing HSCT or having cancer. Accordingly, Applicant has identified species of commensal bacteria that can be combined to design bacterial compositions disclosed herein. Detailed disclosure of the bacterial species and the functional features of interest are provided in the present disclosure.

### I. Bacterial (Microbiome) Compositions

Bacteria, including one or more OTU or species of bacteria, discovered to be associated with certain functional features (e.g., those described herein) can be used to design therapeutic compositions or designed compositions (DEs) for treating and/or preventing a range of diseases and disorders, such as an infection or GvHD, for example, following HSCT in a subject. Such compositions can include material directly derived from feces of healthy humans or such compositions can include material fermented from bacterial cultures, including a biologically pure culture. The designed compositions comprising material directly derived from human feces can, in some aspects, contain spore-forming bacteria (SFB) derived from human feces as the sole type of bacteria present in the composition. In some aspects, such designed compositions can comprise spores as the sole type of bacteria present in the composition (healthy human spore product; HHSP). Collectively, SFB and HHSP are referred to herein as “spore compositions.”

In some aspects, one or more bacteria associated with an improvement in a disease or disorder (e.g., an infection or GvHD, such as that observed with HSC transplantation) can be combined to produce the designed compositions disclosed herein. In certain aspects, one or more bacteria associated with certain functional features of interest (e.g., those described herein) can be combined in the bacterial compositions disclosed herein. By combining different bacterial species disclosed herein, the designed compositions disclosed herein can target different biological pathways. Not to be bound by any particular theory, such ability allows the designed compositions disclosed herein to be useful for the treatment of a wide range of diseases and disorders, e.g., those associated with infection, GvHD, or mucositis, e.g., in a subject undergoing HSCT, as well as other diseases and disorders related to immune suppression described herein. Species in a designed composition can be spore-formers (in some cases, in spore form), non-spore formers, or a combination thereof. Species in a designed composition can include material directly derived from feces of healthy humans or such compositions can include material fermented from bacterial cultures, including a biologically pure culture. Collectively, spore compositions and designed compositions are referred to herein as “microbiome compositions.”

Provided herein are bacteria and combinations of bacteria useful for treating and/or preventing one or more signs or symptoms of a disease or disorder associated with dysbiosis of the gastrointestinal microbiome, e.g., infection or GvHD following HSCT. In general, such compositions include one or more of the bacteria described herein and/or one more of the bacteria described herein as exhibiting one or more of the functional features of interest disclosed herein (e.g., reduced morbidity and mortality in HSCT patients or having one or more features associated with reduced morbidity and mortality in HSCT patients).

In some aspects, the amount, level, identity, presence, and/or ratio of bacteria in the microbiome (e.g., gastrointestinal microbiome) of a subject is manipulated to treat, prevent, delay, or ameliorate one or more signs or symptoms of a disease or disorder associated with dysbiosis of the gastrointestinal microbiome (e.g., infection or GvHD following HSCT). In some aspects, the amount, level, identity, presence, and/or ratio of bacteria in the microbiome (e.g., gastrointestinal microbiome) of a subject is manipulated to treat, prevent, delay, or ameliorate one or more signs or symptoms of a disease or disorder described herein.

The term “microbial engraftment” or “engraftment” refers to the establishment of OTUs (bacterial species or strains) comprising a therapeutic microbial composition, e.g., a bacterial composition, in a target niche that are absent or undetectable in a treated subject prior to treatment. The microbes comprising the engrafted ecology are present in the therapeutic microbial composition and establish as constituents of the subject's microbial ecology. Engrafted OTUs can establish for a transient period of time, or demonstrate long-term stability in the microbial ecology that populates the subject post treatment with a therapeutic microbial composition. Without committing to any theory, the drug product (i.e., bacterial compositions disclosed herein) may catalyze a shift from a dysbiotic ecology to one representative of a healthy state, either by engraftment of drug product species, promoting ecological conditions favorable for the growth of non-product commensal microbes present in the patient (augmentation), or both.

As used herein, engraftment is indicated by one or more of the following outputs: (i) strain level engraftment, (ii) species-level population engraftment, (iii) species-level subject engraftment, and (iv) putative engraftment. “Strain level engraftment” is determined using any relevant method known in the art. In some aspects, strain level engraftment is determined using an assay in which single nucleotide variant (SNV) frequencies unique to the drug product composition are used to determine whether strains of species detected in treated subjects are significantly more similar to strains in the composition compared to strains of species detected in subjects prior to treatment. Strain level engraftment is measured on a per-subject and per-species basis. Non-limiting examples of other methods of determining strain level engraftment include the use of probes, e.g., NanoString nCounter probes that can be targeted to unique regions of the strain genome, relative to other known genomic sequences of the same species, or compared to metagenomics datasets from healthy subjects; or specific PCR probes for the particular species or strain of interest. “Species-level population engraftment” refers to significantly increased prevalence (p<=0.05) of a species in treated subjects relative to non-treated subjects at any post-treatment time point as measured with a Fisher's exact test, with the requirement that the species was not detected in treated subjects prior to treatment but was detected in the composition. Species-level population engraftment is a population-level measure and requires a significant (p<=0.05) difference across the population treated with a particular regimen compared to placebo. “Species-level subject engraftment” refers to the detection of a species present in the HHSP in a subject post-treatment when said species was not detected pre-treatment in that subject. “Putative engraftment” refers to significantly increased prevalence (p<=0.05) of a species in treated subjects relative to non-treated subjects at any post-treatment time point as measured with a Fisher's exact test. The putative engraftment further requires that the species was detected in the drug product composition and may or may not be present in the treated subject prior to treatment. “Putative engraftment” is a population level statistic. Putative engraftment can be further evaluated using strain level metrics for engraftment.

In some embodiments, the term engraftment can be further divided into long-term engraftment and transient engraftment. “Long-term engraftment” refers to the ability of bacterial species or strains disclosed herein to durably reside in the gastrointestinal tracts of subjects after treatment. Such species or strains are described herein as “long-term engrafter” (LTE). In some embodiments, long-term engrafters continue to be present in the subject (e.g., in the gastrointestinal tract) for about 4 weeks, about 8 weeks, about 12 weeks or longer after the start of dosing of a bacterial composition disclosed herein. “Transient engraftment” refers to the ability of bacterial species or strains (e.g., those disclosed herein) to reside in the gastrointestinal tracts of subjects after treatment, but are only detected in the fecal samples of subjects for a limited period of time. In some embodiments, if bacteria or combinations of bacteria are detected in the fecal sample of a subject, it is generally believed that those bacteria or combinations of bacteria remain present within the gastrointestinal tract. Such species or strains are described herein as “transient engrafter” (TE). In some embodiments, transient-engrafters are detected at one or more time points and not detected at another time point. In some embodiments, transient-engrafters are no longer detected in the subject (e.g., no longer detected in the fecal sample of the subject) about 1 week, about 2 weeks, or about 4 weeks after the start of dosing (i.e., administering a bacterial composition disclosed herein).

It is a key feature of a microbiome composition (e.g., designed compositions) as provided herein that one or more species or OTUs of bacteria in the microbiome composition engraft in a subject treated with the composition, e.g., a subject that responds to the treatment by an improvement in at least one sign or symptom of the disease being treated. In some embodiments, a microbiome composition disclosed herein comprises one or more species or OTUs of bacteria that are long-term engrafters. In other embodiments, a microbiome composition comprises one or more species or OTUs of bacteria that are transient engrafters. In certain embodiments, a microbiome composition comprises both long-term engrafters and transient engrafters. In certain embodiments, a bacterial composition disclosed herein comprises two, three, four, five, six, seven, eight, nine, ten or more long-term engrafters. In some embodiments, a bacterial composition comprises two, three, four, five, six, seven, eight, nine, ten or more transient engrafters. In further embodiments, a bacterial composition disclosed herein comprises three or more transient engrafters and/or three or more long-term engrafters, four or more transient engrafters and/or four or more long-term engrafters, five or more transient engrafters and/or four or more long-term engrafters, six or more transient engrafters and/or four or more long-term engrafters, seven or more transient engrafters and/or four or more long-term engrafters, eight or more transient engrafters and/or four or more long-term engrafters, nine or more transient engrafters and/or four or more long-term engrafters, or ten or more transient engrafters and/or four or more long-term engrafters. In any such embodiments, the bacterial composition disclosed herein can also include one, two, three, four, five, six, seven, eight, nine, ten or more species not defined as long-term or transient engrafters. In further embodiments, a bacterial composition disclosed herein comprises ten or more transient engrafters and/or four or more long-term engrafters and/or two or more species not defined as either.

As used herein, “augmentation” refers to the establishment or significant increase of a population of microbes, or selected species or OTUs, that are (i) absent or undetectable (as determined by the use of known and/or specified genomic or microbiological techniques) in an administered therapeutic microbiome composition, (ii) absent, undetectable, or present at low frequencies in the host niche (as example: gastrointestinal tract (GI tract), skin, anterior-nares, or vagina) before treatment with the microbiome composition compared to after treatment with the microbiome composition, and (iii) are found in the host (subject) after the administration of the microbiome composition or are significantly increased after treatment, for instance about 2-fold, about 5-fold, about 1×10², about 1×10³, about 1×10⁴, about 1×10⁵, about 1×10⁶, about 1×10⁷ fold, or greater than 1×10⁸ fold, in cases where they were present at low frequencies. Microbes comprising an augmented population can be derived from exogenous sources such as food and the environment or grow out from micro-niches within the host where they reside at low frequency. In some aspects of the invention, after treatment with a microbiome composition as provided herein, one or more species or OTUs of bacteria are augmented in the treated subject, e.g., a subject that responds to the treatment by an improvement in at least one sign or symptom of the disease being treated.

Without committing to any theory, administration of a therapeutic microbiome composition may induce a shift in the target niche, e.g., the GI tract, that promotes favorable conditions for the growth of certain commensal microbes causing them to increase in abundance, i.e., they are augmented. In the absence of treatment with a therapeutic microbiome composition, although the host may be exposed to or harbor these commensal microbes, higher abundance and functions and benefits associated with those microbes are not observed or are less frequently observed in a population treated with the microbiome composition.

In some embodiments, a bacterial composition comprises a population of bacteria that has been purified from a biological material (e.g., fecal materials, such as feces or materials isolated from the various segments of the small and large intestines) obtained from a mammalian donor subject (e.g., a healthy human). In some embodiments, the biological material (e.g., fecal material) is obtained from multiple donors (e.g., 2, 3, 4, 5, 6, 7, 8, 9, 10, 15, 20, 25, 30, 35, 40, 45, 50, 75, 100, 200, 300, 400, 500, 750, 1000, or from greater than 1000 donors), and the materials are pooled prior to purification or after purification of the desired bacteria. In other embodiments, the biological material (sample) can be obtained from a single donor subject at multiple times and two or more samples pooled, e.g., 2, 3, 4, 5, 6, 7, 8, 9, 10, 15, 20, 25, 30, 32, 35, 40, 45, 48, 50, 100 samples from a single donor. Methods of making such preparations include treatment of the feces with chloroform, acetone, ethanol, and the like, e.g., see PCT/US2014/014745 and U.S. Pat. No. 9,011,834, which are incorporated herein by reference in their entirety.

In some embodiments, a microbiome composition derived from bacterial cultures, or that is or was derived from feces, is depleted in residual habitat products. “Residual habitat products” refers to material derived from the habitat of a microbiota within or on a human or animal excluding the microbiota. An individual's microbiota is in, for example, feces in the gastrointestinal tract, on the skin itself, in saliva, mucus of the respiratory tract, or secretions of the genitourinary tract, all of which contain biological and other matter associated with the microbial community. “Substantially free of residual habitat products” means that the bacterial composition contains a reduced amount of the biological matter associated with the microbial environment on or in the human or animal subject and is about 100% free, about 99% free, about 98% free, about 97% free, about 96% free, or about 95% free of any contaminating biological matter associated with the microbial community or the contaminating matter is below a level of detection. Residual habitat products can include abiotic materials (including undigested food) or it can include unwanted microorganisms. Substantially free of residual habitat products can also mean that the bacterial composition contains no detectable cells from a human or animal and that only microbial cells are detectable. In some embodiments, substantially free of residual habitat products can mean that the bacterial composition contains no detectable viral (including bacterial viruses (i.e., phage)), fungal, mycoplasmal contaminants. In other embodiments, it means that fewer than about 1×10⁻²%, about 1×10⁻³%, about 1×10⁻⁴%, about 1×10⁻⁵%, about 1×10⁻⁶%, about 1×10⁻⁷%, about 1×10⁻⁸% of the viable cells in the bacterial composition are human or animal, as compared to microbial cells. There are multiple ways to accomplish reduced presence of residual habitat products, none of which are limiting. Thus, contamination can be reduced by isolating desired constituents through multiple steps of streaking to single colonies on solid media until replicate (such as, but not limited to, two) streaks from serial single colonies have shown only a single colony morphology. Alternatively, reduction of contamination can be accomplished by multiple rounds of serial dilutions to single desired cells (e.g., a dilution of about 10⁻⁸ or about 10⁻⁹), such as through multiple 10-fold serial dilutions. This can further be confirmed by showing that multiple isolated colonies have similar cell shapes and Gram staining behavior. Other methods for confirming adequate reduction of residual habitat products include genetic analysis (e.g., PCR, DNA sequencing), serology and antigen analysis, enzymatic and metabolic analysis, and methods using instrumentation such as flow cytometry with reagents that distinguish desired constituents from contaminants. In some embodiments, in a microbiome composition disclosed herein, the bacterial material is substantially composed of viable bacterial spores as the live component. In some embodiments, in a microbiome composition disclosed herein, the bacterial mixture is substantially composed of viable bacteria in the vegetative-state as the live component. In some embodiments, in a microbiome composition disclosed herein, the bacterial mixture is composed of viable bacterial spores and viable bacteria in the vegetative state as the live component.

As used herein, the term “spore” or “endospore” refers to an entity, particularly a bacterial entity, which is in a dormant, non-vegetative and non-reproductive stage. Spores are generally resistant to environmental stress such as radiation, desiccation, enzymatic treatment, temperature variation, nutrient deprivation, oxygen, and chemical disinfectants. In some embodiments, a spore or spore population is resistant to 50% ethanol.

A “spore population” refers to a plurality of spores present in a composition. Synonymous terms used herein include spore composition, spore preparation, ethanol treated spore fraction and spore ecology. A spore population can be purified from a fecal donation, e.g., via ethanol or heat treatment, or a density gradient separation or any combination of methods described herein to increase the purity, potency and/or concentration of spores in a sample. Alternatively, a spore population can be derived through culture methods starting from isolated spore former species or spore former OTUs or from a mixture of such species, either in vegetative or spore form.

In some embodiments, the spore preparation comprises spore forming species wherein residual non-spore forming species have been inactivated by chemical or physical treatments including ethanol, detergent, heat, sonication, and the like; or wherein the non-spore forming species have been removed from the spore preparation by various separations steps including density gradients, centrifugation, filtration and/or chromatography; or wherein inactivation and separation methods are combined to make the spore preparation. In yet another embodiment, the spore preparation comprises spore forming species that are enriched over viable non-spore formers or vegetative forms of spore formers. In this embodiment, spores are enriched by about 2-fold, about 5-fold, about 10-fold, about 50-fold, about 100-fold, about 1000-fold, about 10,000-fold or greater than about 10,000-fold compared to all vegetative forms of bacteria. In yet another embodiment, the spores in the spore preparation undergo partial germination during processing and formulation such that the final composition comprises spores and vegetative bacteria derived from spore forming species.

The term “germinant” refers to a material or composition or physical-chemical process capable of inducing vegetative growth of a bacterium that is in a dormant spore form, or group of bacteria in the spore form, either directly or indirectly in a host organism and/or in vitro.

The term “sporulation induction agent” refers to a material or physical-chemical process that is capable of inducing sporulation in a bacterium, either directly or indirectly, in a host organism and/or in vitro.

The term “increase production of bacterial spores” includes an activity or a sporulation induction agent. “Production” in this context includes conversion of vegetative bacterial cells into spores and augmentation of the rate of such conversion, as well as decreasing the germination of bacteria in spore form, decreasing the rate of spore decay in vivo, or ex vivo, or to increasing the total output of spores (e.g., via an increase in volumetric output of fecal material).

In some embodiments, the preparation of a spore composition includes suspending a sample in ethanol, e.g., at least about 30%, at least about 40%, at least about 50%, at least about 55%, at least about 60%, at least about 65%, at least about 70%, at least about 75%, at least about 80%, at least about 85%, at least about 90%, at least about 95%, or at least about 100%. In some cases, the preparation of a spore composition includes suspending a sample in about 30 to about 100% ethanol, about 40 to about 80% ethanol, about 50 to about 80% ethanol, about 30% ethanol, about 40% ethanol, about 50% ethanol, about 55% ethanol, about 60% ethanol, about 65% ethanol, about 70% ethanol, about 75% ethanol, about 80% ethanol, about 85% ethanol, about 90% ethanol, about 95% ethanol, or about 100%.

As used herein, the terms “purify”, “purified” and “purifying” refer to the state of a population (e.g., a plurality of known or unknown amount and/or concentration) of desired bacteria or bacterial spores, that have undergone one or more processes of purification, e.g., a selection or an enrichment of the desired bacterium and/or bacterial spores, or alternatively a removal or reduction of residual habitat products as described herein. In some embodiments, a purified population has no detectable undesired activity or, alternatively, the level or amount of the undesired activity is at or below an acceptable level or amount. In other embodiments, a purified population has an amount and/or concentration of desired bacteria or bacterial spores, e.g., in general or of selected species, at or above an acceptable amount and/or concentration. In other embodiments, the ratio of desired-to-undesired activity (e.g., spores compared to vegetative bacteria), has changed by about 2-fold, about 5-fold, about 10-fold, about 30-fold, about 100-fold, about 300-fold, about 1×10⁴, about 1×10⁵, about 1×10⁶, about 1×10⁷, about 1×10⁸, or greater than about 1×10⁸. In other embodiments, a purified population of bacterial spores is enriched as compared to the starting material (e.g., a fecal material) from which the population is obtained. This enrichment can be by about 10%, about 20%, about 30%, about 40%, about 50%, about 60%, about 70%, about 80%, about 90%, about 95%, about 96%, about 97%, about 98%, about 99%, about 99.9%, about 99.99%, about 99.999%, about 99.9999%, about 99.99999%, or greater than about 99.999999% as compared to the starting material.

In some embodiments, a purified population of bacteria has reduced or undetectable levels of one or more pathogens (e.g., pathogenic bacteria, viruses, or fungi), or one or more pathogenic activities, such as toxicity, an ability to cause infection of the mammalian recipient subject, an undesired immunomodulatory activity, an autoimmune response, a metabolic response, or an inflammatory response or a neurological response. In some embodiments, the pathogen or pathogenic activity of the bacteria is reduced by at least about 5%, at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99% compared to the reference pathogen or bacteria. In some embodiments, a purified population of bacteria has reduced sensory components as compared to fecal matter, such as reduced odor, taste, appearance, and umami.

In some embodiments, a bacterial composition disclosed herein is substantially free of residual habitat products and/or substantially free of a detectable level of a pathogenic material (e.g., contains no detectable viral (including bacterial viruses (i.e., phage)), fungal, mycoplasmal, or toxoplasmal contaminants, or eukaryotic parasites, such as a helminth; or has an acceptable level of the foregoing. In some embodiments, a bacterial composition is substantially free of acellular material (e.g., DNA, viral coat material, or non-viable bacterial material).

### Designed Compositions (DEs)

Applicant has discovered that certain families, genera, species, and OTUs of bacteria are associated with an improvement (e.g., reduction of risk of infection or GvHD) of a disease or disorder associated with dysbiosis of the gastrointestinal microbiome (e.g., infection or GvHD following HSCT). Furthermore, some of those families, genera, species, and OTUs were associated with engraftment. In addition, some families, genera, species, and OTUs were not present and/or not detected in a subject suffering from a disease or disorder associated with dysbiosis of the gastrointestinal tract (e.g., in a patient suffering from recurrence of C. difficile infection or ulcerative colitis) and were augmented in a subject whose disease state was improved after treatment with an HHSP or a DE. Such bacteria that are associated with improvement in a subject are useful in compositions for treating a disease or disorder associated with dysbiosis (e.g., infection or GvHD following HSCT). Furthermore, applicant has discovered that certain species are positively or negatively associated with an improvement in disease or disorder associated with dysbiosis without intervention from a microbiome composition. In general, such negatively associated species are not included in a composition useful for treating such diseases. In other aspects, such positively associated species may be included in a composition useful for treating such diseases. Applicants have further identified families, genera, species, and OTUs of bacteria that exhibit certain functional features that can be useful in treating a wide range of diseases and disorders, including those associated with dysbiosis of the gastrointestinal tract (e.g., infection or GvHD following HSCT).

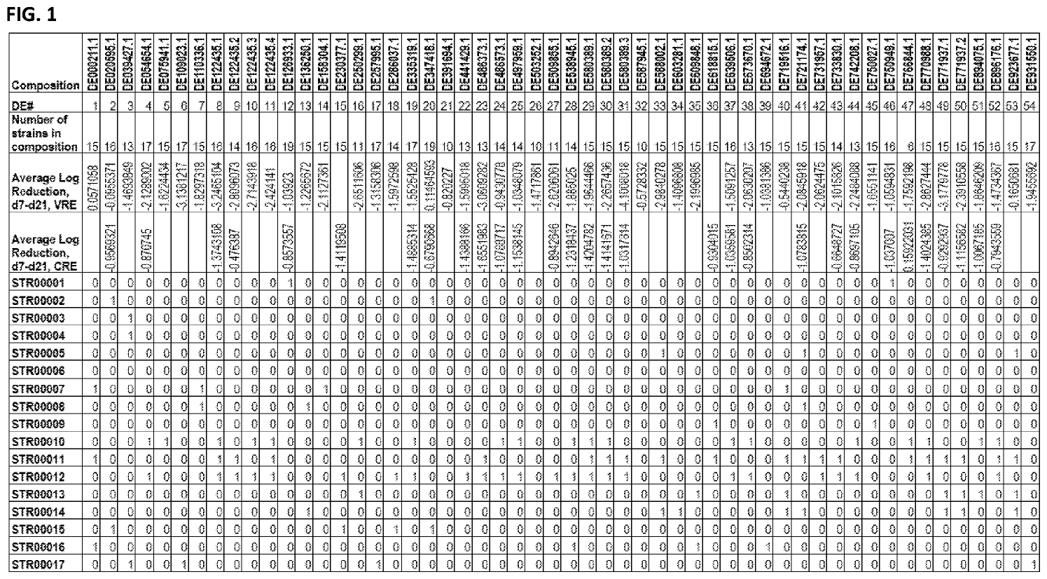

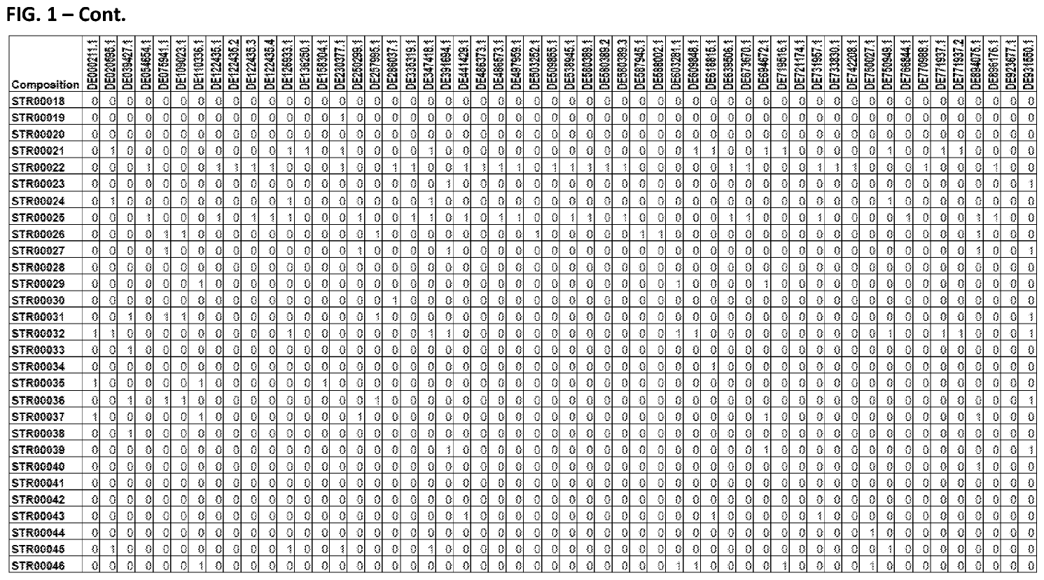

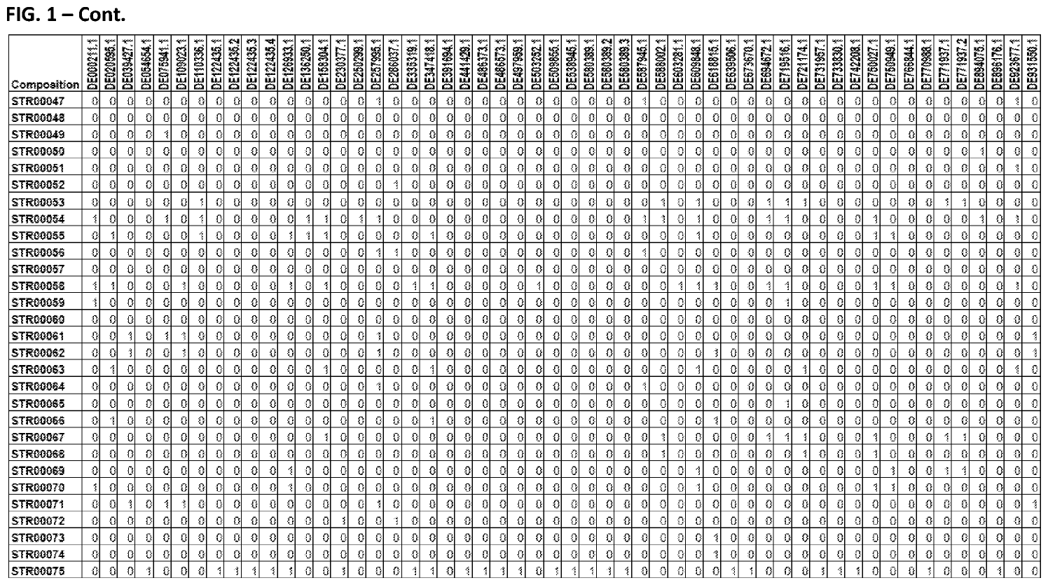

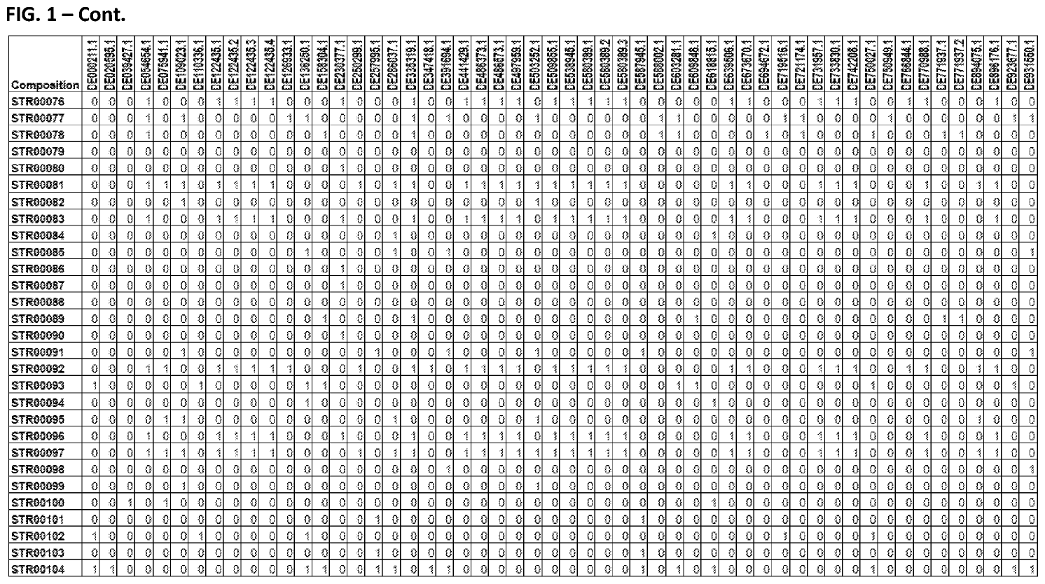

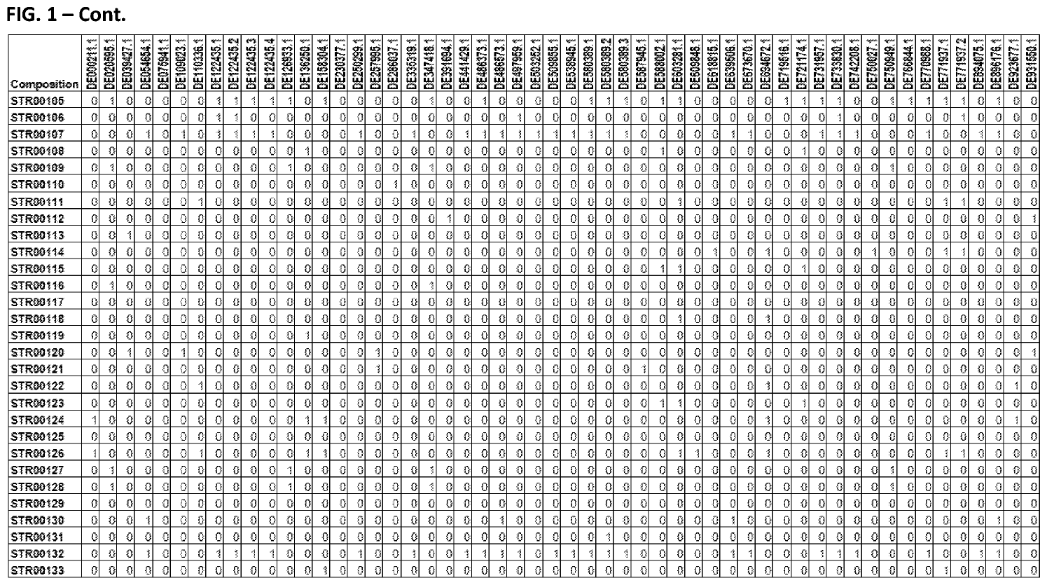

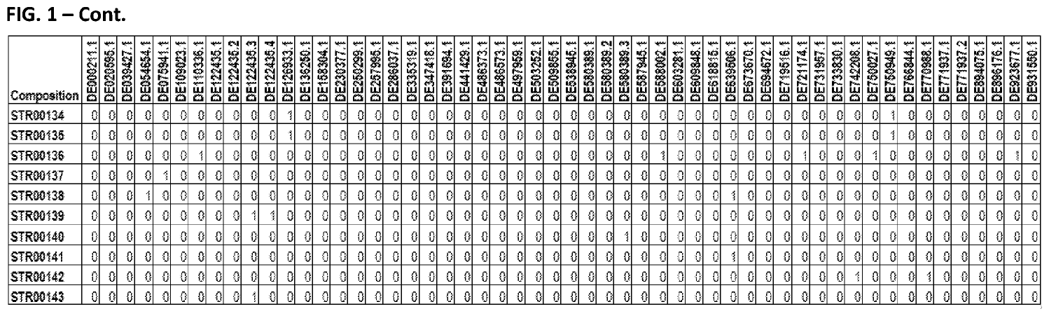

Accordingly, disclosed herein are microbiome compositions that have been designed to exhibit certain features (and as such are designed compositions such as DE8, DE10, DE11, or DE23 recited in FIG. 1). Non-limiting examples of such features include: (i) capable of engrafting (long-term and/or transient) when administered to a subject, (ii) capable of having anti-inflammatory activity in epithelial cells (e.g., inhibiting TNF-α-driven IL-8 secretion in epithelial cells in vitro, ability to down-modulate expression of inflammatory genes (e.g., CXCL1, CXCL2, CXCL3, CXCL11, ICAM1)), (iii) not capable of inducing pro-inflammatory activity, (iv) capable of producing a secondary bile acid (e.g., 7α-dehydroxylase and bile salt hydrolase activity), (v) capable of producing a tryptophan metabolite (e.g., indole, 3-methyl indole, indolepropionic acid), (vi) capable of restoring epithelial integrity as determined by a primary epithelial cell monolayer barrier integrity assay, (vii) capable of being associated with reduction of risk of infection or GvHD following HSCT, (viii) capable of not being associated with clinical non-remission of infection or GvHD following HSCT, (ix) capable of producing a short-chain fatty acid (e.g., butyrate, propionate), (x) capable of inhibiting a HDAC activity, (xi) capable of producing a medium-chain fatty acid (e.g., valerate, hexanoate), (xii) capable of expressing catalase activity, (xiii) capable of having alpha-fucosidase activity, (xiv) capable of inducing Wnt activation, (xv) capable of producing a B vitamin (e.g., thiamin (B1) and/or pyridoxamine (B6)), (xvi) capable of reducing fecal calprotectin level, (xvii) not capable of activating a toll-like receptor pathway (e.g., TLR4 or TLR5), (xviii) capable of activating a toll-like receptor pathway (e.g., TLR2), (xix) capable of restoring colonization resistance, (xx) capable of a broad range of carbon source utilization; (xxi) capable of reducing VRE pathogen carriage, (xxii) capable of reducing CRE pathogen carriage, (xxiii) capable of coadministration with a carrier or excipient described herein, without substantially decreasing the therapeutic benefit of the administered species, (xxiv) capable of being associated with the healthy human gut microbiota, (xxv) capable of not being associated with toxin and hemolysin genes associated with Clostridial pathogens and no significant cytopathic effects in vitro, (xxvi) susceptible to multiple clinically relevant antibiotics, (xxvii) capable of not being associated with genes that are both likely responsible for the observed antibiotic resistances and transmissible, (xxxviii) capable of inhibiting epithelial cell apoptosis, (xxix) capable of down-modulating one or more genes induced in IFN-γ treated colonic organoids (e.g., those associated with inflammatory chemokine signaling, NF-κB signaling, TNF family signaling, type I interferon signaling, type II interferon signaling, TLR signaling, lymphocyte trafficking, Th17 cell differentiation, Th2 differentiation, apoptosis, inflammasomes, autophagy, oxidative stress, MEW class I and II antigen presentation, complement, mTor, nod-like receptor signaling, PI3K signaling, or combinations thereof), (xxx) capable of reducing the expression of one or more inhibitory receptors (e.g., TIGIT, TIM-3, or LAG-3) on CD8+ T cells, (xxxi) capable of increasing expression of one or more genes/proteins associated with CD8+ T cell activation and/or function (e.g., CD45RO, CD69, IL-24, TNF-α, perforin, or IFN-γ), (xxxii) capable of enhancing the ability of CD8+ T cells to kill tumor cells, (xxxiii) capable of enhancing the efficacy of an immune checkpoint inhibitor therapy, (xxxiv) capable of promoting the recruitment of CD8+ T cells to tumors, (xxxv) capable of inducing an anti-inflammatory IL-10-skewed IL-10/IL-6 cytokine ratio in macrophages, (xxxvi) capable of inducing less inflammatory responses but similar pathogen defense responses in macrophages than a donor-derived spore-based composition, (xxxvii) capable of increasing the amount of anti-inflammatory mediators in (e.g., IL-1 receptor antagonists (IL-1RA), IL-4, IL-6, IL-10, IL-11, IL-13, TGF-β), (xxxviii) capable of reducing colonic inflammation, (xxxix) capable of treating and/or preventing a disease or disorder, such as those associated with dysbiosis of a gastrointestinal tract, (xl) capable of increasing the diversity of the gastrointestinal microbiome in a subject, (xli) capable of improving mucosal and/or epithelial barrier integrity in a subject compared to a reference control (e.g., untreated patients or the subject prior to treatment), (xlii) capable of promoting mucosal healing, (xliii) capable of reducing incidence of infection, (xliv) capable of reducing the need for antibiotics in a subject, (xlv) capable of increasing the probability of survival in a subject, (xlvi) capable of reducing the risk of relapse of primary cancer, (xlvii) capable of reducing the abundance of a biomarker of infection in the stool of a subject, (xlviii) capable of increasing the abundance of a biomarker of an administered species in the stool of a subject, (xlix) capable of targeted delivery of most (e.g., 70%, 75%, 80%, 85%, 90%, 95%, 96%, 97%, 98%, 99%, 99.5%, 99.6%, 99.7%, 99.8%, or 99.9% of the administered species relative to the number of colony forming units administered) or all of the administered species to the intestines of the subject (e.g., through encapsulation, or through coating one or more components of a dosage form with an enteric polymer), (l) capable of a therapeutic benefit following a single administration of a composition or pharmaceutical composition described herein to a subject, (li) capable of coadministration with an additional agent described herein, without substantially decreasing the therapeutic benefit of the administered species, (lii) capable of increasing the Treg:Th1 or Treg:Th17 ratios on the lamina propria of mice, or any combination thereof. Non-limiting examples of designed compositions are described, e.g., in FIG. 1. In some embodiments, a designed composition disclosed herein comprises one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, fifty, fifty-one, fifty-two, fifty-three, or all of the above features. In certain embodiments, a designed composition of the present disclosure can comprise features that target multiple biological pathways, such that the same composition can be used to treat a wide range of diseases and disorders. e.g., in FIG. 1. In some embodiments, a designed composition disclosed herein comprises one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, fifty, fifty-one, fifty-two, or all of the above features. In certain embodiments, a designed composition of the present disclosure such as DE122435.3 can comprise features that target multiple biological pathways, such that the same composition can be used to treat a wide range of diseases and disorders.

In some embodiments, a bacterial composition disclosed herein (such as DE8, DE10, DE11, or DE23 recited in FIG. 1) comprises one or more features selected from (i) capable of engrafting (long-term and/or transient) when administered to a subject, (ii) capable of having anti-inflammatory activity (e.g., inhibiting TNF-α-driven IL-8 secretion in epithelial cells in vitro, ability to downmodulate expression of inflammatory genes (e.g., CXCL1, CXCL2, CXCL3, CXCL11, ICAM1)), (iii) not capable of inducing pro-inflammatory activity, (iv) capable of producing a secondary bile acid (7α-dehydroxylase and bile salt hydrolase activity), (v) capable of producing a tryptophan metabolite (e.g., indole, 3-methyl indole, indolepropionic acid), (vi) capable of restoring epithelial integrity as determined by a primary epithelial cell monolayer barrier integrity assay, (vii) capable of being associated with reduction of risk of infection or GvHD following HSCT, (viii) capable of not being associated with clinical non-remission of infection or GvHD following HSCT, (ix) capable of producing a short-chain fatty acid (e.g., butyrate, propionate), (x) capable of inhibiting a HDAC activity, (xi) capable of producing a medium-chain fatty acid (e.g., valerate, hexanoate), (xii) capable of expressing catalase activity, (xiii) capable of having alpha-fucosidase activity, (xiv) capable of inducing Wnt activation, (xv) capable of producing a B vitamin (e.g., thiamin (B1) and/or pyridoxamine (B6)), (xvi) capable of reducing fecal calprotectin level, (xvii) not capable of activating a toll-like receptor pathway (e.g., TLR4 or TLR5), (xviii) capable of activating a toll-like receptor pathway (e.g., TLR2), (xix) capable of restoring colonization resistance, (xx) capable of a broad range of carbon source utilization; (xxi) capable of reducing VRE pathogen carriage, (xxii) capable of reducing CRE pathogen carriage, (xxiii) capable of reducing colonic inflammation, (xxiv) capable of being associated with the healthy human gut microbiota, (xxv) capable of not being associated with toxin and hemolysin genes associated with Clostridial pathogens and no significant cytopathic effects in vitro, (xxvi) susceptible to multiple clinically relevant antibiotics, (xxvii) capable of not being associated with genes that are both likely responsible for the observed antibiotic resistances and transmissible, (xxviii) capable of inhibiting epithelial cell apoptosis, (xxix) capable of down-modulating one or more genes induced in IFN-γ treated colonic organoids (e.g., those associated with inflammatory chemokine signaling, NF-κB signaling, TNF family signaling, type I interferon signaling, type II interferon signaling, TLR signaling, lymphocyte trafficking, Th17 cell differentiation, Th1 differentiation, Th2 differentiation, apoptosis, inflammasomes, autophagy, oxidative stress, MHC class I and II antigen presentation, complement, mTor, nod-like receptor signaling, PI3K signaling, or combinations thereof), (xxx) capable of reducing the expression of one or more inhibitory receptors (e.g., TIGIT, TIM-3, or LAG-3) on T cells, (xxxi) capable of increasing expression of one or more genes/proteins associated with T cell activation and/or function (e.g., CD45RO, CD69, IL-24, TNF-α, perforin, or IFN-γ), (xxxii) capable of enhancing the ability of CD8+ T cells to kill tumor cells, (xxxiii) capable of enhancing the efficacy of an immune checkpoint inhibitor therapy, (xxxiv) capable of promoting the recruitment of CD8+ T cells to tumors, (xxxv) capable of inducing an anti-inflammatory IL-10-skewed IL-10/IL-6 cytokine ratio in macrophages; (xxxvi) capable of inducing less inflammatory responses but similar pathogen defense responses in macrophages than a donor-derived spore-based composition (i.e., a spore-based composition), (xxxvii) capable of producing IL-18, or any combination thereof.

In some embodiments, a bacterial composition disclosed herein (such as DE8, DE10, DE11, or DE23 recited in FIG. 1) comprises one or more features selected from: (i) ability to utilize a carbon source used by a pathogenic organism, such as but not limited to Enterococcus and Enterobacteriaceae species and ESKAPE pathogens (including: Enterococcus faecium, Staphylococcus aureus, Klebsiella pneumoniae, Acinetobacter baumannii, Pseudomonas aeruginosa, and Enterobacter spp.), Enterococcus species including, but not limited to, Enterococcus faecalis and Enterococcus faecium, Enterobacteriaceae species including, but not limited to, Klebsiella pneumonia, or such species that are resistant to vancomycin or carbapenems, drug resistant or multi-drug resistant (MDROs) including VRE, CRE (Klebsiella pneumonia, Klebsiella oxytoca, Klebsiella aerogenes, Enterococcus species), extended spectrum beta-lactamase (ESBLs) producing bacteria (E. coli, Klebsiella species), or methicillin-resistant Staphylococcus aureus (MRSA); (ii) ability to engraft when administered to a subject; (iii) ability to produce short-chain fatty acids; (iv) ability to produce medium-chain fatty acids; (v) ability to produce tryptophan metabolites; (vi) ability to inhibit histone deacetylase (HDAC) activity; (vii) ability to decrease IL-8 secretion in intestinal epithelial cells (IECs) treated with TNF-α; (viii) lack of induction of IL8 secretion in intestinal epithelial cells (IECs) in the absence of TNF-α; and combinations thereof. In some aspects, bacterial species with pro-inflammatory activity (e.g., able to induce IL-8 secretion in IECs) are specifically excluded from bacterial compositions disclosed.

In some embodiments, a bacterial composition disclosed herein (such as DE122435.3) comprises one or more features selected from: (1) reducing VRE and CRE carriage and restore colonization resistance in the GI tract of mice; (2) protecting the epithelial barrier from cytokine-mediated inflammatory damage (e.g., IFN-γ mediated); (3) reducing inflammation in the epithelial barrier, as measured by IL-8 secretion and modulation of inflammatory pathway gene expression in vitro, and in the colonic lamina propria of mice, e.g., as measured through an increased ratio of Treg cells to pro-inflammatory Th1 and Th17 cells, or a combination thereof.

In some embodiments, a bacterial composition disclosed herein (such as DE8, DE10, DE11, or DE23 recited in FIG. 1) comprises one or more features selected from: (1) capable of having anti-inflammatory activity in epithelial cells (e.g., inhibiting TNF-α-driven IL-8 secretion in epithelial cells in vitro, ability to down-modulate expression of inflammatory genes (e.g., CXCL1, CXCL2, CXCL3, CXCL11, ICAM1)), (2) not capable of inducing pro-inflammatory activity, (3) capable of producing a secondary bile acid (e.g., 7α-dehydroxylase and bile salt hydrolase activity), (4) capable of restoring epithelial integrity as determined by a primary epithelial cell monolayer barrier integrity assay, (5) capable of producing a short-chain fatty acid (e.g., butyrate, propionate), (6) capable of inhibiting a HDAC activity, (7) capable of producing a medium-chain fatty acid (e.g., valerate, hexanoate), (8) capable of restoring colonization resistance, (9) capable of reducing VRE pathogen carriage, (10) capable of reducing CRE pathogen carriage, (11) capable of inhibiting epithelial cell apoptosis, (12) capable of down-modulating one or more genes induced in IFN-γ treated colonic organoids (e.g., those associated with inflammatory chemokine signaling, NF-κB signaling, TNF family signaling, type I interferon signaling, type II interferon signaling, TLR signaling, lymphocyte trafficking, Th17 cell differentiation, Th2 differentiation, apoptosis, inflammasomes, autophagy, oxidative stress, MEW class I and II antigen presentation, complement, mTor, nod-like receptor signaling, PI3K signaling, or combinations thereof), (13) capable of reducing the expression of one or more inhibitory receptors (e.g., TIGIT, TIM-3, or LAG-3) on CD8+ T cells, (14) capable of increasing expression of one or more genes/proteins associated with CD8+ T cell activation and/or function (e.g., CD45RO, CD69, IL-24, TNF-α, perforin, or IFN-γ), (15) capable of enhancing the ability of CD8+ T cells to kill tumor cells, (16) capable of enhancing the efficacy of an immune checkpoint inhibitor therapy, (17) capable of increasing the Treg:Th1 or Treg:Th17 ratios on the lamina propria of mice, or any combination thereof.

In some embodiments, a bacterial composition disclosed herein (such as DE8, DE10, DE11, or DE23 recited in FIG. 1) comprises one or more features selected from: (1) capable of having anti-inflammatory activity in epithelial cells (e.g., inhibiting TNF-α-driven IL-8 secretion in epithelial cells in vitro, ability to down-modulate expression of inflammatory genes (e.g., CXCL1, CXCL2, CXCL3, CXCL11, ICAM1)), (2) not capable of inducing pro-inflammatory activity, (3) capable of restoring epithelial integrity as determined by a primary epithelial cell monolayer barrier integrity assay, (4) capable of inhibiting epithelial cell apoptosis, (5) capable of down-modulating one or more genes induced in IFN-γ treated colonic organoids (e.g., those associated with inflammatory chemokine signaling, NF-κB signaling, TNF family signaling, type I interferon signaling, type II interferon signaling, TLR signaling, lymphocyte trafficking, Th17 cell differentiation, Th2 differentiation, apoptosis, inflammasomes, autophagy, oxidative stress, MHC class I and II antigen presentation, complement, mTor, nod-like receptor signaling, PI3K signaling, or combinations thereof), (6) capable of reducing the expression of one or more inhibitory receptors (e.g., TIGIT, TIM-3, or LAG-3) on CD8+ T cells, (7) capable of increasing expression of one or more genes/proteins associated with CD8+ T cell activation and/or function (e.g., CD45RO, CD69, IL-24, TNF-α, perform, or IFN-γ), (8) capable of enhancing the ability of CD8+ T cells to kill tumor cells, (9) capable of enhancing the efficacy of an immune checkpoint inhibitor therapy, (10) capable of increasing the Treg:Th1 or Treg:Th17 ratios on the lamina propria of mice, or any combination thereof.

In some embodiments, a bacterial composition disclosed herein (such as DE8, DE10, DE11, or DE23 recited in FIG. 1) comprises one or more features selected from: (1) capable of having anti-inflammatory activity in epithelial cells (e.g., inhibiting TNF-α-driven IL-8 secretion in epithelial cells in vitro, ability to down-modulate expression of inflammatory genes (e.g., CXCL1, CXCL2, CXCL3, CXCL11, ICAM1)), (2) not capable of inducing pro-inflammatory activity, (3) capable of producing a secondary bile acid (e.g., 7α-dehydroxylase and bile salt hydrolase activity), (4) capable of restoring epithelial integrity as determined by a primary epithelial cell monolayer barrier integrity assay, (5) capable of producing a short-chain fatty acid (e.g., butyrate, propionate), (6) capable of inhibiting a HDAC activity, (7) capable of producing a medium-chain fatty acid (e.g., valerate, hexanoate), (8) capable of restoring colonization resistance, (9) capable of reducing VRE pathogen carriage, (10) capable of reducing CRE pathogen carriage, or any combination thereof.

In some embodiments, a bacterial composition disclosed herein (such as DE8, DE10, DE11, or DE23 recited in FIG. 1) comprises one or more features selected from: (1) capable of restoring colonization resistance, (2) capable of reducing VRE pathogen carriage, (3) capable of reducing CRE pathogen carriage, (4) capable of inhibiting epithelial cell apoptosis, or any combination thereof.

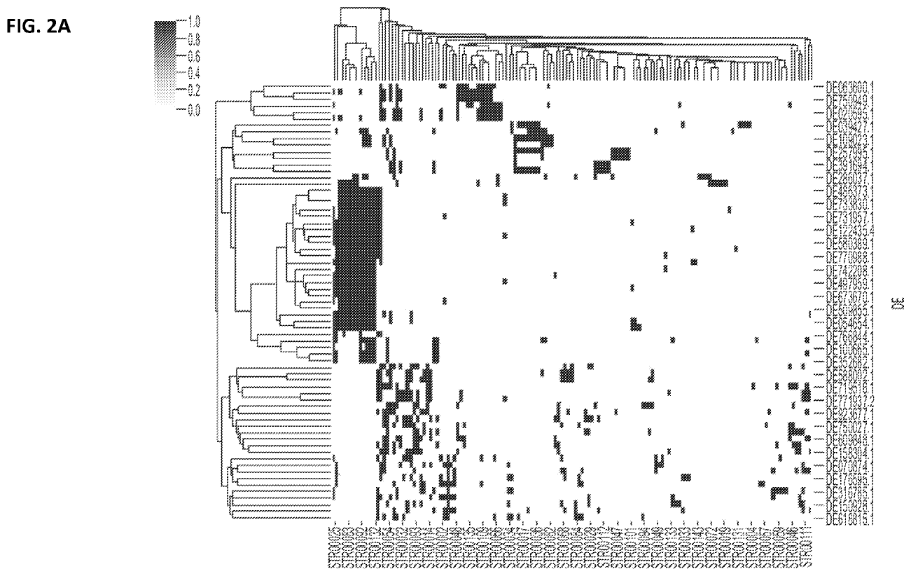

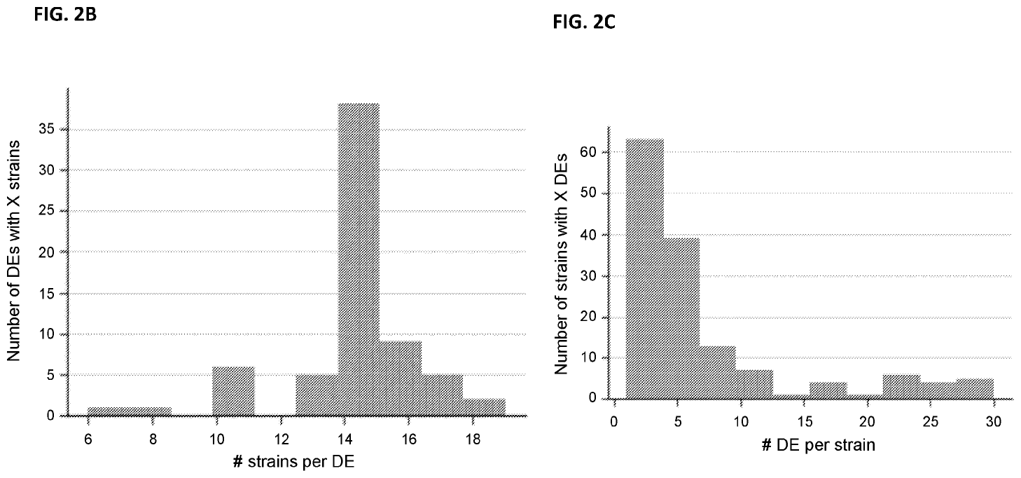

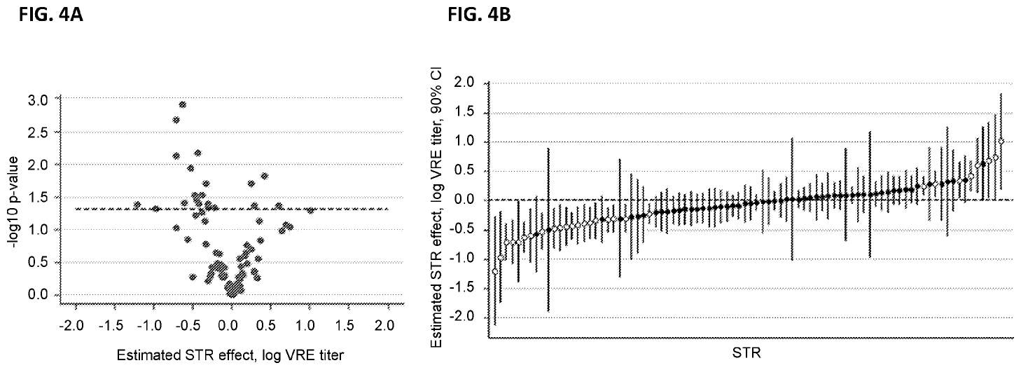

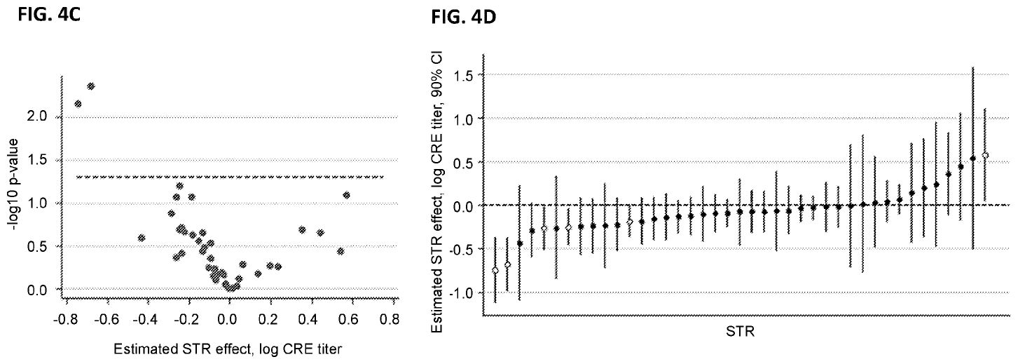

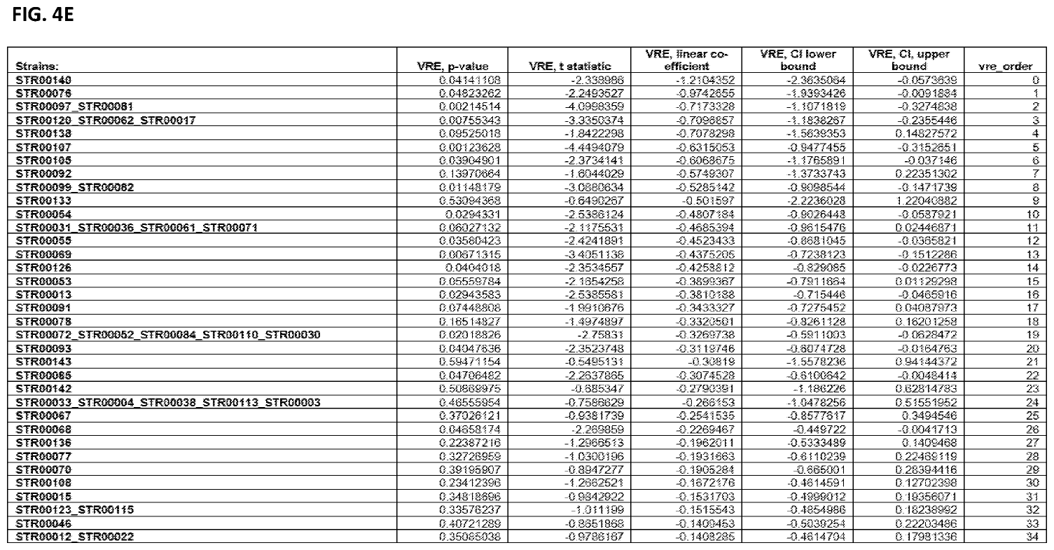

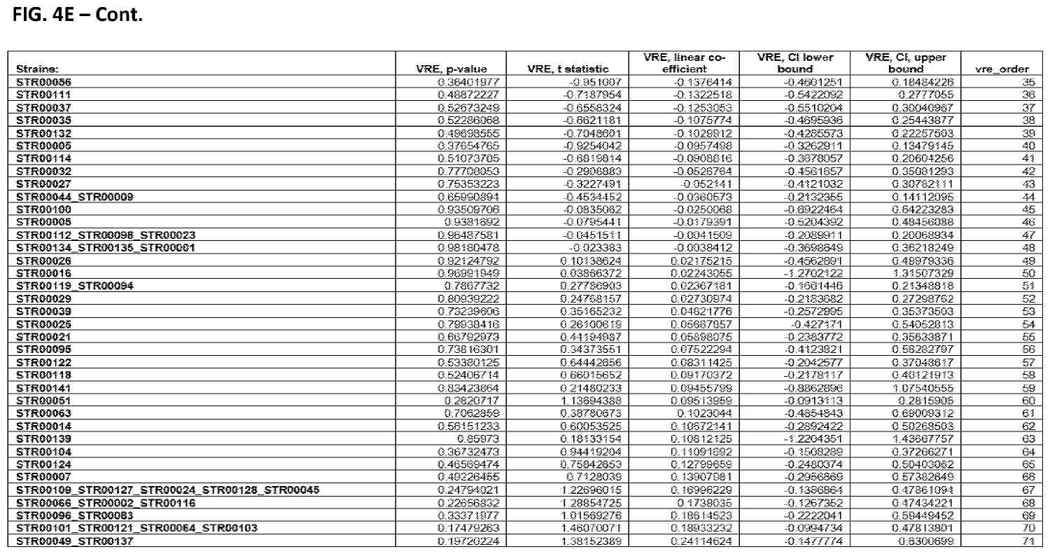

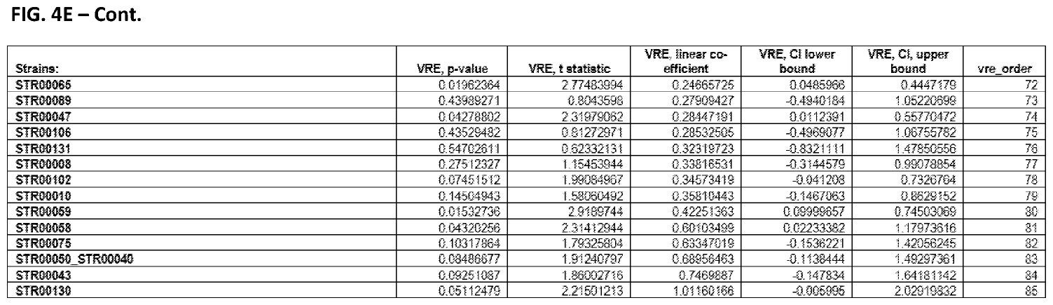

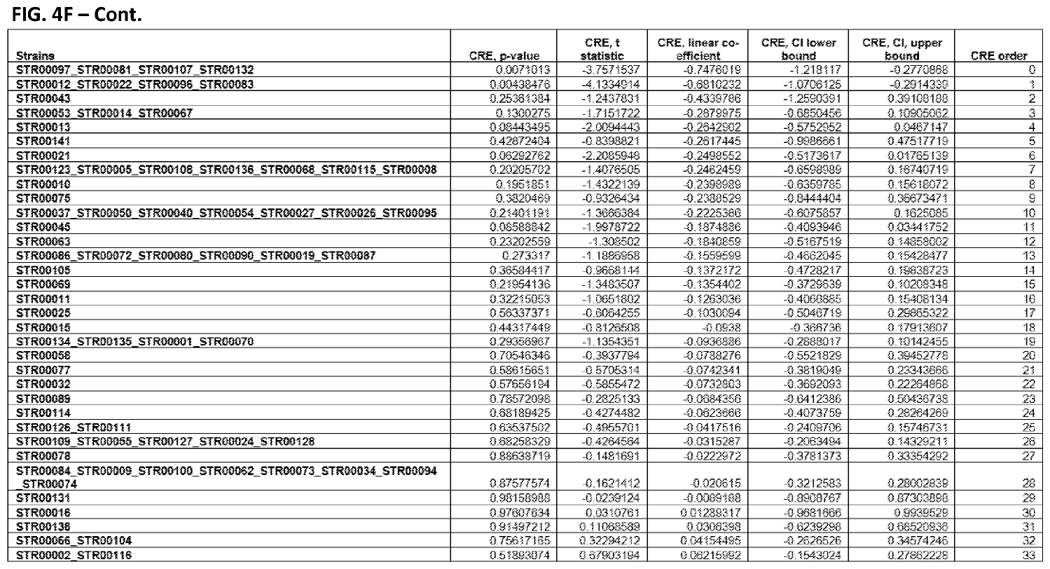

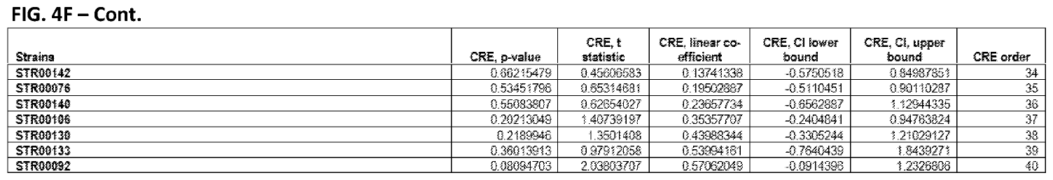

In some embodiments, the bacteria in a microbiome composition comprise one or more families, genera, species, or OTUs that are increased in the GI microbiome of a patient suffering from a disease or disorder associated with dysbiosis of the gastrointestinal tract (e.g., patients having infection or GvHD following HSCT) or population of patients prior to treatment with a complex microbiome composition, e.g., an HHSP or DE composition, and increased in a subject or a population of subjects after treatment with an HHSP or DE composition. In some embodiments, a bacterial composition disclosed herein comprises selected families, genera, species, or OTUs of bacteria. In general, the bacteria are commensal bacteria initially derived from, for example, a GI tract, typically the GI tract of a human, isolated and grown into pure cultures that can be used in a DE. These bacteria are selected for desired properties as described herein and used in designed composition. In some embodiments, a bacterial composition (e.g., designed compositions disclosed herein) comprises more than two types of bacteria. Accordingly, in some embodiments, a bacterial composition of the present disclosure comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or at least 21, 22, 23, 24, 25, 26, 27, 28, 29 30, 31, 32, 33, 34, 35, 36, 37, 38, 39, or at least 40, at least 50, or greater than 50 types of bacteria, as defined by species or operational taxonomic unit (OTU), or otherwise as provided herein. The bacteria in a composition may be present in approximately equal amounts of viable bacteria or each family, genus, species of OTU. In other embodiments of the invention, the bacteria are present in varying amounts in the composition. Non-limiting examples of bacterial species that can be used in designing the microbiome compositions disclosed herein are provided in Tables 1-3, FIG. 1-2A, FIG. 4E, and/or FIG. 4F.

In some embodiments, the bacteria in a microbiome composition disclosed herein are from a family, genus, species, or OTU depleted in a subject suffering from a disease or disorder, such as those associated with a dysbiosis (e.g., patients who become dysbiotic during HSCT and/or having infection or GvHD following HSCT) and/or typically present only at low levels or are absent in patients diagnosed with a disease or disorder, such as those associated with dysbiosis (e.g., infection or GvHD following HSCT). In some embodiments, a bacterial composition includes one or more additional bacteria that are present with high frequency in a population of healthy humans or subjects with a disease or disorder associated with dysbiosis (e.g., patients having infection or GvHD following HSCT) but who are not exhibiting symptoms associated with active disease. In some embodiments, a bacterial composition does not include one or more bacteria that are pathogenic or pathobiont species or OTU and/or bacteria that are present in an overabundance as observed on or before commencing HSCT.

In some embodiments, a bacterial composition disclosed herein comprises one or more bacteria from the family Ruminococcaceae, Lachnospiraceae, Sutterellaceae, Clostridiaceae, Erysipelotrichaceae, Bacteroidaceae, Akkermansiaceae, Peptostreptococcaceae, Eubacteriaceae, Clostridiales Family XIII, or Desulfovibrionaceae. In some embodiments, a bacterial composition can comprise at least one, two, three, four, five, six, seven, or all of the families listed.

In some embodiments, a bacterial composition comprises bacteria having at least about 97%, e.g., at least about 97%, at least about 97.5%, at least about 98%, at least about 98.5%, at least about 99%, identity to a 16S rDNA sequence (e.g., a full length or variable region of a 16S DNA sequence) to one or more of the following bacterial species: Eubacterium maltosivorans, Clostridium aldenense, Clostridium bolteae, Clostridium glycyrrhizinilyticum, Clostridium hylemonae, Clostridium innocuum, Clostridium lavalense, Clostridium scindens, Clostridium spiroforme, Clostridium symbiosum, Eubacterium rectale, Ruminococcus gnavus, Ruminococcus torques, Absiella dolichum, Agathobaculum desmolans, Akkermansia muciniphila, Alistipes finegoldii, Alistipes shahii, Anaerofustis stercorihominis, Anaeromassilibacillus senegalensis, Anaerostipes caccae, Anaerotruncus colihominis, Bacteroides caccae, Faecalibacterium prausnitzii, Faecalicatena contorta, Faecalicatena orotica, Flavonifractor plautii, Gemmiger formicilis, Harryflintia acetispora, Holdemania filiformis, Holdemania massiliensis, Intestinimonas butyriciproducens, Lachnospira pectinoschiza, Lachnospiraceae bacterium 5 1 57FAA, Lactobacillus fermentum, Lactonifactor longoviformis, Longibaculum muris, Longicatena caecimuris, Murimonas intestina, Oscillibacter ruminantium, Bacteroides eggerthii, Bacteroides faecis, Bacteroides intestinalis, Bacteroides koreensis, Bacteroides kribbi, Bacteroides salyersiae, Bacteroides uniformis, Bacteroides vulgatus, Bacteroides xylanisolvens, Barnesiella intestinihominis, Bifidobacterium dentium, Bifidobacterium longum, Bifidobacterium stercoris, Blautia coccoides, Blautia hominis, Blautia hydrogenotrophica, Blautia luti, Blautia obeum, Blautia producta, Blautia wexlerae, Butyricimonas faecihominis, Cellulosilyticum lentocellum, Clostridium butyricum, Ruthenibacterium lactatiformans, Sellimonas intestinalis, Shigella flexneri, Terrisporobacter mayombei, Terrisporobacter petrolearius, Turicibacter sanguinis, Tyzzerella nexilis, Clostridium disporicum, Clostridium subterminale, Clostridium tertium, Collinsella aerofaciens, Coprococcus comes, Coprococcus eutactus, Dorea longicatena, Drancourtella massiliensis, Eggerthella lenta, Eisenbergiella tayi, Emergencia timonensis, Erysipelatoclostridium ramosum, Eubacterium callanderi, Paeniclostridium sordellii, Parabacteroides distasonis, Parabacteroides merdae, Paraclostridium bifermentans, Peptostreptococcus stomatis, Robinsoniella peoriensis, Romboutsia timonensis, Roseburia intestinalis, Roseburia inulinivorans, Ruminococcus albus, Ruminococcus bromii, Ruminococcus faecis, or Ruminococcus lactaris. In some embodiments, a bacterial composition can comprise at least one, two, three, four, five, six, seven, eight, nine, ten, eleven, twelve, thirteen, fourteen, fifteen, sixteen, seventeen, eighteen, nineteen, twenty, twenty-five, thirty, thirty-five, forty, forty-five, fifty, or all of the species listed.

In some embodiments, a bacterial composition comprises bacteria having at least about 97%, e.g., at least about 97%, at least about 97.5%, at least about 98%, at least about 98.5%, at least about 99%, identity to a 16S rDNA sequence (e.g., a full length or variable region of a 16S DNA sequence) to one or more of the following bacterial species: Absiella dolichum, Agathobaculum desmolans, Akkermansia muciniphila, Alistipes finegoldii, Alistipes onderdonkii, Alistipes putredinis, Alistipes senegalensis, Alistipes shahii, Alistipes timonensis, Anaerofustis stercorihominis, Anaeromassilibacillus senegalensis, Anaerostipes caccae, Anaerotruncus colihominis, Atlantibacter hermannii, Atlantibacter subterranea, Bacteroides caccae, Bacteroides cellulosilyticus, Bacteroides eggerthii, Bacteroides faecichinchillae, Bacteroides faecis, Bacteroides finegoldii, Bacteroides intestinalis, Bacteroides koreensis, Bacteroides kribbi, Bacteroides oleiciplenus, Bacteroides ovatus, Bacteroides rodentium, Bacteroides salyersiae, Bacteroides stercorirosoris, Bacteroides thetaiotaomicron, Bacteroides uniformis, Bacteroides vulgatus, Bacteroides xylanisolvens, Barnesiella intestinihominis, Bifidobacterium adolescentis, Bifidobacterium catenulatum, Bifidobacterium dentium, Bifidobacterium faecale, Bifidobacterium kashiwanohense, Bifidobacterium longum, Bifidobacterium pseudocatenulatum, Bifidobacterium ruminantium, Bifidobacterium stercoris, Blautia coccoides, Blautia hansenii, Blautia hominis, Blautia hydrogenotrophica, Blautia luti, Blautia marasmi, Blautia obeum, Blautia producta, Blautia stercoris, Blautia wexlerae, Brenneria alni, Butyricimonas faecihominis, Cedecea lapagei, Cellulosilyticum lentocellum, Citrobacter amalonaticus, Citrobacter farmers, Citrobacter koseri, Citrobacter sedlakii, Citrobacter youngae, Clostridium aldenense, Clostridium asparagiforme, Clostridium beijerinckii, Clostridium bolteae, Clostridium butyricum, Clostridium carnis, Clostridium celatum, Clostridium chauvoei, Clostridium chromiireducens, Clostridium citroniae, Clostridium clostridioforme, Clostridium cocleatum, Clostridium dakarense, Clostridium diolis, Clostridium disporicum, Clostridium glycyrrhizinilyticum, Clostridium hylemonae, Clostridium innocuum, Clostridium lavalense, Clostridium paraputrificum, Clostridium puniceum, Clostridium quinii, Clostridium saccharobutylicum, Clostridium saccharoperbutylacetonicum, Clostridium sartagoforme, Clostridium saudiense, Clostridium scindens, Clostridium septicum, Clostridium spiroforme, Clostridium subterminale, Clostridium sulfidigenes, Clostridium symbiosum, Clostridium tertium, Clostridium thiosulfatireducens, Collinsella aerofaciens, Coprococcus comes, Coprococcus eutactus, Cronobacter condimenti, Cronobacter muytjensii, Cronobacter sakazakii, Dorea longicatena, Drancourtella massiliensis, Eggerthella lenta, Eisenbergiella massiliensis, Eisenbergiella tayi, Emergencia timonensis, Enterobacter asburiae, Enterobacter bugandensis, Enterobacter cloacae, Enterobacter hormaechei, Enterobacter tabaci, Erysipelatoclostridium ramosum, Escherichia albertii, Escherichia coli, Escherichia fergusonii, Escherichia marmotae, Eubacterium callanderi, Eubacterium limosum, Eubacterium maltosivorans, Eubacterium rectale, Eubacterium tenue, Faecalibacterium prausnitzii, Faecalicatena contorta, Faecalicatena fissicatena, Faecalicatena orotica, Flavonifractor plautii, Gemmiger formicilis, Harryflintia acetispora, Holdemania filiformis, Holdemania massiliensis, Intestinibacter bartlettii, Intestinimonas butyriciproducens, Kosakonia cowanii, Kosakonia oryzendophytica, Kosakonia oryziphila, Kosakonia pseudosacchari, Kosakonia sacchari, Lachnoclostridium pacaense, Lachnospira pectinoschiza, Lactobacillus fermentum, Lactobacillus gorillae, Lactonifactor longoviformis, Longibaculum muris, Longicatena caecimuris, Metakosakonia massiliensis, Mixta theicola, Murimonas intestina, Oscillibacter ruminantium, Paeniclostridium ghonii, Paeniclostridium sordellii, Pantoea beijingensis, Parabacteroides distasonis, Parabacteroides johnsonii, Parabacteroides merdae, Paraclostridium benzoelyticum, Paraclostridium bifermentans, Pectobacterium carotovorum, Peptostreptococcus anaerobius, Peptostreptococcus stomatis, Phytobacter ursingii, Pseudescherichia vulneris, Pseudocitrobacter anthropi, Pseudocitrobacter faecalis, Pseudoflavonifractor capillosus, Pseudoflavonifractor phocaeensis, Raoultella planticola, Robinsoniella peoriensis, Romboutsia ilealis, Romboutsia lituseburensis, Romboutsia sedimentorum, Romboutsia timonensis, Roseburia faecis, Roseburia hominis, Roseburia intestinalis, Roseburia inulinivorans, Ruminococcus albus, Ruminococcus bromii, Ruminococcus faecis, Ruminococcus gnavus, Ruminococcus lactaris, Ruminococcus torques, Ruthenibacterium lactatiformans, Salmonella bongori, Salmonella enterica, Sellimonas intestinalis, Shigella boydii, Shigella dysenteriae, Shigella flexneri, Shigella sonnei, Subdoligranulum variabile, Terrisporobacter glycolicus, Terrisporobacter mayombei, Terrisporobacter petrolearius, Trabulsiella odontotermitis, Turicibacter sanguinis, Tyzzerella nexilis, or Yokenella regensburgei. In some embodiments, a bacterial composition can comprise at least one, two, three, four, five, six, seven, eight, nine, ten, eleven, twelve, thirteen, fourteen, fifteen, sixteen, seventeen, eighteen, nineteen, twenty, twenty-five, thirty, thirty-five, forty, forty-five, fifty, or all of the species listed.

In some embodiments, a bacterial composition comprises bacteria having at least about 97%, e.g., at least 97%, at least about 97.5%, at least about 98%, at least about 98.5%, at least about 99%, identity to a 16S rDNA sequence (e.g., a full length or variable region of a 16S DNA sequence) to one or more of the following bacterial species: Agathobaculum desmolans, Akkermansia muciniphila, Alistipes finegoldii, Alistipes shahii, Anaerofustis stercorihominis, Anaeromassilibacillus senegalensis, Anaerostipes caccae, Anaerotruncus colihominis, Bacteroides caccae, Bacteroides eggerthii, Bacteroides faecis, Bacteroides intestinalis, Bacteroides koreensis, Bacteroides salyersiae, Bacteroides uniformis, Bacteroides vulgatus, Bacteroides xylanisolvens, Barnesiella intestinihominis, Bifidobacterium dentium, Bifidobacterium longum, Bifidobacterium stercoris, Blautia coccoides, Blautia hominis, Blautia hydrogenotrophica, Blautia luti, Blautia marasmi, Blautia obeum, Blautia producta, Blautia wexlerae, Butyricimonas faecihominis, Cellulosilyticum lentocellum, Clostridium aldenense, Clostridium asparagiforme, Clostridium bolteae, Clostridium butyricum, Clostridium citroniae, Clostridium clostridioforme, Clostridium cocleatum, Clostridium disporicum, Clostridium hylemonae, Clostridium innocuum, Clostridium lavalense, Clostridium scindens, Clostridium spiroforme, Clostridium subterminale, Clostridium symbiosum, Clostridium tertium, Collinsella aerofaciens, Coprococcus comes, Dorea longicatena, Drancourtella massiliensis, Eggerthella lenta, Eisenbergiella massiliensis, Eisenbergiella tayi, Emergencia timonensis, Erysipelatoclostridium ramosum, Eubacterium callanderi, Eubacterium limosum, Eubacterium maltosivorans, Eubacterium rectale, Faecalibacterium prausnitzii, Faecalicatena contorta, Faecalicatena fissicatena, Faecalicatena orotica, Flavonifractor plautii, Gemmiger formicilis, Harryflintia acetispora, Holdemania filiformis, Holdemania massiliensis, Intestinimonas butyriciproducens, Lachnoclostridium pacaense, Lactobacillus fermentum, Lactonifactor longoviformis, Longicatena caecimuris, Murimonas intestina, Oscillibacter ruminantium, Paeniclostridium sordellii, Parabacteroides distasonis, Parabacteroides merdae, Paraclostridium bifermentans, Peptostreptococcus stomatis, Pseudoflavonifractor capillosus, Pseudoflavonifractor phocaeensis, Robinsoniella peoriensis, Romboutsia timonensis, Roseburia intestinalis, Roseburia inulinivorans, Ruminococcus albus, Ruminococcus bromii, Ruminococcus faeces, Ruminococcus gnavus, Ruminococcus lactaris, Ruminococcus torques, Ruthenibacterium lactatiformans, Sellimonas intestinalis, Terrisporobacter mayombei, Terrisporobacter petrolearius, or Turicibacter sanguinis. In some embodiments, a bacterial composition can comprise at least one, two, three, four, five, six, seven, eight, nine, ten, eleven, twelve, thirteen, fourteen, fifteen, sixteen, seventeen, eighteen, nineteen, twenty, twenty-five, thirty, thirty-five, forty, forty-five, fifty, or all of the species listed.

In some embodiments, a bacterial composition comprises bacteria having at least about 97%, e.g., at least about 97%, at least about 97.5%, at least about 98%, at least about 98.5%, at least about 99%, identity to a 16S rDNA sequence (e.g., a full length or variable region of a 16S DNA sequence) to one or more of the following bacterial species: Agathobaculum desmolans, Akkermansia muciniphila, Alistipes finegoldii, Alistipes onderdonkii, Alistipes putredinis, Alistipes senegalensis, Alistipes shahii, Alistipes timonensis, Anaerofustis stercorihominis, Anaeromassilibacillus senegalensis, Anaerostipes caccae, Anaerotruncus colihominis, Bacteroides caccae, Bacteroides cellulosilyticus, Bacteroides eggerthii, Bacteroides faecichinchillae, Bacteroides faecis, Bacteroides finegoldii, Bacteroides intestinalis, Bacteroides koreensis, Bacteroides kribbi, Bacteroides oleiciplenus, Bacteroides ovatus, Bacteroides rodentium, Bacteroides salyersiae, Bacteroides stercorirosoris, Bacteroides thetaiotaomicron, Bacteroides uniformis, Bacteroides vulgatus, Bacteroides xylanisolvens, Barnesiella intestinihominis, Bifidobacterium adolescentis, Bifidobacterium catenulatum, Bifidobacterium dentium, Bifidobacterium faecale, Bifidobacterium kashiwanohense, Bifidobacterium longum, Bifidobacterium pseudocatenulatum, Bifidobacterium ruminantium, Bifidobacterium stercoris, Blautia coccoides, Blautia hansenii, Blautia hominis, Blautia hydrogenotrophica, Blautia luti, Blautia marasmi, Blautia obeum, Blautia producta, Blautia stercoris, Blautia wexlerae, Butyricimonas faecihominis, Cellulosilyticum lentocellum, Clostridium aldenense, Clostridium asparagiforme, Clostridium beijerinckii, Clostridium bolteae, Clostridium butyricum, Clostridium carnis, Clostridium celatum, Clostridium chauvoei, Clostridium chromiireducens, Clostridium citroniae, Clostridium clostridioforme, Clostridium cocleatum, Clostridium dakarense, Clostridium diolis, Clostridium disporicum, Clostridium hylemonae, Clostridium innocuum, Clostridium lavalense, Clostridium paraputrificum, Clostridium puniceum, Clostridium quinii, Clostridium saccharobutylicum, Clostridium saccharoperbutylacetonicum, Clostridium sartagoforme, Clostridium saudiense, Clostridium scindens, Clostridium septicum, Clostridium spiroforme, Clostridium subterminale, Clostridium sulfidigenes, Clostridium symbiosum, Clostridium tertium, Clostridium thiosulfatireducens, Collinsella aerofaciens, Coprococcus comes, Dorea longicatena, Drancourtella massiliensis, Eggerthella lenta, Eisenbergiella massiliensis, Eisenbergiella tayi, Emergencia timonensis, Erysipelatoclostridium ramosum, Eubacterium callanderi, Eubacterium limosum, Eubacterium maltosivorans, Eubacterium rectale, Eubacterium tenue, Faecalibacterium prausnitzii, Faecalicatena contorta, Faecalicatena fissicatena, Faecalicatena orotica, Flavonifractor plautii, Gemmiger formicilis, Harryflintia acetispora, Holdemania filiformis, Holdemania massiliensis, Intestinibacter bartlettii, Intestinimonas butyriciproducens, Lachnoclostridium pacaense, Lactobacillus fermentum, Lactobacillus gorillae, Lactonifactor longoviformis, Longicatena caecimuris, Murimonas intestini, Oscillibacter ruminantium, Paeniclostridium ghonii, Paeniclostridium sordellii, Parabacteroides distasonis, Parabacteroides johnsonii, Parabacteroides merdae, Paraclostridium benzoelyticum, Paraclostridium bifermentans, Peptostreptococcus anaerobius, Peptostreptococcus stomatis, Pseudoflavonifractor capillosus, Pseudoflavonifractor phocaeensis, Robinsoniella peoriensis, Romboutsia ilealis, Romboutsia lituseburensis, Romboutsia sedimentorum, Romboutsia timonensis, Roseburia faecis, Roseburia hominis, Roseburia intestinalis, Roseburia inulinivorans, Ruminococcus albus, Ruminococcus bromii, Ruminococcus faecis, Ruminococcus gnavus, Ruminococcus lactaris, Ruminococcus torques, Ruthenibacterium lactatiformans, Sellimonas intestinalis, Subdoligranulum variabile, Terrisporobacter glycolicus, Terrisporobacter mayombei, Terrisporobacter petrolearius, or Turicibacter sanguinis. In some embodiments, a bacterial composition can comprise at least one, two, three, four, five, six, seven, eight, nine, ten, eleven, twelve, thirteen, fourteen, fifteen, sixteen, seventeen, eighteen, nineteen, twenty, twenty-five, thirty, thirty-five, forty, forty-five, fifty, or all of the species listed.

In some embodiments, a bacterial composition comprises one or more of the following bacterial species: Eubacterium maltosivorans, Clostridium aldenense, Clostridium bolteae, Clostridium glycyrrhizinilyticum, Clostridium hylemonae, Clostridium innocuum, Clostridium lavalense, Clostridium scindens, Clostridium spiroforme, Clostridium symbiosum, Eubacterium rectale, Ruminococcus gnavus, Ruminococcus torques, Absiella dolichum, Agathobaculum desmolans, Akkermansia muciniphila, Alistipes finegoldii, Alistipes shahii, Anaerofustis stercorihominis, Anaeromassilibacillus senegalensis, Anaerostipes caccae, Anaerotruncus colihominis, Bacteroides caccae, Faecalibacterium prausnitzii, Faecalicatena contorta, Faecalicatena orotica, Flavonifractor plautii, Gemmiger formicilis, Harryflintia acetispora, Holdemania filiformis, Holdemania massiliensis, Intestinimonas butyriciproducens, Lachnospira pectinoschiza, Lachnospiraceae bacterium 5 1 57FAA, Lactobacillus fermentum, Lactonifactor longoviformis, Longibaculum muris, Longicatena caecimuris, Murimonas intestini, Oscillibacter ruminantium, Bacteroides eggerthii, Bacteroides faecis, Bacteroides intestinalis, Bacteroides koreensis, Bacteroides kribbi, Bacteroides salyersiae, Bacteroides uniformis, Bacteroides vulgatus, Bacteroides xylanisolvens, Barnesiella intestinihominis, Bifidobacterium dentium, Bifidobacterium longum, Bifidobacterium stercoris, Blautia coccoides, Blautia hominis, Blautia hydrogenotrophica, Blautia luti, Blautia obeum, Blautia producta, Blautia wexlerae, Butyricimonas faecihominis, Cellulosilyticum lentocellum, Clostridium butyricum, Ruthenibacterium lactatiformans, Sellimonas intestinalis, Shigella flexneri, Terrisporobacter mayombei, Terrisporobacter petrolearius, Turicibacter sanguinis, Tyzzerella nexilis, Clostridium disporicum, Clostridium subterminale, Clostridium tertium, Collinsella aerofaciens, Coprococcus comes, Coprococcus eutactus, Dorea longicatena, Drancourtella massiliensis, Eggerthella lenta, Eisenbergiella tayi, Emergencia timonensis, Erysipelatoclostridium ramosum, Eubacterium callanderi, Paeniclostridium sordellii, Parabacteroides distasonis, Parabacteroides merdae, Paraclostridium bifermentans, Peptostreptococcus stomatis, Robinsoniella peoriensis, Romboutsia timonensis, Roseburia intestinalis, Roseburia inulinivorans, Ruminococcus albus, Ruminococcus bromii, Ruminococcus faecis, or Ruminococcus lactaris. In some embodiments, a bacterial composition can comprise at least one, two, three, four, five, six, seven, eight, nine, ten, eleven, twelve, thirteen, fourteen, fifteen, sixteen, seventeen, eighteen, nineteen, twenty, twenty-five, thirty, thirty-five, forty, forty-five, fifty, or all of the species listed.

In some embodiments, a bacterial composition comprises one or more of the following bacterial species: Absiella dolichum, Agathobaculum desmolans, Akkermansia muciniphila, Alistipes finegoldii, Alistipes onderdonkii, Alistipes putredinis, Alistipes senegalensis, Alistipes shahii, Alistipes timonensis, Anaerofustis stercorihominis, Anaeromassilibacillus senegalensis, Anaerostipes caccae, Anaerotruncus colihominis, Atlantibacter hermannii, Atlantibacter subterranea, Bacteroides caccae, Bacteroides cellulosilyticus, Bacteroides eggerthii, Bacteroides faecichinchillae, Bacteroides faecis, Bacteroides finegoldii, Bacteroides intestinalis, Bacteroides koreensis, Bacteroides kribbi, Bacteroides oleiciplenus, Bacteroides ovatus, Bacteroides rodentium, Bacteroides salyersiae, Bacteroides stercorirosoris, Bacteroides thetaiotaomicron, Bacteroides uniformis, Bacteroides vulgatus, Bacteroides xylanisolvens, Barnesiella intestinihominis, Bifidobacterium adolescentis, Bifidobacterium catenulatum, Bifidobacterium dentium, Bifidobacterium faecale, Bifidobacterium kashiwanohense, Bifidobacterium longum, Bifidobacterium pseudocatenulatum, Bifidobacterium ruminantium, Bifidobacterium stercoris, Blautia coccoides, Blautia hansenii, Blautia hominis, Blautia hydrogenotrophica, Blautia luti, Blautia marasmi, Blautia obeum, Blautia producta, Blautia stercoris, Blautia wexlerae, Brenneria alni, Butyricimonas faecihominis, Cedecea lapagei, Cellulosilyticum lentocellum, Citrobacter amalonaticus, Citrobacter farmeri, Citrobacter koseri, Citrobacter sedlakii, Citrobacter youngae, Clostridium aldenense, Clostridium asparagiforme, Clostridium beijerinckii, Clostridium bolteae, Clostridium butyricum, Clostridium earths, Clostridium celatum, Clostridium chauvoei, Clostridium chromiireducens, Clostridium citroniae, Clostridium clostridioforme, Clostridium cocleatum, Clostridium dakarense, Clostridium diolis, Clostridium disporicum, Clostridium glycyrrhizinilyticum, Clostridium hylemonae, Clostridium innocuum, Clostridium lavalense, Clostridium paraputrificum, Clostridium puniceum, Clostridium quinii, Clostridium saccharobutylicum, Clostridium saccharoperbutylacetonicum, Clostridium sartagoforme, Clostridium saudiense, Clostridium scindens, Clostridium septicum, Clostridium spiroforme, Clostridium subterminale, Clostridium sulfidigenes, Clostridium symbiosum, Clostridium tertium, Clostridium thiosulfatireducens, Collinsella aerofaciens, Coprococcus comes, Coprococcus eutactus, Cronobacter condimenti, Cronobacter muytjensii, Cronobacter sakazakii, Dorea longicatena, Drancourtella massiliensis, Eggerthella lenta, Eisenbergiella massiliensis, Eisenbergiella tayi, Emergencia timonensis, Enterobacter asburiae, Enterobacter bugandensis, Enterobacter cloacae, Enterobacter hormaechei, Enterobacter tabaci, Erysipelatoclostridium ramosum, Escherichia albertii, Escherichia coli, Escherichia fergusonii, Escherichia marmotae, Eubacterium callanderi, Eubacterium limosum, Eubacterium maltosivorans, Eubacterium rectale, Eubacterium tenue, Faecalibacterium prausnitzii, Faecalicatena contorta, Faecalicatena fissicatena, Faecalicatena orotica, Flavonifractor plautii, Gemmiger formicilis, Harryflintia acetispora, Holdemania filiformis, Holdemania massiliensis, Intestinibacter bartlettii, Intestinimonas butyriciproducens, Kosakonia cowanii, Kosakonia oryzendophytica, Kosakonia oryziphila, Kosakonia pseudosacchari, Kosakonia sacchari, Lachnoclostridium pacaense, Lachnospira pectinoschiza, Lactobacillus fermentum, Lactobacillus gorillae, Lactonifactor longoviformis, Longibaculum muris, Longicatena caecimuris, Metakosakonia massiliensis, Mixta theicola, Murimonas intestini, Oscillibacter ruminantium, Paeniclostridium ghonii, Paeniclostridium sordellii, Pantoea beijingensis, Parabacteroides distasonis, Parabacteroides johnsonii, Parabacteroides merdae, Paraclostridium benzoelyticum, Paraclostridium bifermentans, Pectobacterium carotovorum, Peptostreptococcus anaerobius, Peptostreptococcus stomatis, Phytobacter ursingii, Pseudescherichia vulneris, Pseudocitrobacter anthropi, Pseudocitrobacter faecalis, Pseudoflavonifractor capillosus, Pseudoflavonifractor phocaeensis, Raoultella planticola, Robinsoniella peoriensis, Romboutsia ilealis, Romboutsia lituseburensis, Romboutsia sedimentorum, Romboutsia timonensis, Roseburia faecis, Roseburia hominis, Roseburia intestinalis, Roseburia inulinivorans, Ruminococcus albus, Ruminococcus bromii, Ruminococcus faecis, Ruminococcus gnavus, Ruminococcus lactaris, Ruminococcus torques, Ruthenibacterium lactatiformans, Salmonella bongori, Salmonella enterica, Sellimonas intestinalis, Shigella boydii, Shigella dysenteriae, Shigella flexneri, Shigella sonnei, Subdoligranulum variabile, Terrisporobacter glycolicus, Terrisporobacter mayombei, Terrisporobacter petrolearius, Trabulsiella odontotermitis, Turicibacter sanguinis, Tyzzerella nexilis, or Yokenella regensburgei. In some embodiments, a bacterial composition can comprise at least one, two, three, four, five, six, seven, eight, nine, ten, eleven, twelve, thirteen, fourteen, fifteen, sixteen, seventeen, eighteen, nineteen, twenty, twenty-five, thirty, thirty-five, forty, forty-five, fifty, or all of the species listed.

In some embodiments, a bacterial composition comprises one or more of the following bacterial species: Agathobaculum desmolans, Akkermansia muciniphila, Alistipes finegoldii, Alistipes shahii, Anaerofustis stercorihominis, Anaeromassilibacillus senegalensis, Anaerostipes caccae, Anaerotruncus colihominis, Bacteroides caccae, Bacteroides eggerthii, Bacteroides faecis, Bacteroides intestinalis, Bacteroides koreensis, Bacteroides salyersiae, Bacteroides uniformis, Bacteroides vulgatus, Bacteroides xylanisolvens, Barnesiella intestinihominis, Bifidobacterium dentium, Bifidobacterium longum, Bifidobacterium stercoris, Blautia coccoides, Blautia hominis, Blautia hydrogenotrophica, Blautia luti, Blautia marasmi, Blautia obeum, Blautia producta, Blautia wexlerae, Butyricimonas faecihominis, Cellulosilyticum lentocellum, Clostridium aldenense, Clostridium asparagiforme, Clostridium bolteae, Clostridium butyricum, Clostridium citroniae, Clostridium clostridioforme, Clostridium cocleatum, Clostridium disporicum, Clostridium hylemonae, Clostridium innocuum, Clostridium lavalense, Clostridium scindens, Clostridium spiroforme, Clostridium subterminale, Clostridium symbiosum, Clostridium tertium, Collinsella aerofaciens, Coprococcus comes, Dorea longicatena, Drancourtella massiliensis, Eggerthella lenta, Eisenbergiella massiliensis, Eisenbergiella tayi, Emergencia timonensis, Erysipelatoclostridium ramosum, Eubacterium callanderi, Eubacterium limosum, Eubacterium maltosivorans, Eubacterium rectale, Faecalibacterium prausnitzii, Faecalicatena contorta, Faecalicatena fissicatena, Faecalicatena orotica, Flavonifractor plautii, Gemmiger formicilis, Harryflintia acetispora, Holdemania filiformis, Holdemania massiliensis, Intestinimonas butyriciproducens, Lachnoclostridium pacaense, Lactobacillus fermentum, Lactonifactor longoviformis, Longicatena caecimuris, Murimonas intestini, Oscillibacter ruminantium, Paeniclostridium sordellii, Parabacteroides distasonis, Parabacteroides merdae, Paraclostridium bifermentans, Peptostreptococcus stomatis, Pseudoflavonifractor capillosus, Pseudoflavonifractor phocaeensis, Robinsoniella peoriensis, Romboutsia timonensis, Roseburia intestinalis, Roseburia inulinivorans, Ruminococcus albus, Ruminococcus bromii, Ruminococcus faecis, Ruminococcus gnavus, Ruminococcus lactaris, Ruminococcus torques, Ruthenibacterium lactatiformans, Sellimonas intestinalis, Terrisporobacter mayombei, Terrisporobacter petrolearius, or Turicibacter sanguinis. In some embodiments, a bacterial composition can comprise at least one, two, three, four, five, six, seven, eight, nine, ten, eleven, twelve, thirteen, fourteen, fifteen, sixteen, seventeen, eighteen, nineteen, twenty, twenty-five, thirty, thirty-five, forty, forty-five, fifty, or all of the species listed.

In some embodiments, a bacterial composition comprises one or more of the following bacterial species: Agathobaculum desmolans, Akkermansia muciniphila, Alistipes finegoldii, Alistipes onderdonkii, Alistipes putredinis, Alistipes senegalensis, Alistipes shahii, Alistipes timonensis, Anaerofustis stercorihominis, Anaeromassilibacillus senegalensis, Anaerostipes caccae, Anaerotruncus colihominis, Bacteroides caccae, Bacteroides cellulosilyticus, Bacteroides eggerthii, Bacteroides faecichinchillae, Bacteroides faecis, Bacteroides finegoldii, Bacteroides intestinalis, Bacteroides koreensis, Bacteroides kribbi, Bacteroides oleiciplenus, Bacteroides ovatus, Bacteroides rodentium, Bacteroides salyersiae, Bacteroides stercorirosoris, Bacteroides thetaiotaomicron, Bacteroides uniformis, Bacteroides vulgatus, Bacteroides xylanisolvens, Barnesiella intestinihominis, Bifidobacterium adolescentis, Bifidobacterium catenulatum, Bifidobacterium dentium, Bifidobacterium faecale, Bifidobacterium kashiwanohense, Bifidobacterium longum, Bifidobacterium pseudocatenulatum, Bifidobacterium ruminantium, Bifidobacterium stercoris, Blautia coccoides, Blautia hansenii, Blautia hominis, Blautia hydrogenotrophica, Blautia luti, Blautia marasmi, Blautia obeum, Blautia producta, Blautia stercoris, Blautia wexlerae, Butyricimonas faecihominis, Cellulosilyticum lentocellum, Clostridium aldenense, Clostridium asparagiforme, Clostridium beijerinckii, Clostridium bolteae, Clostridium butyricum, Clostridium earths, Clostridium celatum, Clostridium chauvoei, Clostridium chromiireducens, Clostridium citroniae, Clostridium clostridioforme, Clostridium cocleatum, Clostridium dakarense, Clostridium diolis, Clostridium disporicum, Clostridium hylemonae, Clostridium innocuum, Clostridium lavalense, Clostridium paraputrificum, Clostridium puniceum, Clostridium quinii, Clostridium saccharobutylicum, Clostridium saccharoperbutylacetonicum, Clostridium sartagoforme, Clostridium saudiense, Clostridium scindens, Clostridium septicum, Clostridium spiroforme, Clostridium subterminale, Clostridium sulfidigenes, Clostridium symbiosum, Clostridium tertium, Clostridium thiosulfatireducens, Collinsella aerofaciens, Coprococcus comes, Dorea longicatena, Drancourtella massiliensis, Eggerthella lenta, Eisenbergiella massiliensis, Eisenbergiella tayi, Emergencia timonensis, Erysipelatoclostridium ramosum, Eubacterium callanderi, Eubacterium limosum, Eubacterium maltosivorans, Eubacterium rectale, Eubacterium tenue, Faecalibacterium prausnitzii, Faecalicatena contorta, Faecalicatena fissicatena, Faecalicatena orotica, Flavonifractor plautii, Gemmiger formicilis, Harryflintia acetispora, Holdemania filiformis, Holdemania massiliensis, Intestinibacter bartlettii, Intestinimonas butyriciproducens, Lachnoclostridium pacaense, Lactobacillus fermentum, Lactobacillus gorillae, Lactonifactor longoviformis, Longicatena caecimuris, Murimonas intestini, Oscillibacter ruminantium, Paeniclostridium ghonii, Paeniclostridium sordellii, Parabacteroides distasonis, Parabacteroides johnsonii, Parabacteroides merdae, Paraclostridium benzoelyticum, Paraclostridium bifermentans, Peptostreptococcus anaerobius, Peptostreptococcus stomatis, Pseudoflavonifractor capillosus, Pseudoflavonifractor phocaeensis, Robinsoniella peoriensis, Romboutsia ilealis, Romboutsia lituseburensis, Romboutsia sedimentorum, Romboutsia timonensis, Roseburia faecis, Roseburia hominis, Roseburia intestinalis, Roseburia inulinivorans, Ruminococcus albus, Ruminococcus bromii, Ruminococcus faecis, Ruminococcus gnavus, Ruminococcus lactaris, Ruminococcus torques, Ruthenibacterium lactatiformans, Sellimonas intestinalis, Subdoligranulum variabile, Terrisporobacter glycolicus, Terrisporobacter mayombei, Terrisporobacter petrolearius, or Turicibacter sanguinis. In some embodiments, a bacterial composition can comprise at least one, two, three, four, five, six, seven, eight, nine, ten, eleven, twelve, thirteen, fourteen, fifteen, sixteen, seventeen, eighteen, nineteen, twenty, twenty-five, thirty, thirty-five, forty, forty-five, fifty, or all of the species listed.

In some embodiments, a bacterial composition comprises one or more of the following bacterial species: Anaerotruncus colihominis, Blautia producta, Blautia wexlerae, Clostridium aldenense, Clostridium bolteae, Clostridium innocuum, Faecalicatena orotica, Erysipelatoclostridium ramosum, Eisenbergiella tayi, Emergencia timonensis, Eubacterium maltosivorans, Flavonifractor plautii, Murimonas intestini, Blautia obeum, Dorea longicatena, Clostridium scindens. In some embodiments, one or more of the bacteria in a composition has at least about 97% identity, e.g., at least about 97%, at least about 97.5%, at least about 98%, at least about 98.5%, at least 99% identity, to a 16S rDNA of the foregoing species. In some embodiments, a bacterial composition can comprise at least one, two, three, four, five, six, seven, eight, nine, ten, eleven, twelve, thirteen, fourteen, fifteen, or all of the bacterial species listed.

In some embodiments, a bacterial composition comprises one or more of the following bacterial species: Anaerotruncus colihominis, Blautia coccoides, Blautia hominis, Blautia luti, Blautia marasmi, Blautia obeum, Blautia producta, Blautia stercorin, Blautia wexlerae, Clostridium aldenense, Clostridium bolteae, Clostridium innocuum, Dorea longicatena, Eisenbergiella massiliensis, Eisenbergiella tayi, Emergencia timonensis, Erysipelatoclostridium ramosum, Eubacterium callanderi, Eubacterium limosum, Faecalicatena contorta, Faecalicatena fissicatena, Faecalicatena orotica, Flavonifractor plautii, Murimonas intestini, or Pseudoflavonifractor phocaeensis. In some embodiments, one or more of the bacteria in a composition has at least about 97% identity, e.g., at least about 97%, at least about 97.5%, at least about 98%, at least about 98.5%, at least about 99% identity, to a 16S rDNA of the foregoing species. In some embodiments, a bacterial composition can comprise at least one, two, three, four, five, six, seven, eight, nine, ten, eleven, twelve, thirteen, fourteen, fifteen, or all of the bacterial species listed.

In some embodiments, a bacterial composition comprises one or more of the following bacterial species: Anaerotruncus colihominis, Blautia coccoides, Blautia hominis, Blautia luti, Blautia marasmi, Blautia obeum, Blautia producta, Blautia stercoris, Blautia wexlerae, Clostridium aldenense, Clostridium bolteae, Clostridium innocuum, Clostridium scindens, Dorea longicatena, Eisenbergiella massiliensis, Eisenbergiella tayi, Emergencia timonensis, Erysipelatoclostridium ramosum, Eubacterium callanderi, Eubacterium limosum, Faecalicatena contorta, Faecalicatena fissicatena, Faecalicatena orotica, Flavonifractor plautii, Murimonas intestini, or Pseudoflavonifractor phocaeensis. In some embodiments, one or more of the bacteria in a composition has at least about 97% identity, e.g., at least about 97%, at least about 97.5%, at least about 98%, at least about 98.5%, at least about 99% identity, to a 16S rDNA of the foregoing species. In some embodiments, a bacterial composition can comprise at least one, two, three, four, five, six, seven, eight, nine, ten, eleven, twelve, thirteen, fourteen, fifteen, or all of the bacterial species listed.

In some embodiments, a bacterial composition comprises one or more of the following bacterial species: Bacteroides salyersiae, Bacteroides vulgatus, Bifidobacterium longum, Blautia obeum, Clostridium asparagiforme, Clostridium bolteae, Clostridium innocuum, Clostridium lavalense, Clostridium scindens, Dorea longicatena, Emergencia timonensis, Faecalicatena orotica, Gemmiger formicilis, Intestinibacter bartlettii, Intestinimonas butyriciproducens, Romboutsia ilealis, Romboutsia timonensis, Roseburia faecis, Roseburia hominis, Roseburia intestinalis, Ruminococcus faecis, Subdoligranulum variabile, Terrisporobacter glycolicus, Terrisporobacter mayombei, or Terrisporobacter petrolearius. In some embodiments, one or more of the bacteria in a composition has at least about 97% identity, e.g., at least about 97%, at least about 97.5%, at least about 98%, at least about 98.5%, at least about 99% identity, to a 16S rDNA of the foregoing species. In some embodiments, a bacterial composition can comprise at least one, two, three, four, five, six, seven, eight, nine, ten, eleven, twelve, thirteen, fourteen, fifteen, or all of the bacterial species listed.

In some embodiments, a bacterial composition comprises one or more of the following bacterial species: Agathobaculum desmolans, Akkermansia muciniphila, Alistipes finegoldii, Alistipes onderdonkii, Alistipes putredinis, Alistipes senegalensis, Alistipes shahii, Alistipes timonensis, Anaerotruncus colihominis, Bacteroides caccae, Bacteroides cellulosilyticus, Bacteroides faecis, Bacteroides finegoldii, Bacteroides intestinalis, Bacteroides oleiciplenus, Bacteroides stercorirosoris, Bacteroides thetaiotaomicron, Bifidobacterium adolescentis, Bifidobacterium catenulatum, Bifidobacterium dentium, Bifidobacterium faecale, Bifidobacterium kashiwanohense, Bifidobacterium longum, Bifidobacterium pseudocatenulatum, Bifidobacterium ruminantium, Bifidobacterium stercoris, Blautia coccoides, Blautia hominis, Blautia marasmi, Blautia producta, Blautia stercoris, Clostridium aldenense, Clostridium subterminale, Clostridium sulfidigenes, Clostridium thiosulfatireducens, Emergencia timonensis, Faecalibacterium prausnitzii, Intestinibacter bartlettii, Intestinimonas butyriciproducens, Roseburia faecis, Roseburia hominis, Roseburia intestinalis, Terrisporobacter glycolicus, Terrisporobacter mayombei, or Terrisporobacter petrolearius. In some embodiments, one or more of the bacteria in a composition has at least about 97% identity, e.g., at least about 97%, at least about 97.5%, at least about 98%, at least about 98.5%, at least about 99% identity, to a 16S rDNA of the foregoing species. In some embodiments, a bacterial composition can comprise at least one, two, three, four, five, six, seven, eight, nine, ten, eleven, twelve, thirteen, fourteen, fifteen, or all of the bacterial species listed.

In some embodiments, a bacterial composition comprises one or more of the following bacterial species: Anaerotruncus colihominis, Blautia coccoides, Blautia hominis, Blautia marasmi, Blautia obeum, Blautia producta, Blautia stercoris, Clostridium aldenense, Clostridium bolteae, Clostridium innocuum, Dorea longicatena, Eisenbergiella massiliensis, Eisenbergiella tayi, Emergencia timonensis, Erysipelatoclostridium ramosum, Faecalicatena contorta, Faecalicatena fissicatena, Faecalicatena orotica, Flavonifractor plautii, Murimonas intestini, or Pseudoflavonifractor phocaeensis. In some embodiments, one or more of the bacteria in a composition has at least about 97% identity, e.g., at least about 97%, at least about 97.5%, at least about 98%, at least about 98.5%, at least about 99% identity, to a 16S rDNA of the foregoing species. In some embodiments, a bacterial composition can comprise at least one, two, three, four, five, six, seven, eight, nine, ten, eleven, twelve, thirteen, fourteen, fifteen, or all of the bacterial species listed.

In some embodiments, a bacterial composition comprises one or more of the following bacterial species: Anaerotruncus colihominis, Blautia hominis, Blautia luti, Blautia obeum, Clostridium aldenense, Clostridium bolteae, Clostridium innocuum, Dorea longicatena, Eisenbergiella tayi, Emergencia timonensis, Erysipelatoclostridium ramosum, Eubacterium callanderi, Faecalicatena orotica, Flavonifractor plautii, or Murimonas intestini. In some embodiments, one or more of the bacteria in a composition has at least about 97% identity, e.g., at least about 97%, at least about 97.5%, at least about 98%, at least about 98.5%, at least about 99% identity, to a 16S rDNA of the foregoing species. In some embodiments, a bacterial composition can comprise at least one, two, three, four, five, six, seven, eight, nine, ten, eleven, twelve, thirteen, fourteen, fifteen, or all of the bacterial species listed.

In some embodiments, a bacterial composition comprises one or more of the following bacterial species: Anaerotruncus colihominis, Blautia hominis, Blautia obeum, Blautia wexlerae, Clostridium aldenense, Clostridium bolteae, Clostridium innocuum, Clostridium scindens, Dorea longicatena, Eisenbergiella tayi, Emergencia timonensis, Erysipelatoclostridium ramosum, Eubacterium callanderi, Faecalicatena orotica, Flavonifractor plautii, or Murimonas intestini. In some embodiments, one or more of the bacteria in a composition has at least about 97% identity, e.g., at least about 97%, at least about 97.5%, at least about 98%, at least about 98.5%, at least about 99% identity, to a 16S rDNA of the foregoing species. In some embodiments, a bacterial composition can comprise at least one, two, three, four, five, six, seven, eight, nine, ten, eleven, twelve, thirteen, fourteen, fifteen, or all of the bacterial species listed.

In some embodiments, a bacterial composition comprises one or more of the following bacterial species: Bacteroides salyersiae, Bacteroides vulgatus, Bifidobacterium longum, Blautia obeum, Clostridium innocuum, Clostridium lavalense, Clostridium scindens, Dorea longicatena, Emergencia timonensis, Faecalicatena orotica, Gemmiger formicilis, Intestinimonas butyriciproducens, Roseburia intestinalis, Ruminococcus faecis, Terrisporobacter mayombei, or Terrisporobacter petrolearius. In some embodiments, one or more of the bacteria in a composition has at least about 97% identity, e.g., at least about 97%, at least about 97.5%, at least about 98%, at least about 98.5%, at least about 99% identity, to a 16S rDNA of the foregoing species. In some embodiments, a bacterial composition can comprise at least one, two, three, four, five, six, seven, eight, nine, ten, eleven, twelve, thirteen, fourteen, fifteen, or all of the bacterial species listed.

In some embodiments, a bacterial composition comprises one or more of the following bacterial species: Agathobaculum desmolans, Akkermansia muciniphila, Alistipes finegoldii, Anaerotruncus colihominis, Bacteroides caccae, Bacteroides faecis, Bacteroides intestinalis, Bifidobacterium longum, Bifidobacterium stercoris, Blautia hominis, Clostridium aldenense, Clostridium subterminale, Emergencia timonensis, Faecalibacterium prausnitzii, Intestinimonas butyriciproducens, Roseburia intestinalis, Terrisporobacter mayombei, or Terrisporobacter petrolearius. In some embodiments, one or more of the bacteria in a composition has at least about 97% identity, e.g., at least about 97%, at least about 97.5%, at least about 98%, at least about 98.5%, at least about 99% identity, to a 16S rDNA of the foregoing species. In some embodiments, a bacterial composition can comprise at least one, two, three, four, five, six, seven, eight, nine, ten, eleven, twelve, thirteen, fourteen, fifteen, or all of the bacterial species listed.

In some embodiments, a bacterial composition comprises one or more of the following bacterial species: Anaerotruncus colihominis, Blautia hominis, Blautia obeum, Clostridium aldenense, Clostridium bolteae, Clostridium innocuum, Dorea longicatena, Eisenbergiella tayi, Emergencia timonensis, Erysipelatoclostridium ramosum, Faecalicatena orotica, Flavonifractor plautii, or Murimonas intestini. In some embodiments, one or more of the bacteria in a composition has at least about 97% identity, e.g., at least about 97%, at least about 97.5%, at least about 98%, at least about 98.5%, at least about 99% identity, to a 16S rDNA of the foregoing species. In some embodiments, a bacterial composition can comprise at least one, two, three, four, five, six, seven, eight, nine, ten, eleven, twelve, thirteen, fourteen, fifteen, or all of the bacterial species listed.

In some embodiments, a bacterial composition comprises one or more of the following bacterial species: Anaerotruncus colihominis, Blautia hominis, Blautia obeum, Clostridium aldenense, Clostridium bolteae, Clostridium innocuum, Dorea longicatena, Eisenbergiella tayi, Emergencia timonensis, Erysipelatoclostridium ramosum, Faecalicatena orotica, Flavonifractor plautii, or Murimonas intestini. In some embodiments, one or more of the bacteria in a composition has at least about 97% identity, e.g., at least about 97%, at least about 97.5%, at least about 98%, at least about 98.5%, at least about 99% identity, to a 16S rDNA of the foregoing species. In some embodiments, a bacterial composition can comprise at least one, two, three, four, five, six, seven, eight, nine, ten, eleven, twelve, thirteen, fourteen, fifteen, or all of the bacterial species listed.

In some embodiments, a bacterial composition comprises one or more of the following bacterial species: Anaerotruncus colihominis, Blautia coccoides, Blautia hominis, Blautia luti, Blautia marasmi, Blautia producta, Blautia stercoris, Blautia wexlerae, Clostridium aldenense, Clostridium bolteae, Clostridium innocuum, Eisenbergiella massiliensis, Eisenbergiella tayi, Emergencia timonensis, Erysipelatoclostridium ramosum, Eubacterium callanderi, Eubacterium limosum, Faecalicatena contorta, Faecalicatena fissicatena, Faecalicatena orotica, Flavonifractor plautii, Murimonas intestini, or Pseudoflavonifractor phocaeensis. In some embodiments, one or more of the bacteria in a composition has at least about 97% identity, e.g., at least about 97%, at least about 97.5%, at least about 98%, at least about 98.5%, at least about 99% identity, to a 16S rDNA of the foregoing species. In some embodiments, a bacterial composition can comprise at least one, two, three, four, five, six, seven, eight, nine, ten, eleven, twelve, thirteen, fourteen, fifteen, or all of the bacterial species listed.

In some embodiments, a bacterial composition comprises one or more of the following bacterial species: Anaerotruncus colihominis, Blautia coccoides, Blautia hominis, Blautia luti, Blautia marasmi, Blautia obeum, Blautia producta, Blautia stercoris, Blautia wexlerae, Clostridium aldenense, Clostridium bolteae, Clostridium innocuum, Dorea longicatena, Eisenbergiella massiliensis, Eisenbergiella tayi, Emergencia timonensis, Erysipelatoclostridium ramosum, Eubacterium callanderi, Eubacterium limosum, Faecalicatena contorta, Faecalicatena fissicatena, Faecalicatena orotica, Flavonifractor plautii, Murimonas intestini, or Pseudoflavonifractor phocaeensis. In some embodiments, one or more of the bacteria in a composition has at least about 97% identity, e.g., at least about 97%, at least about 97.5%, at least about 98%, at least about 98.5%, at least about 99% identity, to a 16S rDNA of the foregoing species. In some embodiments, a bacterial composition can comprise at least one, two, three, four, five, six, seven, eight, nine, ten, eleven, twelve, thirteen, fourteen, fifteen, or all of the bacterial species listed.

In some embodiments, a bacterial composition comprises one or more of the following bacterial species: Anaerotruncus colihominis, Blautia coccoides, Blautia hominis, Blautia luti, Blautia marasmi, Blautia obeum, Blautia producta, Blautia stercoris, Blautia wexlerae, Clostridium aldenense, Clostridium bolteae, Clostridium innocuum, Dorea longicatena, Eisenbergiella massiliensis, Eisenbergiella tayi, Emergencia timonensis, Erysipelatoclostridium ramosum, Eubacterium callanderi, Eubacterium limosum, Faecalicatena contorta, Faecalicatena fissicatena, Faecalicatena orotica, Flavonifractor plautii, Murimonas intestini, Pseudoflavonifractor phocaeensis. In some embodiments, one or more of the bacteria in a composition has at least about 97% identity, e.g., at least about 97%, at least about 97.5%, at least about 98%, at least about 98.5%, at least about 99% identity, to a 16S rDNA of the foregoing species. In some embodiments, a bacterial composition can comprise at least one, two, three, four, five, six, seven, eight, nine, ten, eleven, twelve, thirteen, fourteen, fifteen, or all of the bacterial species listed.

In some embodiments, a bacterial composition comprises one or more of the following bacterial species: Agathobaculum desmolans, Anaerotruncus colihominis, Blautia hominis, Blautia wexlerae, Clostridium aldenense, Clostridium bolteae, Clostridium innocuum, Eisenbergiella tayi, Emergencia timonensis, Erysipelatoclostridium ramosum, Eubacterium callanderi, Faecalicatena orotica, Flavonifractor plautii, Murimonas intestini, Roseburia intestinalis, Terrisporobacter mayombei, or Terrisporobacter petrolearius. In some embodiments, one or more of the bacteria in a composition has at least about 97% identity, e.g., at least about 97%, at least about 97.5%, at least about 98%, at least about 98.5%, at least about 99% identity, to a 16S rDNA of the foregoing species. In some embodiments, a bacterial composition can comprise at least one, two, three, four, five, six, seven, eight, nine, ten, eleven, twelve, thirteen, fourteen, fifteen, or all of the bacterial species listed.

In some embodiments, a bacterial composition comprises one or more of the following bacterial species: Anaerotruncus colihominis, Blautia hominis, Blautia wexlerae, Clostridium aldenense, Clostridium bolteae, Clostridium innocuum, Eisenbergiella tayi, Emergencia timonensis, Erysipelatoclostridium ramosum, Eubacterium callanderi, Faecalicatena orotica, Flavonifractor plautii, or Murimonas intestini. In some embodiments, one or more of the bacteria in a composition has at least about 97% identity, e.g., at least about 97%, at least about 97.5%, at least about 98%, at least about 98.5%, at least about 99% identity, to a 16S rDNA of the foregoing species. In some embodiments, a bacterial composition can comprise at least one, two, three, four, five, six, seven, eight, nine, ten, eleven, twelve, thirteen, fourteen, fifteen, or all of the bacterial species listed.

In some embodiments, a bacterial composition comprises one or more of the following bacterial species: Anaerotruncus colihominis, Blautia hominis, Blautia obeum, Blautia wexlerae, Clostridium aldenense, Clostridium bolteae, Clostridium innocuum, Dorea longicatena, Eisenbergiella tayi, Emergencia timonensis, Erysipelatoclostridium ramosum, Eubacterium callanderi, Faecalicatena orotica, Flavonifractor plautii, or Murimonas intestini. In some embodiments, one or more of the bacteria in a composition has at least about 97% identity, e.g., at least about 97%, at least about 97.5%, at least about 98%, at least about 98.5%, at least about 99% identity, to a 16S rDNA of the foregoing species. In some embodiments, a bacterial composition can comprise at least one, two, three, four, five, six, seven, eight, nine, ten, eleven, twelve, thirteen, fourteen, fifteen, or all of the bacterial species listed.

In some embodiments, a bacterial composition comprises one or more of the following bacterial species: Anaerotruncus colihominis, Blautia hominis, Blautia obeum, Blautia wexlerae, Clostridium aldenense, Clostridium bolteae, Clostridium innocuum, Dorea longicatena, Eisenbergiella tayi, Emergencia timonensis, Erysipelatoclostridium ramosum, Eubacterium callanderi, Faecalicatena orotica, Flavonifractor plautii, or Murimonas intestini. In some embodiments, one or more of the bacteria in a composition has at least about 97% identity, e.g., at least about 97%, at least about 97.5%, at least about 98%, at least about 98.5%, at least about 99% identity, to a 16S rDNA of the foregoing species. In some embodiments, a bacterial composition can comprise at least one, two, three, four, five, six, seven, eight, nine, ten, eleven, twelve, thirteen, fourteen, fifteen, or all of the bacterial species listed.

In some embodiments, a bacterial composition (e.g., designed composition) disclosed herein comprises one or more of the bacterial species disclosed in Tables 1-3, FIG. 1-2A, FIG. 4E, and/or FIG. 4F.

In some embodiments, a bacterial composition of the present disclosure comprises one or more bacteria comprising a 16S rDNA sequence that is at least about 85%, at least about 90%, at least about 95%, at least about 96%, at least about 97%, at least about 97.5%, at least about 98%, at least about 98.5%, at least about 99%, at least about 99.5%, or about 100% identical to a 16S rDNA sequence set forth in SEQ ID NOs: 1-352.

The term “16S sequencing” or “16S rDNA” or “16S” refers to sequence derived by characterizing the nucleotides that comprise the 16S ribosomal RNA gene(s). The bacterial 16S rDNA is approximately 1500 nucleotides in full length and can be referred to fragments ranging from a few nucleotides to full length of the 16S rDNA. 16S rDNA is used in reconstructing the evolutionary relationships and sequence similarity of one bacterial isolate to another using phylogenetic approaches. 16S sequences are used for phylogenetic reconstruction as they are in general highly conserved, but contain specific hypervariable regions that harbor sufficient nucleotide diversity to differentiate genera and species of most bacteria.

The term “V1-V9 regions” of the 16S rRNA refers to the first through ninth hypervariable regions of the 16S rRNA gene that are used for genetic typing of bacterial samples. These regions in bacteria are defined by nucleotides 69-99, 137-242, 433-497, 576-682, 822-879, 986-1043, 1117-1173, 1243-1294 and 1435-1465 respectively using numbering based on the E. coli system of nomenclature. Brosius et al., Complete nucleotide sequence of a 16S ribosomal RNA gene from Escherichia coli, PNAS (1978). In some embodiments, a sequence comprising at least one of the V1, V2, V3, V4, V5, V6, V7, V8, and V9 regions is used to characterize an OTU. In some embodiments, a sequence comprising at least all of the V1, V2, V3, V4, V5, V6, V7, V8, and V9 regions is used to characterize an OTU. In some embodiments, a sequence comprising the V1, V2, and V3 regions is used to characterize an OTU. In another embodiment, a sequence comprising the V3, V4, and V5 regions is used to characterize an OTU. In another embodiment, a sequence comprising the V3, and V4 regions is used to characterize an OTU. In another embodiment, a sequence comprising the V4, and V5 regions is used to characterize an OTU. In another embodiment, a sequence comprising the V4 region is used to characterize an OTU. A person of ordinary skill in the art can identify the specific hypervariable regions of a candidate 16S rRNA by comparing the candidate sequence in question to a reference sequence and identifying the hypervariable regions based on similarity to the reference hypervariable regions, or alternatively, one can employ Whole Genome Shotgun (WGS) sequence characterization of microbes or a microbial community.

In some embodiments, a bacterial composition disclosed herein (e.g., designed compositions) comprises both a spore-forming bacteria and a non-spore-forming bacteria. In some embodiments, a bacterial composition comprises only spore-forming bacteria. In some cases, the bacteria of the compositions are in spore form.

Applicant has also discovered that certain bacterial species are associated with exacerbation or non-improvement of at least one sign or symptom of a disease or disorder associated with dysbiosis of the gastrointestinal microbiome (e.g., infection, GvHD, mucositis). The presence of such species in a bacterial composition can be undesirable. Accordingly, in some embodiments, a bacterial composition (e.g., designed compositions) does not include one or more of the following bacterial species: Klebsiella pneumoniaea, Enterococcus faecium, Enterococcus faecalis, Bifidobacterium dentium, Dialister invisus, Prevotella copri, Veillonella atypica, Veillonella dispar, Veillonella parvula, or Veillonella ratti. In certain embodiments, a bacterial composition does not include one or more bacteria that has at least about 97%, e.g., about 99% identity, to a 16S rDNA of the foregoing species. In some embodiments, a bacterial composition does not include at least one, two, three, four, five, six, seven, eight, nine, or all of the species listed.

In some embodiments, a bacterial composition of the present disclosure does not comprise one or more bacteria comprising a 16S rDNA sequence that is at least about 85%, at least about 90%, at least about 95%, at least about 96%, at least about 97%, at least about 97.5%, at least 98%, at least about 98.5%, at least about 99%, at least about 99.5%, or about 100% identical to a 16S rDNA sequence set forth in SEQ ID NO: 1-352.

As described supra, bacteria that are beneficial for the treatment of a disease or disorder associated with dysbiosis (e.g., infection or GvHD following HSCT) are associated with certain biological functions. Accordingly, in some embodiments, types of bacteria present in a bacterial composition disclosed herein (e.g., designed compositions) are associated with certain biological functions, which are useful in treating, preventing, delaying, or ameliorating one or more signs or symptoms associated with a disease or disorder disclosed herein (e.g., infection or GvHD following HSCT). Non-limiting examples of relevant functional features are further described below.

### Functional Features

In some embodiments of the invention, a microbiome composition disclosed herein (e.g., designed compositions such as DE122435.3) is a composition that includes bacteria that can carry out certain functions identified herein as being useful for treating and/or preventing a disease or disorder associated with dysbiosis (e.g., infection or GvHD following HSCT). In certain embodiments, bacterial species that are useful for the present disclosure comprises one or more of the following features: (1) capable of engrafting (long-term and/or transient) when administered to a subject; (2) capable of having anti-inflammatory (e.g., inhibiting TNF-α-driven IL-8 secretion in epithelial cells in vitro, ability to downmodulate expression of inflammatory genes (e.g., CXCL1, CXCL2, CXCL3, CXCL11, ICAM1)); (3) not capable of inducing pro-inflammatory activity (e.g., does not induce IL-8 production by IECs); (4) capable of producing secondary bile acids (e.g., 7α-dehydroxylase and bile salt hydrolase activity); (5) capable of producing tryptophan metabolites (e.g., indole, 3-methyl indole, indolepropionic acid); (6) capable of restoring epithelial integrity, as determined by a primary epithelial cell monolayer barrier integrity assay; (7) capable of being associated with reduction of risk of infection or GvHD following HSCT; (8) capable of not being associated with clinical non-remission of infection or GvHD following HSCT; (9) capable of producing short-chain fatty acids (e.g., butyrate, propionate); (10) capable of inhibiting HDAC activity; (11) capable of producing a medium-chain fatty acid (e.g., valerate, hexanoate); (12) capable of expressing catalase activity; (13) capable of having alpha-fucosidase activity; (14) capable of inducing Wnt activation; (15) capable of producing B vitamins (e.g., thiamin (B1) and/or pyridoxamine (B6)); (16) capable of reducing fecal calprotectin level; (17) not capable of activating a toll-like receptor pathway (e.g., TLR4 or TLR5); (18) capable of activating a toll-like receptor pathway (e.g., TLR2); (19) capable of restoring colonization resistance; (20) capable of a broad range of carbon source utilization; (21) capable of reducing VRE pathogen carriage, (22) capable of reducing CRE pathogen carriage; (23) capable of reducing colonic inflammation; (24) capable of being associated with the healthy human gut microbiota; (25) capable of not being associated with toxin and hemolysin genes associated with Clostridial pathogens and no significant cytopathic effects in vitro; (26) susceptible to multiple clinically relevant antibiotics; (27) capable of not being associated with genes that are both likely responsible for the observed antibiotic resistances and transmissible, (28) capable of inhibiting epithelial cell apoptosis; (29) capable of downmodulating one or more genes induced in IFN-γ treated colonic organoids (e.g., those associated with inflammatory chemokine signaling, NF-κB signaling, TNF family signaling, type I interferon signaling, type II interferon signaling, TLR signaling, lymphocyte trafficking, Th17 cell differentiation, Th2 differentiation, apoptosis, inflammasomes, autophagy, oxidative stress, MEW class I and II antigen presentation, complement, mTor, nod-like receptor signaling, PI3K signaling, or combinations thereof), (30) capable of reducing the expression of one or more inhibitory receptors (e.g., TIGIT, TIM-3, or LAG-3) on T cells, (31) capable of increasing expression of one or more genes/proteins associated with T cell activation and/or function (e.g., CD45RO, CD69, IL-24, TNF-α, perforin, or IFN-γ), (32) capable of enhancing the ability of CD8+ T cells to kill tumor cells, (33) capable of enhancing the efficacy of an immune checkpoint inhibitor therapy, (34) capable of promoting the recruitment of CD8+ T cells to tumors, (35) capable of inducing an anti-inflammatory IL-10-skewed IL-10/IL-6 cytokine ratio in macrophages; (36) capable of inducing less inflammatory responses but similar pathogen defense responses in macrophages than a donor-derived spore-based composition (i.e., a spore-based composition), (37) capable of producing IL-18, or combinations thereof. In certain embodiments, species that are useful for the present disclosure comprises one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, or all of the above features.

Additional disclosure relating to exemplary functional features are provided below.

### Engraftment

As described supra, a key feature of the bacterial compositions disclosed herein is the ability of one or more bacterial species (or OTUs of bacteria) included in the compositions to engraft in a subject when administered to the subject. Accordingly, Applicant has identified bacteria and combinations of bacteria that are capable of engrafting when administered to a subject. Not to be bound by any one theory, engraftment of bacteria and combinations of bacteria disclosed herein can repopulate the gastrointestinal microbiome of a subject. In some embodiments, once engrafted, bacteria and combinations of bacteria disclosed herein prevent (e.g., by outcompeting for growth nutrients) the growth of non-commensal microbes (e.g., pathogenic bacteria, such as Clostridium difficile, ESKAPE pathogens (including: Enterococcus faecium, Staphylococcus aureus, Klebsiella pneumoniae, Acinetobacter baumannii, Pseudomonas aeruginosa, and Enterobacter spp.), Enterococcus species including but not limited to Enterococcus faecalis and Enterococcus faecium, Enterobacteriaceae species including but not limited to Klebsiella pneumonia, or such species that are resistant to vancomycin or carbapenems, drug resistant and multi-drug resistant organisms (MDROs) including VRE, CRE (including Klebsiella pneumoniae, Klebsiella oxytoca, Klebsiella aerogenes, Enterococcus species), drug resistant multi-drug resistant Enterobacteriaceae, and extended spectrum beta-lactamase (ESBLs) producing bacteria (including E. coli, Klebsiella species), or methicillin-resistant Staphylococcus aureus (MRSA)) that may result in local (e.g. GI) infection, or systemic (e.g. bloodstream or tissue) infection in the host, or promote inflammatory responses directly or indirectly (e.g. mucositis, GvHD). In further embodiments, once engrafted, bacteria and combinations of bacteria disclosed herein can promote or augment the growth of other commensal bacteria within the subject. In further embodiments, the engrafting bacteria and combinations of bacteria can produce various factors (e.g., tryptophan metabolites, fatty acids, secondary bile acids) or exert other functions (e.g., those disclosed herein) to help treat and/or prevent one or more symptoms associated with a disease or disorder disclosed herein.

Whether bacteria or combinations of bacteria are capable of engrafting can be determined by various methods known in the art. Subject samples can first be collected (e.g., by whole stool samples, rectal swaps, tissue biopsies, or mucosal samples) before and/or after administration of bacteria or combinations of bacteria. Subsequently, these samples can be characterized to identify the bacteria or combinations of bacteria. Administered bacterial strains can be identified in samples based on genotypic, phenotypic, and other molecular properties of the strains, for example: a) the sequence of certain genes (e.g., 16S rRNA sequence) b) the presence and/or sequence identity of one or more regions of DNA (i.e., linear segments) that are rarely present in other strains, rarely present in other microbiome samples, rarely present in the target patient population, or absent from the microbiome of the particular subject(s) before administration of the bacteria, c) DNA variants including SNVs, insertions and deletions (i.e., indels), structural variation, gene copy number variation, or other DNA variants that are rarely present in other strains, rarely present in other microbiome samples, rarely present in the target patient population, or absent from the microbiome of the particular subject(s) before administration of the bacteria, d) other identifying phenotypic, genomic, proteomic, metabolomic or other properties of the administered strains. Molecular technologies used to identify administered bacteria or combinations of bacteria include but are not limited various DNA sequencing technologies including PCR and qPCR, amplicon sequencing, whole genome sequencing, shotgun metagenomic sequencing; other molecular technologies can be used included but not limited to microarray, multiplexed molecular barcode (e.g., available from NanoString Technologies), and mass spectrometry. Bioinformatic methods used to analyze these data may include sequence alignment and mapping, genome or metagenome assembly, or other methods. Microbiological and culturing methods can also be used to identify and characterize strains. These mentioned methods of identification and characterization of administered bacteria or combinations of bacteria can be used alone or in combination.

In some embodiments, one or more of the bacterial species included in the bacterial compositions disclosed herein are capable of engrafting when administered to a subject. In certain embodiments, each of the bacterial species included in a bacterial composition is capable of engrafting. In some embodiments, the bacteria and combinations of bacteria that are capable of engrafting are long-term engrafters. In certain embodiments, the bacteria and combinations of bacteria that are capable of engrafting are transient engrafters. In some embodiments, the bacterial compositions disclosed herein (e.g., designed compositions) comprise one or more long-term engrafters and one or more transient engrafters. In certain embodiments, a bacterial composition disclosed herein comprises two, three, four, five, six, seven, eight, nine, ten or more long-term engrafters. In some embodiments, a bacterial composition comprises two, three, four, five, six, seven, eight, nine, ten or more transient engrafters. In any such embodiments, the bacterial composition disclosed herein can also include one, two, three, four, five, six, seven, eight, nine, ten or more species not defined as long-term or transient engrafters. In further embodiments, a bacterial composition disclosed herein comprises three or more transient engrafters and/or three or more long-term engrafters, four or more transient engrafters and/or four or more long-term engrafters, five or more transient engrafters and/or four or more long-term engrafters, six or more transient engrafters and/or four or more long-term engrafters, seven or more transient engrafters and/or four or more long-term engrafters, eight or more transient engrafters and/or four or more long-term engrafters, nine or more transient engrafters and/or four or more long-term engrafters, or ten or more transient engrafters and/or four or more long-term engrafters. In any such embodiments, the bacterial composition disclosed herein can also include one, two, three, four, five, six, seven, eight, nine, ten or more species not defined as long-term or transient engrafters. In further embodiments, a bacterial composition disclosed herein comprises ten or more transient engrafters and/or four or more long-term engrafters and/or two or more species not defined as either. Non-limiting examples of long-term engrafters and/or transient engrafters that can be used with the present disclosure are provided in Table 1.

### Bile Acids

Applicant has discovered that certain secondary bile acids are associated with the treatment and/or prevention of a disease or disorder, such as those associated with a dysbiosis (e.g., infection or GvHD following HSCT). The term “bile acids” refers to a family of molecules, composed of a steroid structure with four rings, a five or eight carbon side-chain terminating in a carboxylic acid joined at the 17-position of the steroid scaffold, and the presence and orientation of different numbers of hydroxy groups. Depending on the tissue, the structure of the bile acids can vary. For instance, upon their synthesis in the liver, the bile acids are conjugated to either taurine or glycine residues (“conjugated primary bile acids” also known as bile salts) and subsequently excreted and stored in the gall bladder. During digestion, the conjugated primary bile acids are then secreted into the intestinal lumen. In some embodiments, the primary conjugated bile acids are glycocholic acid (gCA), taurocholic acid (tCA), glycochenodeoxycholic acid (gCDCA), or taurochenodeoxycholic acid (tCDCA).

Within the intestinal lumen, the resident intestinal bacteria express enzymes (e.g., bile salt hydrolase (BSH)), which deconjugate the conjugated primary bile acids to produce “primary bile acids.” In some embodiments, the primary bile acids comprise cholic acid (CA) or chenodeoxycholic acid (CDCA). Primary bile acids are then further processed (via enzymes, such as hydroxysteroid dehydrogenase (HSDH) or 7α-dehydroxylase) to become “secondary bile acids.” Accordingly, in some aspects, the phrase “capable of producing a secondary bile acid” comprises the ability to deconjugate primary bile acids to produce the secondary bile acids. In some embodiments, the secondary bile acids comprise deoxycholic acid (DCA), (3 or 12)-oxo-deoxycholic acid, (3 or 12)-iso-deoxycholic acid, (3, 7 or 12)-oxo-cholic acid, (3, 7 or 12)-iso-cholic acid, lithocholic acid (LCA), oxo-LCA, iso-LCA, (3 or 7)-oxo-chenodeoxy cholic acid, or (3 or 7)-iso-chenodeoxy cholic acid.

The secondary bile acids produced in the intestinal lumen can circulate back to the liver, where they are reconjugated to become “conjugated secondary bile acids.” In some embodiments, the secondary conjugated bile acids of the present disclosure comprise (3 or 12)-glyco-iso-deoxycholic acid, (3 or 12)-tauro-iso-deoxycholic acid, glyco-deoxycholic acid, tauro-deoxycholic acid, (3, 7 or 12)-glyco-iso-cholic acid, (3, 7 or 12)-tauro-iso-cholic acid, sulfo-lithocholic acid, glyco-sulfo-lithocholic acid, tauro-sulfo-lithocholic acid, (3 or 7)-glyco-iso-chenodeoxycholic acid, (3 or 7)-tauro-iso-chenodeoxycholic acid, (3 or 7)-glyco-oxo-chenodeoxycholic acid, or (3 or 7)-tauro-oxo-chenodeoxycholic acid.

In some embodiments, one or more of the bacterial species that can be used in constructing the designed compositions disclosed herein comprise an enzyme involved in secondary bile acid production. In certain embodiments, the enzyme comprises BSH or HSDH. In some embodiments, a bacterial species useful for the present disclosure comprises both BSH and HSDH. Accordingly, in some embodiments, bacteria and combinations of bacteria disclosed herein can increase the level of a bile acid (e.g., a secondary bile acid, e.g., deoxycholic acid (DCA), 3-α-12-oxo-deoxycholic acid, 3-α-7-oxo-deoxycholic acid, 3-α-12-α-7-oxo-deoxycholic acid, 3-α-7-α-12-oxo-deoxycholic acid, 3-β-12-α-deoxycholic acid (3-isodeoxycholic acid), 7-α-3-oxo-chenodeoxycholic acid, lithocholic acid (LCA), 3-oxo LCA, oxo-LCA, iso-LCA, urso-deoxycholic acid (UDCA), and combinations thereof) in a subject.

In some embodiments, the level of a secondary bile acid is increased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, compared to a corresponding level in a reference sample. In some embodiments, the reference sample is a biological sample (e.g., fecal sample) obtained from a subject prior to the administration of a bacterial composition disclosed herein. In other embodiments, the reference sample is a biological sample (e.g., fecal sample) obtained from a subject with an active symptom of a disease or disorder, such as those associated with dysbiosis (e.g., infection or GvHD following HSCT).

In some embodiments, the increase in the level of a secondary bile acid can reduce the level of a pro-inflammatory mediators (e.g., TNF-α or IL-8) produced by activated cells (e.g., LPS-stimulated monocytes, LPS-stimulated PBMCs, or TNF-α-stimulated intestinal epithelial cells). In some embodiments, the increase in the level of a secondary bile acid is correlated with increased populations of anti-inflammatory T regulatory cells, involved in the suppression of GvHD, in the periphery or colon. In some embodiments, the increase in the level of a secondary bile acid can protect epithelial cell viability, reduce mortality in murine models of GvHD, and reduce non-relapse mortality in some HSCT patients with post-transplant liver complications.

In certain embodiments, the amount of pro-inflammatory mediators produced by activated cells is decreased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, compared to a reference sample (e.g., activated cells not treated with increased concentration of a secondary bile acid). In some embodiments, the level of anti-inflammatory mediators produced is increased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a reference sample (e.g., activated cells not treated with increased concentration of a secondary bile acid).

In some embodiments, reducing the level of certain secondary bile acids can be important in the effective treatment of a disease or disorder disclosed herein. Accordingly, in certain embodiments, bacteria and combinations of bacteria that are useful for the present disclosure are capable of reducing the level of a secondary bile acid in a subject. In some embodiments, the level of a secondary bile acid is reduced by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, compared to a corresponding level in a reference sample. In some embodiments, the reference sample is a biological sample (e.g., fecal sample) obtained from a subject prior to the administration of a bacterial composition disclosed herein. In other embodiments, the reference sample is a biological sample (e.g., fecal sample) obtained from a subject with an active symptom of a disease or disorder, such as those associated with dysbiosis (e.g., infection or GvHD following HSCT).

### Anti-Inflammatory Activity

Applicant has identified bacteria and combinations of bacteria that are capable of exhibiting anti-inflammatory activity when administered to a subject. As used herein, the term “anti-inflammatory activity” refers to the ability to prevent and/or reduce inflammation. The term “inflammation” or “pro-inflammatory” refers to the complex biological response of an individual's immune system to harmful stimuli, such as pathogens, damaged cells, or irritants, and includes secretion of pro-inflammatory mediators, such as pro-inflammatory cytokines, i.e., cytokines which are produced predominantly by activated immune cells, such as macrophages and dendritic cells, and are involved in the amplification of inflammatory reactions.

Without being limited to any one particular theory, the anti-inflammatory activity observed with the bacteria and combinations of bacteria disclosed herein can be related to the other functional aspects of the bacteria or combinations of bacteria. For example, in some embodiments, the anti-inflammatory activity is related to the ability of the bacteria or combinations of bacteria to produce a secondary bile acid, a tryptophan metabolite, a short-chain fatty acid, inhibit HDAC inhibition, inhibit TNF-α-driven IL-8 secretion in epithelial cells in vitro, inhibit IFNγ-driven induction of inflammatory and apoptosis pathways in human primary colonic organoids in vitro and/or stimulate IL-10 production by macrophages in vitro. In some aspects, the anti-inflammatory activity (e.g., as demonstrated by IL-8 secretion) is improved compared to a bacterial spore preparation from healthy human donor stool (see, e.g., Example 6). Accordingly, in some embodiments, the bacteria and combinations of bacteria that have anti-inflammatory activity have one or more of the following features: (i) capable of producing a short-chain fatty acid, (ii) capable of inhibiting histone deacetylase (HDAC) activity, (iii) capable of inhibiting TNF-α-driven IL-8 secretion in epithelial cells in vitro, (iv) capable of inhibiting NF-kB and NF-kB target genes, (v) capable of downmodulating one or more genes induced in IFN-γ treated colonic organoids (e.g., those associated with inflammatory chemokine signaling, NF-κB signaling, TNF family signaling, type I interferon signaling, type II interferon signaling, TLR signaling, lymphocyte trafficking, Th17 cell differentiation, Th2 differentiation, apoptosis, inflammasomes, autophagy, oxidative stress, WIC class I and II antigen presentation, complement, mTor, nod-like receptor signaling, PI3K signaling, or combinations thereof), (vi) capable of inducing anti-inflammatory IL-10 production in macrophages in vitro; or (vii) capable of downmodulating expression of one or more inflammatory genes (e.g., CXCL1, CXCL2, CXCL3, CXCL11, ICAM1), (viii) capable of inducing regulatory T cells (Tregs), (ix) capable of inducing a higher ratio of Tregs compared to Th1 and/or Th17 cells in the colonic lamina propria, or any combination thereof. In some aspects, designed composition DE122435.3 downregulates expression of chemokines CXCL1, CXCL2, CXCL5, and CXCL8. Whether bacteria or combinations of bacteria have anti-inflammatory activity can be measured using assays known in the art, including methods to measure metabolites like short-chain fatty acids (e.g., MS, LC-MS, GS-MS, LC-MS/MS), methods of measuring gene expression at the RNA and/or protein level (e.g., multiplexed bead-based (e.g., available from Luminex) cytokine panels, microarray, multiplexed molecular barcode (e.g., available from NanoString Technologies), and RNA-sequencing).

As described herein, designed composition DE122435.3 elicits significantly reduced pro-inflammatory cytokine or transcriptional changes in human macrophages while also maintaining key functionality for innate defense and thus is an improvement over natural communities (i.e., spore-prep compositions from healthy human donor stool).

In some embodiments, the anti-inflammatory activity of the bacteria and combinations of bacteria disclosed herein can reduce the amount of pro-inflammatory mediators produced and/or present in a subject (e.g., suffering from a disease or disorder disclosed herein). In certain embodiments, the amount of pro-inflammatory mediators produced and/or present in the subject is decreased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, compared to a reference sample. In some embodiments, the reference sample is a biological sample obtained from a subject prior to the administration of a bacterial composition disclosed herein. In other embodiments, the reference sample is a biological sample obtained from a subject with an active symptom of a disease or disorder, such as those associated with dysbiosis (e.g., infection or GvHD following HSCT).

In some embodiments, the anti-inflammatory activity of the bacteria and combinations of bacteria disclosed herein can increase the amount of anti-inflammatory mediators in a subject. Non-limiting examples of anti-inflammatory mediators include, but are not limited to, IL-1 receptor antagonists (IL-1RA), IL-4, IL-10, IL-11, IL-13, TGF-0, and combinations thereof. In certain embodiments, the bacteria and combinations of bacteria that are capable of exhibiting anti-inflammatory activity can increase the amount of anti-inflammatory mediators in a subject by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, compared to a reference sample. In some embodiments, the reference sample is a biological sample obtained from a subject prior to the administration of a bacterial composition disclosed herein. In other embodiments, the reference sample is a biological sample obtained from a subject with an active symptom of a disease or disorder, such as those associated with dysbiosis (e.g., infection or GvHD following HSCT).

### Tryptophan Metabolism and Aryl Hydrocarbon Receptor

As used herein, the term “tryptophan” refers to the essential amino acid tryptophan, which is an α-amino acid and has a chemical formula of C₁₁H₁₂N₂O₂. Besides its use in protein synthesis, tryptophan is important in a number of pathways leading to the production of, for example, serotonin (5-hydroxytryptamine), melatonin, kynurenines, and tryptamine. Tryptophan and its metabolites can affect, for example, immunosuppression, immune function, cancer, inflammatory disease, epithelial barrier function, and infection.

Certain tryptophan pathway products have been shown to function as aryl hydrocarbon receptor (Ahr) agonists. The metabolites include, for example, indole, indole-3 aldehyde, indole-3 acetate, indole-3 propionic acid, indole, 3-methylindole, indole-3 acetaldehyde, indole-3 acetonitrile, 6-formylindolo[3,2-b]carbazole (FICZ), and tryptamine. Ahr plays a role in controlling the differentiation and activity of specific T cell subpopulations. It reportedly can influence adaptive immune responses through its effects on both T cells and antigen presenting cells (APCs). Ahr is thought to be involved in development and maintenance of CD4+ T regulatory cells (Tregs) as well as FoxP3-IL-10+ CD4+ Tr1, and induction of Th17 cells. Ahr also alters cytokine expression by Type 3 innate lymphoid cells (ILC3s). These cellular effects include increased production of IL-22. AhR induction by Trp metabolites has been reported to enhance epithelial barrier integrity and ameliorate colitis in in vivo models.

In some embodiments, bacteria or combinations of bacteria disclosed herein can increase the level of a tryptophan metabolite in a subject. In some embodiments, tryptophan metabolite comprises indole, 3-methyl indole, indoleacrylate, or any combination thereof. In certain embodiments, bacteria or combination of bacteria disclosed herein can increase the level of indole and/or 3-methylindole in the subject.

In some embodiments, the level of a tryptophan metabolite is increased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, compared to a corresponding level in a reference sample. In some embodiments, the reference sample is a biological sample (e.g., fecal sample) obtained from a subject prior to the administration of a bacterial composition disclosed herein. In other embodiments, the reference sample is a biological sample (e.g., fecal sample) obtained from a subject with an active symptom of a disease or disorder, such as those associated with dysbiosis (e.g., infection or GvHD following HSCT).

In some embodiments, reducing the level of a tryptophan metabolite in a subject might be useful in treating a disease or disorder. Accordingly, in certain embodiments, bacteria and combinations of bacteria disclosed herein are capable of reducing the level of a tryptophan metabolite in a subject. In some embodiments, the level of a tryptophan metabolite is reduced by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, compared to a corresponding level in a reference sample. In some embodiments, the reference sample is a biological sample (e.g., fecal sample) obtained from a subject prior to the administration of a bacterial composition disclosed herein. In other embodiments, the reference sample is a biological sample (e.g., fecal sample) obtained from a subject with an active symptom of a disease or disorder, such as those associated with dysbiosis.

### Fatty Acids

Applicant has identified bacteria and combinations of bacteria that are capable of producing certain fatty acids in a subject. In some embodiments, fatty acids comprise short-chain fatty acids. In other embodiments, fatty acids comprise medium-chain fatty acids. As used herein, the term “short-chain fatty acids” refer to fatty acids with less than six carbon atoms. Non-limiting examples of short-chain fatty acids include formate, acetate, propionate, butyrate, isobutryate, valerate, isovalerate, and combinations thereof. In certain embodiments, short-chain fatty acid comprises acetate, propionate, butyrate, or combinations thereof. As used herein, the term “medium-chain fatty acids” refer to fatty acids with aliphatic tails of 5 to 12 carbon atoms, which can form medium-chain triglycerides. Non-limiting examples of medium-chain fatty acids include pentanoate (valerate), hexanoate, oxtanoate, decanoate, dodecanoate, and combinations thereof. In some embodiments, medium-chain fatty acid comprises hexanoate.

In some embodiments, bacteria or combination of bacteria disclosed herein increases the level of a short-chain fatty acid in a subject. In certain embodiments, short-chain fatty acid comprises formate, acetate, propionate, butyrate, isobutryate, valerate, isovalerate, or any combination thereof. In some embodiments, the short-chain fatty acid comprises propionate, butyrate, acetate, or combinations thereof. In some embodiments, the level of a short-chain fatty acid in the subject is increased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, compared to a corresponding level in a reference sample. In some embodiments, the reference sample is a biological sample (e.g., fecal sample) obtained from a subject prior to the administration of a bacterial composition disclosed herein. In other embodiments, the reference sample is a biological sample (e.g., fecal sample) obtained from a subject with an active symptom of a disease or disorder, such as those associated with dysbiosis (e.g., infection or GvHD following HSCT).

In some embodiments, bacteria or combination of bacteria disclosed herein increases the level of a medium-chain fatty acid in a subject. In certain embodiments, the medium-chain fatty acid comprises hexanoate. In some embodiments, the level of a medium-chain fatty acid in the subject is increased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, compared to a corresponding level in a reference sample. In some embodiments, the reference sample is a biological sample (e.g., fecal sample) obtained from a subject prior to the administration of a bacterial composition disclosed herein. In other embodiments, the reference sample is a biological sample (e.g., fecal sample) obtained from a subject with an active symptom of a disease or disorder, such as those associated with dysbiosis (e.g., infection or GvHD following HSCT).

### Inhibition of Histone Deacetylase (HDAC) Activity

Histone deacetylases (HDACs) are a family of enzymes that can remove acetyl residues from specific sites in the N-terminal end of histones, which are part of the DNA chromatin structure in eukaryotic cells. The steady state of histone acetylation is a result of the balance of acetylation by histone acetyltransferase (HAT) enzymes and deacetylation by HDACs. When HDACs are inhibited but HATs activity continues, histones become hyperacetylated, thus disrupting high order chromatin structure and stimulating transcription by RNA polymerase III. The effect of HDAC inhibition in gene expression is not generalized, as only 2% of mammalian genes are affected by HDAC inhibition.

Some short chain fatty acids (SCFAs) produced by the intestinal human microbiome are HDAC inhibitors. Butyrate in particular has been identified as an HDAC inhibitor in vitro and in vivo, leading to the accumulation of hyperacetylated histones H3 and H4 (Candido et al., 1978 Cell 14:105-113; Boffa et al. 1978 J Biol Chem 253:3364-3366; Vidali et al. 1978 Proc Natl Acad Sci USA 75:2239-2243; Davie. 2003 J Nutrition 133:2485S-2493S). Other SCFAs, such as propionate, isobutyrate, isovalerate, valerate, lactate, and acetate, can also inhibit histone deacetylation, although reportedly less effectively than butyrate (Sealy and Chalkley. 1978 Cell 14:115-121; Latham et al. Nucl Acids Res 40:4794-4803, Waldecker et al. 2008 J Nutr Biochem 19:587-593). Certain therapeutic effects of butyrate are reportedly mediated, at least in part, by inhibition of HDAC s.

In some embodiments, bacteria and combinations of bacteria disclosed herein are capable of inhibiting (or reducing) HDAC activity. In some embodiments, bacteria and combinations of bacteria disclosed herein can inhibit (or reduce) HDAC activity in a subject by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, compared to a reference sample. In some embodiments, the reference sample is a biological sample obtained from a subject prior to the administration of a bacterial composition disclosed herein. In other embodiments, the reference sample is a biological sample obtained from a subject with an active symptom of a disease or disorder, such as those associated with dysbiosis (e.g., infection or GvHD following HSCT).

### Protection of Epithelial Barrier

Intestinal epithelial cells are the first line of defense against gut pathogens. They form a physical barrier between luminal contents (microbiome and dietary antigens) and the underlying host immune system. A complex network of intercellular junction proteins and cytoskeletal proteins function together in maintaining a tight impermeable barrier (Buckley & Turner, Cold Spring Harbor perspectives in biology, 10(1), a029314, 2018). Notably, junction proteins like tight junction proteins, claudins and occludins play critical roles in maintaining an intact barrier (Buckley & Turner, Cold Spring Harbor perspectives in biology, 10(1), a029314, 2018).

A variety of microbial byproducts are beneficial for the barrier. For example, short-chain fatty acids (SCFAs) have been shown to improve barrier function by regulating tight junction protein assembly (Peng, et al., The Journal of nutrition, 139(9), 1619-1625, 2008). The SCFA butyrate has been shown to improve barrier function by stabilizing the hypoxia-inducible transcription factor, HIF-1α. Stable HIF-1α, in turn, strengthens the barrier by yet uncharacterized mechanisms (Kelly, at al., Cell host & microbe, 17(5), 662-671, 2005). Butyrate downregulates expression of a pore-forming protein, claudin-2. Claudin-2 is associated with a ‘diarrhetic’ phenotype, where increased expression leads to a leakier barrier. Thus, butyrate-mediated downregulation of claudin-2 strengthens the barrier (Zheng, et al., Journal of immunology, 199(8), 2976-2984, 2017). Furthermore, tryptophan catabolites also have roles in bolstering the barrier. Specifically, indole has been shown both in vivo and in vitro to enhance barrier function (Shimada, et al., PLoS ONE 8(11): e80604, 2013; Bansal, et al., PNAS, 107(1), 228-233, 2010).

In some embodiments, bacteria and combinations of bacteria disclosed herein are capable of reducing damage to an epithelial barrier. In some embodiments, bacteria and combinations of bacteria disclosed herein are capable of reducing damage to an epithelial barrier in a subject in a subject by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, compared to a reference. In some embodiments, bacteria and combinations of bacteria disclosed herein are capable of improving epithelial barrier status in the gastrointestinal tract of the subject. In some embodiments, bacteria and combinations disclosed herein are capable of improving epithelial barrier status by at least 5%, at least 10%, at least 20%, at least 30%, at least 40%, at least 50%, at least 60%, at least 70%, at least 80%, or at least 90% compared to a reference. In some embodiments, the reference is a biological sample obtained from a subject prior to the administration of a bacterial composition disclosed herein. In other embodiments, the reference is a biological sample obtained from a subject with an active symptom of a disease or disorder, such as those associated with dysbiosis (e.g., infection or GvHD following HSCT).

### Other Functional Features

As described supra, in addition to the specific functions detailed above, in some embodiments, bacteria or combinations of bacteria disclosed herein can further comprise one or more of the following functional features: (i) capable of inducing Wnt activation, (ii) capable of producing B vitamins (e.g., thiamin (B1) and pyridoxamine (B6)), (iii) capable of reducing fecal calprotectin level, (iv) capable of restoring colonization resistance, (v) capable of a broad range of carbon source utilization; (vi) capable of reducing VRE pathogen carriage, (vii) capable of reducing CRE pathogen carriage, (viii) capable of reducing colonic inflammation, (ix) susceptible to multiple clinically relevant antibiotics, (x) capable of downmodulating one or more genes induced in IFN-γ treated colonic organoids (e.g., those associated with inflammatory chemokine signaling, NF-κB signaling, TNF family signaling, type I interferon signaling, type II interferon signaling, TLR signaling, lymphocyte trafficking, Th17 cell differentiation, Th2 differentiation, apoptosis, inflammasomes, autophagy, oxidative stress, MHC class I and II antigen presentation, complement, mTor, nod-like receptor signaling, PI3K signaling, or combinations thereof), (xi) capable of reducing the expression of one or more inhibitory receptors (e.g., TIGIT, TIM-3, or LAG-3) on T cells, (xii) capable of increasing expression of one or more genes/proteins associated with T cell activation and/or function (e.g., CD45RO, CD69, IL-24, TNF-α, perforin, or IFN-γ), (xiii) capable of enhancing the ability of CD8+ T cells to kill tumor cells, (xiv) capable of enhancing the efficacy of an immune checkpoint inhibitor therapy, (xv) capable of promoting the recruitment of CD8+ T cells to tumors, (xvi) capable of inhibiting induction of epithelial apoptosis, (xvii) capable of restoring epithelial integrity, as determined by a primary epithelial cell monolayer barrier integrity assay, (xviii) capable of being associated with reduction of risk of infection or GvHD following HSCT, (xix) capable of not being associated with clinical non-remission of infection or GvHD following HSCT, (xx) capable of expressing catalase activity, (xxi) capable of having alpha-fucosidase activity, (xxii) capable of being associated with the healthy human gut microbiota, (xxiii) capable of producing IL-18, (xxiv) capable of inducing an anti-inflammatory IL-10-skewed IL-10/IL-6 cytokine ratio in macrophages; (xxv) capable of producing IL-18, or (xxvii) any combination thereof. In further embodiments, bacteria or combinations of bacteria disclosed herein are not capable of activating a toll-like receptor pathway (e.g., TLR4 or TLR5). In certain embodiments, bacteria or combinations of bacteria disclosed herein are capable of activating a toll-like receptor pathway (e.g., TLR2).

The levels of any of the biological molecules (e.g., those described above) in a subject suffering from a disease or disorder disclosed herein (can be measured as described in the present disclosure (see, e.g., Examples) or by any other methods known in the art.

In some embodiments, a bacterial composition of the present disclosure (e.g., designed compositions) comprises one or more bacteria that are capable of forming spores (i.e., spore-forming bacteria). Accordingly, in some embodiments, a bacterial composition comprises a purified population of bacteria, wherein the bacteria are in the form of spores. In some embodiments, all the bacteria are in the form of spores. In other embodiments, some of the bacteria are in the form of spores, while other bacteria are not in the form of spores (i.e., vegetative-state). In some embodiments, the bacterial composition comprises a purified population of spore-forming bacteria, wherein the bacteria are all in the vegetative-state.

In some embodiments, a bacterial composition comprises a population of bacteria that are sensitive to one or more antibiotics that can be used in a human. In some embodiments, bacteria of the composition are resistant to one or more antibiotics that are used to prophylactically treat patients with a disease or disorder, such as those associated with dysbiosis of the gastrointestinal tract (e.g., infection or GvHD following HSCT). Such antibiotics include, but are not limited to, β-lactams, vancomycin, aminoglycosides, fluoroquinolones, and carbapenems.

In some embodiments, a strain of an OTU useful for the present disclosure (e.g., an OTU disclosed herein) can be obtained from a public biological resource center such as the ATCC (atcc.org), the DSMZ (dsmz.de), or the Riken BioResource Center (en.brc.riken.jp). Methods for determining sequence identity are known in the art.

In some embodiments, the composition is a designed composition. Non-limiting examples of designed compositions are provided in FIGS. 1 and 2A. The exemplary DEs disclosed herein were designed to capture key functional and phylogenetic attributes as described herein.

### II. Formulations

Further provided herein are formulations for administration to humans and other subjects in need thereof (e.g., subject suffering from a disease or disorder disclosed herein). Generally, a bacterial composition as described herein is combined with additional active and/or inactive materials to produce a formulation. In some embodiments, a bacterial composition is formulated in a unit dosage form, each dosage form containing, e.g., from about 10² to about 10⁹ CFUs, for example, about 10⁴ to about 10⁸ CFUs. As described herein, in some aspects, all the bacteria of a composition described herein can be formulated as spores. In some aspects, the spores can be formulated as lyophilized spores. In some aspects, the spores can be suspended in a cryoprotectant, for example glycerol. In some aspects, all the bacteria of a composition described herein can be vegetative cells. In some aspects, a bacterial composition described herein can comprise a mixture of spores and vegetative bacteria. In some aspects, bacterial spores and vegetative bacteria can be formulated at the same dosage. In some aspects, bacterial spores and vegetative bacteria can be formulated at different dosages. For example, when a bacterial composition comprises both bacterial spores and vegetative bacteria, the vegetative bacteria can be formulated at a higher dose compared to the bacterial spores. In some aspects, the bacterial spores are formulated at a higher dose compared to the vegetative bacteria. In other embodiments, a bacterial composition is formulated in a multi-dose format. Formulations and methods of formulating that can be used with the bacterial compositions described herein are described in WO2020118054, which is incorporated by reference herein in its entirety.

The formulations disclosed herein can be effective over a wide dosage range and is generally administered in a pharmaceutically effective amount.

The term “effective dose” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

As used herein, the term “dosage” can refer to the total number of colony forming units (CFUs) of each individual species or strain; or can refer to the total number of microorganisms in the dose. It is understood in the art that determining the number of organisms in a dosage is not exact and can depend on the method used to determine the number of organisms present. If the composition includes spores, for example, the number of spores in a composition can be determined using any suitable methods known in the art, e.g., a dipicolinic acid assay (Fichtel et al., FEMS Microbiol Ecol 61: 522-532 (2007)), or a spore forming colony unit (SCFU) assay. Effective doses can be extrapolated from dose-response curves derived from in vitro or animal model test systems.

As used herein, the term “unit dosage forms” or “dosage unit forms” refers to physically discrete units suitable as unitary dosages for human subjects and other mammals, each unit containing a predetermined quantity of active component calculated to produce the desired therapeutic effect, in association with a suitable pharmaceutical excipient. For instance, in some aspects, a unit dosage form can be in the form of a solid (e.g., capsules, tablets, caplets, pills, troches, lozenges, powders, and granules). In some aspects, a unit dosage form can be in the form of a liquid (e.g., liquid suspension). In some cases, more than one unit dosage form (e.g., two separate capsules or one capsule and a liquid suspension) constitutes a dose. For example, a single dose can be one unit dosage form, two dosage unit forms, three dosage unit forms, four unit dosage forms, five unit dosage forms, or more. In some cases, the number of unit dosage forms constituting a single dose is 1, 2, 3, 4, 5, 6, 7, 8, 9, 10, 11, 12, 13, 14, 15, 20, 25, or 30 unit dosage forms. A single dose can be, e.g., about 10³ to about 10⁹ CFUs, for example, about 10⁴ to about 10⁸ CFUs. In some embodiments, a dose is 1, 2, 3, or 4 capsules containing a total of between about 10² and about 10⁸ CFUs in the dose. In the case of a single dose having multiple dosage forms, the dosage forms are generally delivered within a prescribed period, e.g., within 1 hour, 2 hours, 5 hours, 10 hours, 15 hours, or 24 hours.

In some embodiments, a formulation described herein comprises at least one carbohydrate. A “carbohydrate” refers to a sugar or polymer of sugars. The terms “saccharide,” “polysaccharide,” “carbohydrate,” and “oligosaccharide” can be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate can be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates can contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose (e.g., 2′-fluororibose, deoxyribose, and hexose). Carbohydrates can exist in many different forms, for example, conformers, cyclic forms, acyclic forms, stereoisomers, tautomers, anomers, and isomers.

In some embodiments, a formulation described herein comprises at least one lipid. As used herein a “lipid” includes fats, oils, triglycerides, cholesterol, phospholipids, fatty acids in any form including free fatty acids. Fats, oils and fatty acids can be saturated, unsaturated (cis or trans) or partially unsaturated (cis or trans). In some embodiments the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0). In some embodiments, the formulation comprises at least one modified lipid, for example a lipid that has been modified by cooking.

In some embodiments, a formulation described herein comprises at least one supplemental mineral or mineral source. Examples of minerals include, without limitation: chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In some embodiments, a formulation described herein comprises at least one supplemental vitamin. The at least one vitamin can be fat-soluble or water-soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin.

In some embodiments, a formulation described herein comprises an excipient. Non-limiting examples of suitable excipients include a buffering agent, a diluent, a preservative, a stabilizer, a binder, a compaction agent, a lubricant, a dispersion enhancer, a disintegration agent, a flavoring agent, a sweetener, a coloring agent, a glidant, and an anti-adherent.

In some embodiments, the excipient is a buffering agent. Non-limiting examples of suitable buffering agents include sodium citrate, magnesium carbonate, magnesium bicarbonate, calcium carbonate, and calcium bicarbonate.

In some embodiments, the excipient serves as a diluent. In such embodiments, the excipient can be a solid, semi-solid, or liquid material that acts as a vehicle, carrier, or medium for the active component (e.g., bacteria of the formulation disclosed herein). Thus, a formulation can be in the form of, e.g., a tablet, pill, powder, lozenge, sachet, cachet, elixir, suspension, emulsion, solution, syrup, aerosol (as a solid or in a liquid medium), ointment containing, for example, up to 10% by weight of the active component, soft capsule, hard capsule, gel-cap, tablet, suppository, solution, or packaged powder. In some cases, maximizing delivery of viable bacteria is enhanced by including gastro-resistant polymers, adhesion enhancers, or controlled release enhancers in a formulation.

In some embodiments, the excipient comprises a preservative. Non-limiting examples of suitable preservatives include antioxidants, such as alpha-tocopherol and ascorbate, and antimicrobials, such as parabens, chlorobutanol, and phenol.

In some embodiments, a formulation described herein comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

In some embodiments, a formulation described herein comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments, a formulation described herein comprises a dispersion enhancer as an excipient. Non-limiting examples of suitable dispersants include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments, a formulation described herein comprises a disintegrant as an excipient. In some embodiments, the disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some embodiments, the disintegrant is an effervescent disintegrant. Non-limiting examples of suitable effervescent disintegrants include sodium bicarbonate in combination with citric acid, and sodium bicarbonate in combination with tartaric acid.

In some embodiments, the excipient comprises a flavoring agent. Flavoring agents can be chosen from synthetic flavor oils and flavoring aromatics; natural oils; extracts from plants, leaves, flowers, and fruits; and combinations thereof. In some embodiments, the flavoring agent is selected from cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments, the excipient comprises a sweetener. Non-limiting examples of suitable sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia rebaudiana (Stevioside); chloro derivatives of sucrose such as sucralose; and sugar alcohols such as sorbitol, mannitol, sylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

In some embodiments, a formulation described herein comprises a coloring agent. Non-limiting examples of suitable color agents include food, drug and cosmetic colors (FD&C), drug and cosmetic colors (D&C), and external drug and cosmetic colors (Ext. D&C). The coloring agents can be used as dyes or their corresponding lakes.

Additional suitable excipients include, for example, saline, phosphate buffered saline (PBS), cocoa butter, polyethylene glycol, polyalcohols (e.g., glycerol, sorbitol, or mannitol) and prebiotic oligosaccharides such as inulin, Crystalean® starch, or dextrin. Excipients can also be selected to account, at least in part, for the ability of the OTUs in a particular composition to withstand gastric pH (if being delivered orally or directly to the GI tract) and/or bile acids, or other conditions encountered by the formulation upon delivery to a subject (e.g., an ulcerative colitis patient).

The weight fraction of the excipient or combination of excipients in the formulation is usually about 99% or less, such as about 95% or less, about 90% or less, about 85% or less, about 80% or less, about 75% or less, about 70% or less, about 65% or less, about 60% or less, about 55% or less, 50% or less, about 45% or less, about 40% or less, about 35% or less, about 30% or less, about 25% or less, about 20% or less, about 15% or less, about 10% or less, about 5% or less, about 2% or less, or about 1% or less of the total weight of the formulation.

In preparing a formulation of the present disclosure, the formulation can be milled to provide the appropriate particle size prior to combining with the other ingredients, e.g., those described herein. In some embodiments, a bacterial composition is formulated so as to provide quick, sustained, or delayed release of the active component after administration to a subject, for example, for release in the colon, by employing methods and forms known in the art.

The bacterial compositions disclosed herein can be formulated into a variety of forms and administered by a number of different means. A bacterial composition can be administered orally, rectally, topically (e.g., ear instillation), nasally, intravaginally, or parenterally, in formulations containing conventionally acceptable carriers, adjuvants, and vehicles as desired. The term “parenteral” as used herein includes subcutaneous, intravenous, intramuscular, or intrasternal injection and infusion techniques. In an exemplary embodiment, the bacterial composition (e.g., formulated as described herein) is administered orally.

Solid dosage forms for oral administration include capsules, tablets, caplets, pills, troches, lozenges, powders, and granules. A capsule typically comprises a core material comprising a bacterial composition (e.g., that which has been formulated as described herein) and a shell wall that encapsulates the core material. In some embodiments the core material comprises at least one of a solid, a liquid, and an emulsion. In some embodiments the shell wall material comprises at least one of a soft gelatin, a hard gelatin, and a polymer. Suitable polymers include, but are not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, such as those formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). In some embodiments at least one polymer functions as taste-masking agents.

Tablets, pills, and the like can be compressed, multiply compressed, multiply layered, and/or coated. The coating can be single or multiple. In some embodiments, the coating material comprises at least one of a saccharide, a polysaccharide, and glycoproteins extracted from at least one of a plant, a fungus, and a microbe. Non-limiting examples include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum. In some embodiments the coating material comprises a protein. In some embodiments the coating material comprises at least one of a fat and an oil. In some embodiments the at least one of a fat and an oil is high temperature melting. In some embodiments the at least one of a fat and an oil is hydrogenated or partially hydrogenated. In some embodiments the at least one of a fat and an oil is derived from a plant. In some embodiments the at least one of a fat and an oil comprises at least one of glycerides, free fatty acids, and fatty acid esters. In some embodiments the coating material comprises at least one edible wax. The edible wax can be derived from animals, insects, or plants. Non-limiting examples include beeswax, lanolin, bayberry wax, carnauba wax, and rice bran wax.

In some embodiments, a tablet or pill comprises an inner component surrounding the composition (e.g., which can be formulated as described herein) and an outer component, the latter serving as an envelope over the former. The two components can be separated by an enteric coating layer that can resist disintegration in the stomach and permits the inner component to pass intact into the duodenum or to be delayed in release.

Alternatively, powders or granules embodying a bacterial composition (e.g., which can be formulated as described herein) disclosed herein can be incorporated into a food product. In some embodiments, the food product is a drink for oral administration. Non-limiting examples of a suitable drink include fruit juice, a fruit drink, an artificially flavored drink, an artificially sweetened drink, a carbonated beverage, a sports drink, a liquid diary product, a shake, an alcoholic beverage, a caffeinated beverage, infant formula and so forth. Other suitable means for oral administration include aqueous and nonaqueous solutions, emulsions, suspensions and solutions and/or suspensions reconstituted from non-effervescent granules, containing at least one of suitable solvents, preservatives, emulsifying agents, suspending agents, diluents, sweeteners, coloring agents, and flavoring agents.

In some embodiments, the food product is a solid foodstuff. Suitable examples of a solid foodstuff include without limitation a food bar, a snack bar, a cookie, a brownie, a muffin, a cracker, an ice cream bar, a frozen yogurt bar, and the like.

In some embodiments, a bacterial composition disclosed herein (e.g., which can be formulated as described herein) is incorporated into a therapeutic food. In some embodiments, the therapeutic food is a ready-to-use food that optionally contains some or all essential macronutrients and micronutrients. In some embodiments, a bacterial composition disclosed herein (e.g., which can be formulated as described herein) is incorporated into a supplementary food that is designed to be blended into an existing meal. In some embodiments, the supplemental food contains some or all essential macronutrients and micronutrients. In some embodiments, a bacterial composition disclosed herein (e.g., which can be formulated as described herein) is blended with or added to an existing food to fortify the food's protein nutrition. Examples include food staples (grain, salt, sugar, cooking oil, margarine), beverages (coffee, tea, soda, beer, liquor, sports drinks), snacks, sweets and other foods.

In some embodiments, the formulations are filled into gelatin capsules for oral administration. An example of an appropriate capsule is a 250 mg gelatin capsule containing from 10 (up to 100 mg) of lyophilized powder (10⁸ to 10¹¹ bacteria), 160 mg microcrystalline cellulose, 77.5 mg gelatin, and 2.5 mg magnesium stearate. In other embodiments, from about 10⁵ to about 10¹² bacteria can be used, about 10⁵ to about 10⁷, about 10⁶ to about 10⁷, or about 10⁸ to about 10¹⁰, with attendant adjustments of the excipients if necessary. In further embodiments, an enteric-coated capsule or tablet or with a buffering or protective composition can be used. The use of enteric polymers (such as those used to coat a capsule or tablet described herein) can be useful when formulating a bacterial composition disclosed herein for oral administration. In certain embodiments, the enteric polymers allow for more efficient delivery of the bacterial compositions disclosed herein to a subject's gastrointestinal tract. In some embodiments, the enteric-coated capsule or tablet release their contents (i.e., bacteria or combinations of bacteria disclosed herein) when the pH becomes alkaline after the enteric-coated capsule or tablet passes through the stomach. When a pH sensitive composition (e.g., enteric polymers) is used for formulating the bacterial composition, the pH sensitive composition is a polymer whose pH threshold of the decomposition of the composition is about 6.8 to about 7.5. In some aspects, the pH threshold range can be lower, e.g., about 5.5 or about 6.0, or higher, e.g., about 7.0 or about 8.0. Such a numeric value range is a range where the pH shifts toward the alkaline side at a distal portion of the stomach, and hence is a suitable range for use in the delivery to the colon. Thus, the pH threshold range can be about 5.0 to about 8.0, about 5.5 to about 8.0, about 6.0 to about 8.0, about 6.5 to about 8.0, about 5.0 to about 7.5, about 5.5 to about 7.5, about 6.0 to about 7.5, about 6.5 to about 7.5, about to about 7.0, about 5.5 to about 7.0, about 6.0 to about 7.0, about 6.5 to about 7.0, or a range between any two preceding values.

Moreover, an approach to improving delivery of a bacterial composition disclosed herein (e.g., which can be formulated as described herein) to the colon specifically can include a composition which ensures the delivery to the gastrointestinal tract by delaying the release of the contents by approximately 3 to 5 hours, which corresponds to the small intestinal transit time. In some aspects, the delayed release of the contents of the formulation is about 1 to about 8 hours, about 1 to about 7 hours, about 1 to about 6 hours, about 1 to about 5 hours, about 1 to about 4 hours, about 1 to about 3 hours, about 1 to about 2 hours, about 2 to 8 hours, about 2 to about 7 hours, about 2 to about 6 hours, about 2 to about 5 hours, about 2 to about 4 hours, about 2 to about 3 hours, about 3 to 8 hours, about 3 to about 7 hours, about 3 to 6 hours, about 3 to about 5 hours, about 3 to about 4 hours, about 4 to about 8 hours, about 4 to about 7 hours, about 4 to about 6 hours, about 4 to about 5 hours, about 5 to about 8 hours, about 5 to about 7 hours, about to about 6 hours, about 6 to about 8 hours, about 6 to about 7 hours, about 7 to about 8 hours, or a range between any two preceding values. In an example of formulating a pharmaceutical preparation comprising the composition for delaying the release, a hydrogel is used as a shell. The hydrogel is hydrated and swells upon contact with gastrointestinal fluid, so that the contents are effectively released. Furthermore the delayed release dosage units include drug-containing compositions having a material which coats or selectively coats a drug. Examples of such a selective coating material include in vivo degradable polymers, gradually hydrolyzable polymers, gradually water-soluble polymers, and/or enzyme degradable polymers. A preferred coating material for efficiently delaying the release is not particularly limited, and examples thereof include cellulose-based polymers such as hydroxypropyl cellulose, acrylic acid polymers and copolymers such as methacrylic acid polymers and copolymers, and vinyl polymers and copolymers such as polyvinylpyrrolidone.

Additional compositions that target delivery to the colon include bioadhesive compositions which specifically adhere to the colonic mucosal membrane (for example, a polymer described in the specification of U.S. Pat. No. 6,368,586), and compositions into which a protease inhibitor is incorporated for protecting particularly a bacterial composition disclosed herein (e.g., which can be formulated as described herein) in the gastrointestinal tracts from decomposition due to an activity of a protease.

An additional colon-delivery mechanism is via pressure change, such that the contents are released from the colon by generation of gas in bacterial fermentation at a distal portion of the stomach. Such pressure-change is not particularly limited, and a more specific example thereof is a capsule which has contents dispersed in a suppository base and which is coated with a hydrophobic polymer (for example, ethyl cellulose).

A further composition for delivery to the colon includes, for example, a bacterial composition disclosed herein (e.g., which can be formulated as described herein) comprising a component that is sensitive to an enzyme (for example, a carbohydrate hydrolase or a carbohydrate reductase) present in the colon. Such a composition is not particularly limited, and more specific examples thereof include compositions that use food components such as non-starch polysaccharides, amylose, xanthan gum, and azopolymers.

In some embodiments, a bacterial composition disclosed herein is formulated with a germinant to enhance engraftment or efficacy. In some embodiments, a bacterial composition is formulated or administered with a prebiotic substance to enhance engraftment or efficacy.

In some embodiments, the number of bacteria of each type can be present in the same level or amount or in different levels or amounts. For example, in a bacterial composition (e.g., which can be formulated as described herein) with two types of bacteria, the bacteria can be present in from about a 1:10,000 ratio to about a 1:1 ratio, from about a 1:10,000 ratio to about a 1:1,000 ratio, from about a 1:1,000 ratio to about a 1:100 ratio, from about a 1:100 ratio to about a 1:50 ratio, from about a 1:50 ratio to about a 1:20 ratio, from about a 1:20 ratio to about a 1:10 ratio, from about a 1:10 ratio to about a 1:1 ratio, or a range between any two preceding values. For bacterial compositions (e.g., which can be formulated as described herein) comprising at least three types of bacteria, the ratio of type of bacteria can be chosen pairwise from ratios for bacterial compositions with two types of bacteria. For example, in a bacterial composition (e.g., which can be formulated as described herein) comprising bacteria A, B, and C, at least one of the ratio between bacteria A and B, the ratio between bacteria B and C, and the ratio between bacteria A and C can be chosen, independently, from the pairwise combinations above.

### III. Methods of Treating a Subject

The compositions and formulations disclosed herein can be used for the treatment and/or prevention of a disease or disorder, such as those associated with dysbiosis of a gastrointestinal tract (e.g., infection or GvHD following HSCT), e.g., by ameliorating one or more signs or symptoms of the disease. The bacterial compositions disclosed herein (e.g., which can be formulated as described herein) can also be useful for the treatment of diseases or disorders, including but not limited to acute leukemia (ALL), acute myelogenous leukemia (AML), multiple myeloma and lymphomas (NHL and HD), MDS (combined with MPNs) and related cancers that require HSCT, mucositis, infections including but not limited to blood and tissue infection with ESKAPE pathogens (including: Enterococcus faecium, Staphylococcus aureus, Klebsiella pneumoniae, Acinetobacter baumannii, Pseudomonas aeruginosa, and Enterobacter spp.), with Enterococcus species including but not limited to Enterococcus faecalis and Enterococcus faecium, Enterobacteriaceae species including but not limited to Klebsiella pneumonia, or such species that are resistant to vancomycin or carbapenems, with drug resistant or multi-drug resistant organisms (MDROs) including VRE, CRE (including Klebsiella pneumoniae, Klebsiella oxytoca, Klebsiella aerogenes, Enterococcus species), with drug resistant or multi-drug resistant Enterobacteriaceae, with extended spectrum beta-lactamase (ESBLs) producing bacteria (including E. coli, Klebsiella species), or with methicillin-resistant Staphylococcus aureus (MRSA), patients at risk for infectious disease, including for example, patients in intensive care units and long-term care, treatment and prevention of C. difficile infection (CDI), as well as recurrent CDI, immune check point inhibitor-induced (ICI) colitis, viral infection and reactivation, and fungal infection, including gastrointestinal infection and systemic, e.g., candidemia, etc.). The bacterial compositions (e.g., which can be formulated as described herein) disclosed herein can further be useful for treatment of diseases or disorders, including those described in International Publication No. WO 2019/227085, which is incorporated by reference herein.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease or enhancing overall survival. Treating can include reducing at least one sign or symptom associated with a disease or disorder disclosed herein, e.g., infection or GvHD following HSCT. Treatment can be of a subject having a disease or a subject who does not have a disease (e.g., for prophylaxis). It is understood that “preventing” can mean reducing the risk of disease or reducing the rate of relapse.

In some embodiments, treatment with a bacterial composition described herein is associated with at least one of the following: (i) an increase in the diversity of the gastrointestinal (GI) microbiome in a subject, (ii) a reduction in GI inflammation in a subject, (iii) improvement in mucosal and/or epithelial barrier integrity in a subject compared to a reference control (e.g., untreated patients or the subject prior to treatment), (iv) promotion of mucosal healing, (v) reduction in incidence of infection, (vi) reduction in the use of antibiotics, (vii) increase in the probability of survival, (viii) reduction in relapse of primary cancer, and (ix) other improvements of at least one sign or symptom of a disease or disorder disclosed herein. In some embodiments, improvement associated with an increase in the diversity of the gastrointestinal (GI) microbiome in a subject includes improvement measured by species domination, including, for example, by pathobionts, drug resistant organisms, or MDROs, or as measured by an increase in diversity of species such as in the number of species (e.g., species richness) and/or species distribution (e.g., even skew of distribution). Such improvements can also include, for example, improvements detected via biomarkers, such as a decrease or increase in the level of certain biological molecules (e.g., fecal calprotectin, secondary bile acids, tryptophan metabolites, or short-chain and medium-chain fatty acids) following treatment. Formulations disclosed herein (e.g., comprising a designed bacterial composition) can be used to treat any disease or disorder associated with a dysbiosis of the gastrointestinal tract. Non-limiting examples of such diseases or disorders are provided throughout the present disclosure.

Formulations as described herein are useful for administration to a subject, e.g., a mammal, such as a human in need of treatment, e.g., to prevent or treat a disease or disorder disclosed herein or a sign or symptom of a disease or disorder disclosed herein or to prevent recurrence of a disease or disorder disclosed herein. In some embodiments, the mammalian subject is a human subject. In some embodiments, the human subject (e.g., patient) has one or more signs or symptoms of a disease or disorder, such as those associated with infection (including, but not limited to, blood stream infection, sepsis, tissue infection, invasive infection, viral infection or reactivation, and gastrointestinal infection including but not limited to C. difficile, graft-versus-host-disease (GvHD) including acute or chronic GvHD, relapse of cancer, or mucositis). A therapeutically effective treatment using a formulation provided herein can ameliorate one or more of such signs and symptoms of a disease or disorder disclosed herein. In some embodiments, the signs and symptoms of a disease or disorder disclosed can be febrile neutropenia, defined as a temperature ≥38.0° C. (100.4° F.) concurrent with absolute neutrophil count (ANC)<500 cells/mm³ in the absence of an identified infectious agent.

Efficacy of a treatment can be determined by evaluating signs and/or symptoms and according to whether induction of improvement and/or maintenance of an improved condition is achieved, e.g., for at least about 1 week, at least about two weeks, at least about three weeks, at least about four weeks, at least about 8 weeks, or at least about 12 weeks.

Other indicators of efficacy of a therapeutic composition and/or method for treating a disease or disorder, such as those associated with dysbiosis include engraftment of at least one bacterial species or OTU identified in a microbiome composition, for example, at about 2 weeks, about 3 weeks, about 4 weeks, about 5 weeks, about 6 weeks, about 7 weeks, about 8 weeks, about 9 weeks, about 10 weeks, about 11 weeks, about 12 weeks, or longer after initial dosing with the microbiome composition. In some embodiments, the indicators of efficacy of a therapeutic composition and/or method for treating a disease or disorder, such as those associated with dysbiosis include the reduced abundance of at least one species or OTU identified herein (e.g., ESKAPE pathogens including: Enterococcus faecium, Staphylococcus aureus, Klebsiella pneumoniae, Acinetobacter baumannii, Pseudomonas aeruginosa, and Enterobacter spp. or pathogens related to gastrointestinal infections, including Clostridioides difficile, viral, and parasitic infections), for example, at about 2 weeks, about 3 weeks, about 4 weeks, about 5 weeks, about 6 weeks, about 7 weeks, about 8 weeks, about 9 weeks, about 10 weeks, about 11 weeks, about 12 weeks, or longer after initial dosing with the microbiome composition. In some embodiments, the indicators of efficacy of a therapeutic composition and/or method for treating a disease or disorder, such as those associated with dysbiosis include the reduced abundance of at least one species or OTU identified herein (e.g., Enterococcus spp. and Enterobacteriaceae spp. in stool, drug resistant or multi-drug resistant organisms (MDROs) including vancomycin-resistant enterococci (VRE), carbapenem-resistant Enterobacteriaceae (CRE), drug resistant or multi-drug resistant Enterobacteriaceae, methicillin resistant Staphylococcus aureus (MRSA), and organisms that produce extended-spectrum beta-lactamase (ESBL)), for example, at about 2 weeks, about 3 weeks, about 4 weeks, about 5 weeks, about 6 weeks, about 7 weeks, about 8 weeks, about 9 weeks, about 10 weeks, about 11 weeks, about 12 weeks, or longer after initial dosing with the microbiome composition.

In some embodiments, treatment with a formulation disclosed herein can improve a dysbiosis, including, but not limited to, an improvement in the representation of one or more OTUs identified as reduced in a population of subjects suffering from a disease or disorder associated with dysbiosis (e.g., patients following HSCT with active disease). In some embodiments, treatment with a formulation of the present disclosure can reduce the representation of one or more microbial species that are associated with a disease or disorder disclosed herein.

In some embodiments, treatment with a formulation disclosed herein can increase the representation of microbial species that are associated with an improvement (e.g., reducing the risk of infection or GvHD) of a disease or disorder disclosed herein. In some embodiments, the improvement of a disease or disorder can be improvement on overall survival (OS), the incidence and duration of survival endpoints including transplant-related mortality (TRM), relapse free survival (RFS), GvHD-free survival (GFS), and GvHD- and relapse-Free survival (GRFS), the frequency and length of hospitalizations and Intensive Care Unit (ICU) stays, or incidence and severity of chronic Graft-versus-Host Disease (cGvHD). In some embodiments the improvement of a disease or disorder can be assessed through biomarkers comprising the urinary concentration of products of amino acid metabolism such as 3-indoxyl sulfate (3-IS), stool biomarkers, including but not limited to calprotectin or lipocalin, and plasma immunologic mediators during the treatment period including circulating markers of aGvHD, suppression of tumorigenicity 2 (ST2), regenerating islet-derived 3α (REG3α), cytokines, or T cell subsets in plasma at various timepoints through Week 14 (End of Treatment).

In some embodiments, subjects that have undergone or are undergoing transplantation and administered the bacterial compositions or pharmaceutical formulations thereof have i) an increased prevalence in their stool of one or more strains in the bacterial composition, ii) a decreased abundance in their stool of Enterococcus spp., Enterobacteriaceae spp., or both, iii) a decreased incidence of bloodstream infections including but not limited to bacterial infections (VRE, CRE, or ESBL), fungal infections, or combinations thereof, iv) a decreased incidence of gastrointestinal infections including but not limited to Clostridiodes difficile, viral infections or reactivations (including but not limited to norovirus, adenovirus, or rotavirus), parasitic infections (including but not limited to Cryptosporidia), or combinations thereof, v) a decreased incidence of acute GvHD including but not limited to acute GvHD Grades II, III, and IV, vi) a decreased incidence of febrile neutropenia, vii) reduced frequency, length, or both frequency and length of hospitalization stay, or vii) any combination thereof, relative to a reference (e.g., a corresponding reference in a subject that did not receive the composition disclosed herein or corresponding reference in the subject prior to the administration of the composition).

In some embodiments, a subject receives a pretreatment protocol prior to administration of the formulation, wherein the pretreatment protocol prepares the gastrointestinal tract to receive the bacterial composition. In certain embodiments, the pretreatment protocol comprises an oral antibiotic treatment, wherein the antibiotic treatment alters the bacteria in the patient. In specific embodiments, the antibiotic is not absorbed through the gut or minimally bioavailable for systemic distribution. In other embodiments, the pretreatment protocol comprises a colonic cleansing (e.g., enema), wherein the colonic cleansing substantially empties the contents of the patient's colon. As used herein, “substantially emptying the contents of the colon” refers to removal of at least about 75%, at least about 80%, at least about 90%, at least about 95%, or about 100% of the contents of the ordinary volume of colon contents. In certain embodiments, the subject receives more than one pretreatment protocol, e.g., an antibiotic treatment preceding a colon-cleansing protocol.

In some embodiments, a subject does not receive a pretreatment protocol prior to administration of the formulation. Instead, the formulations as described herein are administered to reduce, alleviate, or prevent intestinal dysbiosis associated with subsequent treatment of the subject for a disease or disorder requiring treatment (e.g., allogeneic or autologous HSCT). In some embodiments, a subject receives a pretreatment protocol prior to administration of the formulations as described herein and the subject receives subsequent treatment for a disease or disorder requiring treatment (e.g., allogeneic or autologous HSCT) following administration of the formulations as described herein.

In some embodiments, a pretreatment protocol is administered to a subject at least 1 day, 2 days, 3 days, 5 days, 6 days, 7 days, 10 days, or 15 days prior to administration of a formulation described herein. In some embodiments, subsequent treatment of the subject for a disease or disorder requiring treatment (e.g., allogeneic or autologous HSCT) occurs at least 1 day, 2 days, 3 days, 5 days, 6 days, 7 days, 10 days, or 15 days after administration of a formulation described herein. In some embodiments, the subject receives multiple doses of a formulation. In some embodiments, the subject receives multiple doses of a formulation through a course of 1, 2, 3, 4, 5, 6, 7, 8, 9, 10, 11, 12, 13, 14, 15, 16, 17, 18, 19 or 20 days. In some embodiments, the subject receives at least 1, 2, 3, 4, 5, 6, 7, 8, 9 or 10 courses. In some embodiments, the subject has at least one sign or symptom of a disease or disorder, such as those disclosed herein prior to administration of the formulation. In other embodiments, the subject does not exhibit a sign or symptom of a disease or disorder, such as those disclosed herein prior to administration of the formulation, e.g., formulation is administered prophylactically to reduce the risk of a sign or symptom of a disease or disorder, such as those disclosed herein.

In some embodiments, a formulation described herein is administered enterically, in other words, by a route of access to the gastrointestinal tract. This includes oral administration, rectal administration (including enema, suppository, or colonoscopy), by an oral or nasal tube (nasogastric, nasojejunal, oral gastric, or oral jejunal), or any other method known in the art.

In some embodiments, a formulation is administered to at least one region of the gastrointestinal tract, including the mouth, esophagus, stomach, small intestine, large intestine, and rectum. In other embodiments, a formulation is administered to all regions of the gastrointestinal tract. In certain embodiments, a formulation is administered orally in the form of medicaments such as powders, capsules, tablets, gels or liquids. The formulation can also be administered in gel or liquid form by the oral route or through a nasogastric tube, or by the rectal route in a gel or liquid form, by enema or instillation through a colonoscope or by a suppository.

In some embodiments, the bacteria and bacterial compositions are provided in a dosage form. In some embodiments, the dosage form is designed for administration of at least one OTU or combination thereof disclosed herein, wherein the total amount of bacterial composition administered is selected from about 0.1 ng to about 10 g, about 10 ng to about 1 g, about 100 ng to about 0.1 g, about 0.1 mg to about 500 mg, about 1 mg to about 1000 mg, from about 1000 to about 5000 mg, or more. In some embodiments, the bacteria and bacterial compositions are provided in a single dosage form comprising a capsule. In some embodiments, the bacteria and bacterial compositions are provided in a single dosage form comprising at least one capsule, at least two capsules, at least three capsules, at least three capsules, at least four capsules, at least five capsules, at least six capsules, at least seven capsules, at least eight capsules, at least nine capsules, or at least ten capsules. In some embodiments, the capsule comprise a liquid formulation of bacterial spores from a strain delivered at a target dose strength of about 1×10⁶ to about 5×10⁷ colony-forming units (CFUs) per dose. In some embodiments, the capsule comprise a liquid formulation of bacterial spores from 1, 2, 3, 4, 5, 6, 7, 8, 9, 10, 11, 12, 13, 14, 15, 16, 17, 18, 19 or 20 strains delivered at a target dose strength of about 1×10⁶ to about 5×10⁷ colony-forming units (CFUs) per dose. In some embodiments, the capsule comprise a dry powder formulation of bacterial spores from a strain delivered at a target dose strength of about 1×10⁶ to about 5×10⁷ colony-forming units (CFUs) per dose. In some embodiments, the capsule comprise a dry powder formulation of bacterial spores from 1, 2, 3, 4, 5, 6, 7, 8, 9, 10, 11, 12, 13, 14, 15, 16, 17, 18, 19 or 20 strains delivered at a target dose strength of about 1×10⁶ to about 5×10⁷ colony-forming units (CFUs) per dose.

In some embodiments, the treatment period is at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 1 month, at least about 2 months, at least about 3 months, at least about 4 months, at least about 5 months, at least about 6 months, or at least about 1 year. In some embodiments, the treatment period is from about 1 day to 1 week, from about 1 week to 4 weeks, from about 1 month, to 3 months, from about 3 months to 6 months, from about 6 months to 1 year, or for over a year.

In some embodiments, from about 10⁵ and about 10¹² microorganisms total is administered to the patient in a given dosage form. In certain embodiments, an effective amount can be provided in from about 1 to about 500 ml or from about 1 to about 500 grams of the bacterial composition having from about 10⁵ to about 10¹¹ bacteria per ml or per gram, or a capsule, tablet, powder, or suppository having from about 1 mg to about 1000 mg lyophilized powder having from about 10⁵ to about 10¹¹ bacteria. In some embodiments, those receiving acute treatment receive higher doses than those who are receiving chronic administration (such as hospital workers or those admitted into long-term care facilities).

In some embodiments, a formulation described herein is administered once, on a single occasion or on multiple occasions, such as once a day for several days or more than once a day on the day of administration (including twice daily, three times daily, or up to five times daily). In some embodiments, a formulation is administered intermittently according to a set schedule, e.g., once a day, once weekly, or once monthly, or when the patient relapses from clinical improvement of a disease or disorder, such as those disclosed herein, or exhibits a sign or symptoms of a disease or disorder, such as those disclosed herein. In other embodiments, a formulation is administered on a long-term basis to individuals who are at risk for active disease or disorder, such as those disclosed herein or are diagnosed as being at risk for developing a disease or disorder (e.g., have a family history of the diseases or a history of isotretinoin use by the individual).

In some embodiments, a bacterial composition of the present disclosure (e.g., which can be formulated as described herein) is administered with other agents (e.g., anti-microbial agents or prebiotics) as a combination therapy mode. In some embodiments, a bacterial composition of the present disclosure (e.g., which can be formulated as described herein) is administered without other agents (e.g., anti-microbial agents or prebiotics).

In some embodiments, a bacterial composition as described herein (e.g., which can be formulated as described herein) is administered with other agents where the other agent (e.g., anti-microbial agents or prebiotics) is administered before, for example, one day, two days, three days, four days, five days, six days, seven days, or more than seven days, the bacterial composition. In some embodiments, the bacterial composition (e.g., which can be formulated as described herein) is then administered over several days, for example, one day, two days, three days, four days, five days, six days, seven days, eight days, nine days, ten days, or more. In some embodiments, the other agent (e.g., anti-microbial agents or prebiotics) is administered, for example, for one day, two days, three days, four days, five days, six days, seven days, or more than seven days, followed by administration of a bacterial composition as described herein over several days, for example, one day, two days, three days, four days, five days, six days, seven days, eight days, nine days, ten days, or more. In some embodiments, the other agent (e.g., anti-microbial agents or prebiotics) is administered over a period of four days followed by administration of a bacterial composition as described herein (e.g., which can be formulated as described herein) over ten days.

In some embodiments, a bacterial composition as described herein (e.g., which can be formulated as described herein) is administered before HSCT, after HSCT, or after administration of, e.g., anti-bacterial agents, following HSCT, and combinations thereof. In some embodiments, a bacterial composition as described herein is administered before HSCT, after HSCT, and after administration of, e.g., anti-bacterial agents, following HSCT

In some embodiments, a bacterial composition (e.g., which can be formulated as described herein) is included in combination therapy with one or more anti-microbial agents, which include anti-bacterial agents, anti-fungal agents, anti-viral agents and anti-parasitic agents.

Anti-bacterial agents include cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); and carbapenem antibiotics (ertapenem, doripenem, imipenem/cilastatin, and meropenem). In certain aspects, the anti-bacterial agent is vancomycin.

Anti-viral agents include Abacavir, Acyclovir, Adefovir, Amprenavir, Atazanavir, Cidofovir, Darunavir, Delavirdine, Didanosine, Docosanol, Efavirenz, Elvitegravir, Emtricitabine, Enfuvirtide, Etravirine, Famciclovir, Foscarnet, Fomivirsen, Ganciclovir, Indinavir, Idoxuridine, Lamivudine, Lopinavir Maraviroc, MK-2048, Nelfinavir, Nevirapine, Penciclovir, Raltegravir, Rilpivirine, Ritonavir, Saquinavir, Stavudine, Tenofovir Trifluridine, Valaciclovir, Valganciclovir, Vidarabine, Ibacitabine, Amantadine, Oseltamivir, Rimantidine, Tipranavir, Zalcitabine, Zanamivir and Zidovudine.

Examples of antifungal compounds include, but are not limited to polyene antifungals such as natamycin, rimocidin, filipin, nystatin, amphotericin B, candicin, and hamycin; imidazole antifungals such as miconazole, ketoconazole, clotrimazole, econazole, omoconazole, bifonazole, butoconazole, fenticonazole, isoconazole, oxiconazole, sertaconazole, sulconazole, and tioconazole; triazole antifungals such as fluconazole, itraconazole, isavuconazole, ravuconazole, posaconazole, voriconazole, terconazole, and albaconazole; thiazole antifungals such as abafungin; allylamine antifungals such as terbinafine, naftifine, and butenafine; and echinocandin antifungals such as anidulafungin, caspofungin, and micafungin. Other compounds that have antifungal properties include, but are not limited to polygodial, benzoic acid, ciclopirox, tolnaftate, undecylenic acid, flucytosine or 5-fluorocytosine, griseofulvin, and haloprogin.

In some embodiments, a bacterial composition (e.g., which can be formulated as described herein) is included in combination therapy with one or more corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, anti-cholinergic decongestants, mast-cell stabilizers, monoclonal anti-IgE antibodies, vaccines, and combinations thereof.

A prebiotic is a selectively fermented ingredient that allows specific changes, both in the composition and/or activity in the gastrointestinal microbiota that confers benefits upon a treated subject's well-being and health. Prebiotics can include complex carbohydrates, amino acids, peptides, or other nutritional components that allow bacterial composition benefits such as better survival, fitness, enhanced engraftment, enhanced competition with resident bacteria or pathobionts or pathogens. Prebiotics include, but are not limited to, amino acids, biotin, fructooligosaccharide, galactooligosaccharides, inulin, lactulose, mannan oligosaccharides, oligofructose-enriched inulin, oligofructose, oligodextrose, tagatose, trans-galactooligosaccharide, and xylooligosaccharides.

To evaluate a subject, signs or symptoms of an adverse event or disease recurrence are evaluated post-treatment ranging from, e.g., about 1 day to about 6 months after administration of a formulation. One method of evaluation involves obtaining fecal material from the subject and assessment of microbes present in the gastrointestinal tract, e.g., using 16S rDNA or metagenomic shotgun sequencing analysis or other analyses known in the art. Population of the gastrointestinal tract by bacterial species present in the formulation as well as augmentation by commensal microbes not present in the formulation can be used to indicate an improvement in the GI dysbiosis associated with e.g., an infection or GvHD following HSCT, and therefore a decreased risk of an adverse event or a decrease in the severity of an adverse event.

In addition to treating the different inflammatory diseases disclosed herein (e.g., an infection or GvHD following HSCT), applicant has surprisingly discovered that the designed compositions disclosed herein can be also used to treat diseases or disorders that are generally not associated with pro-inflammatory responses. A non-limiting example of such a disease or disorder is cancer. In some embodiments, the bacterial compositions disclosed herein (e.g., designed compositions) can be used to treat certain cancers, e.g., when administered in combination with other anti-cancer agents. Without being limited to any one particular theory, the compositions disclosed herein are designed to have functional features that target multiple biological pathways. In some embodiments, the functional features are important for the treatment of inflammatory diseases. In other embodiments, the functional features are important for the treatment of cancers. In certain embodiments, the functional features are important for the treatment of both inflammatory diseases and cancers. Non-limiting examples of functional features that can be important for the treatment of both inflammatory diseases and cancers include, but are not limited to, inhibition of HDAC activity, production of short-chain fatty acids, production of medium chain fatty acids, production of tryptophan metabolites, protection of epithelial barrier integrity, inhibition of apoptosis (e.g., which is capable of restoring epithelial barrier integrity after transplant preconditioning), downmodulating one or more genes induced in IFN-γ treated colonic organoids (e.g., those associated with inflammatory chemokine signaling, NF-κB signaling, TNF family signaling, type I interferon signaling, type II interferon signaling, TLR signaling, lymphocyte trafficking, Th17 cell differentiation, Th2 differentiation, apoptosis, inflammasomes, autophagy, oxidative stress, MEW class I and II antigen presentation, complement, mTor, nod-like receptor signaling, PI3K signaling, or combinations thereof), production of IL-18, activation of human CD8 T cells by metabolites (e.g., short-chain fatty acids) or macromolecules, activation of antigen presenting cells such as dendritic cells by bacterial antigens, macromolecules and metabolites, or reduced colonic inflammation (e.g., through promoting immune homeostasis in the colon and via upregulation of Tregs and counterbalancing inflammatory cell populations such as Th1, Th17, and activated human CD8 T cells), reducing expression of one or more inhibitory receptors (e.g., TIGIT, TIM-3, or LAG-3) on human CD8+ T cells, increasing expression of one or more genes/proteins associated with T cell activation and/or function (e.g., CD45RO, CD69, IL-24, TNF-α, perforin, or IFN-γ), enhancing the ability of human CD8+ T cells to kill tumor cells, enhancing the efficacy of an immune checkpoint inhibitor, or enabling recruitment of human CD8 T cells to tumors located distally.

In some embodiments, a designed composition disclosed herein is administered in combination with an additional therapeutic agent used for the treatment of cancers. Such additional therapeutic agents can include, for example, chemotherapy drugs, small molecule drugs or antibodies that stimulate the immune response to a given cancer. In some instances, therapeutic compositions can include an immune checkpoint inhibitor, e.g., an anti-PD-1 antibody, an anti-PD-L1 antibody, or an anti-CTLA-4 antibody. Non-limiting examples of other antibodies that can be used in combination with the designed compositions of the present disclosure include an anti-OX40 (also known as CD134, TNFRSF4, ACT35 and/or TXGP1L) antibody, an anti-CD137 antibody, an anti-LAG-3 antibody, or an anti-GITR antibody.

In some embodiments, a designed composition disclosed herein, when administered in combination with an anti-cancer agent (e.g., immune checkpoint inhibitor, e.g., anti-PD-1 antibody or an anti-PD-L1 antibody), can reduce tumor volume in a subject. In certain embodiments, tumor volume is decreased by at least about 10%, at least about 20%, at least about 30%, at least about 40%, at least about 50%, at least about 60%, at least about 70%, at least about 80%, or at least about 90% in the subject, compared to a reference (e.g., tumor volume in the subject prior to the administration or a corresponding subject that did not receive the compositions disclosed herein).

In some embodiments, a designed composition disclosed herein, when administered in combination with an anti-cancer agent (e.g., immune checkpoint inhibitor, e.g., anti-PD-1 antibody or an anti-PD-L1 antibody), can increase the percentage of CD8 T cells and/or CD4 T cells (tumor infiltrating lymphocytes) in the tumor of a subject. In some embodiments, the percentage of CD8 T cells and/or CD4 T cells in the tumor is increased by at least about 5%, at least about 10%, at least about 20%, at least about 30%, at least about 40%, at least about 50%, at least about 60%, at least about 70%, at least about 80%, or at least about 90% in the subject, compared to a reference (e.g., percentage of CD8 T cells and/or CD4 T cells in the tumor in the subject prior to the administration or a corresponding subject that did not receive the compositions disclosed herein). As a result of the increase in the percentage of CD8 T cells, in some embodiments, the ratio of CD8 T cells to regulatory T cells in the tumor is increased, e.g., by at least about 5%, at least about 10%, at least about 20%, at least about 30%, at least about 40%, at least about 50%, at least about 60%, at least about 70%, at least about 80%, or at least about 90% in the subject, compared to a reference (e.g., the ratio of CD8 T cells to regulatory T cells in the tumor in the subject prior to the administration or a corresponding subject that did not receive the compositions disclosed herein).

In some embodiments, a designed composition disclosed herein, when administered in combination with an anti-cancer agent (e.g., immune checkpoint inhibitor, e.g., anti-PD-1 antibody or an anti-PD-L1 antibody), can enhance the ability of CD8+ T cells to kill tumor cells. In certain embodiments, the ability of CD8+ T cells to kill tumor cells is increased by at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, or at least about 500% or more, compared to a reference (e.g., the ability of CD8+ T cells to kill tumor cells in the subject prior to the administration or a corresponding subject that did not receive the compositions disclosed herein).

In some embodiments, a designed composition disclosed herein, when administered in combination with an anti-cancer agent (e.g., immune checkpoint inhibitor, e.g., anti-PD-1 antibody or an anti-PD-L1 antibody), can increase the activation and/or function of T cells (e.g., tumor-specific CD8+ or CD4+ T cells) in the subject. In certain embodiments, the activation and/or function of T cells (e.g., tumor-specific CD8+ or CD4+ T cells) is increased by at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, or at least about 500% or more, compared to a reference (e.g., the activation and/or function of the T cells in the subject prior to the administration or a corresponding subject that did not receive the compositions disclosed herein). As described herein, methods of determining the activation and/or function of T cells (e.g., tumor-specific CD8+ or CD4+ T cells) are known in the art, e.g., by measuring the expression of one or more of the following: CD45RO, CD69, IL-24, TNF-α, perforin, or IFN-γ.

In some embodiments, a designed composition disclosed herein, when administered in combination with an anti-cancer agent (e.g., immune checkpoint inhibitor, e.g., anti-PD-1 antibody or an anti-PD-L1 antibody), can reduce the expression of one or more inhibitory receptors (e.g., TIGIT, TIM-3, or LAG-3) on T cells (e.g., tumor-specific CD8+ or CD4+ T cells). In certain embodiments, the expression of one or more inhibitory receptors is decreased by at least about 10%, at least about 20%, at least about 30%, at least about 40%, at least about 50%, at least about 60%, at least about 70%, at least about 80%, or at least about 90%, compared to a reference (e.g., the expression of the corresponding inhibitory receptors on the T cells in the subject prior to the administration or a corresponding subject that did not receive the compositions disclosed herein).

Non-limiting examples of cancers that can be treated with the present disclosure include squamous cell carcinoma, small-cell lung cancer, non-small cell lung cancer, squamous non-small cell lung cancer (NSCLC), nonsquamous NSCLC, glioma, gastrointestinal cancer, renal cancer (e.g., clear cell carcinoma), ovarian cancer, liver cancer, colorectal cancer, endometrial cancer, kidney cancer (e.g., renal cell carcinoma (RCC)), prostate cancer (e.g., hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, T-cell lymphoma, environmentally-induced cancers including those induced by asbestos, virus-related cancers or cancers of viral origin (e.g., human papilloma virus (HPV-related or -originating tumors)), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CIVIL), undifferentiated AML (MO), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NEIL), B cell hematologic malignancy, e.g., B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukaemia (T-Lbly/T-ALL), peripheral T-cell lymphoma, lymphoblastic lymphoma, post-transplantation lymphoproliferative disorder, true histiocytic lymphoma, primary central nervous system lymphoma, primary effusion lymphoma, B cell lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous, including astrocytoma, schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, as well as any combinations of said cancers. The methods described herein can also be used for treatment of metastatic cancers, unresectable, refractory cancers (e.g., cancers refractory to previous immunotherapy, e.g., with a blocking CTLA-4 or PD-1 antibody), and/or recurrent cancers.

### Additional Information

Certain terms used in the present application are defined as follows. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

As used herein, the term “approximately” or “about,” as applied to one or more values of interest, refers to a value that is similar to a stated reference value and within a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). When the term “approximately” or “about” is applied herein to a particular value, the value without the term “approximately” or “about” is also disclosed herein.

As described herein, any concentration range, percentage range, ratio range, or integer range is to be understood to include the value of any integer within the recited range and, when appropriate, fractions thereof (such as one tenth and one hundredth of an integer), unless otherwise indicated.

As used herein, the term “graft-versus-host-disease” (GvHD) refers to an inflammatory condition that can often occur in a subject after transplantation (e.g., HSCT), particularly where the transplanted tissue and/or cells (also referred to herein as “graft”) are from an allogenic donor. More specifically, GvHD occurs when the donor's immune cells present in the graft attack the recipient's tissues/organs, resulting in the eventual destruction of the tissues/organs if left untreated. Symptoms can vary depending on the type of GvHD. There are two main types of GvHD: (i) acute GvHD and (ii) chronic GvHD.

Acute GvHD typically develops within the first 100 days after transplantation. In contrast, chronic GvHD usually occurs more slowly (e.g., at least 100 days post-transplantation) and can last a lifetime. Acute GvHD (aGvHD) is an inflammatory process affecting different organs, clinically presenting after transplant as maculopapular rash (skin), hyperbilirubinemia and jaundice (liver), anorexia, nausea, and vomiting (upper GI), and watery or bloody diarrhea and crampy abdominal pain (lower GI) (Nassereddine 2017). The diagnosis of aGvHD is based upon clinical features and tissue biopsies, typically within the first several weeks after transplant. Acute GvHD is clinically staged and graded in severity from Grade Ito Grade IV depending on the extent of lower and upper GI, liver, and skin involvement. The International Multi-Center Consortium of Transplant Sites has developed consensus guidelines to standardize data collection around diagnosis and staging of aGvHD. These guidelines have been endorsed by the National Institutes of Health (NIH) and Center for International Blood and Marrow Transplant Research (CIBMTR) (Harris 2016a; Shoemans 2018). Chronic GvHD is characterized by progressive tissue injury leading to fibrosis and susceptibility to infection with symptoms resembling autoimmune collagen vascular diseases (Jagasia 2015). Pathogenesis may involve inflammation, cell-mediated immunity, humoral immunity, and fibrosis. It is differentiated from aGvHD by its clinical features rather than a temporal relationship to HSCT. The 2014 NIH chronic GvHD consensus criteria guidelines, endorsed by the CIBMTR, defined organ-specific scoring and severity grading. In addition, the guidelines provide the recommendation that at least one diagnostic manifestation or one distinctive manifestation plus diagnostic confirmation such as a pertinent biopsy, laboratory test or other test, medical specialist evaluation, or radiographic image in the same or other organ, be required for diagnosis (Jagasia 2015; Schoemans 2018).

In some aspects, acute GvHD can be characterized by selective damage to organs and tissues, including, but not limited to, the liver, skin, mucosa, and gastrointestinal tract. Chronic GVHD can additionally cause damage to the connective tissue, exocrine glands, and lungs. Symptoms of acute GvHD include, but are not limited to, dermatitis, mucositis, hepatitis, jaundice, enteritis, which can lead to diarrhea, nausea, vomiting, cramping, abdominal pain, blood in the stool, and combinations thereof. Non-limiting examples of symptoms associated with chronic GvHD include: dry eyes or mouth, vision changes, mouth ulcers, difficulty swallowing, gum disease and tooth decay, hair loss, nail loss and/or brittleness, sensitivity to spicy or acidic foods, mouth pain, pulmonary symptoms, such as wheezing or shortness of breath, muscle/joint pain or weakness, fatigue, skin rash that is red to purple, skin discoloration, vaginal dryness, loss of appetite, weight loss, abdominal pain, and combinations thereof. Unless indicated otherwise, the term GvHD refers to both acute and chronic GvHD.

As used herein, the term “hematopoietic stem cells” (HSCs) refers to a subset of multipotent stem cells that give rise to all the blood or immune cell types, including myeloid (e.g., monocytes and macrophages, neutrophils, basophils, eosinophils, erythrocytes, megakaryocytes/platelets, dendritic cells, mast cells), and lymphoid lineages (e.g., innate lymphoid cells, T-cells, B-cells, NKT-cells, NK-cells), and having multi-lineage hematopoietic differentiation potential and sustained self-renewal activity. The term “stem cells,” as used herein, refers to cells that retain the ability to renew themselves through mitotic cell division and can differentiate into a diverse range of specialized cell types.

As used herein, the term “hematopoietic stem cell transplantation” (HSCT) refers to the transplantation of multipotent hematopoietic stem cells from a donor to a recipient. In an “autologous” HSC transplantation, the stem cells are isolated from a subject in need of a treatment (e.g., chemotherapy or radiation therapy) and then administered back to the subject after the treatment. Therefore, in the context of autologous HSC transplantation, the terms “donor” and “recipient”/“subject” refer to the same individual. In a “syngenic” HSC transplantation, stem cells are isolated from an identical twin of the subject to be treated and then administered to the subject after treatment (e.g., chemotherapy or radiation therapy). In an “allogenic” HSC transplantation, stem cells are isolated from a healthy donor (e.g., non-identical twin or an individual related or not related to the subject to be treated) and then administered to a different recipient subject after treatment (e.g., chemotherapy or radiation therapy). In such transplantations, the terms “donor” and “subject”/“recipient” refer to different individuals. Unless specified otherwise, the term HSCT is not limited to any specific type of HSCT (e.g., encompasses autologous, syngenic, and allogenic HSCT).

The term “Glade” refers to the OTUs or members of a phylogenetic tree that are downstream of a statistically valid node in a phylogenetic tree. The Glade comprises a set of terminal leaves in the phylogenetic tree that is a distinct monophyletic evolutionary unit and that share some extent of sequence similarity.

The term “microbiota” refers to the ecological community of microorganisms that occur (sustainably or transiently) in and on an animal subject, typically a mammal such as a human, including eukaryotes, archaea, bacteria, and viruses (including bacterial viruses i.e., phage).

The term “microbiome” refers to the genetic content of the communities of microbes that live in and on the human body, both sustainably and transiently, including eukaryotes, archaea, bacteria, and viruses (including bacterial viruses (i.e., phage)), wherein “genetic content” includes genomic DNA, RNA such as ribosomal RNA, the epigenome, plasmids, and all other types of genetic information.

The term “ecological niche” or “niche” refers to the ecological space in which an organism or group of organisms occupies. Niche describes how an organism or population or organisms responds to the distribution of resources, physical parameters (e.g., host tissue space) and competitors (e.g., by growing when resources are abundant, and when predators, parasites and pathogens are scarce) and how it in turn alters those same factors (e.g., limiting access to resources by other organisms, acting as a food source for predators and a consumer of prey).

The term “dysbiosis” refers to a state of the microbiota of the GI tract or other body area in a subject, including mucosal or skin surfaces in which the normal diversity and/or function of the ecological network is disrupted. This unhealthy state can be due to a decrease in diversity, the overgrowth of one or more pathogens or pathobionts, symbiotic organisms able to cause disease only when certain genetic and/or environmental conditions are present in a subject, or the shift to an ecological microbial network that no longer provides an essential function to the host subject, and therefore no longer promotes health.

As used herein, the term “operational taxonomic units” or “OTU” (or plural, “OTUs”) refers to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence can be the 16S rDNA sequence or a portion of the 16S rDNA sequence. In other embodiments, the entire genomes of two entities are sequenced and compared. In another embodiment, select regions such as multilocus sequence tags (MLST), specific genes, or sets of genes can be genetically compared. In 16S embodiments, OTUs that share ≥97% average nucleotide identity across the entire 16S or a variable region of the 16S rDNA, e.g., a V4 region, are considered the same OTU (see, e.g., Claesson M J, Wang Q, O'Sullivan 0, Greene-Diniz R, Cole J R, Ros R P, and O'Toole P W. 2010. Comparison of two next-generation sequencing technologies for resolving highly complex microbiome composition using tandem variable 16S rRNA gene regions. Nucleic Acids Res 38: e200. Konstantinidis K T, Ramette A, and Tiedje J M. 2006. The bacterial species definition in the genomic era. Philos Trans R Soc Lond B Biol Sci 361: 1929-1940). In embodiments involving the complete genome, MLSTs, specific genes, or sets of genes OTUs that share ≥95% average nucleotide identity are considered the same OTU (see, e.g., Achtman M, and Wagner M. 2008. Microbial diversity and the genetic nature of microbial species. Nat. Rev. Microbiol. 6: 431-440. Konstantinidis K T, Ramette A, and Tiedje J M. 2006. The bacterial species definition in the genomic era. Philos Trans R Soc Lond B Biol Sci 361: 1929-1940). OTUs are frequently defined by comparing sequences between organisms. Generally, sequences with less than 95% sequence identity are not considered to form part of the same OTU. In some cases, an OTU is characterized by a combination of nucleotide markers, genes, and/or single nucleotide variants (SNVs). In some cases, the referenced genes are highly conserved genes (e.g., “house-keeping” genes). The features defining an OTU can be a combination of the foregoing. Such characterization employs, e.g., WGS data or a whole genome sequence.

As used herein, the term “phylogenetic tree” refers to a graphical representation of the evolutionary relationships of one genetic sequence to another that is generated using a defined set of phylogenetic reconstruction algorithms (e.g., parsimony, maximum likelihood, or Bayesian). Nodes in the tree represent distinct ancestral sequences and the confidence of any node is provided by a bootstrap or Bayesian posterior probability, which measures branch uncertainty.

Identification of and reference to bacterial species described herein can be found throughout the present disclosure, including the Figures/Drawings, Tables, and Sequence Listing. Where a taxonomic name is used or referenced for a specific bacterium, it is understood that the bacterium may have previously had a different taxonomic name(s) and that one of skill in the art would have resources available to identify and associate previous taxonomic names with those described herein, as used in the art, or both. Such resources include, but are not limited to, Bergey's Manual of Systematics of Archea and Bacteria (1^{st} Ed.); Bergey's Manual of Systematic Bacteriology (2^{nd} Ed.); the online version available at onlinelibrary.wiley.com/doi/book/10.1002/9781118960608; and the National Center for Biotechnology Information (NCBI) database available online at www.ncbi.nlm.nih.gov/taxonomy.

The specification is most thoroughly understood in light of the teachings of the references cited within the specification. The embodiments within the specification provide an illustration of embodiments and should not be construed to limit the scope. The skilled artisan readily recognizes that many other embodiments are encompassed. All publications and patents cited in this disclosure are incorporated by reference in their entirety. To the extent the material incorporated by reference contradicts or is inconsistent with this specification, the specification will supersede any such material. The citation of any references herein is not an admission that such references are prior art.

As used herein, the term “subject” refers to any animal subject including humans, laboratory animals (e.g., primates, rats, mice), livestock (e.g., cows, sheep, goats, pigs, turkeys, and chickens), and household pets (e.g., dogs, cats, and rodents).

The “colonization” of a host organism includes the non-transitory residence of a bacterium or other microscopic organism. In the case of treatment, the host is generally referred to herein as a “subject”, typically a human or other mammal. As used herein, “reducing colonization” of a host subject's gastrointestinal tract (or any other microbiotal niche) by a pathogenic bacterium includes a reduction in the residence time of the pathogen in the gastrointestinal tract as well as a reduction in the number (or concentration) of the pathogen in the gastrointestinal tract or adhered to the luminal surface of the gastrointestinal tract. Measuring reductions of adherent pathogens can be demonstrated, e.g., by a biopsy sample, or reductions can be measured indirectly, e.g., by measuring the pathogenic burden in the stool of a mammalian host.

A “combination” of two or more bacteria includes the physical co-existence of the two bacteria, either in the same material or product or in physically connected products, as well as the temporal co-administration or co-localization of the two bacteria.

A “cytotoxic” activity or bacterium includes the ability to kill a host cell or CD8-related toxicity. A “cytostatic” activity or bacterium includes the ability to inhibit, partially or fully, growth, metabolism, and/or proliferation of a bacterial cell, such as a pathogenic bacterial cell.

To be free of “non-comestible products” means that a bacterial composition or other material provided herein does not have a substantial amount of a non-comestible product, e.g., a product or material that is inedible, harmful or otherwise undesired in a product suitable for administration, e.g., oral administration, to a human subject. Non-comestible products are often found in preparations of bacteria from the prior art.

A “biologically pure culture” is a culture of bacteria in a medium in which only selected viable species are present and no other viable species of microorganisms are detected.

For nucleic acids, the term “substantial homology” indicates that two nucleic acids, or designated sequences thereof, when optimally aligned and compared, are identical, with appropriate nucleotide insertions or deletions, in at least about 80% of the nucleotides, at least about 90% to 95%, or at least about 98% to 99.5% of the nucleotides. Alternatively, substantial homology exists when the segments will hybridize under selective hybridization conditions, to the complement of the strand.

For polypeptides, the term “substantial homology” indicates that two polypeptides, or designated sequences thereof, when optimally aligned and compared, are identical, with appropriate amino acid insertions or deletions, in at least about 80% of the amino acids, at least about 90% to 95%, or at least about 98% to 99.5% of the amino acids.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at worldwideweb.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4: 11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at worldwideweb.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and)(BLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules described herein. BLAST protein searches can be performed with the) (BLAST program, score=50, wordlength=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See worldwideweb.ncbi.nlm.nih.gov. Other methods of determining identity that are known in the art can be used.

The term “patient” includes human and other mammalian subjects that receive either prophylactic or therapeutic treatment.

As used herein, the terms “ug” and “uM” are used interchangeably with “μg” and “μM,” respectively.

Various aspects described herein are described in further detail throughout the specification.

Unless otherwise indicated, all numbers expressing quantities of ingredients, reaction conditions, and so forth used in the specification, including claims, are to be understood as being modified in all instances by the term “about.” Accordingly, unless otherwise indicated to the contrary, the numerical parameters are approximations and can vary depending upon the desired properties sought to be obtained. At the very least, and not as an attempt to limit the application of the doctrine of equivalents to the scope of the claims, each numerical parameter should be construed in light of the number of significant digits and ordinary rounding approaches.

Table A shows “STR” designations for strains described herein, SEQ ID NOs of 16S rDNA sequences encoding the 16S rRNA of each STR, and a reference to the species or strain name sharing high sequence identity with the 16S rDNA sequence for each SEQ ID NO.

TABLE A
SEQ ID NO, STR Designation, and Associated Species

| SEQ ID NO | STR | Species |
|---|---|---|
| SEQ ID NO: 1 | STR00005.1 | Collinsella_aerofaciens_strain_JCM_10188_16S_ribosomal_RNA |
| SEQ ID NO: 2 | STR00003.1 | Bacteroides_uniformis_strain_JCM_5828_16S_ribosomal_RNA |
| SEQ ID NO: 3 | STR00044.1 | Ruminococcus_lactaris_ATCC_29176_16S_ribosomal_RNA |
| SEQ ID NO: 4 | STR00043.1 | Blautia_wexlerae_DSM_19850_16S_ribosomal_RNA |
| SEQ ID NO: 5 | STR00053 | Bifidobacterium_longum_strain_KCTC_3128_16S_ribosomal_RNA |
| SEQ ID NO: 6 | STR00059.1 | Butyricimonas_faecihominis_strain_180-3_16S_ribosomal_RNA |
| SEQ ID NO: 7 | STR00060.1 | [Clostridium]_spiroforme_strain_JCM_1432_16S_ribosomal_RNA |
| SEQ ID NO: 8 | STR00061.1 | Bacteroides_caccae_strain_JCM_9498_16S_ribosomal_RNA |
| SEQ ID NO: 9 | STR00062.1 | Alistipes_finegoldii_strain_AHN_2437_16S_ribosomal_RNA |
| SEQ ID NO: 10 | STR00066.1 | Lactonifactor_longoviformis_strain_ED-Mt61/PYG-s6_16S_ribosomal_RNA |
| SEQ ID NO: 11 | STR00067.1 | Intestinimonas_butyriciproducens_strain_SRB-521-5-I_16S_ribosomal_RNA |
| SEQ ID NO: 12 | STR00065 | Drancourtella_massiliensis_strain_GD1_16S_ribosomal_RNA |
| SEQ ID NO: 13 | STR00069 | Faecalicatena_orotica_strain_JCM_1429_16S_ribosomal_RNA |
| SEQ ID NO: 14 | STR00068.1 | Parabacteroides_distasonis_strain_JCM_5825_16S_ribosomal_RNA |
| SEQ ID NO: 15 | STR00070.1 | Oscillibacter_ruminantium_GH1_16S_ribosomal_RNA |
| SEQ ID NO: 16 | STR00071.1 | Akkermansia_muciniphila_strain_ATCC_BAA-835_16S_ribosomal_RNA |
| SEQ ID NO: 17 | STR00007.1 | Bacteroides_koreensis_strain_YS-aM39_16S_ribosomal_RNA |
| SEQ ID NO: 18 | STR00072 | Clostridium_disporicum_strain_DS1_16S_ribosomal_RNA |
| SEQ ID NO: 19 | STR00075.3 | Flavonifractor_plautii_strain_265_16S_ribosomal_RNA |
| SEQ ID NO: 20 | STR00077.1 | Terrisporobacter_mayombei_strain_SFC-5_16S_ribosomal_RNA |
| SEQ ID NO: 21 | STR00076.5 | Blautia_hominis_strain_KB1_16S_ribosomal_RNA |
| SEQ ID NO: 22 | STR00078.1 | Roseburia_intestinalis_L1-82_16S_ribosomal_RNA |
| SEQ ID NO: 23 | STR00079.1 | Lachnospira_pectinoschiza_strain_150-1_16S_ribosomal_RNA |
| SEQ ID NO: 24 | STR00080.1 | [Eubacterium]_rectale_ATCC_33656_16S_ribosomal_RNA |
| SEQ ID NO: 25 | STR00082.1 | Roseburia_intestinalis_L1-82_16S_ribosomal_RNA |
| SEQ ID NO: 26 | STR00085.1 | Lactonifactor_longoviformis_strain_ED-Mt61/PYG-s6_16S_ribosomal_RNA |
| SEQ ID NO: 27 | STR00086 | Turicibacter_sanguinis_strain_MOL361_16S_ribosomal_RNA |
| SEQ ID NO: 28 | STR00089.1 | Terrisporobacter_petrolearius_strain_LAM0A37_16S_ribosomal_RNA |
| SEQ ID NO: 29 | STR00090 | Cellulosilyticum_lentocellum_DSM_5427_16S_ribosomal_RNA |
| SEQ ID NO: 30 | STR00091.1 | Blautia_hominis_strain_KB1_16S_ribosomal_RNA |
| SEQ ID NO: 31 | STR00092.6 | Erysipelatoclostridium_ramosum_strain_JCM_1298_16S_ribosomal_RNA |
| SEQ ID NO: 32 | STR00094.1 | Romboutsia_timonensis_strain_DR1_16S_ribosomal_RNA |
| SEQ ID NO: 33 | STR00098.1 | Robinsoniella_peoriensis_strain_PPC31_16S_ribosomal_RNA |
| SEQ ID NO: 34 | STR00093.1 | Anaeromassilibacillus_senegalensis_strain_mt9_16S_ribosomal_RNA |
| SEQ ID NO: 35 | STR00096.5 | Faecalicatena_orotica_strain_DSM_1287_16S_ribosomal_RNA |
| SEQ ID NO: 36 | STR00097.3 | Emergencia_timonensis_strain_SN18_16S_ribosomal_RNA |
| SEQ ID NO: 37 | STR00100.1 | Bacteroides_vulgatus_ATCC_8482_16S_ribosomal_RNA |
| SEQ ID NO: 38 | STR00099.1 | Clostridium_subterminale_strain_JCM_1417_16S_ribosomal_RNA |
| SEQ ID NO: 39 | STR00101.1 | Blautia_coccoides_strain_JCM_1395_16S_ribosomal_RNA |
| SEQ ID NO: 40 | STR00108.1 | [Clostridium]_symbiosum_strain_ATCC_14940_16S_ribosomal_RNA |
| SEQ ID NO: 41 | STR00106.1 | [Clostridium]_scindens_strain_ATCC_35704_16S_ribosomal_RNA |
| SEQ ID NO: 42 | STR00105.7 | Dorea_longicatena_strain_111-35_16S_ribosomal_RNA |
| SEQ ID NO: 43 | STR00104.1 | Eisenbergiella_tayi_strain_B086562_16S_ribosomal_RNA |
| SEQ ID NO: 44 | STR00107.5 | [Clostridium]_aldenense_strain_RMA_9741_16S_ribosomal_RNA |
| SEQ ID NO: 45 | STR00109.1 | Intestinimonas_butyriciproducens_strain_SRB-521-5-I_16S_ribosomal_RNA |
| SEQ ID NO: 46 | STR00111.1 | [Clostridium]_lavalense_strain_CCRI-9842_16S_ribosomal_RNA |
| SEQ ID NO: 47 | STR00112.1 | Turicibacter_sanguinis_strain_MOL361_16S_ribosomal_RNA |
| SEQ ID NO: 48 | STR00116.1 | [Ruminococcus]_gnavus_ATCC_29149_16S_ribosomal_RNA |
| SEQ ID NO: 49 | STR00115.1 | Blautia_producta_ATCC_27340_=_DSM_2950_strain_JCM_1471_16S_ribosomal_RNA |
| SEQ ID NO: 50 | STR00113.1 | Barnesiella_intestinihominis_YIT_11860_16S_ribosomal_RNA |
| SEQ ID NO: 51 | STR00114.1 | Bacteroides_salyersiae_strain_JCM_12988_16S_ribosomal_RNA |
| SEQ ID NO: 52 | STR00118.1 | Eubacterium_callanderi_strain_DSM_3662_16S_ribosomal_RNA |
| SEQ ID NO: 53 | STR00121.1 | Anaerostipes_caccae_strain_L1-92_16S_ribosomal_RNA |
| SEQ ID NO: 54 | STR00120.1 | Bifidobacterium_longum_subsp._suillum_strain_Su_851_16S_ribosomal_RNA |
| SEQ ID NO: 55 | STR00125 | Bacteroides_kribbi_strain_R2F3-3-3_16S_ribosomal_RNA |
| SEQ ID NO: 56 | STR00124.1 | Bacteroides_uniformis_strain_JCM_5828_16S_ribosomal_RNA |
| SEQ ID NO: 57 | STR00126.1 | Bacteroides_vulgatus_ATCC_8482_16S_ribosomal_RNA |
| SEQ ID NO: 58 | STR00123.1 | Lactobacillus_fermentum_strain_CIP_102980_16S_ribosomal_RNA |
| SEQ ID NO: 59 | STR00117.1 | Blautia_hominis_strain_KB1_16S_ribosomal_RNA |
| SEQ ID NO: 60 | STR00129.1 | Coprococcus_eutactus_strain_ATCC_27759_16S_ribosomal_RNA |
| SEQ ID NO: 61 | STR00127.1 | [Ruminococcus]_torques_strain_VPI_B2-51_16S_ribosomal_RNA |
| SEQ ID NO: 62 | STR00128.1 | [Ruminococcus]_gnavus_ATCC_29149_16S_ribosomal_RNA |
| SEQ ID NO: 63 | STR00131.1 | Eubacterium_callanderi_strain_DSM_3662_16S_ribosomal_RNA |
| SEQ ID NO: 64 | STR00041.1 | Tyzzerella_nexilis_DSM_1787_16S_ribosomal_RNA |
| SEQ ID NO: 65 | STR00030.1 | Blautia_hominis_strain_KB1_16S_ribosomal_RNA |
| SEQ ID NO: 66 | STR00037.1 | Paraclostridium_bifermentans_strain_JCM_1386_16S_ribosomal_RNA |
| SEQ ID NO: 67 | STR00018.1 | Bacteroides_uniformis_strain_JCM_5828_16S_ribosomal_RNA |
| SEQ ID NO: 68 | STR00017 | Bifidobacterium_stercoris_JCM_15918_strain_Eg1_16S_ribosomal_RNA |
| SEQ ID NO: 69 | STR00039.1 | [Ruminococcus]_gnavus_ATCC_29149_16S_ribosomal_RNA |
| SEQ ID NO: 70 | STR00021.1 | Emergencia_timonensis_strain_SN18_16S_ribosomal_RNA |
| SEQ ID NO: 71 | STR00035.1 | Bacteroides_xylanisolvens_XB1A_16S_ribosomal_RNA |
| SEQ ID NO: 72 | STR00023 | [Clostridium]_symbiosum_strain_ATCC_14940_16S_ribosomal_RNA |
| SEQ ID NO: 73 | STR00020.1 | Erysipelatoclostridium_ramosum_strain_JCM_1298_16S_ribosomal_RNA |
| SEQ ID NO: 74 | STR00031.1 | Bacteroides_intestinalis_strain_341_16S_ribosomal_RNA |
| SEQ ID NO: 75 | STR00042 | Absiella_dolichum_strain_JCM_10413_16S_ribosomal_RNA |
| SEQ ID NO: 76 | STR00038.1 | Bacteroides_eggerthii_strain_JCM_12986_16S_ribosomal_RNA |
| SEQ ID NO: 77 | STR00024.1 | Flavonifractor_plautii_strain_265_16S_ribosomal_RNA |
| SEQ ID NO: 78 | STR00033.1 | Holdemania_filiformis_strain_J1-31B-1_16S_ribosomal_RNA |
| SEQ ID NO: 79 | STR00013.1 | [Clostridium]_innocuum_strain_B-3_16S_ribosomal_RNA |
| SEQ ID NO: 80 | STR00022.5 | [Clostridium]_bolteae_strain_JCM_12243_16S_ribosomal_RNA |
| SEQ ID NO: 81 | STR00027 | [Clostridium]_scindens_strain_ATCC_35704_16S_ribosomal_RNA |
| SEQ ID NO: 82 | STR00036.1 | Bacteroides_faecis_MAJ27_16S_ribosomal_RNA |
| SEQ ID NO: 83 | STR00019 | [Clostridium]_hylemonae_strain_TN-272_16S_ribosomal_RNA |
| SEQ ID NO: 84 | STR00034.1 | [Clostridium]_bolteae_strain_16351_16S_ribosomal_RNA |
| SEQ ID NO: 85 | STR00012.5 | [Clostridium]_innocuum_strain_B-3_16S_ribosomal_RNA |
| SEQ ID NO: 86 | STR00045.1 | Intestinimonas_butyriciproducens_strain_SRB-521-5-I_16S_ribosomal_RNA |
| SEQ ID NO: 87 | STR00047.1 | Cellulosilyticum_lentocellum_DSM_5427_16S_ribosomal_RNA |
| SEQ ID NO: 88 | STR00002.1 | Coprococcus_comes_ATCC_27758_16S_ribosomal_RNA |
| SEQ ID NO: 89 | STR00052.1 | Cellulosilyticum_lentocellum_DSM_5427_16S_ribosomal_RNA |
| SEQ ID NO: 90 | STR00056.1 | Cellulosilyticum_lentocellum_DSM_5427_16S_ribosomal_RNA |
| SEQ ID NO: 91 | STR00048.1 | [Clostridium]_glycyrrhizinilyticum_strain_ZM35_16S_ribosomal_RNA |
| SEQ ID NO: 92 | STR00046.1 | Longicatena_caecimuris_strain_PG-426-CC-2_16S_ribosomal_RNA |
| SEQ ID NO: 93 | STR00049 | Eggerthella_lenta_strain_DSM_2243_16S_ribosomal_RNA |
| SEQ ID NO: 94 | STR00051.1 | [Clostridium]_spiroforme_strain_JCM_1432_16S_ribosomal_RNA |
| SEQ ID NO: 95 | STR00001.1 | [Clostridium]_innocuum_strain_B-3_16S_ribosomal_RNA |
| SEQ ID NO: 96 | STR00054.1 | Clostridium_tertium_strain_795_16S_ribosomal_RNA |
| SEQ ID NO: 97 | STR00134.1 | Blautia_hydrogenotrophica_strain_S5a36_16S_ribosomal_RNA |
| SEQ ID NO: 98 | STR00133 | [Clostridium]_scindens_strain_ATCC_35704_16S_ribosomal_RNA |
| SEQ ID NO: 99 | STR00132.5 | Eisenbergiella_tayi_strain_B086562_16S_ribosomal_RNA |
| SEQ ID NO: 100 | STR00135.1 | Holdemania_massiliensis_AP2_16S_ribosomal_RNA |
| SEQ ID NO: 101 | STR00136.1 | Bacteroides_eggerthii_strain_JCM_12986_16S_ribosomal_RNA |
| SEQ ID NO: 102 | STR00032.1 | Gemmiger_formicilis_strain_X2-56_16S_ribosomal_RNA |
| SEQ ID NO: 103 | STR00137 | Bifidobacterium_dentium_strain_B764_16S_ribosomal_RNA |
| SEQ ID NO: 104 | STR00143.6 | Blautia_obeum_ATCC_29174_16S_ribosomal_RNA |
| SEQ ID NO: 105 | STR00139.4 | [Clostridium]_scindens_strain_ATCC_35704_16S_ribosomal_RNA |
| SEQ ID NO: 106 | STR00016 | Clostridium_butyricum_strain_JCM_1391_16S_ribosomal_RNA |
| SEQ ID NO: 107 | STR00014 | Ruminococcus_faecis_JCM_15917_strain_Eg2_16S_ribosomal_RNA |
| SEQ ID NO: 108 | STR00059 | Butyricimonas_faecihominis_strain_180-3_16S_ribosomal_RNA |
| SEQ ID NO: 109 | STR00085 | Lactonifactor_longoviformis_strain_ED-Mt61/PYG-s6_16S_ribosomal_RNA |
| SEQ ID NO: 110 | STR00005 | Collinsella_aerofaciens_strain_JCM_10188_16S_ribosomal_RNA |
| SEQ ID NO: 111 | STR00101 | Blautia_coccoides_strain_JCM_1395_16S_ribosomal_RNA |
| SEQ ID NO: 112 | STR00006 | Shigella_flexneri_strain_ATCC_29903_16S_ribosomal_RNA |
| SEQ ID NO: 113 | STR00011 | Blautia_obeum_ATCC_29174_16S_ribosomal_RNA |
| SEQ ID NO: 114 | STR00077 | Terrisporobacter_mayombei_strain_SFC-5_16S_ribosomal_RNA |
| SEQ ID NO: 115 | STR00091 | Blautia_hominis_strain_KB1_16S_ribosomal_RNA |
| SEQ ID NO: 116 | STR00067 | Intestinimonas_butyriciproducens_strain_SRB-521-5-I_16S_ribosomal_RNA |
| SEQ ID NO: 117 | STR00079 | Lachnospira_pectinoschiza_strain_150-1_16S_ribosomal_RNA |
| SEQ ID NO: 118 | STR00089 | Terrisporobacter_petrolearius_strain_LAM0A37_16S_ribosomal_RNA |
| SEQ ID NO: 119 | STR00068 | Parabacteroides_distasonis_strain_JCM_5825_16S_ribosomal_RNA |
| SEQ ID NO: 120 | STR00043 | Blautia_wexlerae_DSM_19850_16S_ribosomal_RNA |
| SEQ ID NO: 121 | STR00070 | Oscillibacter_ruminantium_GH1_16S_ribosomal_RNA |
| SEQ ID NO: 122 | STR00044 | Ruminococcus_lactaris_ATCC_29176_16S_ribosomal_RNA |
| SEQ ID NO: 123 | STR00080 | [Eubacterium]_rectale_ATCC_33656_16S_ribosomal_RNA |
| SEQ ID NO: 124 | STR00099 | Clostridium_subterminale_strain_JCM_1417_16S_ribosomal_RNA |
| SEQ ID NO: 125 | STR00063 | Ruthenibacterium_lactatiformans_strain_585-1_16S_ribosomal_RNA |
| SEQ ID NO: 126 | STR00066 | Lactonifactor_longoviformis_strain_ED-Mt61/PYG-s6_16S_ribosomal_RNA |
| SEQ ID NO: 127 | STR00102 | [Clostridium]_bolteae_strain_JCM_12243_16S_ribosomal_RNA |
| SEQ ID NO: 128 | STR00060 | [Clostridium]_spiroforme_strain_JCM_1432_16S_ribosomal_RNA |
| SEQ ID NO: 129 | STR00062 | Alistipes_finegoldii_strain_AHN_2437_16S_ribosomal_RNA |
| SEQ ID NO: 130 | STR00003 | Bacteroides_uniformis_strain_JCM_5828_16S_ribosomal_RNA |
| SEQ ID NO: 131 | STR00061 | Bacteroides_caccae_strain_JCM_9498_16S_ribosomal_RNA |
| SEQ ID NO: 132 | STR00071 | Akkermansia_muciniphila_strain_ATCC_BAA-835_16S_ribosomal_RNA |
| SEQ ID NO: 133 | STR00007 | Bacteroides_koreensis_strain_YS-aM39_16S_ribosomal_RNA |
| SEQ ID NO: 134 | STR00073 | Ruminococcus_albus_strain_JCM_14654_16S_ribosomal_RNA |
| SEQ ID NO: 135 | STR00078 | Roseburia_intestinalis_L1-82_16S_ribosomal_RNA |
| SEQ ID NO: 136 | STR00126 | Bacteroides_vulgatus_ATCC_8482_16S_ribosomal_RNA |
| SEQ ID NO: 137 | STR00018 | Bacteroides_uniformis_strain_JCM_5828_16S_ribosomal_RNA |
| SEQ ID NO: 138 | STR00054 | Clostridium_tertium_strain_795_16S_ribosomal_RNA |
| SEQ ID NO: 139 | STR00038 | Bacteroides_eggerthii_strain_JCM_12986_16S_ribosomal_RNA |
| SEQ ID NO: 140 | STR00030 | Blautia_hominis_strain_KB1_16S_ribosomal_RNA |
| SEQ ID NO: 141 | STR00055 | [Clostridium]_bolteae_strain_JCM_12243_16S_ribosomal_RNA |
| SEQ ID NO: 142 | STR00039 | [Ruminococcus]_gnavus_ATCC_29149_16S_ribosomal_RNA |
| SEQ ID NO: 143 | STR00047 | Cellulosilyticum_lentocellum_DSM_5427_16S_ribosomal_RNA |
| SEQ ID NO: 144 | STR00026 | Faecalibacterium_prausnitzii_strain_ATCC_27768_16S_ribosomal_RNA |
| SEQ ID NO: 145 | STR00136 | Bacteroides_eggerthii_strain_JCM_12986_16S_ribosomal_RNA |
| SEQ ID NO: 146 | STR00020 | Erysipelatoclostridium_ramosum_strain_JCM_1298_16S_ribosomal_RNA |
| SEQ ID NO: 147 | STR00002 | Coprococcus_comes_ATCC_27758_16S_ribosomal_RNA |
| SEQ ID NO: 148 | STR00013 | [Clostridium]_innocuum_strain_B-3_16S_ribosomal_RNA |
| SEQ ID NO: 149 | STR00021 | Emergencia_timonensis_strain_SN18_16S_ribosomal_RNA |
| SEQ ID NO: 150 | STR00034 | [Clostridium]_bolteae_strain_16351_16S_ribosomal_RNA |
| SEQ ID NO: 151 | STR00134 | Blautia_hydrogenotrophica_strain_S5a36_16S_ribosomal_RNA |
| SEQ ID NO: 152 | STR00040 | Ruminococcus_bromii_strain_ATCC_27255_16S_ribosomal_RNA |
| SEQ ID NO: 153 | STR00057 | Longibaculum_muris_strain_MT10-315-CC-1.2-2_16S_ribosomal_RNA |
| SEQ ID NO: 154 | STR00024 | Flavonifractor_plautii_strain_265_16S_ribosomal_RNA |
| SEQ ID NO: 155 | STR00031 | Bacteroides_intestinalis_strain_341_16S_ribosomal_RNA |
| SEQ ID NO: 156 | STR00050 | [Eubacterium]_rectale_ATCC_33656_16S_ribosomal_RNA |
| SEQ ID NO: 157 | STR00001 | [Clostridium]_innocuum_strain_B-3_16S_ribosomal_RNA |
| SEQ ID NO: 158 | STR00048 | [Clostridium]_glycyrrhizinilyticum_strain_ZM35_16S_ribosomal_RNA |
| SEQ ID NO: 159 | STR00035 | Bacteroides_xylanisolvens_XB1A_16S_ribosomal_RNA |
| SEQ ID NO: 160 | STR00052 | Cellulosilyticum_lentocellum_DSM_5427_16S_ribosomal_RNA |
| SEQ ID NO: 161 | STR00046 | Longicatena_caecimuris_strain_PG-426-CC-2_16S_ribosomal_RNA |
| SEQ ID NO: 162 | STR00132.4 | Eisenbergiella_tayi_strain_B086562_16S_ribosomal_RNA |
| SEQ ID NO: 163 | STR00058 | Agathobaculum_desmolans_strain_ATCC_43058_16S_ribosomal_RNA |
| SEQ ID NO: 164 | STR00120 | Bifidobacterium_longum_subsp._suillum_strain_Su_851_16S_ribosomal_RNA |
| SEQ ID NO: 165 | STR00045 | Intestinimonas_butyriciproducens_strain_SRB-521-5-I_16S_ribosomal_RNA |
| SEQ ID NO: 166 | STR00025.5 | Eubacterium_callanderi_strain_DSM_3662_16S_ribosomal_RNA |
| SEQ ID NO: 167 | STR00116 | [Ruminococcus]_gnavus_ATCC_29149_16S_ribosomal_RNA |
| SEQ ID NO: 168 | STR00128 | [Ruminococcus]_gnavus_ATCC_29149_16S_ribosomal_RNA |
| SEQ ID NO: 169 | STR00051 | [Clostridium]_spiroforme_strain_JCM_1432_16S_ribosomal_RNA |
| SEQ ID NO: 170 | STR00037 | Paraclostridium_bifermentans_strain_JCM_1386_16S_ribosomal_RNA |
| SEQ ID NO: 171 | STR00056 | Cellulosilyticum_lentocellum_DSM_5427_16S_ribosomal_RNA |
| SEQ ID NO: 172 | STR00036 | Bacteroides_faecis_MAJ27_16S_ribosomal_RNA |
| SEQ ID NO: 173 | STR00135 | Holdemania_massiliensis_AP2_16S_ribosomal_RNA |
| SEQ ID NO: 174 | STR00124 | Bacteroides_uniformis_strain_JCM_5828_16S_ribosomal_RNA |
| SEQ ID NO: 175 | STR00041 | Tyzzerella_nexilis_DSM_1787_16S_ribosomal_RNA |
| SEQ ID NO: 176 | STR00121 | Anaerostipes_caccae_strain_L1-92_16S_ribosomal_RNA |
| SEQ ID NO: 177 | STR00131 | Eubacterium_callanderi_strain_DSM_3662_16S_ribosomal_RNA |
| SEQ ID NO: 178 | STR00093 | Anaeromassilibacillus_senegalensis_strain_mt9_16S_ribosomal_RNA |
| SEQ ID NO: 179 | STR00100 | Bacteroides_vulgatus_ATCC_8482_16S_ribosomal_RNA |
| SEQ ID NO: 180 | STR00127 | [Ruminococcus]_torques_strain_VPI_B2-51_16S_ribosomal_RNA |
| SEQ ID NO: 181 | STR00082 | Roseburia_intestinalis_L1-82_16S_ribosomal_RNA |
| SEQ ID NO: 182 | STR00123 | Lactobacillus_fermentum_strain_CIP_102980_16S_ribosomal_RNA |
| SEQ ID NO: 183 | STR00092.5 | Erysipelatoclostridium_ramosum_strain_JCM_1298_16S_ribosomal_RNA |
| SEQ ID NO: 184 | STR00098 | Robinsoniella_peoriensis_strain_PPC31_16S_ribosomal_RNA |
| SEQ ID NO: 185 | STR00104 | Eisenbergiella_tayi_strain_B086562_16S_ribosomal_RNA |
| SEQ ID NO: 186 | STR00114 | Bacteroides_salyersiae_strain_JCM_12988_16S_ribosomal_RNA |
| SEQ ID NO: 187 | STR00109 | Intestinimonas_butyriciproducens_strain_SRB-521-5-I_16S_ribosomal_RNA |
| SEQ ID NO: 188 | STR00129 | Coprococcus_eutactus_strain_ATCC_27759_16S_ribosomal_RNA |
| SEQ ID NO: 189 | STR00111 | [Clostridium]_lavalense_strain_CCRI-9842_16S_ribosomal_RNA |
| SEQ ID NO: 190 | STR00108 | [Clostridium]_symbiosum_strain_ATCC_14940_16S_ribosomal_RNA |
| SEQ ID NO: 191 | STR00119 | Turicibacter_sanguinis_strain_MOL361_16S_ribosomal_RNA |
| SEQ ID NO: 192 | STR00113 | Barnesiella_intestinihominis_YIT_11860_16S_ribosomal_RNA |
| SEQ ID NO: 193 | STR00097.4 | Emergencia_timonensis_strain_SN18_16S_ribosomal_RNA |
| SEQ ID NO: 194 | STR00112 | Turicibacter_sanguinis_strain_MOL361_16S_ribosomal_RNA |
| SEQ ID NO: 195 | STR00118 | Eubacterium_callanderi_strain_DSM_3662_16S_ribosomal_RNA |
| SEQ ID NO: 196 | STR00122 | Bacteroides_intestinalis_strain_341_16S_ribosomal_RNA |
| SEQ ID NO: 197 | STR00105.6 | Dorea_longicatena_strain_111-35_16S_ribosomal_RNA |
| SEQ ID NO: 198 | STR00094 | Romboutsia_timonensis_strain_DR1_16S_ribosomal_RNA |
| SEQ ID NO: 199 | STR00106 | [Clostridium]_scindens_strain_ATCC_35704_16S_ribosomal_RNA |
| SEQ ID NO: 200 | STR00084 | Murimonas_intestini_strain_SRB-530-5-H_16S_ribosomal_RNA |
| SEQ ID NO: 201 | STR00115 | Blautia_producta_ATCC_27340_=_DSM_2950_strain_JCM_1471_16S_ribosomal_RNA |
| SEQ ID NO: 202 | STR00117 | Blautia_hominis_strain_KB1_16S_ribosomal_RNA |
| SEQ ID NO: 203 | STR00138 | Peptostreptococcus_stomatis_strain_W2278_16S_ribosomal_RNA |
| SEQ ID NO: 204 | STR00032 | Gemmiger_formicilis_strain_X2-56_16S_ribosomal_RNA |
| SEQ ID NO: 205 | STR00081.3 | Anaerotruncus_colihominis_strain_WAL_14565_16S_ribosomal_RNA |
| SEQ ID NO: 206 | STR00033 | Holdemania_filiformis_strain_J1-31B-1_16S_ribosomal_RNA |
| SEQ ID NO: 207 | STR00142 | Anaerofustis_stercorihominis_strain_WAL_14563_16S_ribosomal_RNA |
| SEQ ID NO: 208 | STR00140 | Blautia_luti_DSM_14534_16S_ribosomal_RNA |
| SEQ ID NO: 209 | STR00004 | Parabacteroides_merdae_strain_JCM_9497_16S_ribosomal_RNA |
| SEQ ID NO: 210 | STR00141 | [Clostridium]_lavalense_strain_CCRI-9842_16S_ribosomal_RNA |
| SEQ ID NO: 211 | STR00008 | Clostridium_tertium_strain_795_16S_ribosomal_RNA |
| SEQ ID NO: 212 | STR00009 | Clostridium_disporicum_strain_DS1_16S_ribosomal_RNA |
| SEQ ID NO: 213 | STR00064 | Harryflintia_acetispora_strain_V20-281a_16S_ribosomal_RNA |
| SEQ ID NO: 214 | STR00028 | Paraclostridium_bifermentans_strain_JCM_1386_16S_ribosomal_RNA |
| SEQ ID NO: 215 | STR00083.5 | Murimonas_intestini_strain_SRB-530-5-H_16S_ribosomal_RNA |
| SEQ ID NO: 216 | STR00130 | [Clostridium]_scindens_strain_ATCC_35704_16S_ribosomal_RNA |
| SEQ ID NO: 217 | STR00090.1 | Cellulosilyticum_lentocellum_DSM_5427_16S_ribosomal_RNA |
| SEQ ID NO: 218 | STR00065.1 | Drancourtella_massiliensis_strain_GD1_16S_ribosomal_RNA |
| SEQ ID NO: 219 | STR00072.1 | Clostridium_disporicum_strain_DS1_16S_ribosomal_RNA |
| SEQ ID NO: 220 | STR00110 | Paeniclostridium_sordellii_strain_JCM_3814_16S_ribosomal_RNA |
| SEQ ID NO: 221 | STR00019.1 | [Clostridium]_hylemonae_strain_TN-272_16S_ribosomal_RNA |
| SEQ ID NO: 222 | STR00010.7 | Blautia_wexlerae_DSM_19850_16S_ribosomal_RNA |
| SEQ ID NO: 223 | STR00053.1 | Bifidobacterium_longum_strain_KCTC_3128_16S_ribosomal_RNA |
| SEQ ID NO: 224 | STR00029 | Alistipes_shahii_WAL_8301_16S_ribosomal_RNA |
| SEQ ID NO: 225 | STR00023.1 | [Clostridium]_symbiosum_strain_ATCC_14940_16S_ribosomal_RNA |
| SEQ ID NO: 226 | STR00075.4 | Flavonifractor_plautii_strain_265_16S_ribosomal_RNA |
| SEQ ID NO: 227 | STR00074 | Roseburia_inulinivorans_DSM_16841_strain_A2-194_16S_ribosomal_RNA |
| SEQ ID NO: 228 | STR00103 | Flavonifractor_plautii_strain_265_16S_ribosomal_RNA |
| SEQ ID NO: 229 | STR00086.1 | Turicibacter_sanguinis_strain_MOL361_16S_ribosomal_RNA |
| SEQ ID NO: 230 | STR00049.1 | Eggerthella_lenta_strain_DSM_2243_16S_ribosomal_RNA |
| SEQ ID NO: 231 | STR00012.6 | [Clostridium]_innocuum_strain_B-3_16S_ribosomal_RNA |
| SEQ ID NO: 232 | STR00015 | Terrisporobacter_petrolearius_strain_LAM0A37_16S_ribosomal_RNA |
| SEQ ID NO: 233 | STR00076.6 | Blautia_hominis_strain_KB1_16S_ribosomal_RNA |
| SEQ ID NO: 234 | STR00087 | Faecalicatena_contorta_strain_DSM_3982_16S_ribosomal_RNA |
| SEQ ID NO: 235 | STR00096.6 | Faecalicatena_orotica_strain_DSM_1287_16S_ribosomal_RNA |
| SEQ ID NO: 236 | STR00095 | Intestinimonas_butyriciproducens_strain_SRB-521-5-I_16S_ribosomal_RNA |
| SEQ ID NO: 237 | STR00088.1 | Turicibacter_sanguinis_strain_MOL361_16S_ribosomal_RNA |
| SEQ ID NO: 238 | STR00088 | Turicibacter_sanguinis_strain_MOL361_16S_ribosomal_RNA |
| SEQ ID NO: 239 | STR00088.2 | Turicibacter_sanguinis_strain_MOL361_16S_ribosomal_RNA |
| SEQ ID NO: 240 | STR00088.3 | Turicibacter_sanguinis_strain_MOL361_16S_ribosomal_RNA |
| SEQ ID NO: 241 | STR00010 | Blautia_wexlerae_DSM_19850_16S_ribosomal_RNA |
| SEQ ID NO: 242 | STR00010.1 | Blautia_wexlerae_DSM_19850_16S_ribosomal_RNA |
| SEQ ID NO: 243 | STR00010.2 | Blautia_wexlerae_DSM_19850_16S_ribosomal_RNA |
| SEQ ID NO: 244 | STR00010.3 | Blautia_wexlerae_DSM_19850_16S_ribosomal_RNA |
| SEQ ID NO: 245 | STR00010.4 | Blautia_wexlerae_DSM_19850_16S_ribosomal_RNA |
| SEQ ID NO: 246 | STR00010.5 | Blautia_wexlerae_DSM_19850_16S_ribosomal_RNA |
| SEQ ID NO: 247 | STR00010.8 | Blautia_wexlerae_DSM_19850_16S_ribosomal_RNA |
| SEQ ID NO: 248 | STR00012 | [Clostridium]_innocuum_strain_B-3_16S_ribosomal_RNA |
| SEQ ID NO: 249 | STR00012.1 | [Clostridium]_innocuum_strain_B-3_16S_ribosomal_RNA |
| SEQ ID NO: 250 | STR00012.2 | [Clostridium]_innocuum_strain_B-3_16S_ribosomal_RNA |
| SEQ ID NO: 251 | STR00012.3 | [Clostridium]_innocuum_strain_B-3_16S_ribosomal_RNA |
| SEQ ID NO: 252 | STR00012.4 | [Clostridium]_innocuum_strain_B-3_16S_ribosomal_RNA |
| SEQ ID NO: 253 | STR00022 | [Clostridium]_bolteae_strain_JCM_12243_16S_ribosomal_RNA |
| SEQ ID NO: 254 | STR00022.1 | [Clostridium]_bolteae_strain_JCM_12243_16S_ribosomal_RNA |
| SEQ ID NO: 255 | STR00022.2 | [Clostridium]_bolteae_strain_JCM_12243_16S_ribosomal_RNA |
| SEQ ID NO: 256 | STR00022.3 | [Clostridium]_bolteae_strain_JCM_12243_16S_ribosomal_RNA |
| SEQ ID NO: 257 | STR00022.4 | [Clostridium]_bolteae_strain_JCM_12243_16S_ribosomal_RNA |
| SEQ ID NO: 258 | STR00025 | Eubacterium_callanderi_strain_DSM_3662_16S_ribosomal_RNA |
| SEQ ID NO: 259 | STR00025.1 | Eubacterium_callanderi_strain_DSM_3662_16S_ribosomal_RNA |
| SEQ ID NO: 260 | STR00025.2 | Eubacterium_callanderi_strain_DSM_3662_16S_ribosomal_RNA |
| SEQ ID NO: 261 | STR00025.3 | Eubacterium_callanderi_strain_DSM_3662_16S_ribosomal_RNA |
| SEQ ID NO: 262 | STR00025.4 | Eubacterium_callanderi_strain_DSM_3662_16S_ribosomal_RNA |
| SEQ ID NO: 263 | STR00075 | Flavonifractor_plautii_strain_265_16S_ribosomal_RNA |
| SEQ ID NO: 264 | STR00075.1 | Flavonifractor_plautii_strain_265_16S_ribosomal_RNA |
| SEQ ID NO: 265 | STR00075.2 | Flavonifractor_plautii_strain_265_16S_ribosomal_RNA |
| SEQ ID NO: 266 | STR00076 | Blautia_hominis_strain_KB1_16S_ribosomal_RNA |
| SEQ ID NO: 267 | STR00076.1 | Blautia_hominis_strain_KB1_16S_ribosomal_RNA |
| SEQ ID NO: 268 | STR00076.2 | Blautia_hominis_strain_KB1_16S_ribosomal_RNA |
| SEQ ID NO: 269 | STR00076.3 | Blautia_hominis_strain_KB1_16S_ribosomal_RNA |
| SEQ ID NO: 270 | STR00076.4 | Blautia_hominis_strain_KB1_16S_ribosomal_RNA |
| SEQ ID NO: 271 | STR00081 | Anaerotruncus_colihominis_strain_WAL_14565_16S_ribosomal_RNA |
| SEQ ID NO: 272 | STR00081.1 | Anaerotruncus_colihominis_strain_WAL_14565_16S_ribosomal_RNA |
| SEQ ID NO: 273 | STR00081.2 | Anaerotruncus_colihominis_strain_WAL_14565_16S_ribosomal_RNA |
| SEQ ID NO: 274 | STR00083 | Murimonas_intestini_strain_SRB-530-5-H_16S_ribosomal_RNA |
| SEQ ID NO: 275 | STR00083.1 | Murimonas_intestini_strain_SRB-530-5-H_16S_ribosomal_RNA |
| SEQ ID NO: 276 | STR00083.2 | Murimonas_intestini_strain_SRB-530-5-H_16S_ribosomal_RNA |
| SEQ ID NO: 277 | STR00083.3 | Murimonas_intestini_strain_SRB-530-5-H_16S_ribosomal_RNA |
| SEQ ID NO: 278 | STR00083.4 | Murimonas_intestini_strain_SRB-530-5-H_16S_ribosomal_RNA |
| SEQ ID NO: 279 | STR00092 | Erysipelatoclostridium_ramosum_strain_JCM_1298_16S_ribosomal_RNA |
| SEQ ID NO: 280 | STR00092.1 | Erysipelatoclostridium_ramosum_strain_JCM_1298_16S_ribosomal_RNA |
| SEQ ID NO: 281 | STR00092.2 | Erysipelatoclostridium_ramosum_strain_JCM_1298_16S_ribosomal_RNA |
| SEQ ID NO: 282 | STR00092.3 | Erysipelatoclostridium_ramosum_strain_JCM_1298_16S_ribosomal_RNA |
| SEQ ID NO: 283 | STR00092.4 | Erysipelatoclostridium_ramosum_strain_JCM_1298_16S_ribosomal_RNA |
| SEQ ID NO: 284 | STR00096 | Faecalicatena_orotica_strain_DSM_1287_16S_ribosomal_RNA |
| SEQ ID NO: 285 | STR00096.1 | Faecalicatena_orotica_strain_DSM_1287_16S_ribosomal_RNA |
| SEQ ID NO: 286 | STR00096.2 | Faecalicatena_orotica_strain_DSM_1287_16S_ribosomal_RNA |
| SEQ ID NO: 287 | STR00096.3 | Faecalicatena_orotica_strain_DSM_1287_16S_ribosomal_RNA |
| SEQ ID NO: 288 | STR00096.4 | Faecalicatena_orotica_strain_DSM_1287_16S_ribosomal_RNA |
| SEQ ID NO: 289 | STR00097.5 | Emergencia_timonensis_strain_SN18_16S_ribosomal_RNA |
| SEQ ID NO: 290 | STR00097.1 | Emergencia_timonensis_strain_SN18_16S_ribosomal_RNA |
| SEQ ID NO: 291 | STR00097.2 | Emergencia_timonensis_strain_SN18_16S_ribosomal_RNA |
| SEQ ID NO: 292 | STR00105 | Dorea_longicatena_strain_111-35_16S_ribosomal_RNA |
| SEQ ID NO: 293 | STR00105.2 | Dorea_longicatena_strain_111-35_16S_ribosomal_RNA |
| SEQ ID NO: 294 | STR00132 | Eisenbergiella_tayi_strain_B086562_16S_ribosomal_RNA |
| SEQ ID NO: 295 | STR00132.1 | Eisenbergiella_tayi_strain_B086562_16S_ribosomal_RNA |
| SEQ ID NO: 296 | STR00105.4 | Dorea_longicatena_strain_111-35_16S_ribosomal_RNA |
| SEQ ID NO: 297 | STR00105.5 | Dorea_longicatena_strain_111-35_16S_ribosomal_RNA |
| SEQ ID NO: 298 | STR00107 | [Clostridium]_aldenense_strain_RMA_9741_16S_ribosomal_RNA |
| SEQ ID NO: 299 | STR00107.1 | [Clostridium]_aldenense_strain_RMA_9741_16S_ribosomal_RNA |
| SEQ ID NO: 300 | STR00107.2 | [Clostridium]_aldenense_strain_RMA_9741_16S_ribosomal_RNA |
| SEQ ID NO: 301 | STR00107.3 | [Clostridium]_aldenense_strain_RMA_9741_16S_ribosomal_RNA |
| SEQ ID NO: 302 | STR00107.4 | [Clostridium]_aldenense_strain_RMA_9741_16S_ribosomal_RNA |
| SEQ ID NO: 303 | STR00132.8 | Eisenbergiella_tayi_strain_B086562_16S_ribosomal_RNA |
| SEQ ID NO: 304 | STR00132.2 | Eisenbergiella_tayi_strain_B086562_16S_ribosomal_RNA |
| SEQ ID NO: 305 | STR00132.3 | Eisenbergiella_tayi_strain_B086562_16S_ribosomal_RNA |
| SEQ ID NO: 306 | STR00132.6 | Eisenbergiella_tayi_strain_B086562_16S_ribosomal_RNA |
| SEQ ID NO: 307 | STR00139 | [Clostridium]_scindens_strain_ATCC_35704_16S_ribosomal_RNA |
| SEQ ID NO: 308 | STR00139.1 | [Clostridium]_scindens_strain_ATCC_35704_16S_ribosomal_RNA |
| SEQ ID NO: 309 | STR00096.8 | Faecalicatena_orotica_strain_DSM_1287_16S_ribosomal_RNA |
| SEQ ID NO: 310 | STR00096.9 | Faecalicatena_orotica_strain_DSM_1287_16S_ribosomal_RNA |
| SEQ ID NO: 311 | STR00139.2 | [Clostridium]_scindens_strain_ATCC_35704_16S_ribosomal_RNA |
| SEQ ID NO: 312 | STR00096.7 | Faecalicatena_orotica_strain_DSM_1287_16S_ribosomal_RNA |
| SEQ ID NO: 313 | STR00096.10 | Faecalicatena_orotica_strain_DSM_1287_16S_ribosomal_RNA |
| SEQ ID NO: 314 | STR00097.6 | Emergencia_timonensis_strain_SN18_16S_ribosomal_RNA |
| SEQ ID NO: 315 | STR00097 | Emergencia_timonensis_strain_SN18_16S_ribosomal_RNA |
| SEQ ID NO: 316 | STR00105.9 | Dorea_longicatena_strain_111-35_16S_ribosomal_RNA |
| SEQ ID NO: 317 | STR00105.1 | Dorea_longicatena_strain_111-35_16S_ribosomal_RNA |
| SEQ ID NO: 318 | STR00105.10 | Dorea_longicatena_strain_111-35_16S_ribosomal_RNA |
| SEQ ID NO: 319 | STR00105.3 | Dorea_longicatena_strain_111-35_16S_ribosomal_RNA |
| SEQ ID NO: 320 | STR00105.11 | Dorea_longicatena_strain_111-35_16S_ribosomal_RNA |
| SEQ ID NO: 321 | STR00105.8 | Dorea_longicatena_strain_111-35_16S_ribosomal_RNA |
| SEQ ID NO: 322 | STR00107.6 | [Clostridium]_aldenense_strain_RMA_9741_16S_ribosomal_RNA |
| SEQ ID NO: 323 | STR00107.7 | [Clostridium]_aldenense_strain_RMA_9741_16S_ribosomal_RNA |
| SEQ ID NO: 324 | STR00107.8 | [Clostridium]_aldenense_strain_RMA_9741_16S_ribosomal_RNA |
| SEQ ID NO: 325 | STR00107.9 | [Clostridium]_aldenense_strain_RMA_9741_16S_ribosomal_RNA |
| SEQ ID NO: 326 | STR00107.10 | [Clostridium]_aldenense_strain_RMA_9741_16S_ribosomal_RNA |
| SEQ ID NO: 327 | STR00132.9 | Eisenbergiella_tayi_strain_B086562_16S_ribosomal_RNA |
| SEQ ID NO: 328 | STR00132.11 | Eisenbergiella_tayi_strain_B086562_16S_ribosomal_RNA |
| SEQ ID NO: 329 | STR00132.10 | Eisenbergiella_tayi_strain_B086562_16S_ribosomal_RNA |
| SEQ ID NO: 330 | STR00132.7 | Eisenbergiella_tayi_strain_B086562_16S_ribosomal_RNA |
| SEQ ID NO: 331 | STR00139.3 | [Clostridium]_scindens_strain_ATCC_35704_16S_ribosomal_RNA |
| SEQ ID NO: 332 | STR00132.12 | [Clostridium]_scindens_strain_ATCC_35704_16S_ribosomal_RNA |
| SEQ ID NO: 333 | STR00139.7 | [Clostridium]_scindens_strain_ATCC_35704_16S_ribosomal_RNA |
| SEQ ID NO: 334 | STR00139.5 | [Clostridium]_scindens_strain_ATCC_35704_16S_ribosomal_RNA |
| SEQ ID NO: 335 | STR00139.6 | [Clostridium]_scindens_strain_ATCC_35704_16S_ribosomal_RNA |
| SEQ ID NO: 336 | STR00143 | Blautia_obeum_ATCC_29174_16S_ribosomal_RNA |
| SEQ ID NO: 337 | STR00143.1 | Blautia_obeum_ATCC_29174_16S_ribosomal_RNA |
| SEQ ID NO: 338 | STR00143.2 | Blautia_obeum_ATCC_29174_16S_ribosomal_RNA |
| SEQ ID NO: 339 | STR00143.3 | Blautia_obeum_ATCC_29174_16S_ribosomal_RNA |
| SEQ ID NO: 340 | STR00143.4 | Blautia_obeum_ATCC_29174_16S_ribosomal_RNA |
| SEQ ID NO: 341 | STR00143.5 | Blautia_obeum_ATCC_29174_16S_ribosomal_RNA |
| SEQ ID NO: 342 | STR00144 | Escherichia fergusonii ATCC 35469 16S ribosomal RNA |
| SEQ ID NO: 343 | STR00145 | Bifidobacterium dentiumSTRain B764 16S ribosomal RNA |
| SEQ ID NO: 344 | STR00146 | Streptococcus parasanguinis ATCC 15912 16S ribosomal RNA |
| SEQ ID NO: 345 | STR00147 | [Clostridium] spiroforme DSM 1552 16S ribosomal RNA |
| SEQ ID NO: 346 | STR00148 | Roseburia hominis A2-183 16S ribosomal RNA |
| SEQ ID NO: 347 | STR00149 | Holdemania filiformisSTRain J1-31B-1 16S ribosomal RNA |
| SEQ ID NO: 348 | STR00150 | [Clostridium] symbiosumSTRain ATCC 14940 16S ribosomal RNA |
| SEQ ID NO: 349 | STR00151 | Oscillibacter valericigenesSTRain Sjm18-20 16S ribosomal RNA |
| SEQ ID NO: 350 | STR00152 | Emergencia timonensisSTRain SN18 16S ribosomal RNA |
| SEQ ID NO: 351 | STR00153 | [Clostridium] leptumSTRain DSM 753 16S ribosomal RNA |
| SEQ ID NO: 352 | STR00154 | Flavonifractor plautiiSTRain 265 16S ribosomal RNA |

### EMBODIMENTS

Embodiment 1. A composition comprising a purified population of bacteria, wherein the purified population of bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1-341.

Embodiment 2. A composition comprising a purified population of bacteria, wherein the purified population of bacteria comprises Eubacterium maltosivorans, Clostridium aldenense, Clostridium bolteae, Clostridium glycyrrhizinilyticum, Clostridium hylemonae, Clostridium innocuum, Clostridium lavalense, Clostridium scindens, Clostridium spiroforme, Clostridium symbiosum, Eubacterium rectale, Ruminococcus gnavus, Ruminococcus torques, Absiella dolichum, Agathobaculum desmolans, Akkermansia muciniphila, Alistipes finegoldii, Alistipes shahii, Anaerofustis stercorihominis, Anaeromassilibacillus senegalensis, Anaerostipes caccae, Anaerotruncus colihominis, Bacteroides caccae, Faecalibacterium prausnitzii, Faecalicatena contorta, Faecalicatena orotica, Flavonifractor plautii, Gemmiger formicilis, Harryflintia acetispora, Holdemania filiformis, Holdemania massiliensis, Intestinimonas butyriciproducens, Lachnospira pectinoschiza, Lachnospiraceae bacterium 5 1 57FAA, Lactobacillus fermentum, Lactonifactor longoviformis, Longibaculum muris, Longicatena caecimuris, Murimonas intestina, Oscillibacter ruminantium, Bacteroides eggerthii, Bacteroides faecis, Bacteroides intestinalis, Bacteroides koreensis, Bacteroides kribbi, Bacteroides salyersiae, Bacteroides uniformis, Bacteroides vulgatus, Bacteroides xylanisolvens, Barnesiella intestinihominis, Bifidobacterium dentium, Bifidobacterium longum, Bifidobacterium stercoris, Blautia coccoides, Blautia hominis, Blautia hydrogenotrophica, Blautia luti, Blautia obeum, Blautia producta, Blautia wexlerae, Butyricimonas faecihominis, Cellulosilyticum lentocellum, Clostridium butyricum, Ruthenibacterium lactatiformans, Sellimonas intestinalis, Shigella flexneri, Terrisporobacter mayombei, Terrisporobacter petrolearius, Turicibacter sanguinis, Tyzzerella nexilis, Clostridium disporicum, Clostridium subterminale, Clostridium tertium, Collinsella aerofaciens, Coprococcus comes, Coprococcus eutactus, Dorea longicatena, Drancourtella massiliensis, Eggerthella lenta, Eisenbergiella tayi, Emergencia timonensis, Erysipelatoclostridium ramosum, Eubacterium callanderi, Paeniclostridium sordellii, Parabacteroides distasonis, Parabacteroides merdae, Paraclostridium bifermentans, Peptostreptococcus stomatis, Robinsoniella peoriensis, Romboutsia timonensis, Roseburia intestinalis, Roseburia inulinivorans, Ruminococcus albus, Ruminococcus bromii, Ruminococcus faecis, Ruminococcus lactaris, or combinations thereof.

Embodiment 3. A composition comprising a purified population of bacteria, wherein the purified population of bacteria comprises a species selected from FIG. 1 or combinations thereof.

Embodiment 4. The composition of any one of embodiments 1 to 3, wherein the purified population of bacteria comprises at least two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, or more bacteria.

Embodiment 5. A composition comprising a purified population of bacteria, wherein the composition comprises the purified population of bacteria selected from the DE1-DE54 recited in FIG. 1

Embodiment 6. The composition of any one of embodiments 1-5, wherein the composition can decrease an infection, including an infection caused by an ESKAPE pathogen (Enterococcus faecium, Staphylococcus aureus, Klebsiella pneumoniae, Acinetobacter baumannii, Pseudomonas aeruginosa, and Enterobacter spp.) Embodiment 7. The composition of any one of embodiments 1 to 6, wherein the purified population of bacteria can decrease the number and/or relative abundance of antibiotic resistant bacteria and/or an ESKAPE pathogen in a gastrointestinal tract of a subject compared to a reference (e.g., composition that does not include one or more of the bacteria disclosed herein).

Embodiment 8. The composition of any one of embodiments 1 to 7, wherein the number of antibiotic resistant bacteria is measured as a colony forming unit per gram of a sample obtained from the subject.

Embodiment 9. The composition of any one of embodiments 1 to 8, wherein:
(i) the antibiotic resistant bacteria comprise vancomycin-resistant Enterococci or carbapenem-resistant Enterobacteriaceae or a combination thereof;
(ii) the ESKAPE pathogen comprises Enterococcus faecium, Staphylococcus aureus, Klebsiella pneumoniae, Acinetobacter baumannii, Pseudomonas aeruginosa, and Enterobacter spp., or a combination thereof; or
(iii) the antibiotic resistant bacteria and ESKAPE pathogen are selected from (i) and (ii).

Embodiment 10. The composition of any one of embodiments 1 to 9, wherein one or more bacteria of the purified population of bacteria can compete for carbon-sources with the antibiotic resistant bacteria.

Embodiment 11. The composition of any one of embodiments 1 to 10, wherein the purified population of bacteria can improve epithelial barrier integrity, reduce inflammation, and/or reduce mucositis in a gastrointestinal tract of a subject compared to a reference (e.g., a corresponding reference in a subject that did not receive the composition disclosed herein or corresponding reference in the subject prior to the administration of the composition).

Embodiment 12. The composition of any one of embodiments 1 to 11, wherein the purified population of bacteria can decrease mortality due to an invasive infection in a subject compared to a reference (e.g., a corresponding reference in a subject that did not receive the composition disclosed herein or corresponding reference in the subject prior to the administration of the composition).

Embodiment 13. The composition of embodiment 12, wherein the invasive infection in the subject is an antibiotic resistant infection.

Embodiment 14. The composition of any one of embodiments 1 to 13, wherein the purified population of bacteria can reduce transplantation-related complications in a subject compared to a reference (e.g., a corresponding reference in a subject that did not receive the composition disclosed herein or corresponding reference in the subject prior to the administration of the composition).

Embodiment 15. The composition of any one of embodiments 1 to 14, wherein the purified population of bacteria can increase the overall survival and/or progression-free survival of a subject compared to a reference (e.g., composition that does not include one or more of the bacteria disclosed herein).

Embodiment 16. The composition of any one of embodiments 1 to 15, wherein the purified population of bacteria can modulate a biological activity, wherein the biological activity comprises short-chain fatty acid production, medium-chain fatty acid production, tryptophan metabolite production, fucosidase activity, Wnt activation, anti-IL-8 activity, carbon source utilization, bile acid metabolism, or combinations thereof.

Embodiment 17. The composition of any one of embodiments 1 to 16, wherein the purified population of bacteria comprises one or more bacteria having one or more features selected from the group consisting of:
(1) capable of reducing VRE and CRE carriage and restore colonization resistance in the GI tract of a mammal;
(2) capable of protecting the epithelial barrier from cytokine-mediated inflammatory damage; and
(3) capable of reducing inflammation in the epithelial barrier, as measured by IL-8 secretion in vitro, and in the colonic lamina propria of mice.

Embodiment 18. The composition of any one of embodiments 1 to 17, wherein the purified population of bacteria comprises one or more bacteria having one or more features selected from the group consisting of:
(i) capable of engrafting when administered to a subject, (ii) capable of having anti-inflammatory activity, (iii) not capable of inducing pro-inflammatory activity, (iv) capable of producing a secondary bile acid (7α-deydroxylase and bile salt hydrolase activity), (v) capable of producing a tryptophan metabolite (e.g., indole, 3-methyl indole, indolepropionic acid), (vi) capable of restoring epithelial integrity as determined by a primary epithelial cell monolayer barrier integrity assay, (vii) capable of being associated with remission of infection or GvHD following HSCT, (viii) capable of not being associated with clinical non-remission of infection or GvHD following HSCT, (ix) capable of producing a short-chain fatty acid (e.g., butyrate, propionate), (x) capable of inhibiting a HDAC activity, (xi) capable of producing a medium-chain fatty acid (e.g., valerate, hexanoate), (xii) capable of expressing catalase activity, (xiii) capable of having alpha-fucosidase activity, (xiv) capable of inducing Wnt activation, (xv) capable of producing a B vitamin, (xvi) capable of reducing fecal calprotectin level, (xviii) not capable of activating a toll-like receptor pathway (e.g., TLR4 or TLR5), (xix) capable of activating a toll-like receptor pathway (e.g., TLR2), or (xx) any combination thereof, (xxi) capable of restoring colonization resistance, (xxii) capable of a broad range of carbon source utilization, (xxiii) capable of reducing VRE pathogen carriage, (xxiv) capable of reducing CRE pathogen carriage; (xxv) capable of reducing expression of claudin-2, (xxvi) capable of being associated with the healthy human gut microbiota, (xxvii) capable of not being associated with toxin and hemolysin genes associated with Clostridial pathogens and no significant cytopathic effects in vitro, (xxviii) susceptible to multiple clinically relevant antibiotics, (xxix) capable of not being associated with genes that are both likely responsible for the observed antibiotic resistances and transmissible, and (xxx) any combination thereof.

Embodiment 19. The composition of embodiment 16, wherein the biological activity is modulated in vivo.

Embodiment 20. The composition of embodiment 16, wherein the biological activity is modulated in vitro (e.g., a culture or a synthetic gastrointestinal system).

Embodiment 21. The composition of any one of embodiments 16 to 20, wherein short-chain fatty acid production is increased by at least 10%, at least 20%, at least 30%, at least 40%, at least 50%, at least 60%, at least 70%, at least 80%, or at least 90% compared to a reference (e.g., composition that does not include one or more of the bacteria disclosed herein).

Embodiment 22. The composition of any one of embodiments 16 to 21, wherein medium-chain fatty acid production is increased by at least 10%, at least 20%, at least 30%, at least 40%, at least 50%, at least 60%, at least 70%, at least 80%, or at least 90% compared to a reference (e.g., composition that does not include one or more of the bacteria disclosed herein).

Embodiment 23. The composition of any one of embodiments 16 to 22, wherein tryptophan metabolite production is increased by at least 10%, at least 20%, at least 30%, at least 40%, at least 50%, at least 60%, at least 70%, at least 80%, or at least 90% compared to a reference (e.g., composition that does not include one or more of the bacteria disclosed herein).

Embodiment 24. The composition of any one of embodiments 16 to 23, wherein fucosidase activity is increased by at least 10%, at least 20%, at least 30%, at least 40%, at least 50%, at least 60%, at least 70%, at least 80%, or at least 90% compared to a reference (e.g., composition that does not include one or more of the bacteria disclosed herein).

Embodiment 25. The composition of any one of embodiments 16 to 24, wherein Wnt activation is increased by at least 10%, at least 20%, at least 30%, at least 40%, at least 50%, at least 60%, at least 70%, at least 80%, or at least 90% compared to a reference (e.g., composition that does not include one or more of the bacteria disclosed herein).

Embodiment 26. The composition of any one of embodiments 16 to 25, wherein anti-IL-8 activity is increased by at least 10%, at least 20%, at least 30%, at least 40%, at least 50%, at least 60%, at least 70%, at least 80%, or at least 90% compared to a reference (e.g., composition that does not include one or more of the bacteria disclosed herein).

Embodiment 27. The composition of any one of embodiments 1 to 26, wherein the purified population of bacteria can augment the number and/or relative abundance of spore-forming bacteria in a microbiome of a subject.

Embodiment 28. The composition of any one of embodiments 1 to 27, wherein the purified population of bacteria can augment the number and/or relative abundance of non-pathogenic, commensal non-spore-forming bacteria in a microbiome of a subject.

Embodiment 29. The composition of any one of embodiments 1 to 28, wherein one or more bacteria of the purified population of bacteria are capable of being engrafted into a subject's microbiome when administered to the subject, wherein said engraftment is long-term or transient engraftment.

Embodiment 30. A pharmaceutical formulation comprising the composition of any one of embodiments 1 to 28 and a pharmaceutically acceptable excipient.

Embodiment 31. The pharmaceutical formulation of embodiment 30, wherein the excipient comprises glycerol.

Embodiment 32. The pharmaceutical formulation of embodiment 30 or 31, wherein the composition is lyophilized.

Embodiment 33. The pharmaceutical formulation of any one of embodiments 30 to 32, wherein the composition is formulated for oral delivery.

Embodiment 34. A method of treating a disease or disorder associated with an allogeneic immune response in a subject in need thereof, comprising administering to the subject an effective amount of a composition of any one of embodiments 1 to 29 or a pharmaceutical formulation of any one of embodiments 30 to 33.

Embodiment 35. The method of embodiment 34, wherein the allogeneic immune response is caused by an allogeneic hematopoietic stem cell transplantation (allo-HSCT) or an allogeneic organ transplantation.

Embodiment 36. The method of embodiment 34 or 35, wherein the disease or disorder associated with an allogenic immune response comprises graft-versus-host-disease (GvHD), viral infection or reactivation, invasive infection, blood stream infection, inflammation, or combinations thereof.

Embodiment 37. The method of embodiment 36, wherein the GvHD comprises an acute graft versus host disease (aGvHD) or a chronic graft versus host disease (cGvHD).

Embodiment 38. The method of any one of embodiments 34 to 37, wherein the subject suffers from a cancer.

Embodiment 39. The method of embodiment 38, wherein the cancer comprises acute myeloid leukemia (AML), acute lymphoblastic leukemia (ALL), myelodysplastic syndromes (MDS), myeloproliferative neoplasms (MPN), or combinations thereof

Embodiment 40. A method of treating, reducing, or alleviating a symptom associated with chemotherapy in a subject in need thereof, comprising administering to the subject an effective amount of a composition of any one of embodiments 1 to 29 or a pharmaceutical formulation of any one of embodiments 30 to 33.

Embodiment 41. The method of embodiment 40, wherein the symptom associated with chemotherapy comprises weight loss or an increase in the level of a proinflammatory mediator within a gastrointestinal tract of the subject.

Embodiment 42. The method of embodiment 41, wherein the weight loss is reduced by at least 10%, at least 20%, at least 30%, at least 40%, at least 50%, at least 60%, at least 70%, at least 80%, or at least 90% compared to a reference (e.g., corresponding value in a subject that did not receive the composition disclosed herein or corresponding value in the subject prior to the administration of the composition).

Embodiment 43. The method of embodiment 41, wherein the level of a proinflammatory mediator is reduced by at least 10%, at least 20%, at least 30%, at least 40%, at least 50%, at least 60%, at least 70%, at least 80%, or at least 90% compared to a reference (e.g., corresponding value in a subject that did not receive the composition disclosed herein or corresponding value in the subject prior to the administration of the composition).

Embodiment 44. The method of any one of embodiments 41 to 43, wherein the proinflammatory mediator comprises IFN-γ, IL-1b, IL-2, IL-6, IL-12, CXCL5, IL-17, CXCL1, VEGF, TNF-α, or combinations thereof.

Embodiment 45. The method of embodiment 44, wherein the level of a proinflammatory T cell is reduced by at least 10%, at least 20%, at least 30%, at least 40%, at least 50%, at least 60%, at least 70%, at least 80%, or at least 90% compared to a reference (e.g., corresponding value in a subject that did not receive the composition disclosed herein or corresponding value in the subject prior to the administration of the composition).

Embodiment 46. The method of embodiment 45, wherein the proinflammatory T cell comprises a CD8⁺ T cell.

Embodiment 47. The method of embodiment 46, wherein the level of an anti-inflammatory T cell is reduced by at least 10%, at least 20%, at least 30%, at least 40%, at least 50%, at least 60%, at least 70%, at least 80%, or at least 90% compared to a reference (e.g., corresponding value in a subject that did not receive the composition disclosed herein or corresponding value in the subject prior to the administration of the composition).

Embodiment 48. The method of embodiment 47, wherein the anti-inflammatory T cell comprises a FOXP3⁺ CD4⁺ T cell.

Embodiment 49. A method of preventing, reducing, or treating rejection in a subject undergoing transplantation (e.g., either HSCT or organ), comprising administering to the subject an effective amount of a composition of any one of embodiments 1 to 29 or a pharmaceutical formulation of any one of embodiments 30 to 33.

Embodiment 50. The method of embodiment 49, wherein the composition or the pharmaceutical formulation is administered to the subject prior to, during, and/or after the transplantation.

Embodiment 51. A method of modulating a biological activity in a subject in need thereof, comprising administering to the subject an effective amount of a composition of any one of embodiments 1 to 29 or a pharmaceutical formulation of any one of embodiments 30 to 33, wherein the biological activity comprises short-chain fatty acid production, medium-chain fatty acid production, tryptophan metabolite production, fucosidase activity, Wnt activation, anti-IL-8 activity, or combinations thereof.

Embodiment 52. The method of embodiment 51, wherein short-chain fatty acid production is increased by at least 10%, at least 20%, at least 30%, at least 40%, at least 50%, at least 60%, at least 70%, at least 80%, or at least 90% compared to a reference (e.g., corresponding activity in a subject that did not receive the composition disclosed herein or corresponding activity in the subject prior to the administration of the composition).

Embodiment 53. The method of embodiment 51, wherein medium-chain fatty acid production is increased by at least 10%, at least 20%, at least 30%, at least 40%, at least 50%, at least 60%, at least 70%, at least 80%, or at least 90% compared to a reference (e.g., corresponding activity in a subject that did not receive the composition disclosed herein or corresponding activity in the subject prior to the administration of the composition).

Embodiment 54. The method of embodiment 51, wherein tryptophan metabolite production is increased by at least 10%, at least 20%, at least 30%, at least 40%, at least 50%, at least 60%, at least 70%, at least 80%, or at least 90% compared to a reference (e.g., corresponding activity in a subject that did not receive the composition disclosed herein or corresponding activity in the subject prior to the administration of the composition).

Embodiment 55. The method of embodiment 51, wherein fucosidase activity is increased by at least 10%, at least 20%, at least 30%, at least 40%, at least 50%, at least 60%, at least 70%, at least 80%, or at least 90% compared to a reference (e.g., corresponding activity in a subject that did not receive the composition disclosed herein or corresponding activity in the subject prior to the administration of the composition).

Embodiment 56. The method of embodiment 51, wherein Wnt activation is increased by at least 10%, at least 20%, at least 30%, at least 40%, at least 50%, at least 60%, at least 70%, at least 80%, or at least 90% compared to a reference (e.g., corresponding activity in a subject that did not receive the composition disclosed herein or corresponding activity in the subject prior to the administration of the composition).

Embodiment 57. The method of embodiment 51, wherein anti-IL-8 activity is increased by at least 10%, at least 20%, at least 30%, at least 40%, at least 50%, at least 60%, at least 70%, at least 80%, or at least 90% compared to a reference (e.g., corresponding activity in a subject that did not receive the composition disclosed herein or corresponding activity in the subject prior to the administration of the composition).

Embodiment 58. A method of decreasing the number and/or relative abundance of antibiotic resistant bacteria in a gastrointestinal tract of a subject in need thereof, comprising administering to the subject an effective amount of a composition of any one of embodiments 1 to 29 or a pharmaceutical formulation of any one of embodiments 30 to 33.

Embodiment 59. The method of embodiment 58, wherein the number and/or abundance of antibiotic resistant bacteria is reduced by at least 10%, at least 20%, at least 30%, at least 40%, at least 50%, at least 60%, at least 70%, at least 80%, or at least 90% compared to a reference (e.g., corresponding number in a subject that did not receive the composition disclosed herein or corresponding number in the subject prior to the administration of the composition).

Embodiment 60. The method of embodiment 58 or 59, wherein the antibiotic resistant bacteria comprise vancomycin-resistant Enterococci or carbapenem-resistant Enterobacteriaceae.

Embodiment 61. A method of improving epithelial barrier status, reducing inflammation, and/or reducing mucositis in a gastrointestinal tract of a subject in need thereof, comprising administering to the subject an effective amount of a composition of any one of embodiments 1 to 29 or a pharmaceutical formulation of any one of embodiments 30 to 33.

Embodiment 62. The method of embodiment 61, wherein the epithelial barrier status in the gastrointestinal tract of the subject is improved by at least 5%, at least 10%, at least 20%, at least 30%, at least 40%, at least 50%, at least 60%, at least 70%, at least 80%, or at least 90% compared to a reference (e.g., corresponding value in a subject that did not receive the composition disclosed herein or corresponding activity in the subject prior to the administration of the composition).

Embodiment 63. The method of embodiment 61, wherein the inflammation in the gastrointestinal tract of the subject is reduced by at least 5%, at least 10%, at least 20%, at least 30%, at least 40%, at least 50%, at least 60%, at least 70%, at least 80%, or at least 90% compared to a reference (e.g., corresponding value in a subject that did not receive the composition disclosed herein or corresponding activity in the subject prior to the administration of the composition).

Embodiment 64. The method of embodiment 61, wherein the mucositis in the gastrointestinal tract of the subject is reduced by at least 5%, at least 10%, at least 20%, at least 30%, at least 40%, at least 50%, at least 60%, at least 70%, at least 80%, or at least 90% compared to a reference (e.g., corresponding value in a subject that did not receive the composition disclosed herein or corresponding activity in the subject prior to the administration of the composition).

Embodiment 65. A method of decreasing mortality due to an invasive infection in a subject in need thereof, comprising administering to the subject an effective amount of a composition of any one of embodiments 1 to 29 or a pharmaceutical formulation of any one of embodiments 30 to 33, wherein the subject is undergoing transplantation.

Embodiment 66. The method of embodiment 65, wherein the invasive infection in the subject is an antibiotic resistant infection.

Embodiment 67. A method of reducing transplantation-related complications in a subject in need thereof, comprising administering to the subject an effective amount of a composition of any one of embodiments 1 to 29 or a pharmaceutical formulation of any one of embodiments 30 to 33, wherein the subject is undergoing transplantation.

Embodiment 68. A method of increasing the overall survival and/or progression-free survival of a subject in need thereof, comprising administering to the subject an effective amount of a composition of any one of embodiments 1 to 29 or a pharmaceutical formulation of any one of embodiments 30 to 33, wherein the subject is undergoing transplantation.

Embodiment 69. The method of any one of embodiments 34 to 68, wherein the subject is undergoing or has undergone transplantation.

Embodiment 70. The method of embodiment 69, wherein the transplantation is an allogeneic hematopoietic stem cell transplantation (allo-HSCT) or an allogeneic organ transplantation.

Embodiment 71. The method of any one of embodiments 34 to 70, wherein the subject suffers from a cancer.

Embodiment 72. The method of embodiment 71, wherein the cancer comprises acute myeloid leukemia (AML), acute lymphoblastic leukemia (ALL), myelodysplastic syndromes (MDS), myeloproliferative neoplasms (MPN), or combinations thereof.

The following examples are offered by way of illustration and not by way of limitation. The contents of all references cited throughout this application are expressly incorporated herein by reference.

### EXAMPLES

### Example 1: Designing Bacterial Compositions and Screening for Functional Properties

In designing the bacterial compositions of the present disclosure, the compositions were constructed to have one or more of the following features: (1) include phylogenetically diverse species to enable reduction of pathogen carriage and restoration of colonization resistance; (2) restore integrity of the gastrointestinal epithelial barrier through provision of microbially associated metabolites; (3) reduce inflammation in the gastrointestinal epithelial barrier and lamina propria. To do so, bacterial species exhibiting one or more of the following properties were considered in designing the bacterial compositions: (i) ability to utilize a carbon source used by a pathogenic organism, such as but not limited to Enterococcus and Enterobacteriaceae species and ESKAPE pathogens (including: Enterococcus faecium, Staphylococcus aureus, Klebsiella pneumoniae, Acinetobacter baumannii, Pseudomonas aeruginosa, and Enterobacter spp.) Enterococcus species including, but not limited to, Enterococcus faecalis and Enterococcus faecium, Enterobacteriaceae species including, but not limited to Klebsiella pneumonia, or such species that are resistant to vancomycin or carbapenems, drug resistant or multi-drug resistant (MDROs) including VRE, CRE (Klebsiella pneumonia, Klebsiella oxytoca, Klebsiella aerogenes, Enterococcus species), extended spectrum beta-lactamase (ESBLs) producing bacteria (E. coli, Klebsiella species), or methicillin-resistant Staphylococcus aureus (MRSA); (ii) ability to engraft when administered to a subject; (iii) ability to produce short-chain fatty acids; (iv) ability to produce medium-chain fatty acids; (v) ability to produce tryptophan metabolites; (vi) ability to inhibit histone deacetylase (HDAC) activity; (vii) ability to decrease IL-8 secretion in intestinal epithelial cells (IECs) treated with TNF-α; (viii) lack of induction of IL8 secretion in intestinal epithelial cells (IECs) in the absence of TNF-α; and (ix) combinations thereof. Bacterial species with pro-inflammatory activity (e.g., able to induce IL-8 secretion in IECs) were specifically excluded. As described further below, individual bacterial strains were evaluated using a panel of in vitro assays for functions supporting one or more of the desired properties noted above.

The criteria resulted in greater than 60 candidate bacterial compositions, including more than 150 species. Candidate compositions were screened in vitro and in vivo for their ability to: (1) reduce VRE and CRE carriage and restore colonization resistance in the GI tract of mice; (2) protect the epithelial barrier from cytokine-mediated inflammatory damage (e.g., IFN-γ mediated); (3) reduce inflammation in the epithelial barrier, as measured by IL-8 secretion and modulation of inflammatory pathway gene expression in vitro, and in the colonic lamina propria of mice, e.g., as measured through an increased ratio of Treg cells to pro-inflammatory Th1 and Th17 cells.

The designed bacterial compositions were mixed in equal ratios at ˜1-5×10⁷ colony forming units (CFU)/mL of vegetative bacteria and ˜1×10⁴-1×10⁵ CFU/mL of spore forming bacteria (when relevant) and frozen in 15% glycerol. For cultivation, the bacterial compositions were thawed, the glycerol was removed and the mix germinated in 0.5% BHIS/Oxgall for 1 hour at room temperature when they contained spore preparations. Compositions containing vegetative bacteria did not undergo germination. The germinant was then washed out and the cultures diluted to a final concentration of 5×10⁷ cfu/mL and plated as biological replicates in a synthetically derived, fecal culture medium 4 (FCM4), that supports growth of many anaerobic gut bacteria.

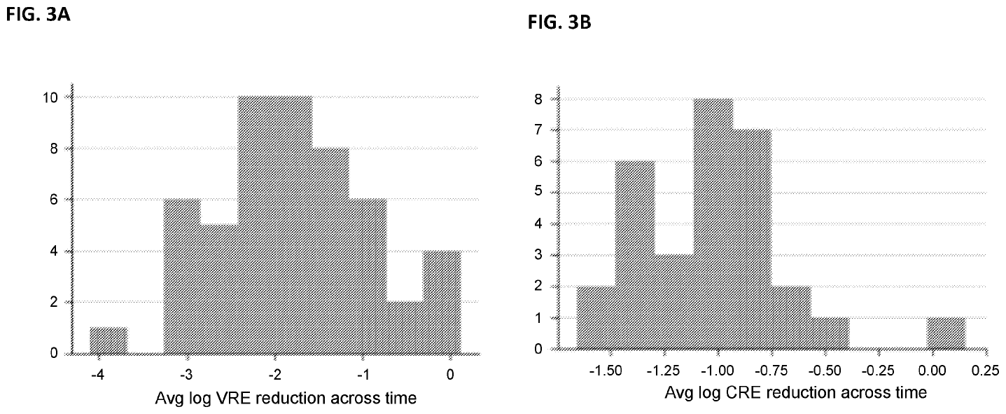

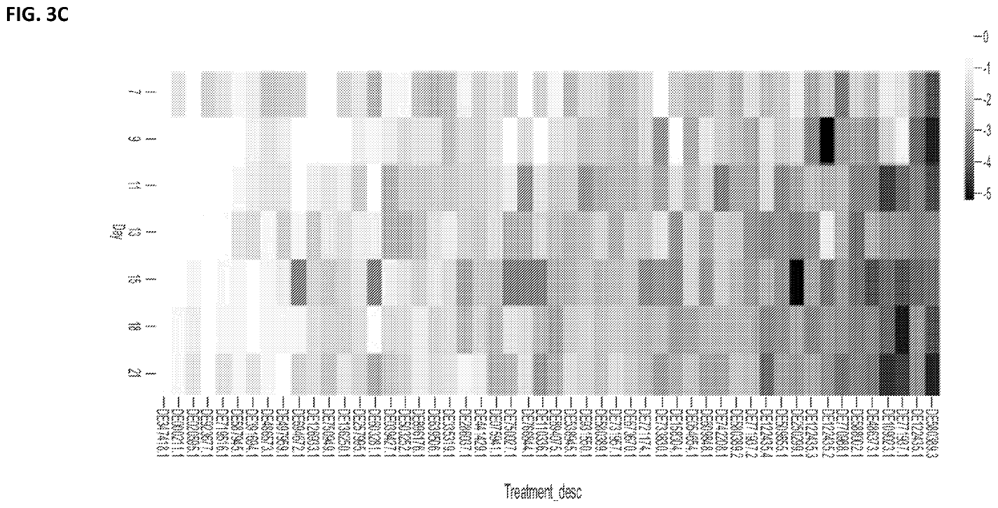

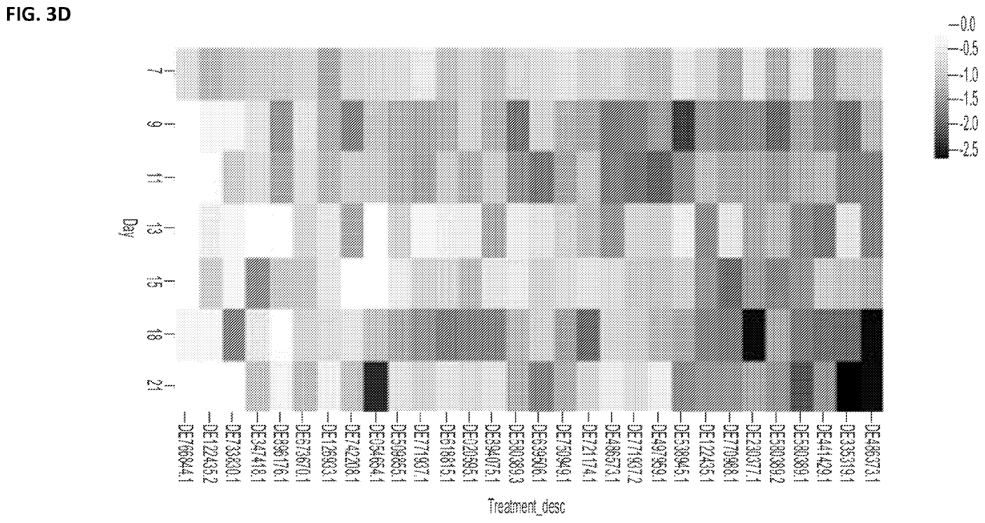

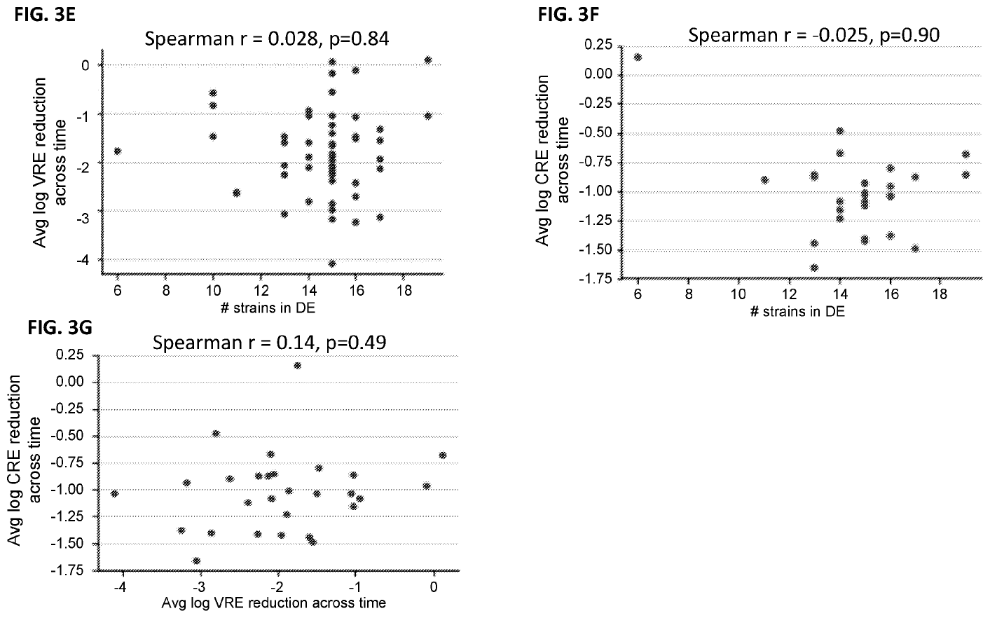

In experiments to evaluate the abilities to reduce VRE and CRE carriage and restore colonization resistance in the gut by the designed bacterial compositions, over 50 unique compositions were tested. The DEs tested and their strain composition are shown in FIG. 1 and the diversity of the DEs tested in FIGS. 2A, 2B and 2C. Briefly, mice were first conditioned with antibiotics to disrupt the native microbiome and impair colonization resistance. Mice were then challenged on Day 0 with either a vancomycin-resistant isolate of Enterococcus faecium (ATCC 700221) or a carbapenem-resistant isolate of Klebsiella pneumoniae to achieve high titer (up to 10¹⁰ CFU/g feces) carriage and intestinal domination by VRE or CRE, respectively. Daily treatment via oral gavage on Days 2, 3, and 4 with the DEs led to a greater than 2 log reduction in VRE or CRE titers in the feces relative to mice treated with vehicle, which maintained high VRE or CRE titers for up to several weeks (FIGS. 3A-3D). Further analysis of the log reduction in VRE or CRE titers by the DEs using the Spearman's correlation test indicated that the number of strains in a DE is not correlated with the VRE (r=0.028, p=0.84) or CRE (r=−0.025, p=0.90) decolonization as shown in FIGS. 3E and 3F, respectively. The Spearman's correlation test of the log reduction in VRE and that in CRE (r=0.14, p=0.49, as shown in FIG. 3G) suggests that the VRE and CRE titer reduction by a DE is not correlated, i.e., VRE and CRE decolonization in the animal models are driven by distinct factors.

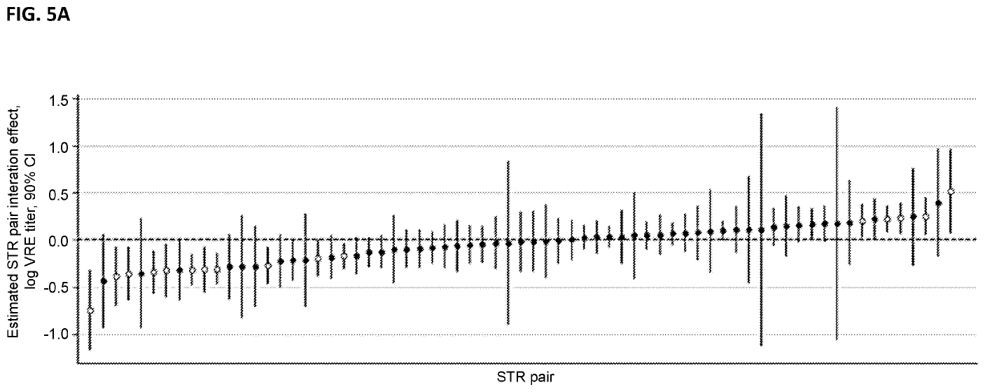

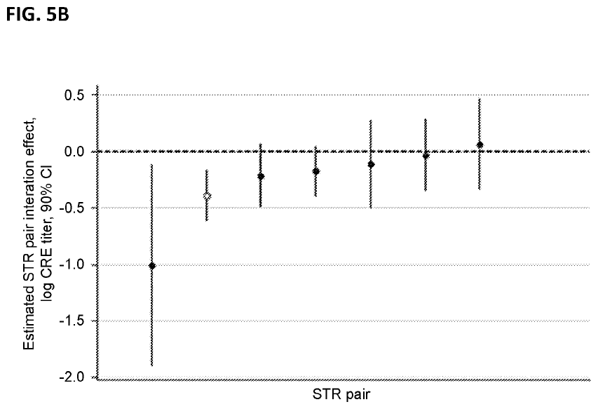

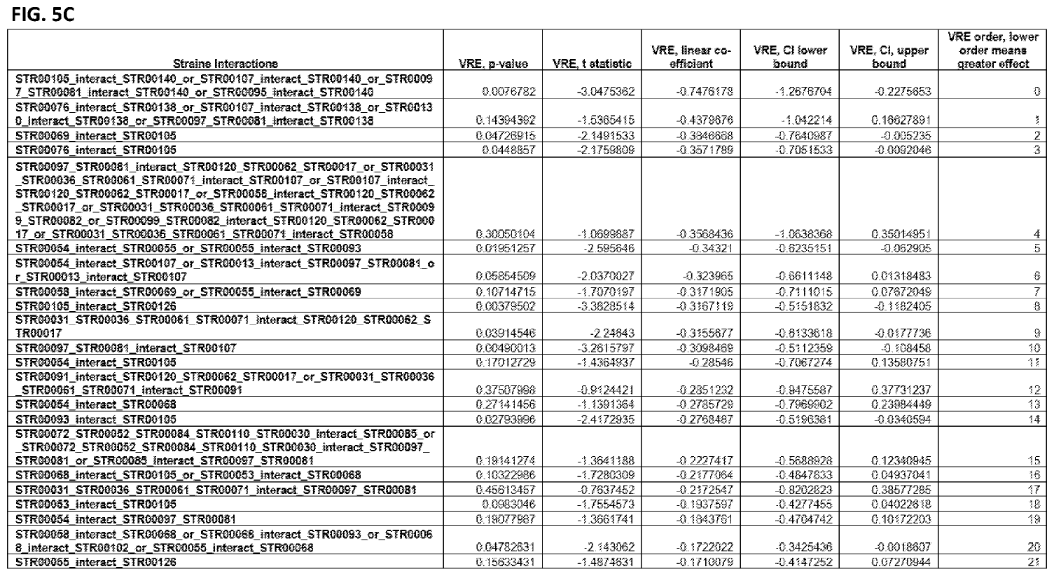

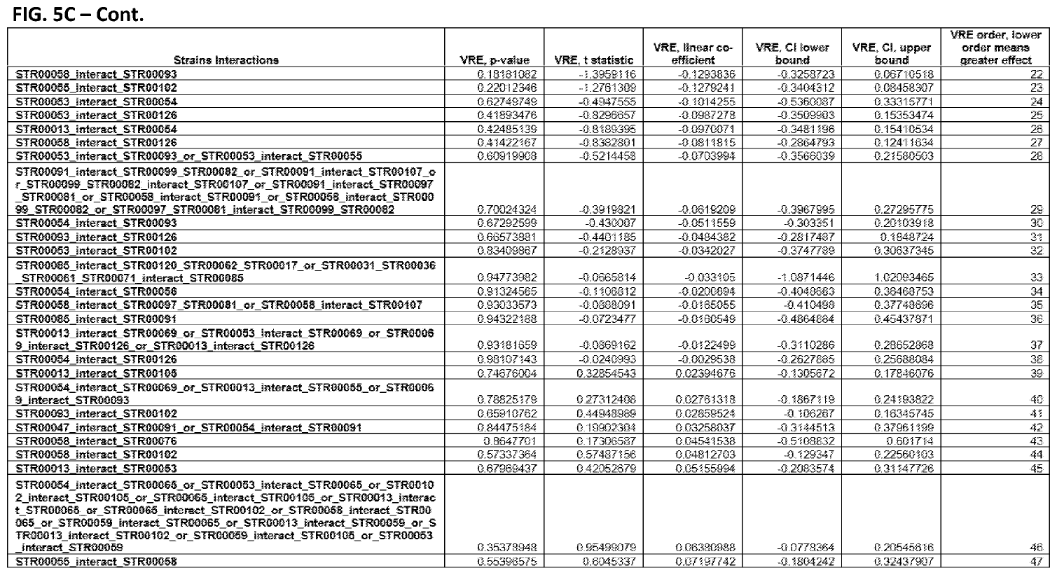

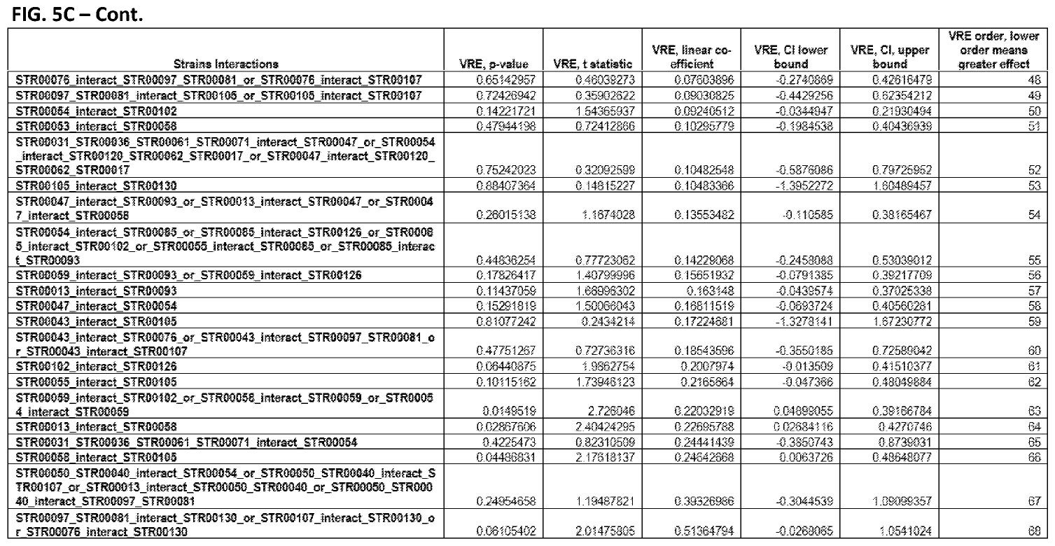

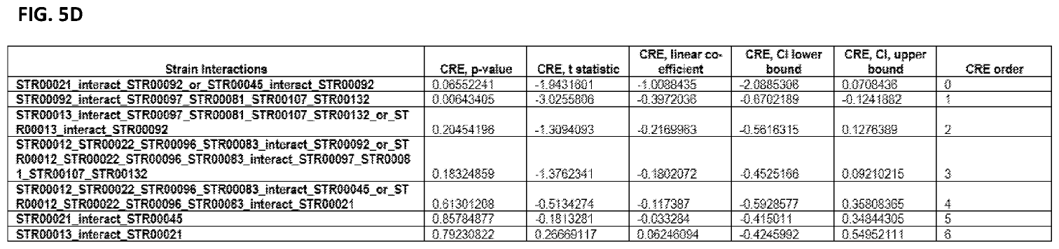

Statistical models were used to assess the effects of adding/removing individual strains in the DEs and the effects of strain interactions (e.g. synergy, antagonism), which usually require a prohibitive number of combinatorial experiments. A single strain additive model was employed to assess the estimated additive effect of each strain on VRE/CRE decolonization, after accounting for the additive effects of other strains, as shown in FIGS. 4A-4F. A strain interaction model was adopted to analyze pairwise (synergistic and antagonistic) interactions between strains on VRE/CRE decolonization, accounting for differences in what would be expected based on single strain additive effects, as shown in FIGS. 5A-5D. Strains and strain combinations with significant effects were selected for candidate DEs.

In experiments where secondary bile acid production by bacterial communities was assayed, FCM4 was supplemented with conjugated primary bile acids (glycocholic acid, taurocholic acid, glycochenodeoxycholic acid and taurochenodeoxycholic acid) at a final concentration of 100 uM. Bacterial cultures were incubated anaerobically at 37° C. for 7 days, after which their biomass was measured by absorbance of 100 μL culture at 600 nm. The remaining culture was centrifuged at 4000 rpm, the supernatants passed through a 0.2 μm filter and used in biochemical and cell-based assays. HDAC inhibition assays, pro-inflammatory assay in IECs, anti-inflammatory assay in IECs, epithelial integrity assay, macrophage and t-cell activation and cytotoxicity assays, determination of SCFAs, MCFAs, and tryptophan metabolites were performed. For determination of bile acid metabolites, 100 μL, of bacterial cell-free supernatant was then extracted with an equal volume of acetonitrile and filtered through a 0.2 μm filter, generating samples for LC-MS analysis. Bile acids were separated using an Agilent 1260 HPLC equipped with a Microsolv bidentate C18 column preceded by a 0.2 μm pre-column filter. Separation was achieved using a water and acetonitrile gradient with 0.1% formic acid at a flow rate of 0.4 ml/minute. Samples were injected at a volume of 5 μL. The HPLC system was coupled to a Bruker Compass™ qTOF mass spectrometer calibrated to a mass range of 50 to 1700 m/z using the Agilent low-mass tuning mix. Each run was additionally calibrated to a reference mass solution injected at the beginning of each run. Bile acids were detected in negative mode and identified by unique m/z and retention times compared to known pure standards. Area under the peak was determined using Bruker data analysis software. Metabolites were quantified using calibration curves generated from pure standards, ranging in concentration from 0.001 μM to 100 μM.

### Engraftment Analysis

Table 1 shows engraftment analysis of bacterial species in a DE (DE122435.3) described herein. In particular, Table 1 identifies engraftment of such species when administered to a human subject suffering from ulcerative colitis (UC) and/or recurrent Clostridium difficile infection (rCDI) and associated efficacy.

TABLE 1
Observed Engraftment And Association with Efficacy of DE122435.3
species (or strains) in previous clinical trials

| | | | Strain | Species Associated |
|---|---|---|---|---|
| | Species Engraftment | | Engraftment | with Efficacy |
| STR | rCDI | UC | CDI | rCDI or UC |
| STR00010-STR00010.7 | x | x | n/a | x |
| STR00012-STR00012.6 | x | x | x | x |
| STR00022-STR00022.5 | | x | x | x |
| STR00025-STR00025.5 | | | n/a | |
| STR00075-STR00075.4 | x | x | x | x |
| STR00076-STR00076.6 | | x | x | |
| STR00081-STR00081.5 | | x | n/a | |
| STR00083-STR00083.5 | | x | x | |
| STR00092-STR00092.6 | | x | n/a | |
| STR00096-STR00096.6 | | | x | |
| STR00097-STR00097.4 | | x | n/a | |
| STR00105-STR00105.7 | x | x | n/a | x |
| STR00107-STR00107.5 | | x | n/a | |
| STR00132-STR00132.5 | | | n/a | |
| STR00139-STR00139.4 | | x | n/a | |
| STR00143-STR00143.6 | x | x | n/a | x |

In Table 1, engraftment of species matching strains in DE122435.3 in subjects receiving HHSPs are noted in the first two columns. A range of STRs is indicated in the first column to identify representative sequences for the strains in DE122435.3. For example, “STR00010-STR00010.7” refers to all of STR00010, STR00010.1, STR00010.2, STR00010.3, STR00010.4, STR00010.5, STR00010.6, and STR00010.7. The same notation is repeated for Tables 2 and 3 below. Engraftment of the strains in subjects in a Phase 1 b trial with a designed composition is noted in the third column (n/a indicates strain not present). In the final association with efficacy column, species associated with non-recurrence of CDI, reduction in Enterobacteriaceae, or remission from UC are noted.

### Carbon Source Utilization

Live biotherapeutic products provide or restore colonization resistance, in part, through direct competition for nutrients, such as carbon (Kamada, 2012). Targeting the carbon sources utilized by pathogenic species, e.g., Klebsiella pneumoniae and Enterococcus faecium, the ability of various strains to utilize 85 different carbon sources was determined in vitro.

Growth was scored by measuring optical density at 600 nm and comparing it to growth in base medium without a test carbon source. Growth was assessed under anaerobic conditions for all organisms. Of the 85 carbon sources tested, the 56 used by K. pneumoniae and/or E. faecium were presented.

All 56 carbon sources used by K. pneumoniae or E. faecium were used by at least one tested strain (Table 2, n.d.=data unavailable, and filled cells indicate utilization of the carbon source). Notably, some tested strains appear to be generalists like K. pneumoniae or E. faecium, using a variety of carbon sources; while some appear to be specialists, utilizing a limited number of carbon sources. This mix of carbon utilization profiles reflects the functional diversity amongst the tested strains and is consistent with a phylogenetically and functionally diverse composition, such as that disclosed herein (e.g., DE122435.3).

TABLE 2
Comparison of carbon source utilization profile of Klebsiella pneumoniae (CRE) and Enterococcus faecium (VRE) to DE122435.3
species. Cells with a “U” indicate utilization of the C-source, based on anaerobic growth detected by optical density
in medium with indicated C-source and cells with a “w” indicates ‘weak’ utilization. Empty cells indicate no
utilization of the C-source, and n.d. indicates no data. Of the 85 carbon sources tested, 56 used by either CRE or VRE are presented.

| | | | STR00010- | STR00012- | STR00022- | STR00025- | STR00075- | STR00076- | STR00081- | STR00083- |
|---|---|---|---|---|---|---|---|---|---|---|
| | CRE | VRE | STR00010.7 | STR00012.6 | STR00022.5 | STR00025.5 | STR00075.4 | STR00076.6 | STR00081.5 | STR00083.5 |
| apple pectin | w | | w | w | | U | U | w | U | |
| pectin | U | U | U | U | U | U | U | U | U | U |
| polygalacturonate | w | w | w | U | w | | U | w | | |
| FOS/inulin | U | U | U | | U | w | | U | U | U |
| Inulin | | w | U | | | | | | w | |
| pullulan | U | | | | | U | | | w | |
| starch | U | | w | | | U | w | | | |
| galactomannan | w | w | | | | | U | | | w |
| glycogen | U | | | | | U | U | | w | w |
| xanthan | w | | | | | | | | | |
| guar | | w | | U | | | U | | U | |
| cyclodextrin | | U | | | | U | | | w | |
| raffinose | U | | U | | U | | | U | U | U |
| cellobiose | U | U | U | U | | | | U | w | U |
| lactose | U | U | U | | | w | | U | U | U |
| maltose | U | U | U | U | U | U | | U | U | U |
| sucrose | U | U | U | | U | w | | U | U | U |
| trehalose | U | U | U | U | U | U | | U | | U |
| arabinose | U | U | U | U | U | U | | U | U | U |
| ribose | U | U | w | U | U | U | U | U | U | U |
| xylose | U | | U | U | U | | | U | U | U |
| fructose | U | U | U | U | U | U | U | U | U | U |
| fucose | U | | | | U | | | U | | U |
| galactose | U | U | U | U | U | | | U | U | U |
| glucose | U | U | U | U | U | U | w | U | U | U |
| mannose | U | U | U | U | U | | | U | U | U |
| rhamnose | U | | | | U | | | U | | w |
| sorbose | U | w | | U | | | U | w | | |
| glucuronate | U | U | n.d. | n.d. | n.d. | | n.d. | n.d. | | n.d. |
| NAG | U | U | | U | U | | | w | U | U |
| glucosamine | U | U | U | n.d. | n.d. | | n.d. | n.d. | U | n.d. |
| mucic acid | U | | | | | | U | | U | |
| glycerol | U | | | | U | | | U | | U |
| erythritol | U | | | | | U | | | | |
| dulcitol | U | | | U | | | | U | | U |
| inositol | w | | | | | | | U | | |
| mannitol | U | U | U | U | | | | U | | w |
| sorbitol | U | U | U | U | | | | U | | U |
| xylitol | w | | U | U | | | | U | U | |
| salicin | U | U | U | U | | | | U | | U |
| esculin | U | U | U | U | | w | | U | U | U |
| arbutin | U | U | U | U | | | | U | | U |
| arginine | w | | | | | | | | | |
| histidine | w | | | | | | | U | | |
| serine | U | | | U | w | | U | U | U | w |
| asparagine | U | | | | | | | w | | |
| aspartic acid | w | | | | | | | | w | |
| tryptophan | U | w | n.d. | w | | | U | | U | |
| succinate | w | | | | | | U | | | |
| pyruvate | U | | | U | w | | U | U | w | U |
| citrate | U | | | | | | | | | |
| fumarate | w | | | | | | | | w | |
| malate | U | | | | | | | w | w | |
| lactate | w | | | | | U | | w | w | |
| ethanolamine | U | | | | | | U | | | |
| mucin | U | w | | | | | U | | | |

| | STR00092- | STR00096- | STR00097- | STR00105- | STR00107- | STR00132- | STR00139- | STR00143- |
|---|---|---|---|---|---|---|---|---|
| | STR00092.6 | STR00096.6 | STR00097.4 | STR00105.7 | STR00107.5 | STR00132.5 | STR00139.4 | STR00143.6 |
| apple pectin | | | | U | | | | w |
| pectin | U | U | w | w | U | | U | U |
| polygalacturonate | w | U | | | | | | w |
| FOS/inulin | w | U | | U | w | U | | U |
| Inulin | | w | w | U | | U | | U |
| pullulan | | U | | | | | | |
| starch | U | w | U | U | U | | | U |
| galactomannan | U | | w | | w | w | | w |
| glycogen | U | w | | | U | | | U |
| xanthan | | w | | | | | | |
| guar | | | U | | | | | |
| cyclodextrin | w | | U | w | w | | | w |
| raffinose | U | U | | U | U | U | | U |
| cellobiose | U | U | | U | | U | | U |
| lactose | U | U | | U | U | U | | U |
| maltose | U | U | U | U | U | U | w | U |
| sucrose | U | U | | U | U | U | U | U |
| trehalose | U | U | | | U | U | | |
| arabinose | | U | U | U | U | U | U | U |
| ribose | w | U | w | w | U | U | U | U |
| xylose | | U | w | | U | U | | U |
| fructose | U | U | | U | U | U | U | U |
| fucose | | U | U | | U | U | U | U |
| galactose | U | U | U | U | U | U | w | U |
| glucose | U | U | U | U | U | U | w | U |
| mannose | U | | U | | U | U | U | U |
| rhamnose | w | U | | | | U | | U |
| sorbose | | | | | | | | U |
| glucuronate | | n.d. | | | U | | | |
| NAG | U | U | U | U | U | U | U | |
| glucosamine | U | n.d. | w | n.d. | U | | U | U |
| mucic acid | w | w | U | w | w | | | |
| glycerol | | w | | U | U | n.d. | U | U |
| erythritol | | | | | | | | |
| dulcitol | | w | U | U | | | | |
| inositol | | U | w | | | | | |
| mannitol | | U | | U | | | U | |
| sorbitol | | U | U | U | U | | w | U |
| xylitol | | U | U | w | | | | U |
| salicin | U | U | w | U | | U | | |
| esculin | U | U | U | n.d. | w | U | U | |
| arbutin | U | U | U | n.d. | | U | | |
| arginine | | | U | | | | | |
| histidine | | | | | | | | |
| serine | | w | U | U | | | | |
| asparagine | | w | | | | | U | |
| aspartic acid | | w | U | | | | U | |
| tryptophan | w | | U | n.d. | w | n.d. | | |
| succinate | | | | | | | | |
| pyruvate | | U | | | w | | U | w |
| citrate | | | | | | | w | |
| fumarate | | | | | | | U | |
| malate | | | | | | | U | |
| lactate | | | | | | | U | |
| ethanolamine | | | U | | | | | |
| mucin | w | | | | | w | | |

### Metabolite Production

Several bacterial metabolites were reported to be dysregulated in the microbiome of subjects with GI barrier disruption and GvHD. As described herein, non-limiting examples of such metabolites include short-chain and medium-chain fatty acids (SCFAs and MCFAs, respectively), tryptophan-derived metabolites, bile acid metabolites, e.g., produced by bacteria with 7-α-dehydroxylation activity, and combinations thereof. Therefore, various strains were evaluated for their ability to produce key SCFAs, tryptophan metabolites, and 7-α-dehydroxylated bile acids in vitro.

Individual strains were grown in vitro and culture supernatants were filtered and analyzed by gas chromatography mass spectrometry for the presence of SCFAs, medium chain fatty acids (MCFAs), and tryptophan metabolites. As shown in Table 3, of the strains present in DE122435.3, nine produced propionate and seven produced butyrate, i.e., two SCFAs with known anti-inflammatory properties. In addition, several of the strains were also able to produce the MCFAs valerate and hexanoate. Several strains produced tryptophan-derived metabolites. Three strains produced significant levels of indole and another three strains produced indole-3-pyruvic acid and/or indole-3-acetic acid, all of which are known to exert anti-inflammatory effects in the gut (Aoki, 2018; Bansal, 2010; Ji, 2020). In Table 3, cells marked with “P” indicates strains that produced the metabolite where marked, empty cells indicate no metabolite production, and cells marked “ND” indicate not determined.

TABLE 3
In vitro Characterization of DE122435.3 species

| | Cell & biochemical | | | Metabolites evaluated by Mass Spectrometry | | | | | | |
|---|---|---|---|---|---|---|---|---|---|---|
| | based assays | | | | | | | | Indole Ac/ | LCA/DCA |
| | | Anti- | Pro- | | | | | | Indole | (7aD |
| Strain | HDACi | inflam. | inflam. | Propionate | Butyrate | Valerate | Hexanoate | Indole | Pyr¹ | activity)² |
| STR00010- | P | P | | P | | | | | | |
| STR00010.7 | | | | | | | | | | |
| STR00012- | P | P | | | P | P | P | | | |
| STR00012.6 | | | | | | | | | | |
| STR00022- | P | | | P | | | | | | |
| STR00022.5 | | | | | | | | | | |
| STR00025- | P | P | | P | P | P | P | | P | |
| STR00025.5 | | | | | | | | | | |
| STR00075- | P | P | | P | P | | P | P | | |
| STR00075.4 | | | | | | | | | | |
| STR00076- | | | | | | | | | | |
| STR00076.6 | | | | | | | | | | |
| STR00081- | P | P | | | P | P | P | P | | |
| STR00081.5 | | | | | | | | | | |
| STR00083- | P | | | P | | | | ND | ND | |
| STR00083.5 | | | | | | | | | | |
| STR00092- | | | | P | P | | | | P | |
| STR00092.6 | | | | | | | | | | |
| STR00096- | P | | | P | | | | ND | ND | |
| STR00096.6 | | | | | | | | | | |
| STR00097- | P | P | | | P | | | | | |
| STR00097.4 | | | | | | | | | | |
| STR00105- | | | | | | | | | P | |
| STR00105.7 | | | | | | | | | | |
| STR00107- | | | | | | | | P | | |
| STR00107.5 | | | | | | | | | | |
| STR00132- | P | P | | P | P | P | P | | | |
| STR00132.5 | | | | | | | | | | |
| STR00139- | | | | | | | | | | P |
| STR00139.4 | | | | | | | | | | |
| STR00143- | P | | | P | | | | | | |
| STR00143.6 | | | | | | | | | | |

¹Indole-3-pyruvic acid and/or indole-3-acetic acid
²Lithocholic acid/deoxycholic acid (7-α-dehydroxylation activity)
Inhibition of HDAC Activity

Inhibition of HDAC activity in IECs and immune cells is one of the main mechanisms by which bacterial metabolites such as SCFAs suppress inflammatory pathways and enhance anti-inflammatory responses in epithelial and immune cells (Kim, 2018). Therefore, to assess the HDAC inhibition activity of the bacterial strains disclosed herein, a commercially available in vitro chemiluminescent assay was used. supernatants were tested in the HDAC inhibition assay (HDAC-Glo I/II assay kit, Promega #G6422) with HeLa nuclear extract (Promega #G6570) as the source of HDAC enzymes. The HDACi assay was performed with 15 μL bacterial supernatant, 10 μL 1M Tris pH 8, and 75 μL of assay buffer containing HeLa nuclear extract. To promote metabolite binding to HDACs, the HDAC solution was preincubated with the bacterial supernatants for 15 minutes prior to the addition of the developing reagent containing proteases that can cleave the deacetylated peptide from the luminogenic substrate. Release of the luminogenic substrate into solution was quantified by measuring luminescence after 20 minutes. As shown in Table 3 (above), 11 of the species present in DE122435.3 exhibited HDAC inhibition activity as measured in vitro.

### Anti-Inflammatory Activity in Intestinal Epithelial Cells (IECs)

IL-8 level is generally elevated in the inflamed intestinal mucosa of UC patients. Accordingly, the ability to suppress IL-8 induction in intestinal epithelial cells is a relevant readout for identifying bacterial species that can modulate as inflammatory immune response, such as that observed in many infections and GvHD.

Briefly, to assess the anti-inflammatory activity of the bacterial strains disclosed herein, HT29 cells (an epithelial cell line derived from a colorectal carcinoma), cultured in McCoys Medium supplemented with 10% FBS, GlutaMAX and Pen/Strep were plated at a density of 50k cells/well in 96-well format and allowed to grow for 5 days until fully confluent. Culture medium was changed every two days. On day 5, cells were pre-treated for 1 hour with a bacterial metabolite (e.g., butyrate, propionate, or acetate) or with bacterial supernatants (10% in cell culture medium) before exposure to 1.25 ng/ml recombinant human TNF-α (Peprotech). Cells were incubated for 4 hours. Culture supernatants were collected and assayed for human IL-8 protein by ELISA (R&D systems) or AlphaLISA (Perkin Elmer). IL-8 levels of test samples were normalized to inflammatory controls that were 10% blank bacterial culture medium pre-treated samples that were exposed to the 1.25 ng/ml TNF-α. To measure the pro-inflammatory capacity of individual bacterial strains, human IL-8 concentrations were measured in cell culture supernatants treated with 10% bacterial supernatant in the absence of TNF-α stimulation.

The anti-inflammatory activity of the individual bacterial strains present in the DE122435.3 composition is shown in Table 3 (above). Seven of the strains reduced IL-8 secretion in TNF-α stimulated HT29 cells by at least 50%, indicative of anti-inflammatory activity.

Collectively, the above results demonstrate that the bacterial strains disclosed herein, such as those present in the DE122435.3 composition, exhibit one or more properties that could be useful in treating and/or reducing the risk of an infection or GvHD in a HSCT subject.

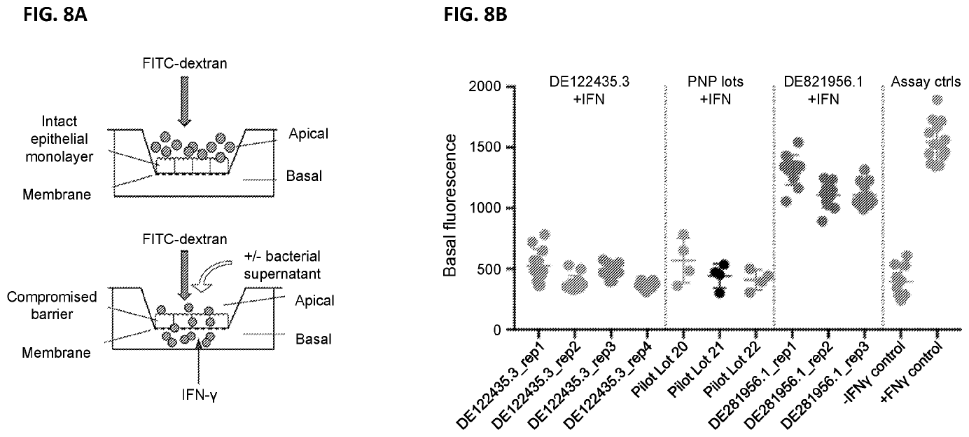

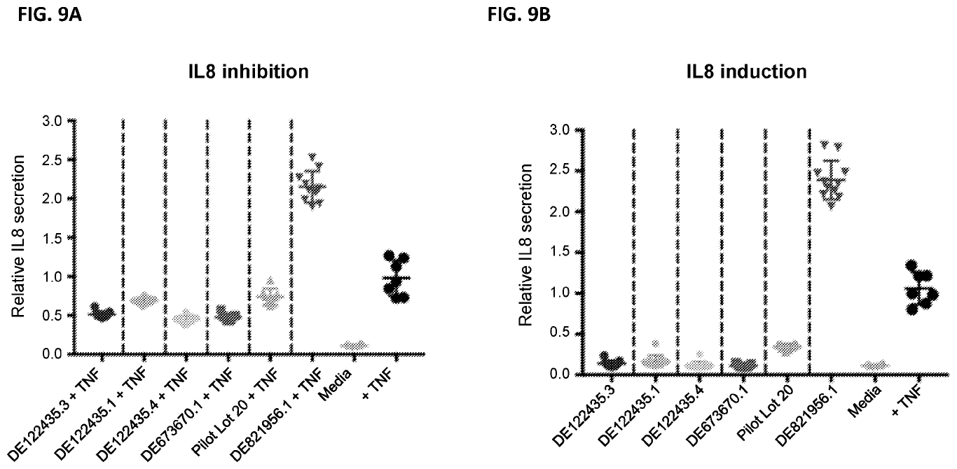

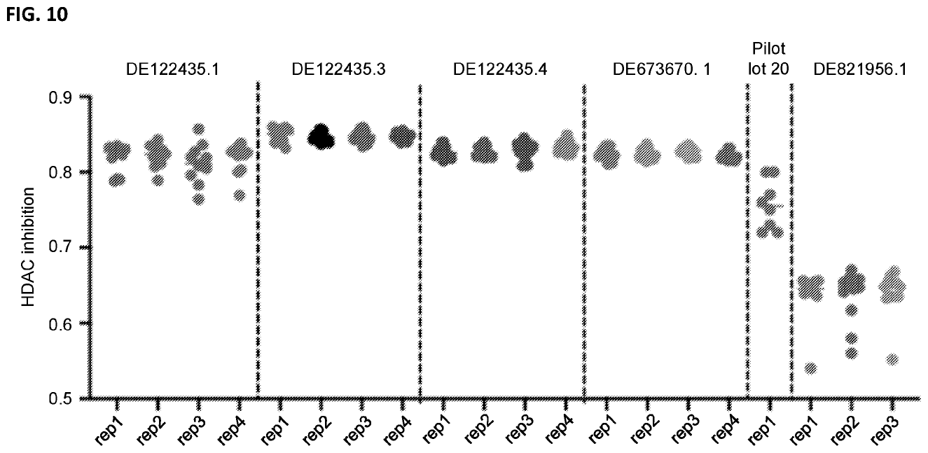

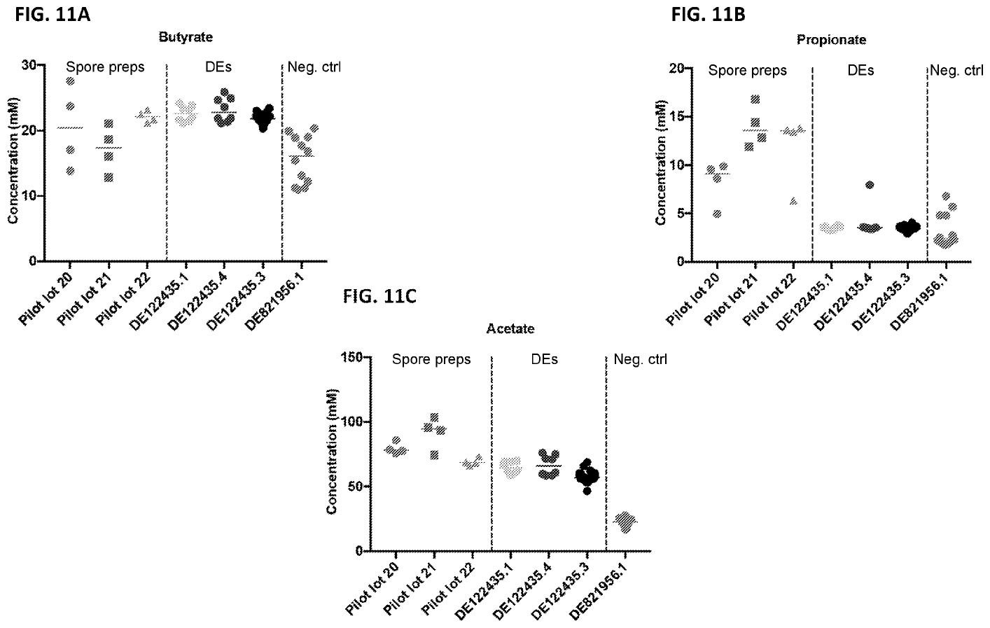

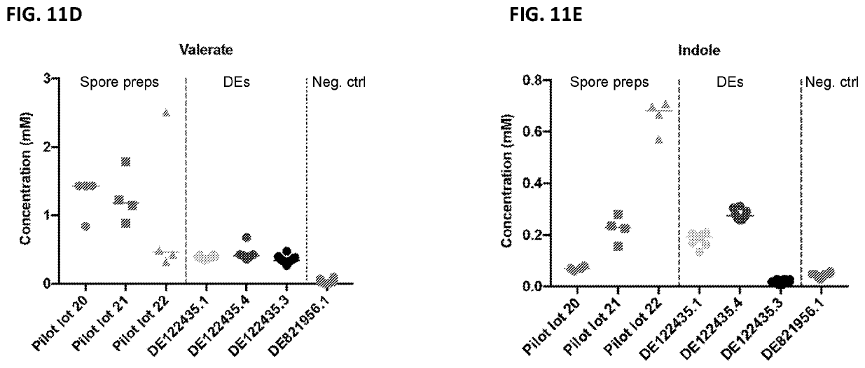

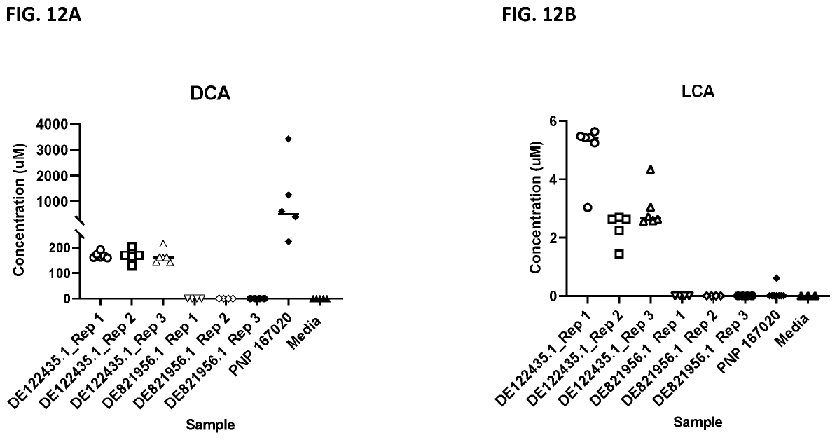

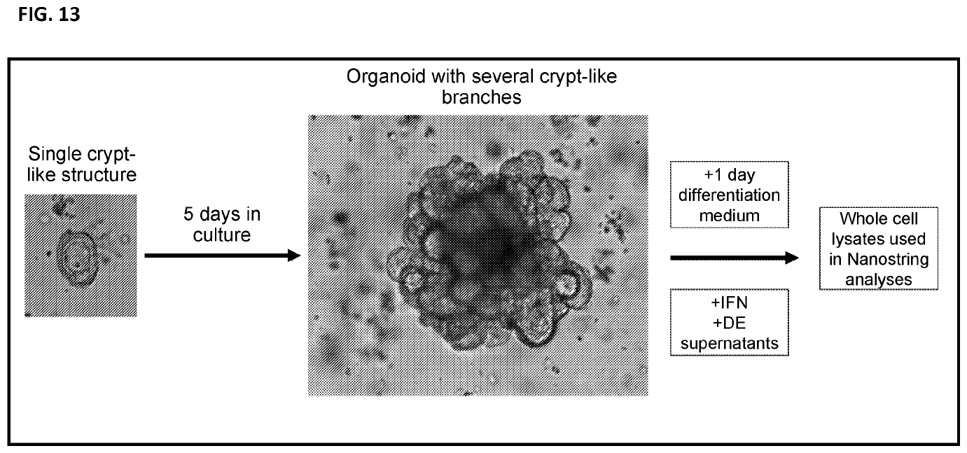

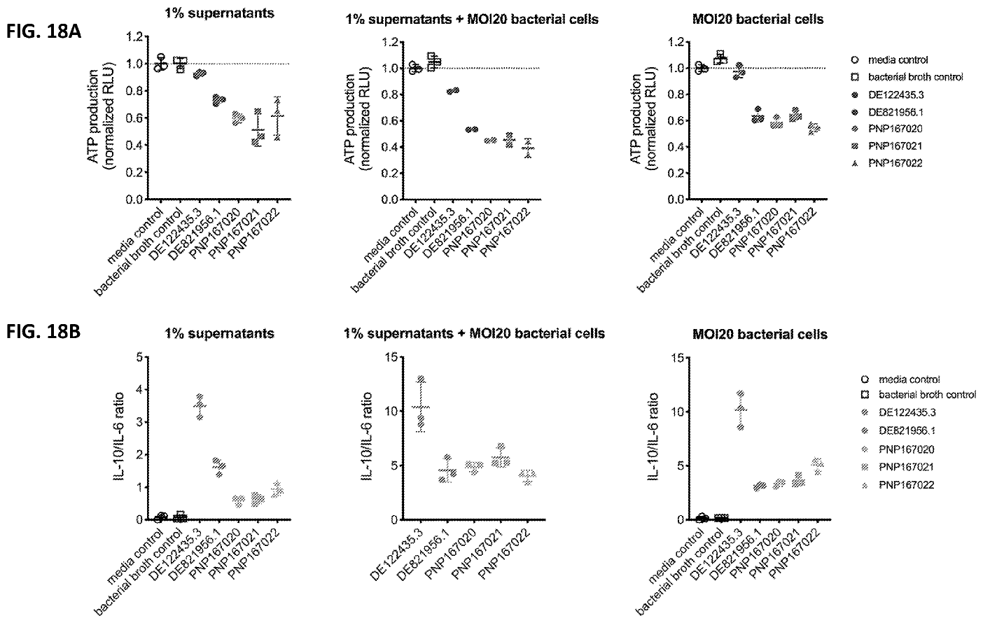

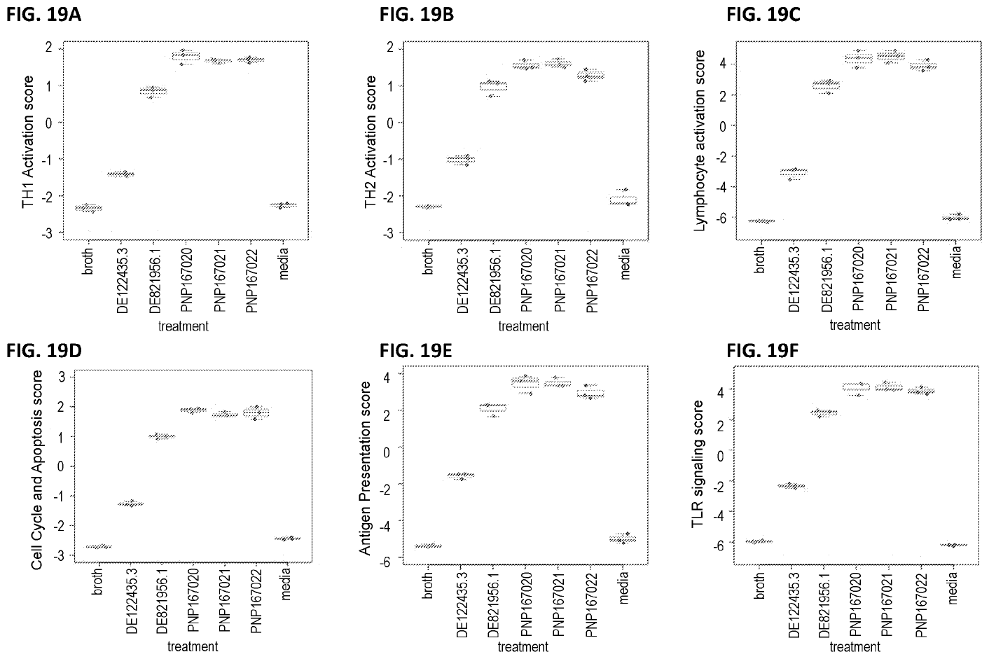

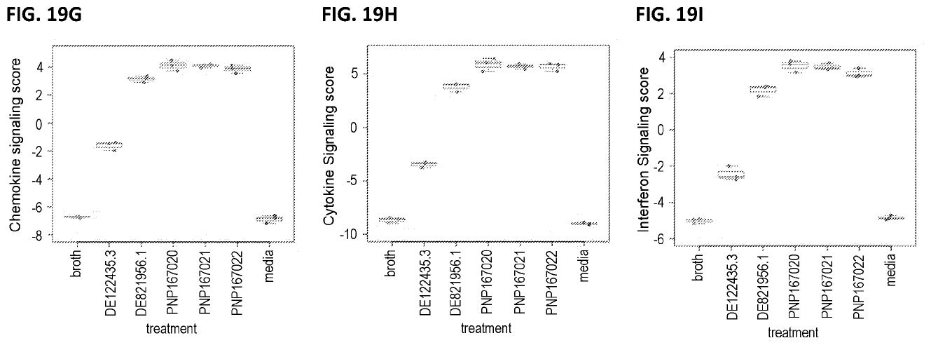

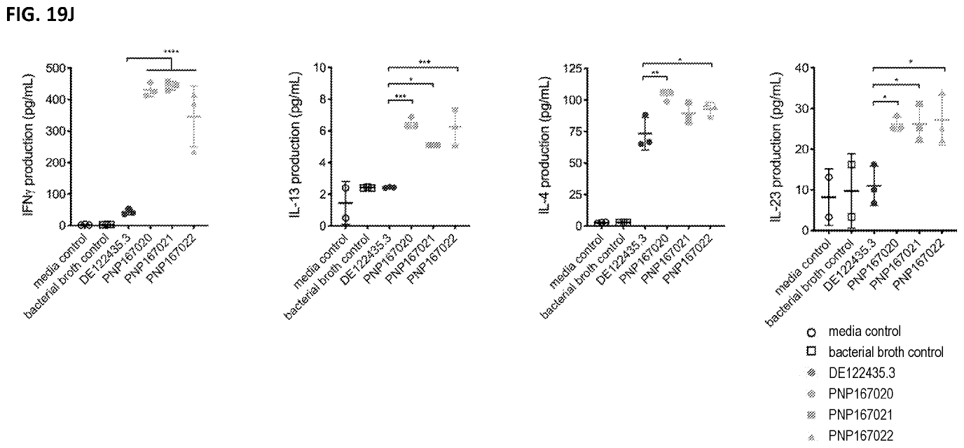

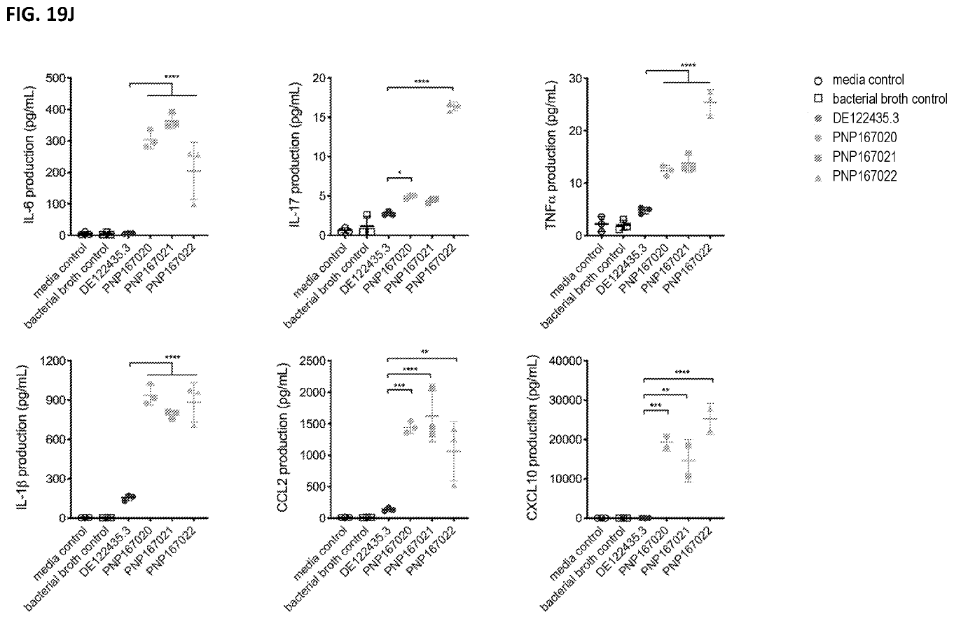

FIGS. 1 and 2A identify the bacterial species included in the different designed compositions. Depending on their bacterial species make-up, the designed bacterial compositions exhibited varying functional activity—see, e.g., FIG. 8B (restoration of epithelial integrity); FIGS. 9A and 9B (anti-inflammatory activity); FIG. 10 (inhibition of HDAC activity); FIGS. 11A-11E (short-chain and medium-chain fatty acid production); FIG. 12 and Table 4 (secondary bile acid production); FIGS. 13, 14A-14P, 15A, 15B, 16A, 16B, and 17 (regulation of genes associated with inflammatory response); and FIGS. 18A, 18B, 19A-J, 20A, and 20B (modulate macrophage phenotype).

### Example 2: In Vivo Analysis of the Effect of DE122435.3 Composition on VRE Colonization

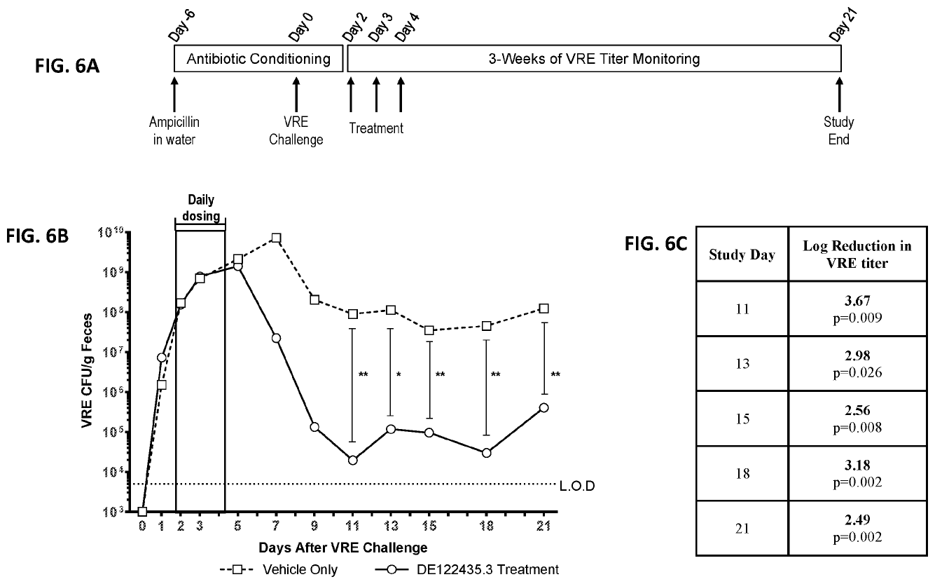

To assess the therapeutic effects of bacterial compositions comprising one or more of the bacterial strains disclosed herein, a mouse model of VRE colonization was used. Briefly, mice were first conditioned with ampicillin in drinking water for eight days to disrupt the native microbiome and impair colonization resistance (see FIG. 6A). On the seventh day of ampicillin treatment (i.e., day 0), the animals were challenged with 1.0×10⁸ colony-forming units (CFU) of Enterococcus faecium (ATCC 700221) by oral gavage. Two days later, ampicillin was removed, and the animals received a dose of either a vehicle control or the DE122435.3 composition daily for three days via oral gavage (i.e., days 2, 3, and 4 post VRE challenge). At various times post VRE challenge, VRE burden (i.e., titer) was quantified in fecal pellets by plating on selective media.

As shown in FIG. 6B, compared to the control animals, mice that received three daily administrations of the DE122435.3 composition had significantly reduced VRE burden. From about day 11 post VRE challenge (i.e., 7 days after the last DE122435.3 administration), animals treated with the DE122435.3 composition had about 2-fold or greater log reduction in VRE titer (see FIG. 6C).

The above result demonstrates that the combination of bacterial strains selected to design the DE122435.3 composition is useful in reducing VRE and thereby, promoting colonization resistance in the gastrointestinal tract.

### Example 3: In Vivo Analysis of the Effect of DE122435.3 Composition on CRE Colonization

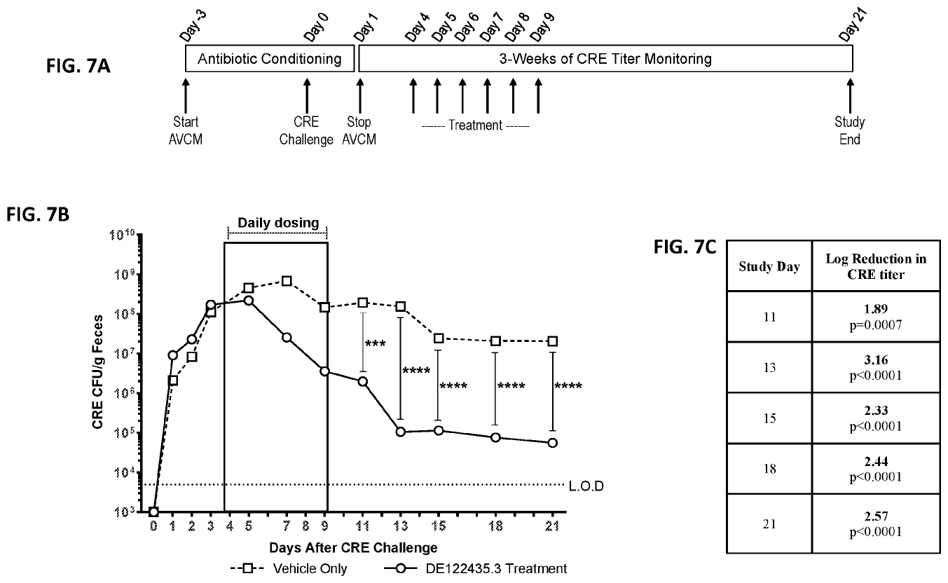

The therapeutic effect of the DE122435.3 composition on CRE colonization was also assessed in a mouse model. As shown in FIG. 7A, mice were conditioned with an antibiotic cocktail containing ampicillin, vancomycin, clindamycin, and metronidazole (“AVCM”) by oral gavage for five days. On the fourth day of AVCM treatment (i.e., day 0), mice were challenged with 1.0×10⁶ CFU of CRE by oral gavage. Two days later, AVCM treatment was stopped. And, starting at day 4 post CRE challenge, the animals received a dose of either the vehicle control or the DE122435.3 composition daily for six days via oral gavage (i.e., days 4-9 post CRE challenge). CRE burden was quantified in fecal pellets by plating on selective media at various time points.

As observed in the VRE animal model, mice that were treated with the DE122435.3 composition had significantly reduced CRE burden compared to the control animals (see FIGS. 7B and 7C). The difference in CRE burden between the two groups began to be apparent after just 3-4 administrations of the DE122435.3 composition.

The above result demonstrates that the combination of bacterial strains present in the DE122435.3 composition is useful in reducing the burden of certain antibiotic resistance pathogens, and thereby, promoting colonization resistance in the gastrointestinal tract.

### Example 4: Assessment of SCFA Production by HDAC Inhibition Assay and Assessment of Bile Acid Production

Short-chain fatty acids (SCFAs) have been described as playing a role in regulating host immunity. Studies have described altered patterns of SCFA in patients of different gastrointestinal diseases, e.g., colitis, and administration of butyrate and propionate have been reported to have therapeutic effects in a colitis animal model. Both in vitro and in vivo, SCFAs have been shown to inhibit histone deacetylate (HDAC) activity, which can then, in turn, regulate many aspects of an immune response (e.g., induction of FoxP3⁺ regulatory T cells). Therefore, bacteria that can produce SCFAs can be useful for the treatment of IBD (e.g., UC) patients.

All bacterial composition frozen stocks were diluted to a final concentration of 5×10⁶ CFU/mL and inoculated as biological replicates in a synthetically derived fecal culture medium 4 (FCM4). FCM4 is composed of complex carbohydrates and mucin in addition to other nutrients that support growth of numerous phylogenetically diverse anaerobic species found in the colon. The medium was supplemented with conjugated bile acids (gCA, tCA, gCDCA and tCDCA) at a final concentration of 100 μM to allow for analysis of microbial bile acid metabolism. Cultures were grown in 96-well deep well plates for a final sample volume of 1.2 mL/well and sealed with adhesive Aeraseal films to allow gas exchange. Bacterial cultures were incubated anaerobically at 37° C. for 7 days, after which cultures were centrifuged at 4000 rpm for 20 minutes, the supernatants passed through a 0.2 μm GHP membrane filter (Pall Corporation, cat #5052).

Microbial supernatants were used for the HDAC inhibition assay (HDAC-Glo I/II assay kit, Promega) and HeLa nuclear extract (Promega) as the source of HDAC enzymes. Assays were performed with 15 μL supernatant, 10 μL 1M Tris pH 8, 75 μL of assay buffer containing diluted HeLa nuclear extract which were preincubated for 15 minutes prior to the addition of developing reagent. Luminescence was measured after 20 minutes. Under these conditions, a sterile supernatant spiked with 15 mM butyrate resulted in 65-75% HDAC inhibition.

As shown in FIG. 10, a number of bacterial compositions were tested for the ability to inhibit HDAC activity. Metabolites produced by three bacterial compositions disclosed herein, DE122435.3, DE122435.1, DE122435.4, strongly inhibit HDAC activity in vitro (>85% HDAC inhibition compared to the no addition control). All three bacterial compositions showed comparable HDAC inhibition to the complex spore pilot lot demonstrating that a rationally designed consortium can exhibit metabolic phenotypes of interest at the levels of a natural complex community. The negative control DE821956.1 in contrast is less potent in inhibiting HDAC activity. Under these conditions (10 mM butyrate), a bacterial metabolite with strong HDAC inhibitory activity, resulted in 91% inhibition.

FIGS. 11A-11E compare SCFA production. FIG. 12 and Table 4 provide a comparison of several properties of the bacterial compositions disclosed herein, including bile acid metabolic activity (i) BSH tCA [for bile salt hydrolase activity on taurocholic acid], (ii) BSH gCA [for bile salt hydrolase activity on glycocholic acid], (iii) BSH CA [for bile salt hydrolase activity on cholic acid], and (iv) 7aD DCA [for 7α-dehydroxylase activity on DCA]. Bile acid activity was measured in in vitro cultures of the different bacterial compositions shown fed the conjugated primary precursors. The primary bile acid products shown include (i) cholic acid (CA) (i.e., BSH byproduct), and (ii) chenodeoxycholic acid (CDCA) (i.e., BSH byproduct). The secondary bile acid products shown include (i) deoxycholic acid (i.e., 7aD byproduct), (ii) lithocholic acid (LCA) (i.e., 7aD byproduct), (iii) oxo-derivatives of CA and CDCA (i.e. HSDH byproducts), and (iv) urso-deoxycholic acid (UDCA, an HSDH byproduct).

TABLE 4
Bile acid metabolite activity shown as amount of primary and secondary bile acid products.

| Bile Acid Information | | Sample | | | |
|---|---|---|---|---|---|
| Metabolite Type | Bile Acid | Media | DE122435.3 | PNP 167020 | DE821956.1 |
| Conjugated | gCA | 432 ± 87.3 | n.d. | n.d. | n.d. |
| primary | tCA | 319.7 ± 65.5 | n.d. | n.d. | n.d. |
| precursors | gCDCA | 1909.8 ± 1986.7 | 0.8 ± 3.5 | n.d. | n.d. |
| | tCDCA | 370.9 ± 57.6 | n.d. | n.d. | n.d. |
| Primary bile | CA | 8.2 ± 5 | 1241.4 ± 558.5 | 43.3 ± 32.1 | >100 |
| acid products | CDCA | n.d. | 120.1 ± 16.8 | 3.2 ± 4.7 | >100 |
| Secondary | DCA | n.d. | 167.2 ± 21.2 | 606.8 ± 412.1 | n.d. |
| bile acid | LCA | n.d. | 3.5 ± 1.4 | 0.5 ± 0.1 | n.d. |
| products | 3-oxo LCA | n.d. | n.d. | n.d. | n.d. |
| | 7-oxo 3a | n.d. | 72.6 ± 4.8 | 18.9 ± 20.2 | 197.7 ± 28.2 |
| | 12-oxo 3a | n.d. | 14.3 ± 2.5 | 33 ± 8.2 | n.d. |
| | 3-oxo 7a | n.d. | 6.4 ± 0.8 | 20.4 ± 8.4 | 20 ± 16.5 |
| | 7-oxo 3a 12a | n.d. | 83 ± 5 | 82.9 ± 49.7 | 83.5 ± 12.7 |
| | 12-oxo 3a 7a | n.d. | 2.5 ± 0.3 | 3.3 ± 2.8 | 7.3 ± 3.3 |
| | 3b 12a (iso-DCA) | n.d. | 61.6 ± 4.3 | 50.9 ± 20.4 | 72.1 ± 12.8 |
| | UDCA | n.d. | 2.6 ± 0.7 | 46 ± 35.1 | 0.7 ± 1.8 |

The above data indicate that the designed bacterial compositions disclosed herein exhibit certain functional attributes (e.g., HDAC inhibition, production of short-chain and medium-chain fatty acids, and bile acid metabolic activity), which can be useful in treating diseases or disorders, such as an infection or GvHD following HSCT.

### Example 5: Barrier Integrity Analysis

As described herein, many of the bacterial strains provided in the present disclosure are capable of producing certain tryptophan metabolites and short chain fatty acids, which are associated with more robust intestinal epithelial barrier and mucosal homeostasis. Disruption of normal barrier function (e.g., due to destruction of tight junctions between epithelial cells and apoptosis induced by chronic inflammation) is an important factor in the pathogenesis of certain diseases or disorders, such as GvHD. Therefore, the ability of bacterial compositions comprising one or more of the bacterial strains disclosed herein to restore barrier integrity was assessed with an in vitro epithelial barrier assay.

As illustrated in FIG. 8A, the assay apparatus has an apical side and a basal side that are separated by a monolayer of epithelial cells on a permeable membrane. The addition of interferon-gamma (IFN-γ) disrupts the tight junctions of the epithelial monolayer and induces apoptosis of epithelial cells (see bottom well). The leakiness of the membrane can be assessed by adding FITC-dextran to the apical side of the apparatus and measuring how rapidly it can pass to the basolateral compartment. A leaky monolayer will allow FITC-dextran to the basal side of the apparatus more quickly than a monolayer with an intact monolayer.

Briefly, the barrier integrity assay was conducted as described below using the DE122435.3 composition. Primary human colon organoid cultures established from isolated colon crypts were grown and expanded in Matrigel® (Corning) and 50% L-cell conditioned medium containing Wnt3a, R-spondin 3 and Noggin (L-WRN) as described by VanDussen et al. containing 10 uM Y-27632 and 10 uM SB43152 (Gut 64:911-920, 2015). Colon organoids were harvested and trypsinized into a suspension containing few cell clusters and seeded onto Matrigel coated transwell inserts (Corning) at a density of 100,000 cells per insert in 50% L-WRN medium supplemented with 10 μM Y-27632 (Millipore Sigma). Epithelial cell monolayers formed over 4-5 days in 50% L-WRN medium. These primarily stem cell population was differentiated into colonocytes by switching the culture medium to 5% L-WRN for 48 hours. After 24 hours of differentiation, a test agent (e.g., bacterial supernatant) was added to apical interface in 100 μL of 5% L-WRN medium and 5-25 ng/ml INFγ (Peprotech), depending on the experiment, was added in 175 μL of 5% L-WRN medium to the basolateral interface and incubated for 48 hours at 37° C. After the 48 hour incubation, colonic epithelial monolayer permeability was assessed by adding 10 μL of 10 ng/ml FITC-Dextran (4 kDa, Sigma) to the apical interface, the organoids were incubated for 1 hour and then 100 μL of medium was collected from the basolateral compartment of each transwell and transferred to a 96 well plate for fluorescence detection.

As shown in FIG. 8B, application of supernatant from DE122435.3 to the apical surface prevented IFN-γ-mediated barrier disruption, as seen by the reduced fluorescence in the basal compartment compared to the IFN-γ control as did three healthy donor spore pilot lots. In contrast, supernatants of a pro-inflammatory composition, DE831956.1 were not protective. These data show that the bacterial composition DE122435.3 led to a significant reduction in barrier permeability.

### Example 6: Anti-Inflammatory Activity with Intestinal Epithelial Cells

Using the methods provided in Example 1 for measuring anti-inflammatory activity of individual bacterial strains disclosed herein, the anti-inflammatory activity of DE122435.3, DE122435.1, and DE122435.4 were evaluated. A consortium of bacterial spores from a healthy human donor (a nonclinical pilot lot 20, hereafter ‘Pilot lot 20’) and a composition with inflammatory properties (DE821956.1) were also included as positive and negative controls, respectively. All bacterial cultures were grown in vitro in complex medium that supports the growth of many anaerobic gut bacteria, mimicking the nutrients found in the colon. Supernatants from these cultures were filtered and applied to HT29 cells which were then treated with TNF-α to induce IL-8 secretion.

As shown in FIG. 9A, DE122435.3, DE122435.1, DE122435.4 and Pilot lot 20 supernatants significantly reduced IL-8 secretion in TNF-α stimulated HT29 cells compared to the TNF-α alone control group. Negative Control DE821956.1 increased IL-8 secretion compared to TNF-α alone, confirming its inflammatory nature.

The above supernatants were also tested in the same IEC assay without TNF-α treatment to assess the ability of the different bacterial compositions to induce IL-8, indicative of proinflammatory activity. As shown in FIG. 9B, DE122435.3, DE122435.1 and DE122435.4 supernatants did not elicit significant IL-8 secretion in the HT29 cells. By contrast, with the Pilot lot 20 composition, more IL-8 secretion was observed than with DE122435.3, DE122435.1 and DE122435.4, demonstrating advantages to the designed compositions compared to a healthy donor-derived spore-based composition. On the other hand, DE821956.1 induced IL-8, as expected, given the pro-inflammatory activity of its strains.

The above results demonstrate the anti-inflammatory nature of the bacterial compositions disclosed herein, suggesting their suitability in treating and/or reducing the risk of diseases or disorders, such as infections and GvHD in HSCT subjects.

### Example 7: Regulation of Cellular Pathways Relevant to Inflammation and GvHD

Bacterial composition-mediated regulation of gene expression pathways relevant to inflammation and GvHD was profiled in human primary epithelial organoids. Primary intestinal epithelial organoids, or “mini-guts” are complex, self-organizing, cell structures formed by Lgr5⁺ adult intestinal stem cells that consist of goblet cells, paneth cells, enteroendocrine cells (Sato et al, Gastroenterology Journal, 141:1762-1772, 2011). Primary human colon organoid cultures established from isolated colon crypts were grown and expanded in Matrigel® (Corning) and 50% L-cell conditioned medium containing Wnt3a, R-spondin 3 and Noggin (L-WRN) as described by VanDussen et al. (Gut 64:911-920, 2015). Colon organoids were grown in 24-well plates for 5 days in 50% L-WRN medium. After 5 days of mini-gut structure formation in 50% L-WRN medium, organoid culture medium was switched to 5% L-WRN medium to induce differentiation of the organoids. After 24 hours in 5% L-WRN medium, organoids were treated with 10% DE supernatants in fresh 5% L-WRN medium supplemented with the inflammatory cytokine, e.g., 12.5 ng/ml human TNFa (Peperotech) or 10 ng/ml IFN- [symbol] . Control conditions include organoids treated with 5% L-WRN+10% bacterial culture medium and 5% L-WRN+10% bacterial culture medium+12.ng/ml human TNFa or IFN- [symbol] . Organoids were incubated in treatment conditions overnight and then collected in Qiagen RLT buffer for RNA analysis. Sample lysates were either purified into RNA using Qiagen RNeasy mini prep kit or lysates were assayed directly on the Nanostring nCounter platform. In some aspects, purified RNA was used to prepare amplified cDNA libraries that were sequenced using an Illumina NovaSeq 6000 instrument. As shown in FIG. 13, organoids were grown from passage in 50% LWRN for 5 days to form robust mini-guts. Following that, medium was switched to 5% LWRN to produce a more differentiated epithelium for 24 hours. DE supernatants were added to the differentiated epithelium and co-treated with/without IFN-γ overnight in 5% L-WRN. Organoids were then harvested for transcript profiling.

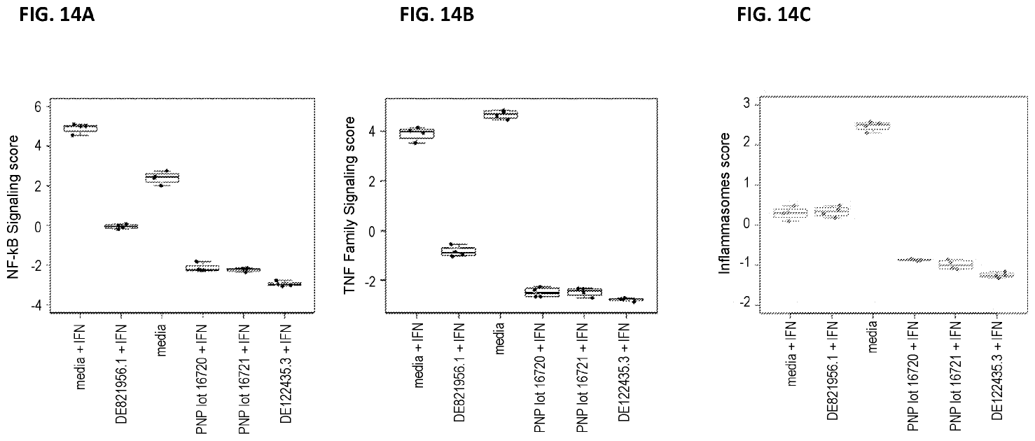

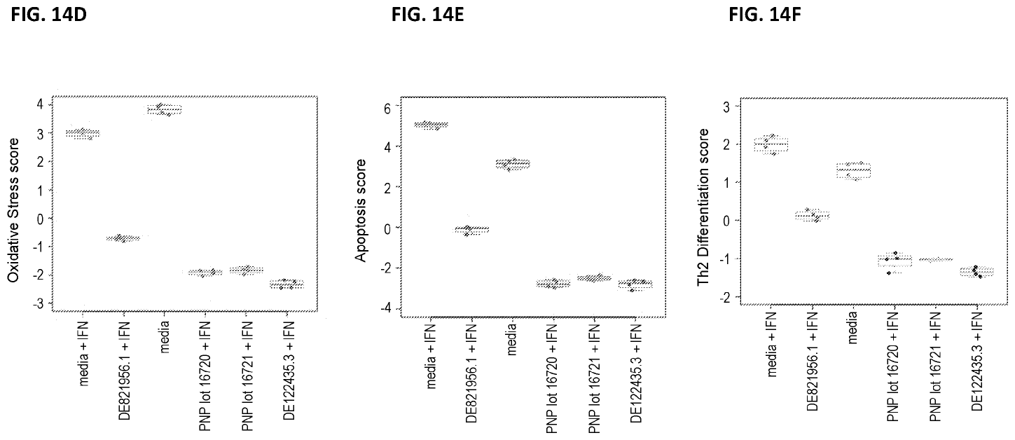

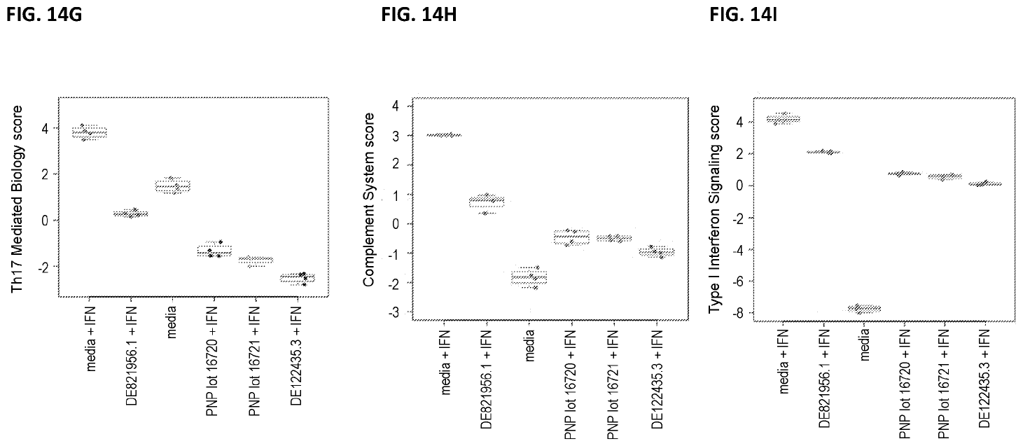

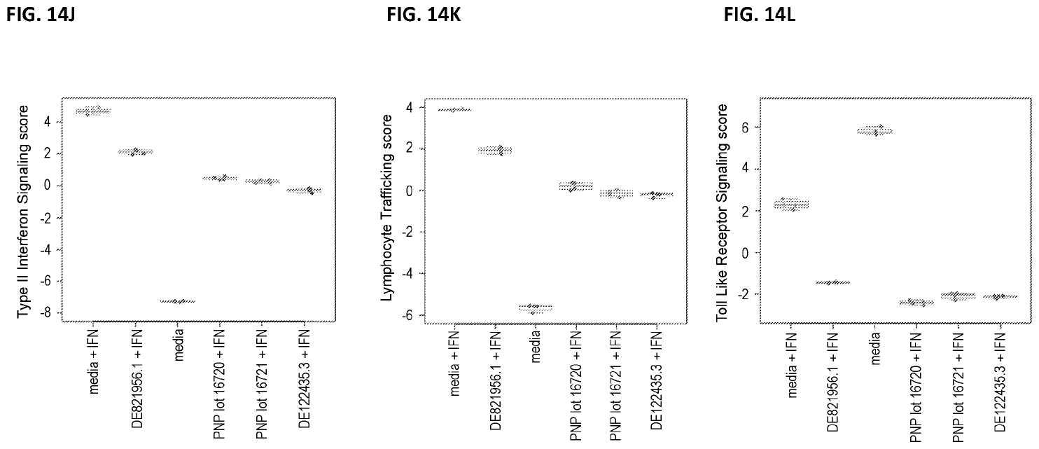

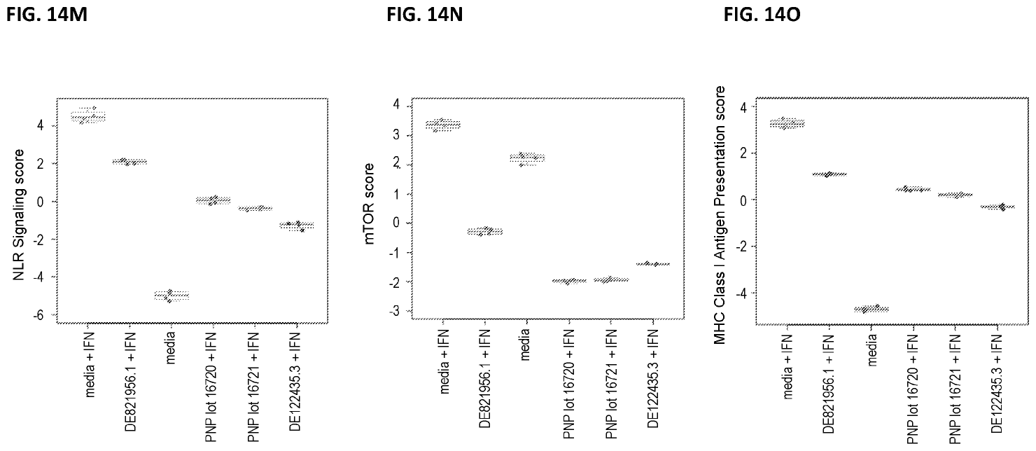

As shown in FIGS. 14A-14P, DE122435.3 and the pilot lots prevented IFN-γ-mediated induction of inflammatory pathways. Co-treatment of colonic organoids with 5% bacterial supernatant in the presence of 10 ng/ml IFNγ decreased expression of many pathways related to inflammation including (1) NF-κB signaling (FIG. 14A), (2) TNF family signaling (FIG. 14B), (3) inflammasomes (FIG. 14C), (4) oxidative stress (FIG. 14D), (5) apoptosis (FIG. 14E), (6) Th2 differentiation (FIG. 14F), (7) Th17 mediated biology (FIG. 14G), (8) complement system (FIG. 14H), (9) type I interferon signaling (FIG. 14I), (10) type II interferon signaling (FIG. 14J), (11) lymphocyte trafficking (FIG. 14K), (12) Toll Like Receptor signaling (FIG. 14L), (13) NLR signaling (FIG. 14M), (14) mTOR (FIG. 14N), (15) MHC Class I antigen presentation (FIG. 14O), and (16) MHC Class II antigen presentation (FIG. 14P). Of particular relevance for GvHD, are pathways related to leukocyte trafficking and T cell activation, as the disease is caused by abnormal T cell activation. Moreover, inhibition of epithelial apoptosis and oxidative stress pathways can play a significant role in enhancing epithelial barrier repair after transplant pre-conditioning.

Chemokines are inducers of leukocyte trafficking and activation, which lead to tissue damage during GvHD. Chemokines are a family of small proteins (about 8-14 kDa) that are classified into four major groups based on the number and spacing of conserved cysteines; the groups include the CC group (CCL1-28), the CXC group (CXCL1-16), the C group (XCL1-2), and the CX3C group (CX3CL1). (Castor et al., Front Pharmacol.3:23 (2012), PMCID: PMC3285883). Particularly, CXCL9 and CXCL10 are involved in T-cell recruitment and increased CXCL10 is seen in both acute and chronic GvHD patient sera. IL-1β neutralization improves survival of mice subject to a GvHD model of irradiation. CX3CL1 is also involved in T-cell recruitment and increased CX3CL1 is observed in GI and mononuclear lamina propria cells of GvHD patients. CCL2 is important for migration of donor cells to target organs during GvHD development and elevated CCL2 associated with migration of donor cells to lungs. Increased CXCL1 and CXCL2 levels are observed in GI tract of mice receiving allogeneic bone marrow transplant.

The interaction between chemokines and one or more members of a family of seven transmembrane domain-containing G-protein-coupled receptors exerts the effects on the pathogenesis of GvHD. In GvHD, downstream signaling of chemokine receptors leads to activation of PI3K, JAK, STAT, and MAPK and thus pro-inflammatory events that are crucial to GvHD development. STAT-3/STAT-1 activation precedes the activation of NF-κB and MAP kinases with the subsequent expression of IRF-1, SOCS-1, and IL-17. NF-κB has a dual role in GVHD development, depending on phase of its expression. STAT-3 phosphorylation acts as a promoter of GVHD inflammation and is regulated by SOCS-3. (Front Pharmacol.3:23, PMCID: PMC3285883). STAT3 phosphorylation is important for allo-T-cell activation in GvHD. Inhibition of STAT3 phosphorylation prevents T-cell activation and proliferation in vitro and GvHD in vivo. (Blood, 112(13): 5254-5258.) Furthermore, a recent study found MHC class II-mediated antigen presentation by epithelial cells initiated GvHD. TLR adaptors MyD88 and TRIF signaling are required for MHC II expression. IFN-γ secreted by IELs (intra-epithelial lymphocytes) increases MHCII expression.

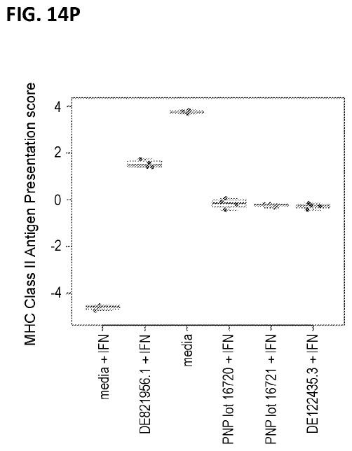

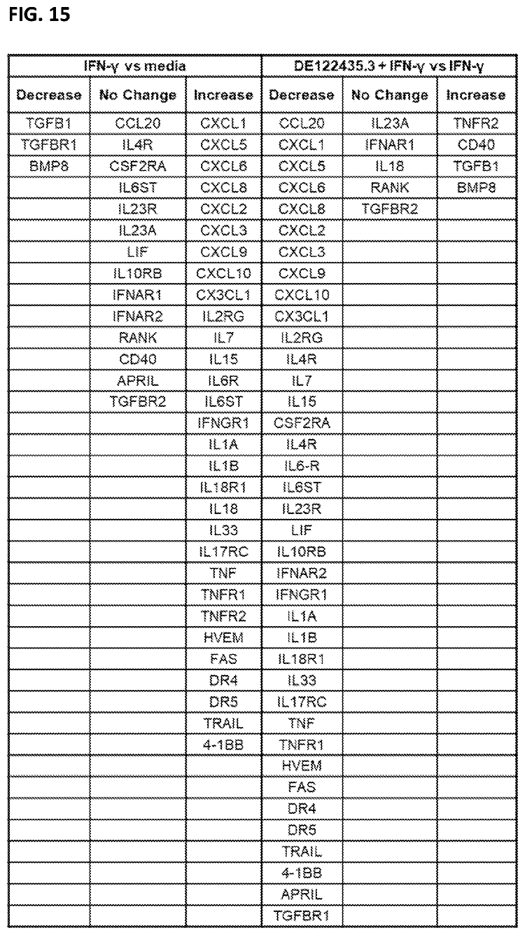

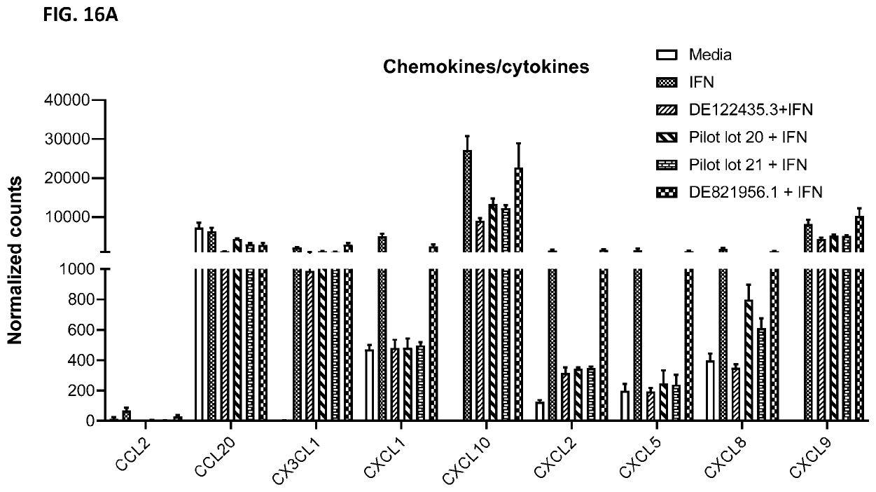

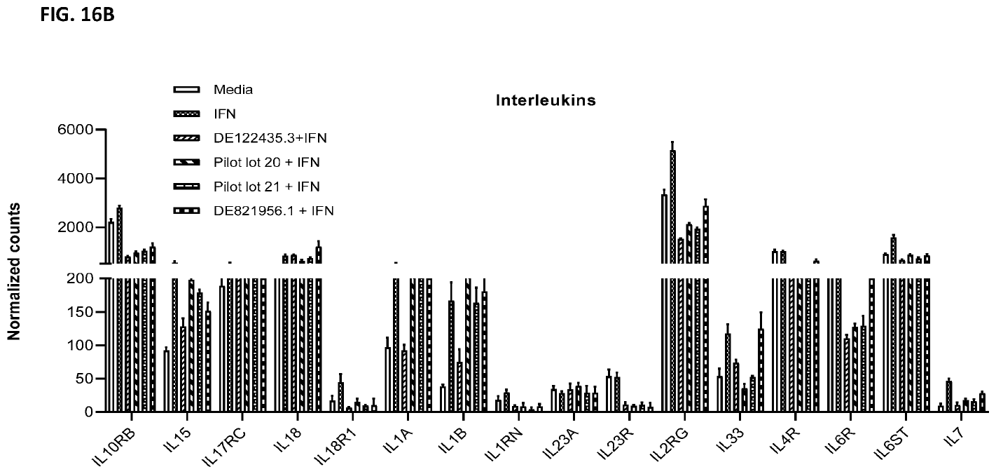

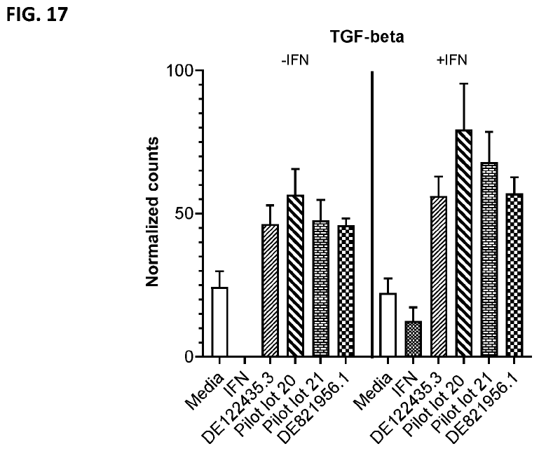

As shown in FIG. 15, IFN-γ induces expression of various chemokines and interleukins, including CXCL1-3, CXCL5, CXCL6, CXCL8-10, IL7, IL15, ILIA, IL1B, among other. Also shown in FIG. 15, inflammatory cytokine expression is downregulated by DE122435.3. As shown in FIG. 16A, chemokines CXCL1, CXCL2, CXCL5, CXCL8 were drastically downregulated by DE122435.3 and pilot lots. As shown in both FIGS. 16A-B, DE122435.3 downregulated inflammatory cytokines and chemokines similar to or slightly better than pilot lots 20/21. Negative control, DE821956.1, was less efficient in inhibiting inflammatory cytokines. And, as shown in FIG. 17, DE122435.3 upregulated TGF-β, a cytokine involved in mucosal healing and with anti-inflammatory properties.

The above results further highlight the anti-inflammatory and epithelial protective nature of the designed bacterial compositions disclosed herein, which can be useful in treating a disease or disorder associated with excessive inflammation, such as that observed in many infections and GvHD after HSCT.

### Example 8: In Vitro Cytokine Induction Profile in Macrophages

To further assess the ability of the designed bacterial compositions disclosed herein to treat a disease or disorder disclosed herein (e.g., GvHD), the ability of the bacterial compositions to modulate macrophage phenotype was assessed. Without committing to any one specific theory, antigen presenting cells, including macrophages, can play a role in activation of T cells, e.g., by producing inflammatory cytokines and/or presenting cognate antigens to the T cells. When such T cells are from an allogenic donor and recognize an antigen in the recipient, the activated T cells can attack the recipient tissues and thereby promote GvHD. Antigen presenting cells (e.g., macrophages) are also key sources of IL-23, a mediator of colonic GvHD. However, study reports have also suggested that certain macrophages exhibit anti-inflammatory properties (e.g., “M2”) and can repress T cell proliferation and activation.

Briefly, to test the above, human monocytic THP-1 cells were differentiated into macrophages with a chemical stimulant as follows. Human macrophages were derived from the THP-1 monocytic cell line (ATCC). THP-1 monocytes were grown in RPMI (Gibco) supplemented with 10% FBS, Pen/Strep, and sodium pyruvate. Cells were differentiated into macrophages by incubation with 25 ug/mL phorbol 12-myristate-13-acetate (PMA, Peprotech) for 24 hours. Cells were grown in 96 well tissue culture treated sterile microtiter plates (Corning) with 100,000 cells seeded per well. Macrophage differentiation is confirmed by quantifying attachment to the tissue culture growth plate (cellular adhesion assay) and expression of macrophage cell surface markers (determined by flow cytometry). The differentiation medium is then replaced with fresh medium, and cells are rested for 24 hours to return the cells to a basal signaling state. Following rest, differentiated macrophages are stimulated with 1% bacterial culture supernatant, a multiplicity of infection (MOI) of 20 bacterial cells (counted via flow cytometry) per macrophage, or a combination of 1% supernatant and MOI20 bacterial cells. After 24 hours of stimulation, culture supernatants were collected for cytokine measurements (Luminex). The cells were harvested for determination of viability or were used to generate cellular lysates for transcriptional analyses. Cell viability was measured via luminescence in an assay that directly measures the presence of cellular ATP (a marker of cell health; CellTiterGlo 2.0, Promega). Assay performance of CellTiterGlo was controlled via an ATP standard curve, and cellular viability was normalized to the respective medium alone (non-stimulated) wells. Quantitation of cytokine production was performed with a ThermoFisher multi-plexed Luminex panel with commercial standards. All analyte standard curves were quality controlled in xPONENT (custom Luminex software), and cytokines were detected above the limit of quantitation of each respective analyte. Transcriptional changes were assessed via NanoString Technologies multiplexed molecular barcode (human myeloid 2.0 panel) with analogous hybridization and sample prep conditions across cellular treatments. Raw probe counts were normalized using nSolver (NanoString Technologies software) with analogous background correction and data normalization across samples. The internal negative and positive controls (commercially provided by NanoString Technologies in each panel) all passed quality control across the samples. Data were plotted and analyzed for statistical significance in GraphPad Prism 8.4.3. As shown in FIG. 18A, differentiated macrophages stimulated with DE122435.3 maintain viability whereas macrophages stimulated with healthy complex natural communities or DE821956.1 exhibit about 40% decrease in viability. Transcriptional profiling of stimulated macrophages shows that DE122435.3 elicits significantly less pro-inflammatory gene expression changes, including Th1 and lymphocyte activation, antigen presentation, cyto- and chemo-kine and interleukin signaling, cell cycle and apoptosis, or TLR signaling than healthy complex communities (PNP167020; PNP167021; and PNP167022) or the negative control inflammatory DE821956.1 (FIGS. 19A-I).

As shown in FIG. 19J, DE122435.3 composition does not elicit the production of pro-inflammatory cytokines known to activate T cells, NK cells, or monocytes or to elicit the production of cytokines shown to be elevated in GvHD patients compared to complex natural communities. These data further demonstrate that, compared to natural communities or an inflammatory composition, the designed bacterial compositions disclosed herein exhibit significantly reduced pro-inflammatory properties that may potentially lead to exacerbation of GvHD.

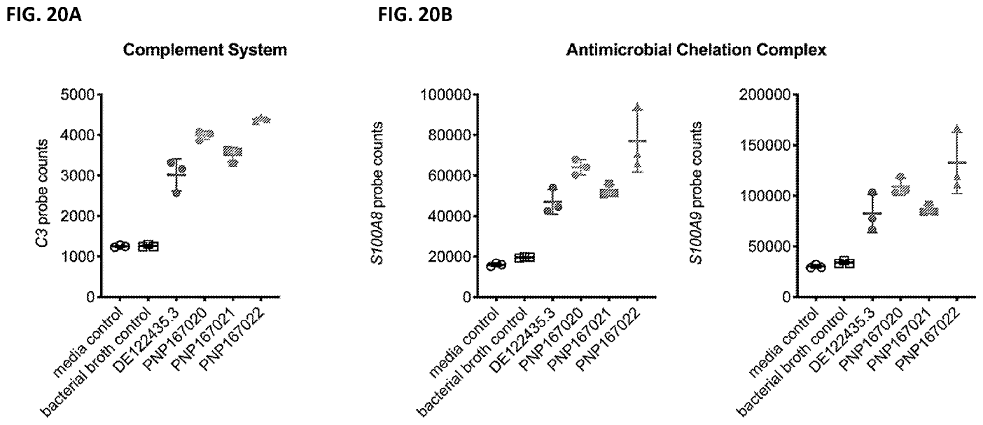

As shown in FIGS. 20A and 20B, macrophages stimulated with DE122435.3 transcriptionally induce key innate immune defenses, including the complement pathway and calprotectin, an antimicrobial chelation complex pathway, critical for pathogen or commensal clearance in the event of barrier breach, at comparable levels of complex healthy bacterial communities. These data demonstrate that designed composition DE122435.3 elicits significantly reduced pro-inflammatory cytokine or transcriptional changes in human macrophages while also maintaining key functionality for innate defense and thus is an improvement over natural communities (i.e., spore-prep compositions).

### Example 9: Therapeutic Effect of the DE122435.1 and DE122435.3 Compositions in a Germ-Free Mouse Model of Immunomodulation

DE122435.1 is another bacterial composition that is related to the DE122435.3 composition described herein. DE122435.1 differs from the DE122435.3 composition by two strains. DE122435.1 is made up of 16 strains, of which 14 are the same strains as in DE122435.3, and 2 strains (STR00011 and STR00106) are replaced with closely related strains.

To evaluate the effect of the DE122435.1 and DE122435.3 compositions on intestinal T cell responses, GF wild-type C57BL/6 mice were colonized with either DE122435.1 or DE821956.1, a bacterial composition known to have proinflammatory properties in vitro, DE122435.3 or DE916091.1, another bacterial composition known to have proinflammatory properties in vitro. After four weeks of colonization, colonic lamina propria lymphocytes were isolated for flow cytometric characterization of CD4+ T cell populations.

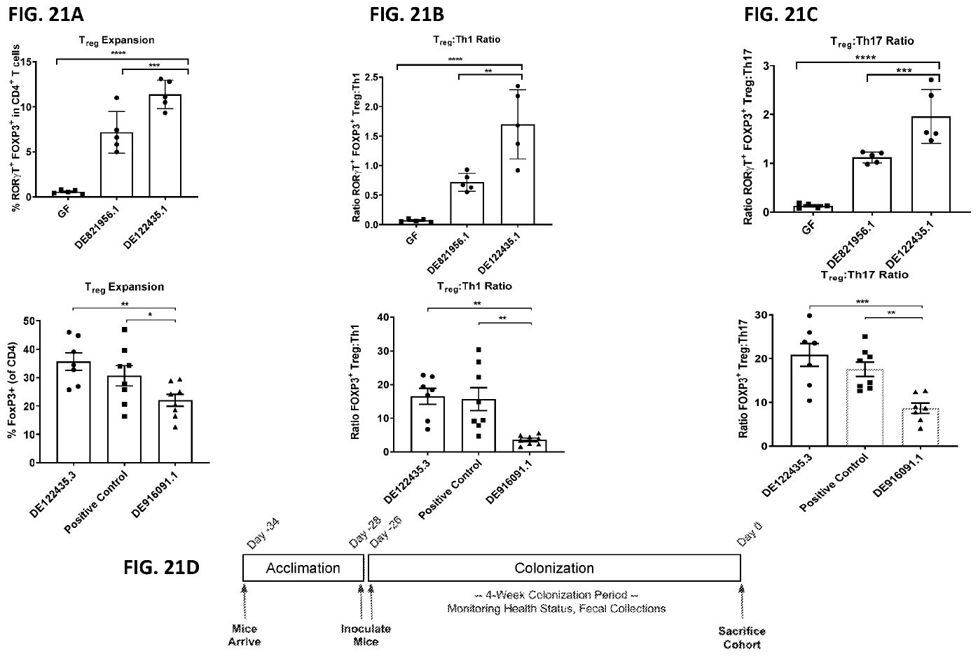

Mice colonized with DE122435.1 or DE122435.3 were found to induce a significant expansion of Foxp3+RORγt+ T cells, which represent a stable regulatory T cell (Treg) lineage known to have a highly anti-inflammatory phenotype in vivo (Yang et al., Mucosal Immunol. 2016 March; 9(2):444-57, 2016), relative to the negative controls DE821956.1 or DE916091.1 colonized mice and germ-free mice (FIG. 21A). Additionally, DE122435.1 or DE122435.3 treatment did not increase the frequency of pro-inflammatory Th1 and Th17 cells; therefore, the ratio of Treg:Th1 (FIG. 21B) and Treg:Th17 (FIG. 21C) were higher in DE122435.1 and DE122435.3 colonized mice as compared to the DE821956.1 and DE916091.1 colonized mice and mice that remained germ-free.

Together, these data show that compositions of the bacterial compositions disclosed herein significantly increased regulatory T cells and the ratio of Treg:Th1 and Treg:Th17 in the colon, both of which are important to promote immune homeostasis and counterbalance inflammatory conditions. Preclinical data shows that DE122435.1 and DE122435.3 can reduce inflammation in the epithelial barrier, as measured in vitro, and in the colonic lamina propria of mice via assessments of Treg cells and pro-inflammatory Th1 and Th17 cells.

### Example 10: In Vivo Tolerability and Pharmacological Summary

Further to Examples 2 and 3 provided above, both the tolerability and pharmacological analysis of the different bacterial compositions disclosed herein were assessed in the VRE and CRE animal models.

Administration of DE122435.3 did not cause mortality or adverse clinical symptoms (lethargy, hunched posture, rectal bleeding, significant weight loss) in numerous studies across multiple mouse models. In the VRE and CRE colonization mouse models, mice receive antibiotic conditioning followed by VRE or CRE challenge. Control arms (no DE122435.3 dosing) do not exhibit any clinical symptoms or mortality. The addition of 3-6 days of daily DE122435.3 dosing by oral gavage in these models (n=34 mice) did not cause any change in clinical observations and no mortality was observed, suggesting that DE122435.3 treatment was well-tolerated. Additionally, DE122435.3 was dosed in GF mice (n=30 mice) and over a 4-6 week colonization period, no mortality or adverse clinical symptoms were observed. Together, these data support that DE122435.3 treatment, at doses found to be efficacious in the VRE and CRE colonization models, is well-tolerated in mice.

DE122435.3 was tested in vivo in VRE and CRE colonization models as well as dosed in GF mice. In these mouse models, DE122435.3 was well-tolerated and demonstrated efficacy by reducing both VRE and CRE titers by more than 99% (2-3 Log) over several weeks. DE122435.3 was screened in vitro in IECs and shown to decrease TNF-α driven secretion of the inflammatory cytokine IL-8, and lack of induction of IL8 secretion in the absence of TNF-α suggesting that DE122435.3 can modulate relevant inflammatory pathways. When the highly related composition DE673670.1 was tested in an in vitro epithelial barrier model, the composition led to a significant reduction in barrier permeability after IFN-γ treatment. Additionally, a GF mouse model of immunomodulation was used to assess the impact of DE122435.3 and the highly related composition, DE122435.1, on immune cell populations in the colon, specifically the ratio of Treg to Th1 and Treg to Th17 cells in the colonic lamina propria. GF mice have dramatically reduced colonic lamina propria CD4+ T cells relative to conventional mice and the colonization of GF mice with bacteria induces rapid expansion and differentiation of CD4+ T cell populations. Among the cells most highly induced by colonization are Th1 and Th17 effector T cells and regulatory T cells (Tregs). Variations in microbial composition can alter the balance of Tregs to Th1 and Th17, inducing either immune homeostasis or an inflammatory environment in the gut. In HSCT, Treg-mediated regulation of Th1 and Th17 inflammatory responses is a crucial factor in alleviating GvHD. Colonization with DE122435.3 and DE122435.1 led to a significantly increased frequency of Tregs and did not increase the frequency of pro-inflammatory Th1 or Th17 effector T cells in the colon compared to mice colonized with an inflammatory composition (DE916091.1). Thus, colonization with DE122435.3 or DE122435.1 resulted in a higher ratio of Tregs to Th1 and Treg to Th17, both of which are important to promote immune homeostasis and counterbalance inflammatory conditions. Given these data, it is anticipated that in addition to significantly reducing VRE and CRE burden in mice (see, e.g., Examples 2 and 3), DE122435.3 will promote immune homeostasis and protect against epithelial barrier damage in vitro.

### Example 11: Analysis of the Anti-Tumor Effect of Designed Bacterial Compositions in Combination with Immune Checkpoint Inhibitor (ICI) Antibodies in a CT26 Tumor Model

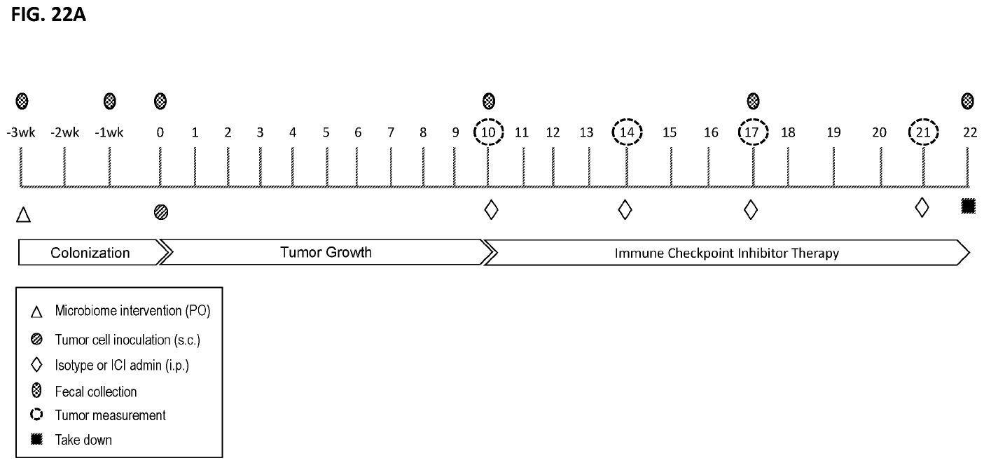

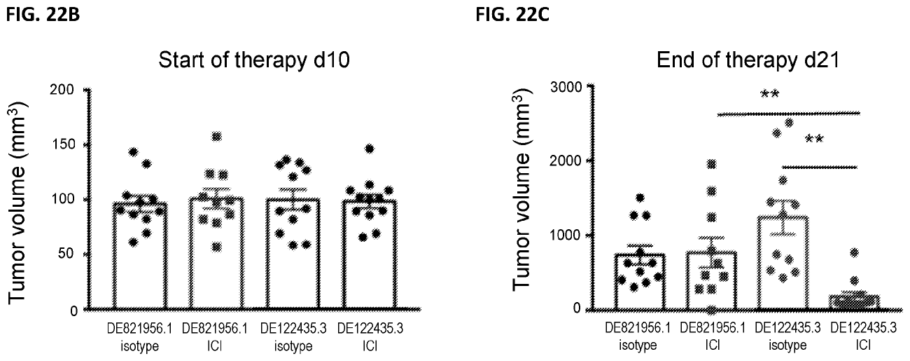

To assess whether the designed compositions disclosed herein could also be useful in treating cancers, a CT26 tumor model was used. Briefly, approximately three weeks prior to tumor inoculation, the DE122435.3 or negative control DE821956.1 composition was administered to the germ-free animals. DE122435.3 was administered once at a dose of 10⁷ per strain (see triangle mark in FIG. 22A) and allowed to colonize the gastrointestinal tract of the animals for 3 weeks. Then, a total of 5×10⁵ of the CT26 tumor cells were transplanted into the animals (via subcutaneous administration) (see circle mark in FIG. 22A). Once the tumors had reached an optimal size, the animals were randomized and further treated with one of the following: (i) isotype antibody or (ii) combination of ICI antibodies (i.e., anti-PD-L1+anti-CTLA-4). The antibodies were administered (via intraperitoneal administration) to the animals at a dose of 200 μg/ml at days 10, 14, 17, and 21 post tumor inoculation (see diamond marks in FIG. 22A). Tumor volume was measured at days 10, 14, 17, and 21 post tumor inoculation. At day 22, the animals were sacrificed and the anti-tumor immune response was assessed in various tissues. Fecal pellets were collected at week −3, week −1, day 0, days 10, 17, and 22 post tumor inoculation for next generation sequencing analysis.

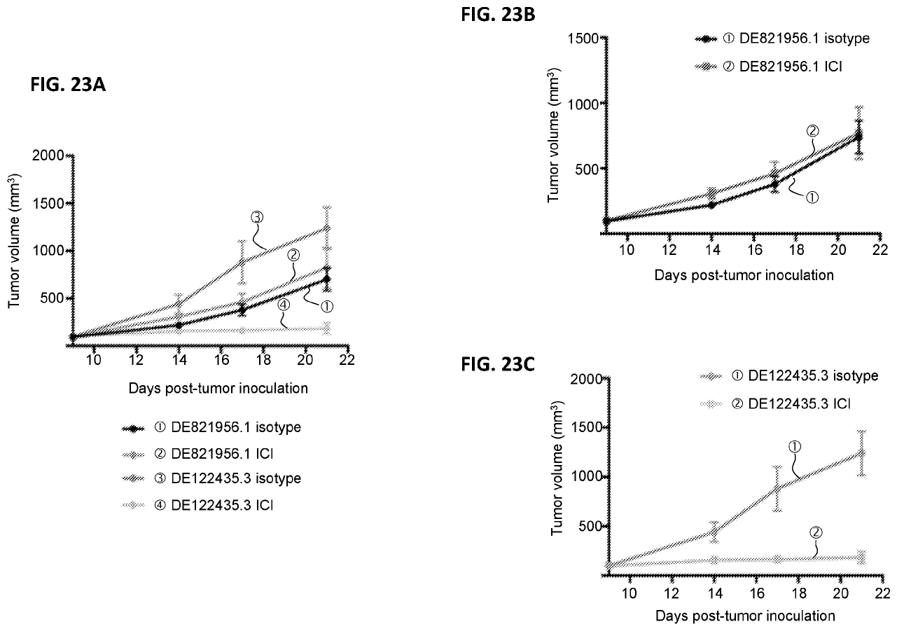

As shown in FIG. 22B, prior to the administration of the ICI antibodies, tumor volume was comparable among the different groups. However, at day 21 post tumor inoculation when the last antibody dose was administered to the animals, animals that received both the DE122435.3 composition and the combo ICI antibodies had greater reduction in tumor volume, compared to the control animals or the isotype groups. The increased reduction in tumor volume was apparent as early as day 14 post tumor inoculation (i.e., when the second dose of the ICI antibodies was administered) (FIGS. 23A, 23B, and 23C). The reduced tumor volume was not solely due to the ICI antibodies, as animals that were colonized with the negative control DE 821956.1 composition and subsequently treated with the ICI antibodies had significantly greater tumor volume (e.g., similar to the control animals).

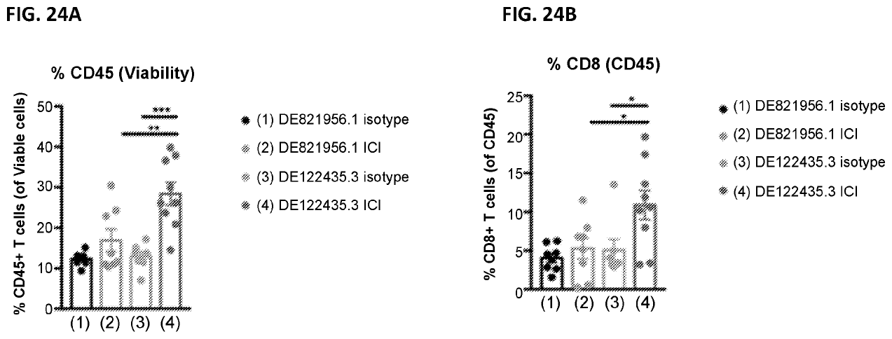

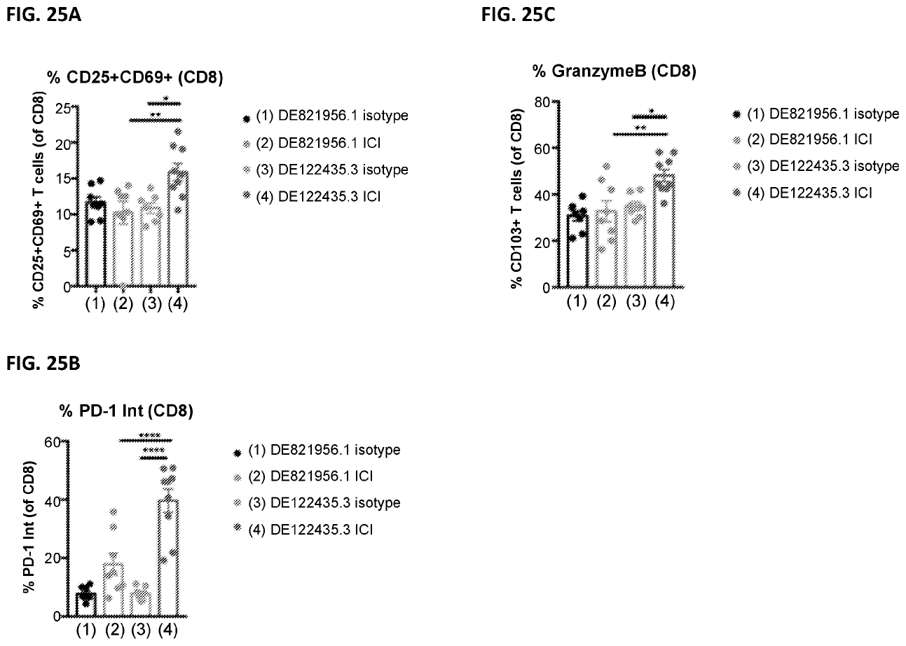

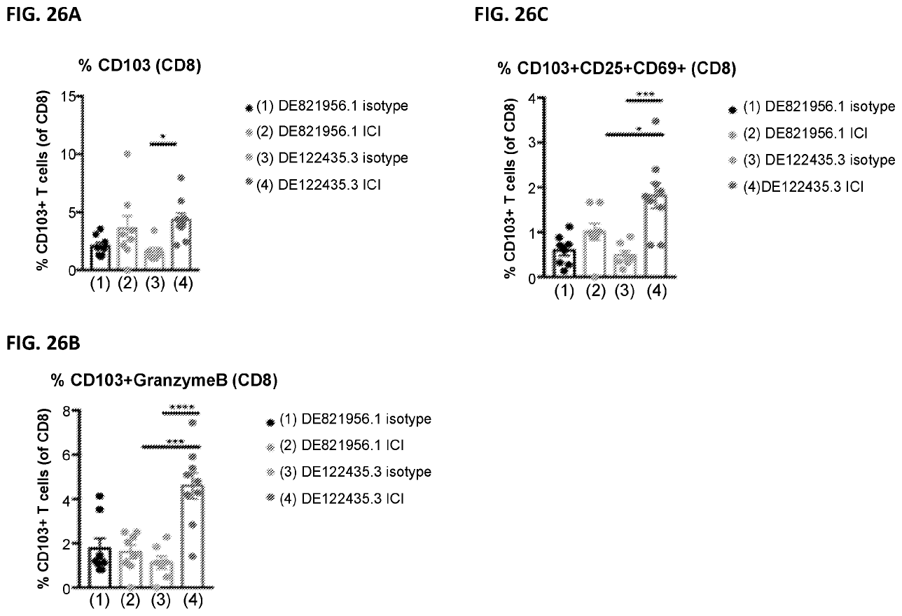

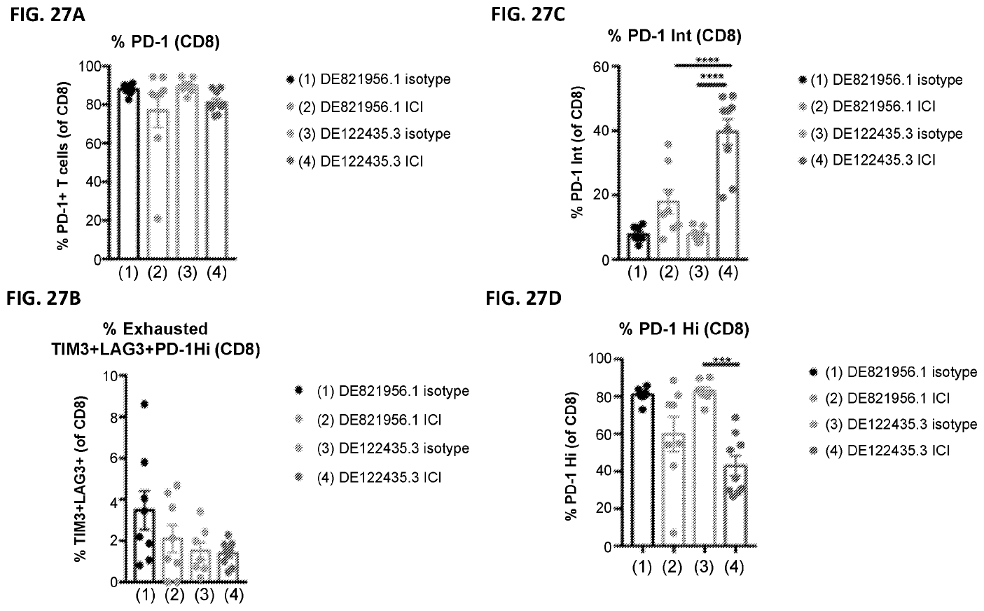

The improved effect on tumor volume was associated with increased T cell immune response in the tumors, resulting in increased frequency of total T cells (i.e., CD45+ cells) and particularly CD8+ T cells (FIGS. 24A and 24B). Moreover, greater percentage of the CD8+ T cells in the tumors of animals that received both the DE122435.3 composition and the ICI antibodies had a greater effector phenotype compared to the other treatment groups. For instance, greater percentage of the CD8+ T cells were CD25+CD69+ and/or expressed intermediate level of PD-1 (i.e., markers for early T cell activation) (FIGS. 25A and 25B). There was also a greater frequency of CD8+ T cells that were GranzymeB+ in the tumors of animals that received both the DE122435.3 composition and the ICI antibodies (FIG. 25C). Similar increase was observed with migratory CD103+CD8+ T cells that expressed either GranzymeB+ or CD25+CD69+ double positives (CD103 is a migratory marker that help direct CD8+ T cells to tumor microenvironment and distinguishes them from tissue resident CD8+ T cells)(FIGS. 26A, 26B, and 26C). In agreement with the enhanced CD8+ T cell immune response described above, the CD8+ T cells present in the tumors of animals that received both the DE122435.3 composition and the ICI antibodies were less exhausted (FIGS. 27A, 27B, 27C, and 27D).

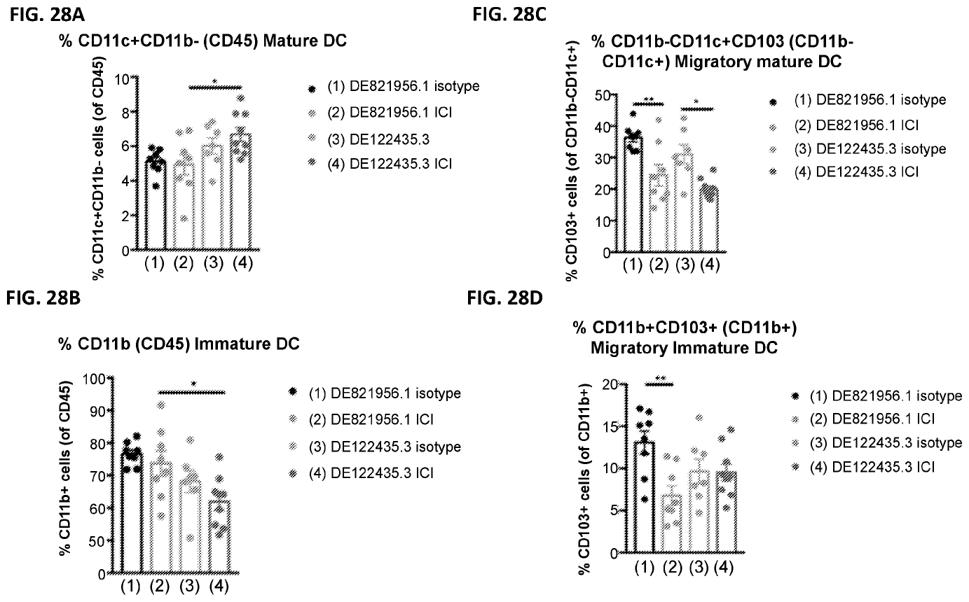

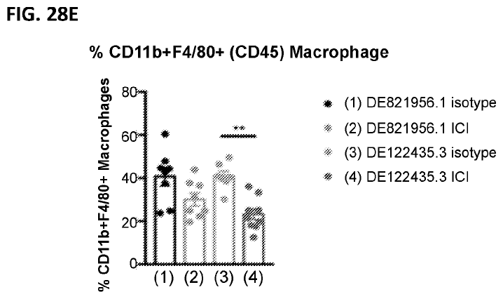

In addition to improved CD8+ T cell immune response, animals that were first colonized with the DE122435.3 composition and then subsequently treated with the ICI antibodies also exhibited a greater innate immune response compared to animals from the other treatment groups. For example, greater percentage of the dendritic cells present in the tumor tissues were of a mature (i.e., activated phenotype) compared to dendritic cells from the other treatment groups (FIGS. 28A, 28B, 28C, and 28D). On the other hand, macrophages in tumor microenvironment occur in two polarization states: M1 (anti-tumor activity) and M2 (aid in tumor growth) with CD11b+F4/80+ macrophages potentially capturing M2 phenotype which is significantly decreased in the tumors of animals that received both the DE122435.3 composition and the ICI antibodies (FIG. 28E). Again, the observed improved effect on innate immune response was not due solely to the administration of the ICI antibodies. Animals that were colonized with the negative control DE 821956.1 composition and then subsequently treated with the ICI antibodies did not exhibit an innate immune response.

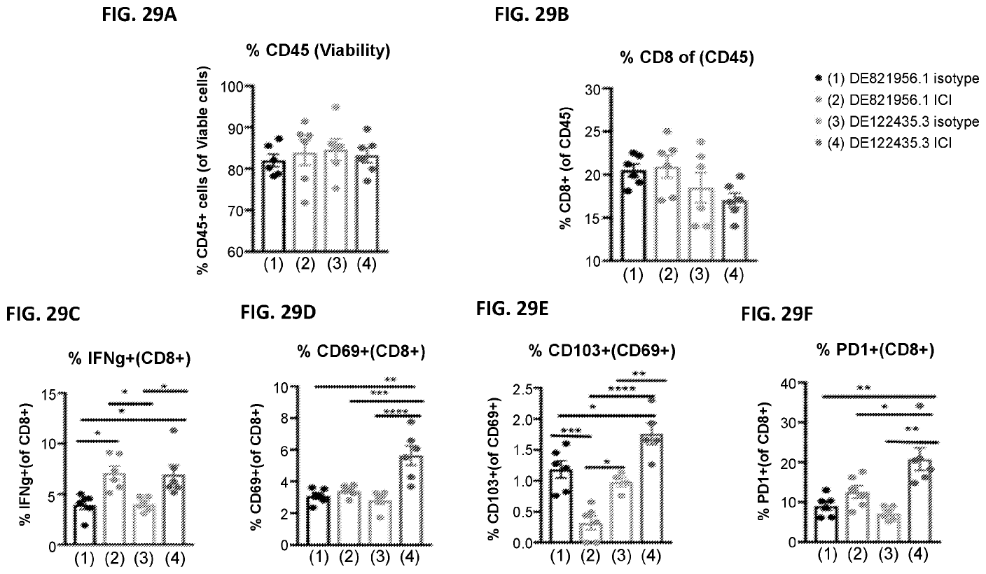

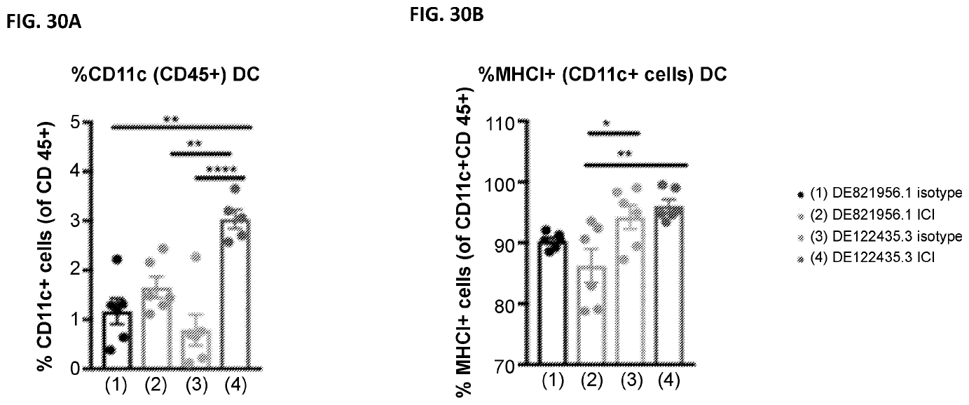

The tumor-draining lymph nodes are pivotal in checkpoint therapy and are often first sites of metastasis. Accordingly, the anti-tumor immune response was also characterized in the tumor-draining lymph nodes of the animals from the different treatment groups. In animals that received both the DE122435.3 composition and the ICI antibodies, there was a modest decrease in the percentage of CD8+ T cells, compared to animals from the different treatment groups (FIG. 29B). However, as observed in the tumor tissues, greater percentage of the CD8+ T cells present in the tumor-draining lymph nodes were of an effector (i.e., activated) phenotype (FIGS. 29C-29F). Similarly, there was not only a greater frequency of total dendritic cells (FIG. 30A) in the tumor-draining lymph nodes but greater percentage of the dendritic cells were of an activated, mature phenotype in animals that received both the DE122435.3 composition and the ICI antibodies (FIG. 30B).

Next, to identify cellular and global pathways related to the effects described above, gene expression analysis of tumor samples of animals from the different treatment groups was carried out using a gene expression panel (available from NanoString Technologies). The genes upregulated and the pathways differentially regulated in the animals treated with DE122435.3 and the combo ICI antibodies or with DE821956.1 composition and the combo ICI antibodies were identified. Preliminary data showed there was a significant difference in gene expression profile between animals that were colonized with the negative control DE821956.1 composition and animals that were colonized with the DE122435.3 composition prior to the administration of the ICI antibodies.

Collectively, the above data demonstrate that when administered in combination with the combo immune checkpoint inhibitors, the DE122435.3 composition can be useful in treating certain cancers. Not to be bound by any one theory, the above data suggests that one or more bacterial strains present in the DE122435.3 composition can enhance the anti-tumor efficacy of immune checkpoint inhibitors (e.g., anti-PD-L1 and/or anti-CTLA-4 antibodies). Moreover, as described supra, cancers are generally not thought to be associated with pro-inflammatory responses, and cancer immunotherapy generally aims to increase host pro-inflammatory responses targeting cancer cells. Therefore, it was not reasonably expected that a bacterial composition designed to have anti-inflammatory properties (i.e., DE122435.3) would be effective for enhancing anti-tumor response. The result further highlights that a bacterial composition can be designed to target multiple immune pathways, and thereby, treat wide range of diseases, including both inflammatory diseases and cancers.

### Example 12: Analysis of the Effect of Designed Compositions on Human CD8 T Cell Activation

As shown in the examples above, when administered in combination with the combo immune checkpoint inhibitors, the designed bacterial compositions described herein (e.g., DE122435.3 composition) can induce the activation and expression of genes that are important in an efficacious anti-tumor immune response (e.g., mediated by CD8 T cells). To further assess the effects of the DE122435.3 composition a human CD8 T cell activation assay was constructed. The in vitro assay mimics in vivo T cell activation from antigen-presenting cells by utilizing the two activation signals CD3 and CD28, bound to a three-dimensional bead similar in size to the antigen-presenting cells.

Briefly, the human primary CD8 T cells were thawed at 37° C. for 24 hr and activated by CD3 and CD28 at 37° C. for 2 days. The cells were treated at 37° C. for 24 hr with bacterial supernatants from different bacterial compositions, including DE122435.3, DE916091.1, and DE821956.1, or negative control bacterial media. The cells were analyzed by flow cytometer for viability and proliferation. The cell lysates were used in gene expression analysis using multiplexed molecular barcode (available from NanoString Technologies) or multiplex panels.

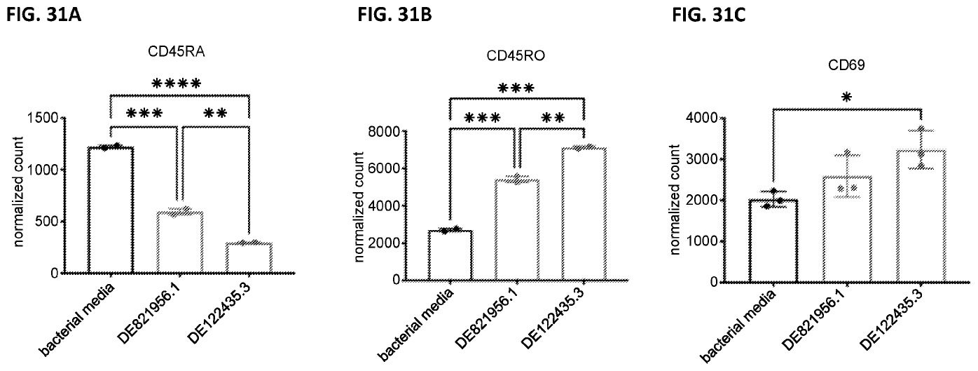

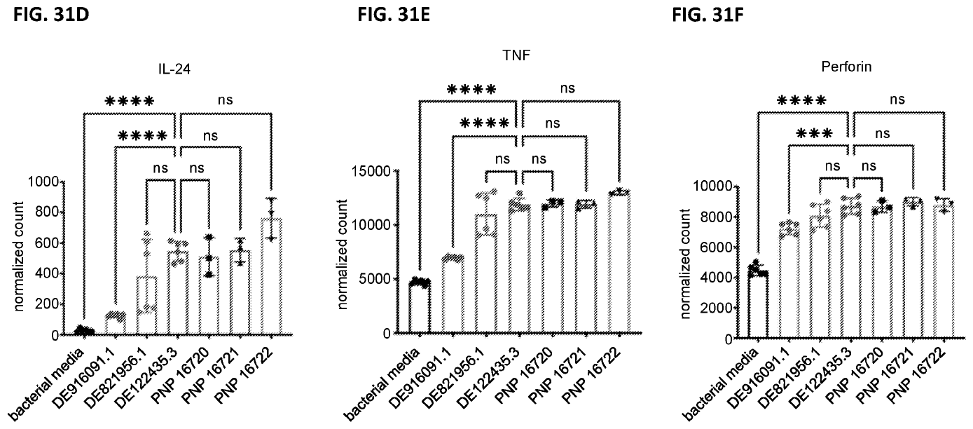

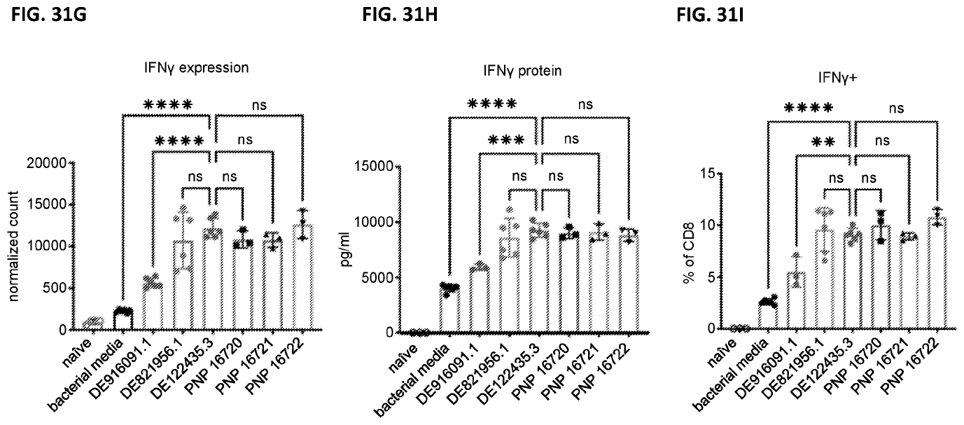

The gene expression was analyzed in the cells treated with the bacterial supernatants and results are shown in Table 5 and FIGS. 31A-31I. As shown in Table 5 below, DE122435.3 supernatants induced signification gene expression changes in T cells, differently from DE821956.1 and the negative control.

TABLE 5
Pathway-level z scores identifying the gene expression changes of the treated T cells.

| | bacterial | bacterial | | | | |
|---|---|---|---|---|---|---|
| | media | media | DE821956.1 | DE821956.1 | DE122435.3 | DE122435.3 |
| Activation | −5.1342 | −5.6094 | 0.7897 | 0.9066 | 4.9715 | 4.0758 |
| Antigen | −1.0659 | −1.3084 | −0.0653 | 0.0024 | 1.3300 | 1.1072 |
| processing & | | | | | | |
| presentation | | | | | | |
| Chemokine | −2.4862 | −2.8808 | 0.7337 | 0.5964 | 2.1137 | 1.9232 |
| Signaling | | | | | | |
| Costimulatory | −1.8123 | −2.3025 | 0.4524 | 0.6192 | 1.6514 | 1.3918 |
| Molecules | | | | | | |
| Cytotoxicity | −2.3316 | −2.3982 | 0.3491 | 0.4137 | 2.0807 | 1.8863 |
| Glycolysis | −0.5747 | −0.6413 | −0.0955 | 0.0792 | 0.7012 | 0.5310 |
| Interactions with | −1.6440 | −1.6779 | 0.0226 | 0.0545 | 1.6857 | 1.5590 |
| Non-lymphoid | | | | | | |
| Cells | | | | | | |
| Interleukin | −3.0890 | −2.8270 | 0.5350 | 0.5133 | 2.6278 | 2.2399 |
| Signaling | | | | | | |
| Oxidative | 0.8769 | 0.0391 | 0.2540 | 0.5228 | −0.8855 | −0.8073 |
| phosphorylation | | | | | | |
| T-cell migration | −1.9986 | −2.0547 | 0.1934 | 0.1443 | 1.9883 | 1.7273 |
| TCR signaling | −2.6098 | −3.0181 | 0.4743 | 0.7160 | 2.4112 | 2.0264 |
| Toxicity | −5.1566 | −5.3018 | 0.8145 | 0.7735 | 4.7381 | 4.1324 |
| Type I interferon | −1.9067 | −1.8725 | 0.1505 | 0.1518 | 1.9064 | 1.5706 |
| signaling | | | | | | |
| Type II interferon | −1.9809 | −2.3533 | 0.2006 | 0.2080 | 2.1395 | 1.7861 |
| signaling | | | | | | |

The bacterial composition stimulated the activation, cytotoxicity and cytokine production by T cells as shown in FIGS. 31A-31I. Particularly as shown in FIGS. 31A-C, treatment with DE122435.3 decreased the expression of CD45RA (a gene highly expressed in naïve T cells and downregulated with activation) and increased the expression of CD45RO and CD69, two markers of T cell activation. Additionally, as shown in FIG. 31D-G, treatment with DE122435.3 resulted in increased expression of the cytokines and cytotoxic molecules IL-24, TNF, perforin and IFNγ, respectively. FIG. 31H and 31I also show the enhanced production of IFNγ protein quantified by multiplexed bead-based (e.g., available from Luminex) and flow cytometry assays, respectively.

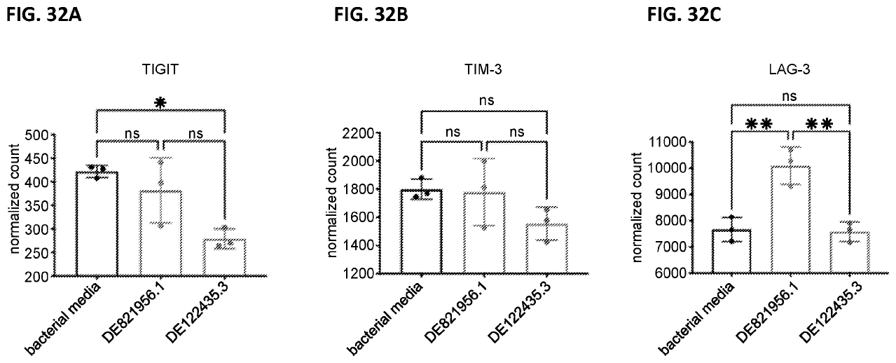

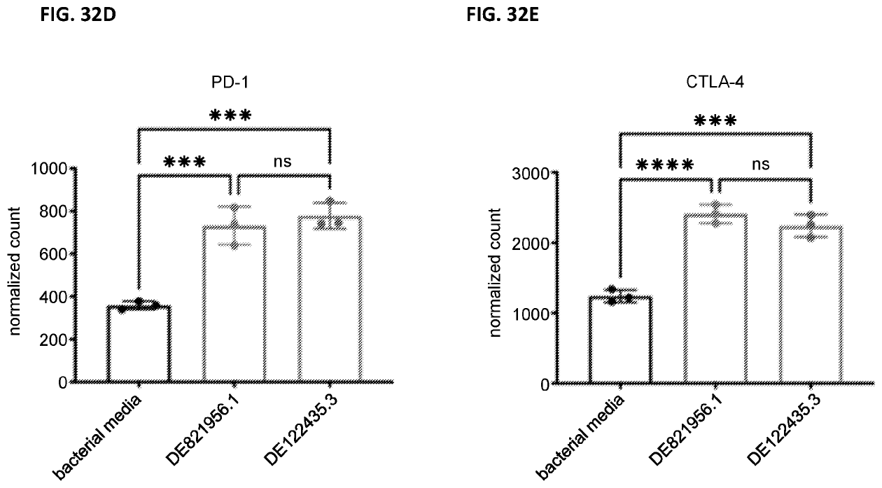

Effects of treatment with DE122435.3 on the expression of T cell inhibitory receptors were also evaluated as shown in FIG. 32A-E. The assays use CD3/CD28 activation that does not require feeder cells (antigen-presenting cells) or antigen. The primary human T cells were treated with bacterial supernatants from compositions including DE122435.3, DE821956.1, or negative control bacterial media. The gene expression of the treated T cells of all treatment groups was quantified by a gene expression panel available from NanoString Technologies. Treatment with DE122435.3 caused a decrease in the inhibitory receptors/exhaustion markers TIGIT and LAG-3 compared to the bacterial media and DE821956.1 controls. Given the induction of activation markers, enhancement of cytokine and perforin production and reduction in some inhibitory receptors, the results show the ability of the bacterial composition to enhance T cell activation and function.

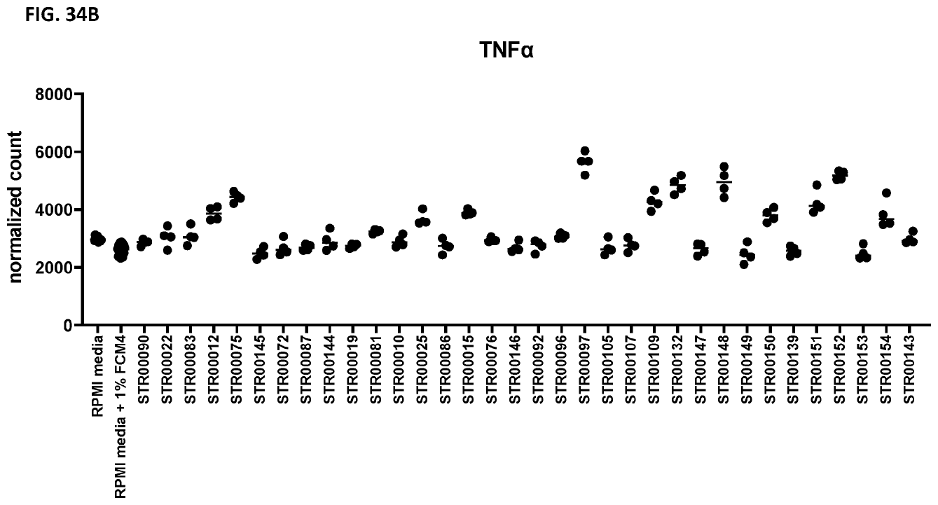

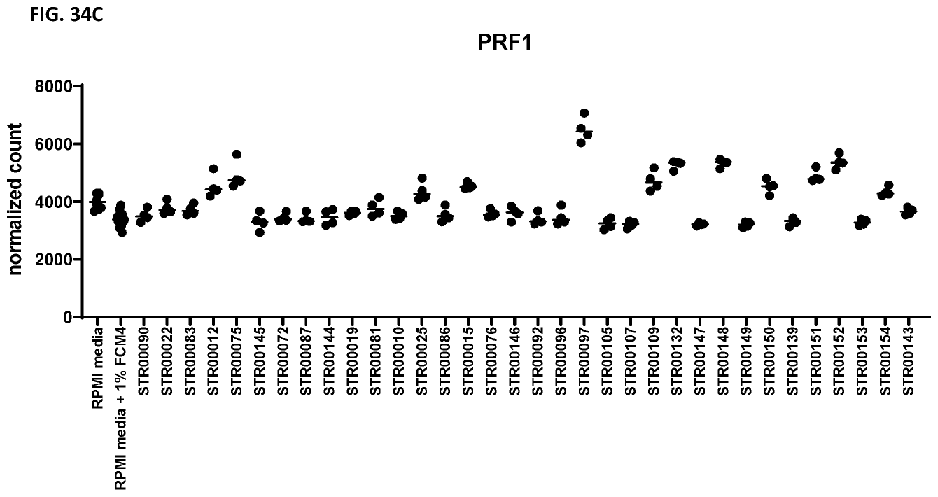

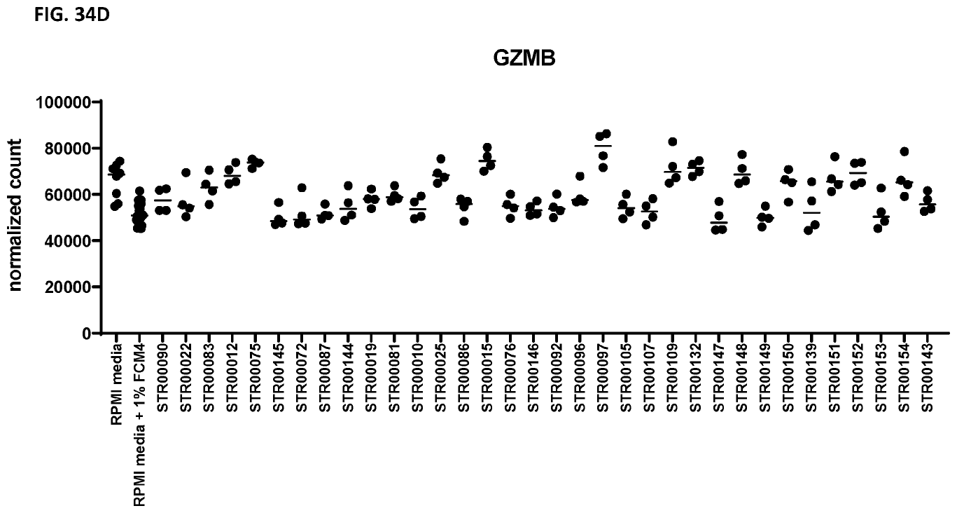

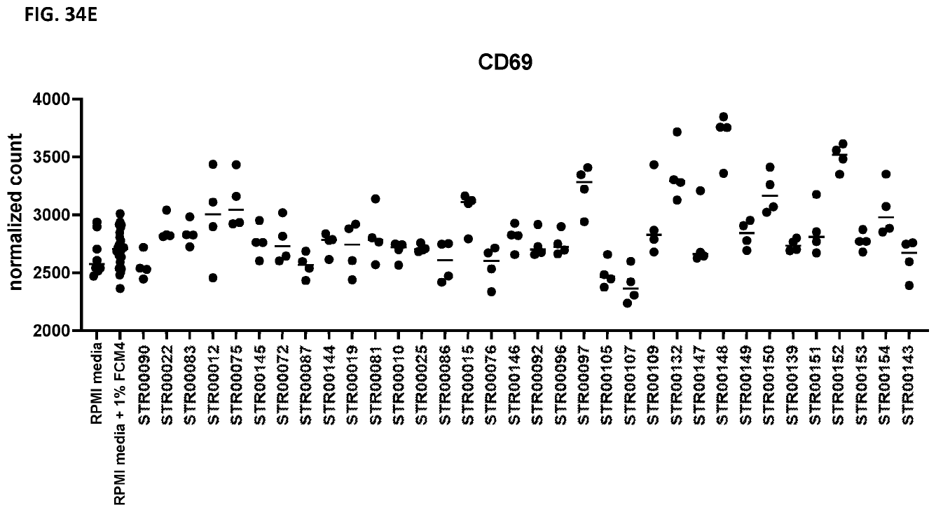

The same assay described above was used to test whether individual bacterial strains can modulate the expression of genes involved in T cell activation and effector function. The results of the Nanostring gene expression assay show that bacterial supernatants from some single strains induce the expression of the CD8 T cell effector cytokines IFNγ and TNFα (FIGS. 34A-34B), cytotoxic molecules perforin and Granzyme B (FIGS. 34C-34D) and the activation marker CD69 (FIG. 34E).

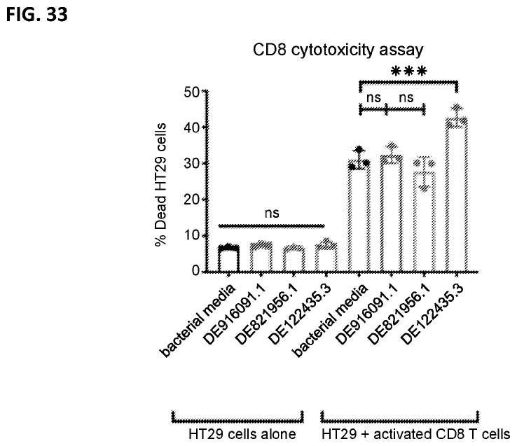

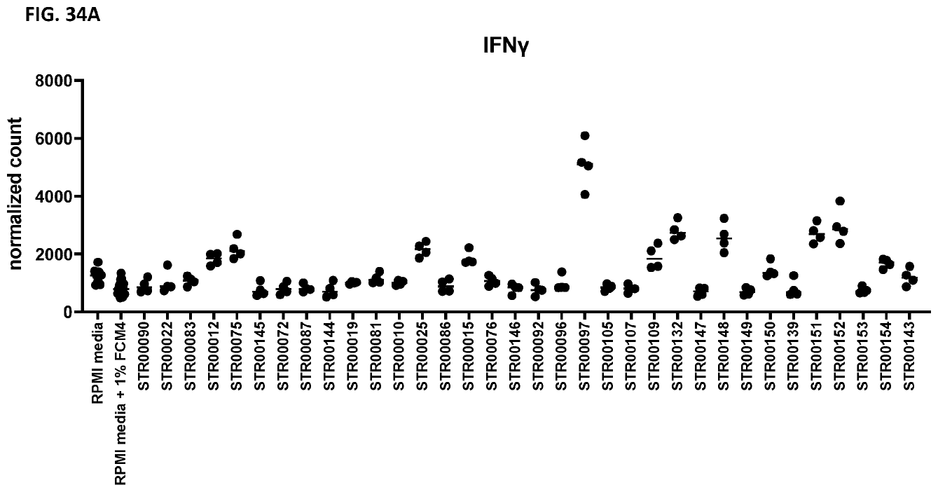

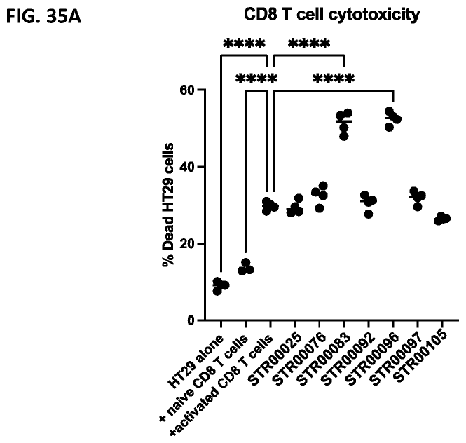

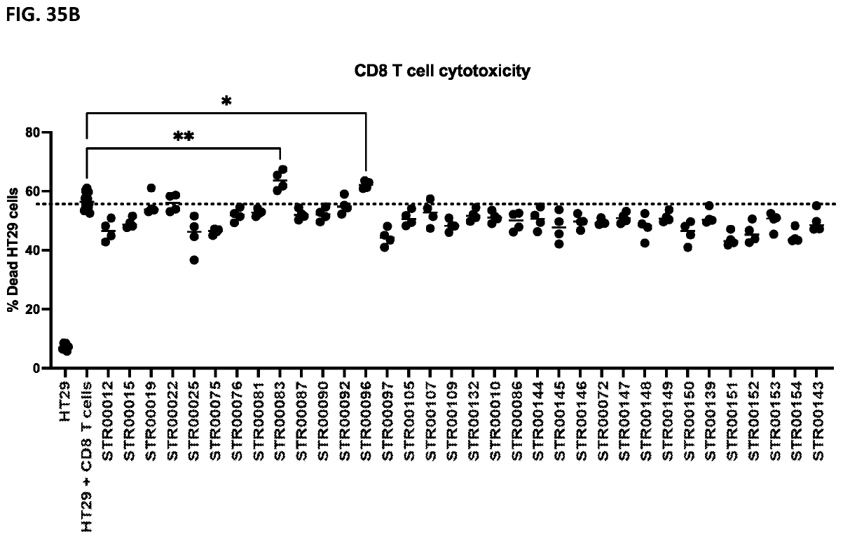

Together, these results showed bacterial compositions as described herein can have direct effects on human CD8+ T cell activation and proliferation. Differentially regulated genes by designed bacterial compositions are involved in several pathways linked to anti-tumor immunity, including cytokine production, cytotoxicity, T cell exhaustion, and immune cell recruitment. To test whether these differences in gene expression translate to enhanced ability of CD8 T cells to kill target cells, a CD8 cytotoxicity assay was developed. CD8 T cells were thawed for 24 hours and activated using beads conjugated to α-CD3 and α-CD28 antibodies for 48 hours as described above. The activated CD8 T cells were subsequently co-cultured with HT29 cells, a colorectal cancer cell line, for another 24 hours in the presence of supernatants of DE122435.3, the negative controls DE916091.1 and DE821956.1 and bacterial media as background. A flow cytometer was used to determine the viability of both the CD8 T cells and the HT29 cells. The results showed that co-culturing activated CD8 T cells with the HT29 target cells in the presence of supernatants of DE122435.3 enhanced the CD8 T cells' cytotoxicity and their ability to kill HT29 target cells compared to the bacterial media background and the negative controls DE916091.1 and DE821956.1 (FIG. 33). The same assay was used to test the ability of single bacterial strains to enhance CD8 T cell cytotoxicity. FIGS. 35A-35B show enhanced killing of HT29 target cells when the CD8 T cells were treated with supernatants from two single bacterial strains.

### Example 13: Analysis of the Effect of Designed Compositions on Systemic Inflammation in 5-FU Induced Murine Mucositis Model

To assess the effect of the designed compositions disclosed herein on circulating inflammatory cytokine levels in response to local GI damage, a chemotherapy-induced murine mucositis model was used with DE486373.1 versus a composition designed to be inflammatory (IgA+, DE916091.1). Briefly, following an acclimation period, germ-free mice were orally dosed with DE486373.1 or DE916091.1. Five weeks post colonization, either 200 mg/kg 5-FU (fluorouracil) or 0.9% saline (vehicle control) was administered intraperitoneally over 3 consecutive days. Body weight was monitored daily and clinical observation was performed twice a week. Fecal pellets were collected for 16Sv4 sequencing. Serum for cytokine analysis and pieces of small intestine/colon were taken for histopathology analysis on necropsy days (day 5 and day 8).

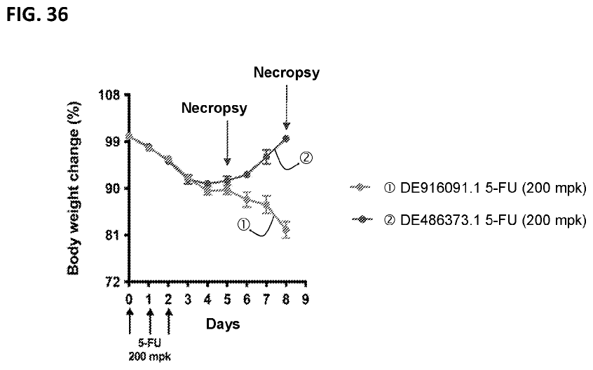

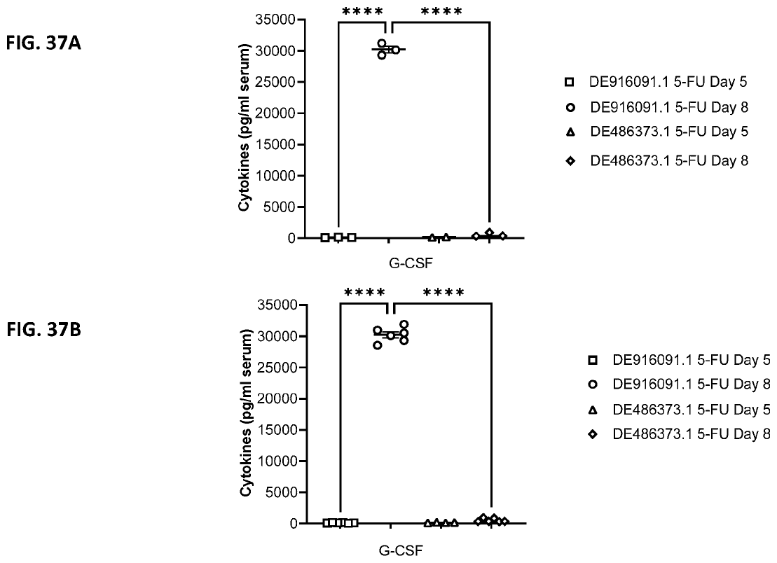

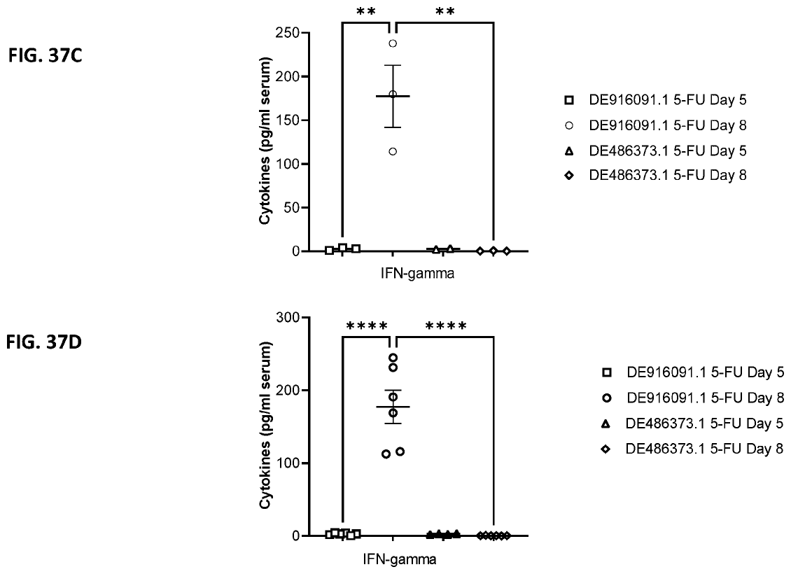

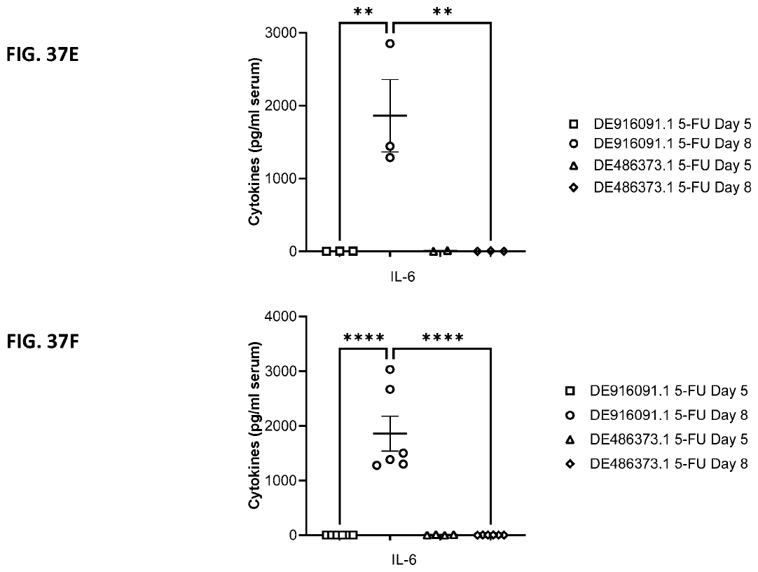

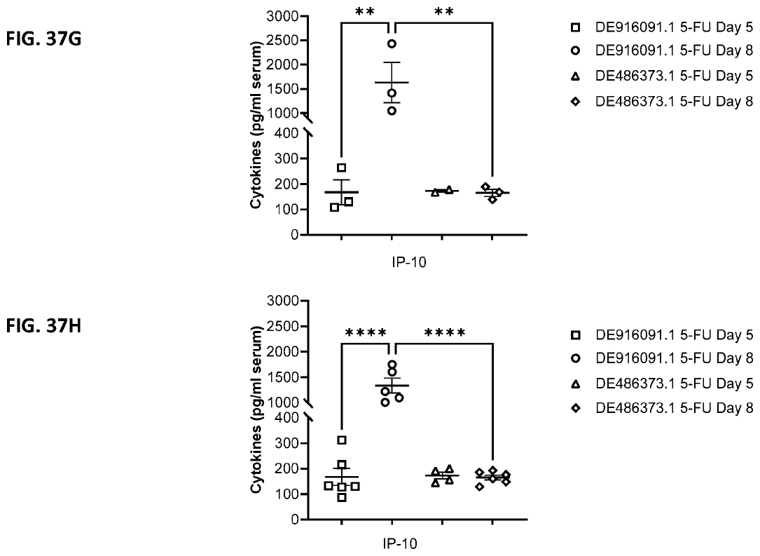

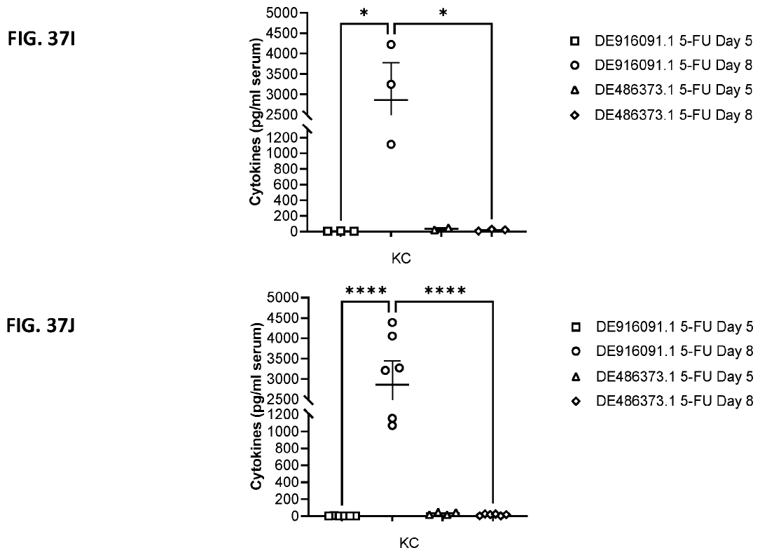

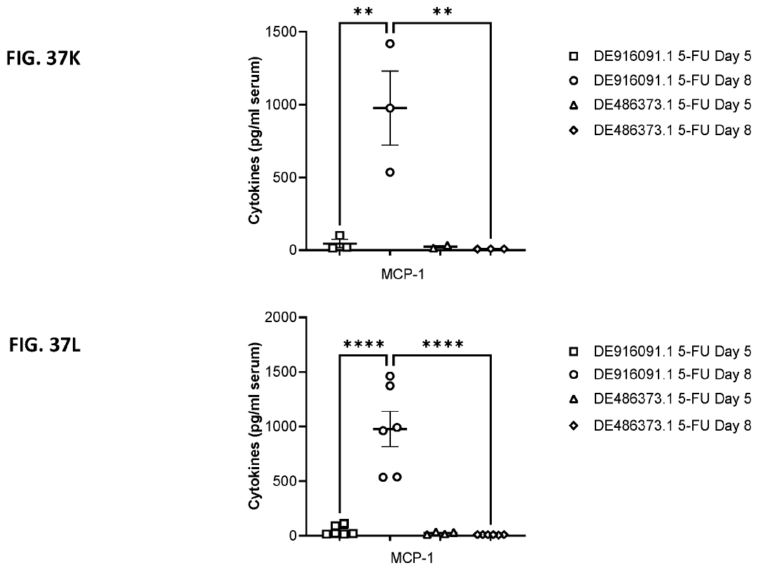

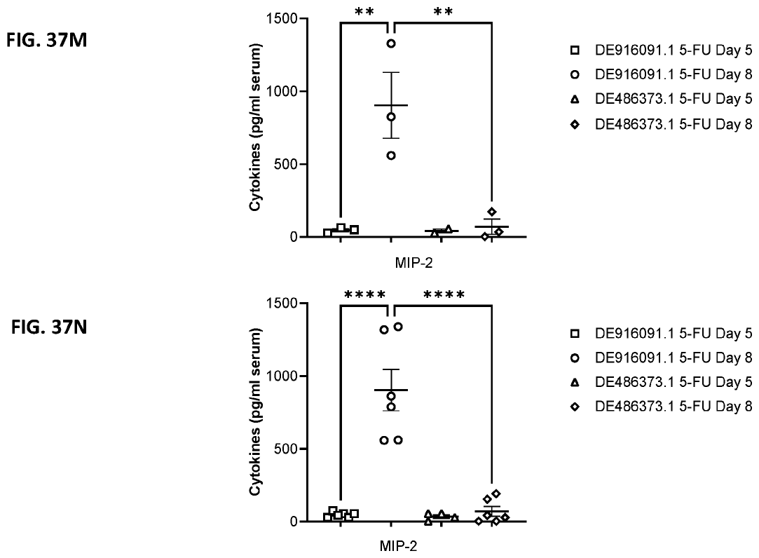

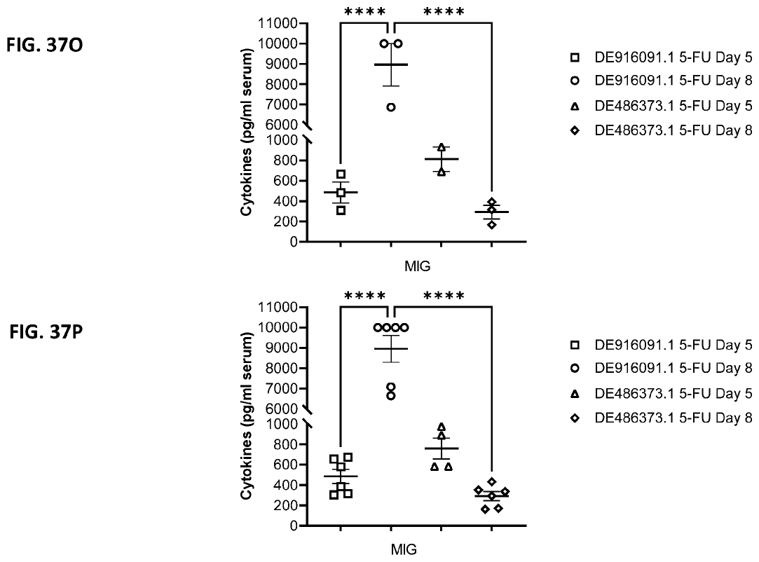

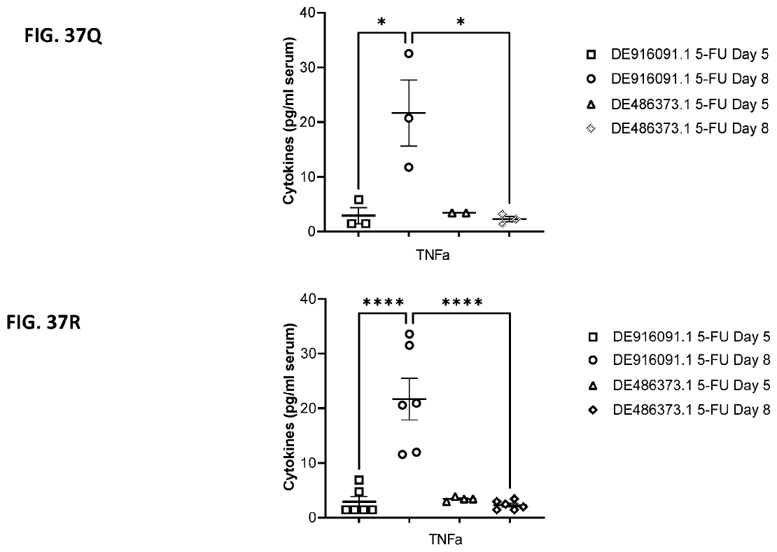

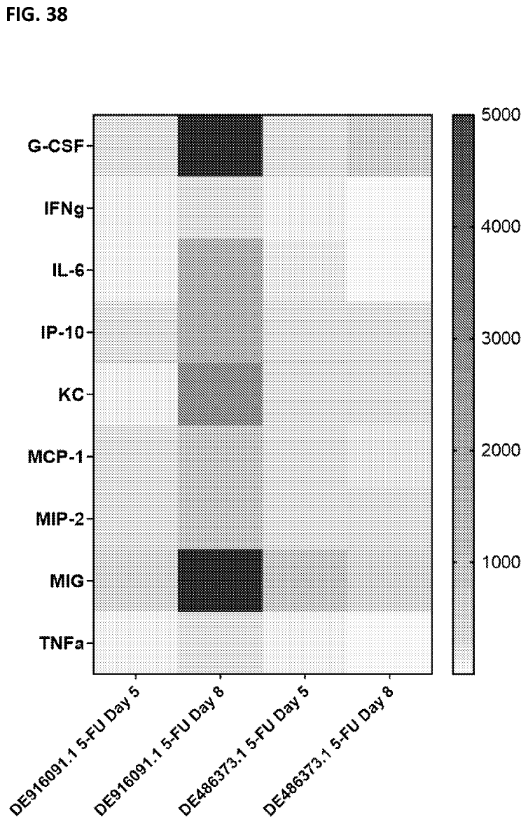

To evaluate the levels of murine cytokines and chemokines in the serum, blood was collected on days 5 and 8 in microcentrifuge tubes, allowed to clot, centrifuged, and serum collected. The Luminex xMAP mouse cytokine/chemokine immunoassay was conducted per the manufacturer's instructions (Milliplex). Results were acquired with a Luminex MagPix instrument and analyzed with xPONENT software (Luminex corporation). Upon analyzing the Luminex data, statistical analysis of G-CSF, IFNγ, IL-6, IP-10, KC, MCP-1, MIP-2, MIG, and TNFα was done using GraphPad Prism to confirm significantly differential levels between the two groups of mice. As compared to DE916091.1, mice colonized with DE486373.1 recovered the initial body weight loss due to 5-FU treatment from day 5 following 5-FU administration (FIG. 36). Across the Luminex panel of 32 cytokines and chemokines, mice colonized with DE486373.1 showed no discernable increase in any analyte measured at days 5 and 8 following 5-FU administration (FIGS. 37A-37R and 38). However, mice colonized with DE916091.1, which was designed as an inflammatory composition, showed a significant increase in proinflammatory factors like G-CSF, IL-6, IFNγ, TNFα, IP-10, KC, MCP-1, MIG and MIP-2 systemically when measured at day 8 compared to day 5 (FIGS. 37A-37R and 38).

These findings suggest that colonization of germ-free mice with DE486373.1 does not increase any circulating inflammatory cytokines analysed in a chemotherapy-induced mucositis model, as compared to colonization with the inflammatory composition DE916091.1. Proinflammatory cytokines such as Granulocyte colony-stimulating factor (G-CSF), Interleukin-6 (IL-6), Interferon-gamma (IFNγ), and tumor necrosis factor alpha (TNFα) are known to impair epithelial barrier function. On the other hand, chemokines such as CXCL1 (KC), CXCL2 (MIP-2) and CXCL9 (MIG) have been shown to recruit neutrophils, monocytes/macrophages or activated T cells to the site of infection/inflammation. Furthermore, these circulatory factors play a critical role in the damage and regeneration phases of the small intestine upon 5-FU chemotherapy. Thus, DE486373.1 ameliorates the systemic inflammation resulting from gut epithelial barrier damage caused by 5-FU (FIG. 38).

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

The claims in the instant application are different than those of the parent application or other related applications. The Applicant therefore rescinds any disclaimer of claim scope made in the parent application or any predecessor application in relation to the instant application. The Examiner is therefore advised that any such previous disclaimer and the cited references that it was made to avoid, may need to be revisited. Further, the Examiner is also reminded that any disclaimer made in the instant application should not be read into or against the parent application.

What is claimed is:

1. A composition comprising a purified population of bacteria, wherein the purified population of bacteria comprises two or more bacteria, wherein the first purified population of bacteria have a 16S rDNA sequence that is at least 97%, identical to a 16S rDNA sequence set forth in SEQ ID NOs: 19, 21, 31, 35, 36, 42, 44, 80, 85, 99, 104, 105, 162, 166, 183, 193, 197, 205, 215, 222, 226, 231, 233, 235, and 241-341, and wherein the second purified population of bacteria are:
(i) capable of reducing VRE and CRE carriage and restore colonization resistance in the GI tract of a mammal as compared to a reference (e.g., composition that does not include one or more of the bacteria in the composition);
(ii) capable of protecting the epithelial barrier from cytokine-mediated inflammatory damage;
(iii) capable of reducing inflammation in the epithelial barrier, as measured by IL-8 secretion and/or modulation of inflammatory pathway gene expression in vitro or in the colonic lamina propria of mice as compared to a reference (e.g., composition that does not include one or more of the bacteria in the composition);
or a combination of any of (i), (ii), and (iii).

2. A composition comprising a purified population of bacteria, wherein the purified population of bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1-352.

3. A composition comprising a purified population of bacteria, wherein the purified population of bacteria comprises Eubacterium maltosivorans, Clostridium aldenense, Clostridium bolteae, Clostridium glycyrrhizinilyticum, Clostridium hylemonae, Clostridium innocuum, Clostridium lavalense, Clostridium scindens, Clostridium spiroforme, Clostridium symbiosum, Eubacterium rectale, Ruminococcus gnavus, Ruminococcus torques, Absiella dolichum, Agathobaculum desmolans, Akkermansia muciniphila, Alistipes finegoldii, Alistipes shahii, Anaerofustis stercorihominis, Anaeromassilibacillus senegalensis, Anaerostipes caccae, Anaerotruncus colihominis, Bacteroides caccae, Faecalibacterium prausnitzii, Faecalicatena contorta, Faecalicatena orotica, Flavonifractor plautii, Gemmiger formicilis, Harryflintia acetispora, Holdemania filiformis, Holdemania massiliensis, Intestinimonas butyriciproducens, Lachnospira pectinoschiza, Lachnospiraceae bacterium 5 1 57FAA, Lactobacillus fermentum, Lactonifactor longoviformis, Longibaculum muris, Longicatena caecimuris, Murimonas intestina, Oscillibacter ruminantium, Bacteroides eggerthii, Bacteroides faecis, Bacteroides intestinalis, Bacteroides koreensis, Bacteroides kribbi, Bacteroides salyersiae, Bacteroides uniformis, Bacteroides vulgatus, Bacteroides xylanisolvens, Barnesiella intestinihominis, Bifidobacterium dentium, Bifidobacterium longum, Bifidobacterium stercoris, Blautia coccoides, Blautia hominis, Blautia hydrogenotrophica, Blautia luti, Blautia obeum, Blautia producta, Blautia wexlerae, Butyricimonas faecihominis, Cellulosilyticum lentocellum, Clostridium butyricum, Ruthenibacterium lactatiformans, Sellimonas intestinalis, Shigella flexneri, Terrisporobacter mayombei, Terrisporobacter petrolearius, Turicibacter sanguinis, Tyzzerella nexilis, Clostridium disporicum, Clostridium subterminale, Clostridium tertium, Collinsella aerofaciens, Coprococcus comes, Coprococcus eutactus, Dorea longicatena, Drancourtella massiliensis, Eggerthella lenta, Eisenbergiella tayi, Emergencia timonensis, Erysipelatoclostridium ramosum, Eubacterium callanderi, Paeniclostridium sordellii, Parabacteroides distasonis, Parabacteroides merdae, Paraclostridium bifermentans, Peptostreptococcus stomatis, Robinsoniella peoriensis, Romboutsia timonensis, Roseburia intestinalis, Roseburia inulinivorans, Ruminococcus albus, Ruminococcus bromii, Ruminococcus faecis, Ruminococcus lactaris, or combinations thereof.

4. A composition comprising a purified population of bacteria, wherein the purified population of bacteria comprises a species selected from FIG. 1 or combinations thereof.

5. The composition of any one of claims 1 to 4, wherein the purified population of bacteria comprises at least two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, or more bacteria.

6. A composition comprising a purified population of bacteria, wherein the composition comprises the purified population of bacteria selected from DE1-DE54 recited in FIG. 1.

7. The composition of claim 6, wherein the composition comprises the purified population of bacteria selected from DE8, DE10, DE11, or DE23 recited in FIG. 1.

8. The composition of claim 6 or 7, wherein the composition comprises the purified population of bacteria from DE10 or DE8.

9. The composition of any one of claims 1-8, wherein the composition decreases an infection, including an infection caused by an ESKAPE pathogen (including Enterococcus faecium, Staphylococcus aureus, Klebsiella pneumoniae, Acinetobacter baumannii, Pseudomonas aeruginosa, and Enterobacter spp.) compared to a reference (e.g., a composition that does not include one or more of the bacteria disclosed herein).

10. The composition of any one of claims 1-9, wherein the composition decreases an infection, including an infection caused by an Enterococcus species including an Enterococcus species selected from Enterococcus faecalis and Enterococcus faecium compared to a reference (e.g., a composition that does not include one or more of the bacteria disclosed herein).

11. The composition of any one of claims 1-9, wherein the composition decreases an infection, including an infection caused by an Enterobacteriaceae species including Klebsiella pneumonia compared to a reference (e.g., a composition that does not include one or more of the bacteria disclosed herein).

12. The composition of any one of claims 1-9, wherein the composition decreases an infection, including an infection caused by a bacterial species resistant to vancomycin or carbapenems compared to a reference (e.g., a composition that does not include one or more of the bacteria disclosed herein).

13. The composition of any one of claims 1-9, wherein the composition decreases an infection, including an infection caused by a drug resistant or multi-drug resistant (MDROs) bacteria including a vancomycin-resistant Enterococcus spp. selected from Enterococcus faecalis and Enterococcus faecium, a carbapenem-resistant Enterobacteriaceae spp. selected from Klebsiella pneumonia, Klebsiella oxytoca, Klebsiella aerogenes, and Enterobacter spp., an extended spectrum beta-lactamase (ESBL) selected from E. coli and Klebsiella species, a methicillin-resistant Staphylococcus aureus (MRSA), or combinations thereof, compared to a reference (e.g., a composition that does not include one or more of the bacteria disclosed herein).

14. The composition of any one of claims 1 to 13, wherein the purified population of bacteria decreases the number and/or relative abundance of antibiotic resistant bacteria and/or an ESKAPE pathogen in a gastrointestinal tract of a subject compared to a reference (e.g., a composition that does not include one or more of the bacteria disclosed herein).

15. The composition of any one of claims 1 to 14, wherein the number of antibiotic resistant bacteria is measured as a colony forming unit per gram of a sample obtained from the subject.

16. The composition of any one of claims 1 to 15, wherein:
(i) the antibiotic resistant bacteria comprise vancomycin-resistant Enterococci or carbapenem-resistant Enterobacteriaceae or a combination thereof;
(ii) the antibiotic resistant bacteria comprise a drug resistant or multi-drug resistant (MDROs) bacteria including a vancomycin-resistant Enterococcus spp. selected from Enterococcus faecalis and Enterococcus faecium, a carbapenem-resistant Enterobacteriaceae spp. selected from Klebsiella pneumonia, Klebsiella oxytoca, Klebsiella aerogenes, and Enterobacter spp., an extended spectrum beta-lactamase (ESBL) selected from E. coli and Klebsiella species, a methicillin-resistant Staphylococcus aureus (MRSA), or combinations thereof;
(iii) the ESKAPE pathogen comprises Enterococcus faecium, Staphylococcus aureus, Klebsiella pneumoniae, Acinetobacter baumannii, Pseudomonas aeruginosa, and Enterobacter spp., or a combination thereof; or
(iv) the antibiotic resistant bacteria and ESKAPE pathogen are selected from (i) and (iii), (ii) and (iii), or (i), (ii), and (iii).

17. The composition of any one of claims 1 to 16, wherein one or more bacteria of the purified population of bacteria competes for carbon-sources with the antibiotic resistant bacteria.

18. The composition of any one of claims 1 to 16, wherein the purified population of bacteria improves epithelial barrier integrity, reduces inflammation, and/or reduces mucositis in a gastrointestinal tract of a subject compared to a reference (e.g., a composition that does not include one or more of the bacteria disclosed herein, a corresponding reference in a subject that did not receive the composition disclosed herein, or a corresponding reference in the subject prior to the administration of the composition).

19. The composition of any one of claims 1 to 18, wherein the purified population of bacteria decreases mortality due to an invasive infection in a subject compared to a reference (e.g., a composition that does not include one or more of the bacteria disclosed herein, a corresponding reference in a subject that did not receive the composition disclosed herein, or a corresponding reference in the subject prior to the administration of the composition).

20. The composition of claim 19, wherein the invasive infection in the subject is an antibiotic resistant infection.

21. The composition of any one of claims 1 to 20, wherein the purified population of bacteria reduces transplantation-related complications in a subject compared to a reference (e.g., a composition that does not include one or more of the bacteria disclosed herein, a corresponding reference in a subject that did not receive the composition disclosed herein, or a corresponding reference in the subject prior to the administration of the composition).

22. The composition of any one of claims 1 to 21, wherein the purified population of bacteria increases the overall survival and/or progression-free survival of a subject compared to a reference (e.g., a corresponding reference in a subject that did not receive the composition disclosed herein or corresponding reference in the subject prior to the administration of the composition).

23. The composition of any one of claims 1 to 22, wherein the purified population of bacteria modulates a biological activity, wherein the biological activity comprises short-chain fatty acid production, medium-chain fatty acid production, tryptophan metabolite production, fucosidase activity, Wnt activation, anti-IL-8 activity, carbon source utilization, bile acid metabolism, or combinations thereof.

24. The composition of any one of claims 2 to 23, wherein the purified population of bacteria comprises one or more bacteria having one or more features selected from the group consisting of:
(i) capable of reducing VRE and CRE carriage and restore colonization resistance in the GI tract of a mammal;
(ii) capable of protecting the epithelial barrier from cytokine-mediated inflammatory damage;
(iiii) capable of reducing inflammation in the epithelial barrier, as measured by IL-8 secretion in vitro, and in the colonic lamina propria of mice;
and combinations thereof.

25. The composition of any one of claims 1 to 24, wherein the purified population of bacteria comprises one or more bacteria having one or more features selected from:
(i) capable of engrafting (long-term and/or transient) when administered to a subject, (ii) capable of having anti-inflammatory activity (e.g., inhibiting TNF-α-driven IL-8 secretion in epithelial cells in vitro, ability to down-modulate expression of inflammatory genes (e.g., CXCL1, CXCL2, CXCL3, CXCL11, ICAM1)), (iii) not capable of inducing pro-inflammatory activity, (iv) capable of producing a secondary bile acid (e.g., 7α-dehydroxylase and bile salt hydrolase activity), (v) capable of producing a tryptophan metabolite (e.g., indole, 3-methyl indole, indolepropionic acid), (vi) capable of restoring epithelial integrity as determined by a primary epithelial cell monolayer barrier integrity assay, (vii) capable of being associated with reduction of risk of infection or GvHD following HSCT, (viii) capable of not being associated with clinical non-remission of infection or GvHD following HSCT, (ix) capable of producing a short-chain fatty acid (e.g., butyrate, propionate), (x) capable of inhibiting a HDAC activity, (xi) capable of producing a medium-chain fatty acid (e.g., valerate, hexanoate), (xii) capable of expressing catalase activity, (xiii) capable of having alpha-fucosidase activity, (xiv) capable of inducing Wnt activation, (xv) capable of producing a B vitamin (e.g., thiamin (B1) and/or pyridoxamine (B6)), (xvi) capable of reducing fecal calprotectin level, (xvii) not capable of activating a toll-like receptor pathway (e.g., TLR4 or TLR5), (xviii) capable of activating a toll-like receptor pathway (e.g., TLR2), (xix) capable of restoring colonization resistance, (xx) capable of a broad range of carbon source utilization; (xxi) capable of reducing VRE pathogen carriage, (xxii) capable of reducing CRE pathogen carriage, (xxiii) capable of reducing expression of claudin-2, (xxiv) capable of being associated with the healthy human gut microbiota, (xxv) capable of not being associated with toxin and hemolysin genes associated with Clostridial pathogens and no significant cytopathic effects in vitro, (xxvi) susceptible to multiple clinically relevant antibiotics, (xxvii) capable of not being associated with genes that are both likely responsible for the observed antibiotic resistances and transmissible, (xxxviii) capable of inhibiting epithelial cell apoptosis, (xxix) capable of down-modulating one or more genes induced in IFN-γ treated colonic organoids (e.g., those associated with inflammatory chemokine signaling, NF-κB signaling, TNF family signaling, type I interferon signaling, type II interferon signaling, TLR signaling, lymphocyte trafficking, Th17 cell differentiation, Th1 differentiation, Th2 differentiation, apoptosis, inflammasomes, autophagy, oxidative stress, MHC class I and II antigen presentation, complement, mTor, nod-like receptor signaling, PI3K signaling, or combinations thereof), (xxx) capable of reducing the expression of one or more inhibitory receptors (e.g., TIGIT, TIM-3, or LAG-3) on CD8+ T cells, (xxxi) capable of increasing expression of one or more genes/proteins associated with CD8+ T cell activation and/or function (e.g., CD45RO, CD69, IL-24, TNF-α, perforin, or IFN-γ), (xxxii) capable of enhancing the ability of CD8+ T cells to kill tumor cells, (xxxiii) capable of enhancing the efficacy of an immune checkpoint inhibitor therapy, (xxxiv) capable of promoting the recruitment of CD8+ T cells to tumors, (xxxv) capable of inducing an anti-inflammatory IL-10-skewed IL-10/IL-6 cytokine ratio in macrophages, (xxxvi) capable of inducing less inflammatory responses but similar pathogen defense responses in macrophages than a donor-derived spore-based composition (i.e., a spore-based composition), (xxxvii) capable of increasing the amount of anti-inflammatory mediators in (e.g., IL-1 receptor antagonists (IL-1RA), IL-4, IL-10, IL-11, IL-13, TGF-β), (xxxviii) capable of reducing colonic inflammation, (xxxix) capable of treating and/or preventing a disease or disorder, such as those associated with dysbiosis of a gastrointestinal tract, (xl) capable of increasing the diversity of the gastrointestinal microbiome in a subject, (xli) capable of improving mucosal and/or epithelial barrier integrity in a subject compared to a reference control (e.g., untreated patients or the subject prior to treatment), (xlii) capable of promoting mucosal healing, (xliii) capable of reducing incidence of infection, (xliv) capable of reducing the need for antibiotics in a subject, (xlv) capable of increasing the probability of survival in a subject, (xlvi) capable of reducing the risk of relapse of primary cancer, (xlvii) capable of reducing the abundance of a biomarker of infection in the stool of a subject, (xlviii) capable of increasing the abundance of a biomarker of an administered species in the stool of a subject, (xlix) capable of targeted delivery of most (e.g., 70%, 75%, 80%, 85%, 90%, 95%, 96%, 97%, 98%, 99%, 99.5%, 99.6%, 99.7%, 99.8%, or 99.9% of the administered species relative to the number of colony forming units administered) or all of the administered species to the intestines of the subject (e.g., through encapsulation, or through coating one or more components of a dosage form with an enteric polymer), (l) capable of a therapeutic benefit following a single administration of a composition or pharmaceutical composition described herein to a subject, (li) capable of coadministration with an additional agent described herein, without substantially decreasing the therapeutic benefit of the administered species, (lii) capable of coadministration with a carrier or excipient described herein, without substantially decreasing the therapeutic benefit of the administered species, (liii), or any combination thereof.

26. The composition of claim 25, wherein the biological activity is modulated in vivo.

27. The composition of claim 25, wherein the biological activity is modulated in vitro (e.g., a culture or a synthetic gastrointestinal system).

28. The composition of any one of claims 1 to 24, wherein the purified population of bacteria comprises one or more bacteria having one or more features selected from:
(i) capable of utilizing a carbon source used by a pathogenic organism, such as but not limited to Enterococcus and Enterobacteriaceae species and ESKAPE pathogens (including: Enterococcus faecium, Staphylococcus aureus, Klebsiella pneumoniae, Acinetobacter baumannii, Pseudomonas aeruginosa, and Enterobacter spp.), Enterococcus species including, but not limited to, Enterococcus faecalis and Enterococcus faecium, Enterobacteriaceae species including, but not limited to, Klebsiella pneumonia, or such species that are resistant to vancomycin or carbapenems, drug resistant or multi-drug resistant (MDROs) including VRE, CRE (Klebsiella pneumonia, Klebsiella oxytoca, Klebsiella aerogenes, Enterococcus species), extended spectrum beta-lactamase (ESBLs) producing bacteria (E. coli, Klebsiella species), or methicillin-resistant Staphylococcus aureus (MRSA); (ii) capable of engrafting when administered to a subject; (iii) capable of producing short-chain fatty acids; (iv) capable of producing medium-chain fatty acids; (v) capable of producing tryptophan metabolites; (vi) capable of inhibiting histone deacetylase (HDAC) activity; (vii) capable of decreasing IL-8 secretion in intestinal epithelial cells (IECs) treated with TNF-α; (viii) lack of induction of IL8 secretion in intestinal epithelial cells (IECs) in the absence of TNF-α; and combinations thereof.

29. The composition of any one of claims 1 to 24, wherein the purified population of bacteria comprises one or more bacteria having one or more features selected from:
(i) capable of having anti-inflammatory activity in epithelial cells, (ii) not capable of inducing pro-inflammatory activity, (iii) capable of producing a secondary bile acid, (iv) capable of producing a tryptophan metabolite, (v) capable of restoring epithelial integrity, (vi) capable of producing a short-chain fatty acid, (vii) capable of inhibiting a HDAC activity, (viii) capable of producing a medium-chain fatty acid, (ix) capable of restoring colonization resistance, (x) capable of reducing VRE pathogen carriage, (xi) capable of reducing CRE pathogen carriage, (xii) capable of inhibiting epithelial cell apoptosis, (xiii) capable of down-modulating one or more genes induced in IFN-γ treated colonic organoids, (xiv) capable of reducing the expression of one or more inhibitory receptors, (xv) capable of increasing expression of one or more genes/proteins associated with CD8+ T cell activation and/or function, (xvi) capable of enhancing the ability of CD8+ T cells to kill tumor cells, (xvii) capable of enhancing the efficacy of an immune checkpoint inhibitor therapy, (xviii) capable of inducing an anti-inflammatory IL-10-skewed IL-10/IL-6 cytokine ratio in macrophages, (xix) capable of inducing less inflammatory responses but similar pathogen defense responses in macrophages than a natural composition, (xx) capable of reducing colonic inflammation, (xxi) capable of treating and/or preventing a disease or disorder, such as those associated with dysbiosis of a gastrointestinal tract, (xxii) capable of increasing the Treg:Th1 or Treg:Th17 ratios on the lamina propria of mice, and combinations thereof.

30. The composition of any one of claims 1 to 24, wherein the purified population of bacteria comprises one or more bacteria having one or more features selected from:
(i) capable of having anti-inflammatory activity in epithelial cells (e.g., inhibiting TNF-α-driven IL-8 secretion in epithelial cells in vitro, ability to down-modulate expression of inflammatory genes (e.g., CXCL1, CXCL2, CXCL3, CXCL11, ICAM1)), (ii) not capable of inducing pro-inflammatory activity, (iii) capable of restoring epithelial integrity as determined by a primary epithelial cell monolayer barrier integrity assay, (iv) capable of inhibiting epithelial cell apoptosis, (v) capable of down-modulating one or more genes induced in IFN-γ treated colonic organoids (e.g., those associated with inflammatory chemokine signaling, NF-κB signaling, TNF family signaling, type I interferon signaling, type II interferon signaling, TLR signaling, lymphocyte trafficking, Th17 cell differentiation, Th2 differentiation, apoptosis, inflammasomes, autophagy, oxidative stress, MHC class I and II antigen presentation, complement, mTor, nod-like receptor signaling, PI3K signaling, or combinations thereof), (vi) capable of reducing the expression of one or more inhibitory receptors (e.g., TIGIT, TIM-3, or LAG-3) on CD8+ T cells, (vii) capable of increasing expression of one or more genes/proteins associated with CD8+ T cell activation and/or function (e.g., CD45RO, CD69, IL-24, TNF-α, perforin, or IFN-γ), (viii) capable of enhancing the ability of CD8+ T cells to kill tumor cells, (ix) capable of enhancing the efficacy of an immune checkpoint inhibitor therapy, (x) capable of increasing the Treg:Th1 or Treg:Th17 ratios on the lamina propria of mice, and combinations thereof.

31. The composition of any one of claims 1 to 24, wherein the composition:
(i) increases frequency of Tregs and does not increase frequency of Th1 or Th17 effector T cells in the colon of a subject compared to a reference (e.g., a corresponding reference in a subject that did not receive the composition disclosed herein or corresponding reference in the subject prior to the administration of the composition),
(ii) the ratio of Tregs to Th1 effector T cells or Tregs to Th17 effector T cells in the colon of a subject compared to a reference (e.g., a corresponding reference in a subject that did not receive the composition disclosed herein or corresponding reference in the subject prior to the administration of the composition),
or (i) and (ii).

32. The composition of any one of claims 1 to 31, wherein the purified population of bacteria augments the number and/or relative abundance of spore-forming bacteria in a microbiome of a subject.

33. The composition of any one of claims 1 to 32, wherein the purified population of bacteria augments the number and/or relative abundance of non-pathogenic, commensal non-spore-forming bacteria in a microbiome of a subject.

34. The composition of any one of claims 1 to 33, wherein one or more bacteria of the purified population of bacteria are capable of being engrafted into a subject's microbiome when administered to the subject, wherein said engraftment is long-term or transient engraftment.

35. A pharmaceutical formulation comprising the composition of any one of claims 1 to 34 and a pharmaceutically acceptable excipient.

36. The pharmaceutical formulation of claim 35, wherein the excipient comprises glycerol.

37. The pharmaceutical formulation of claim 35 or 36, wherein the composition is lyophilized.

38. The pharmaceutical formulation of any one of claims 35 to 37, wherein the composition is formulated for oral delivery.

39. The pharmaceutical formulation of any one of claims 35 to 38, wherein the composition is encapsulated.

40. A method of treating a disease or disorder associated with an allogeneic immune response in a subject in need thereof, comprising administering to the subject an effective amount of the composition of any one of claims 1 to 34 or the pharmaceutical formulation of any one of claims 35 to 39.

41. The method of claim 40, wherein the allogeneic immune response is caused by an allogeneic hematopoietic stem cell transplantation (allo-HSCT) or an allogeneic organ transplantation.

42. The method of claim 40 or 41, wherein the disease or disorder associated with an allogenic immune response comprises graft-versus-host-disease (GvHD), viral infection or reactivation, invasive infection, blood stream infection, inflammation, or combinations thereof.

43. The method of claim 42, wherein the GvHD comprises an acute graft versus host disease (aGvHD) or a chronic graft versus host disease (cGvHD).

44. The method of any one of claims 40 to 43, wherein the subject suffers from a cancer.

45. The method of claim 44, wherein the cancer comprises acute myeloid leukemia (AML), acute lymphoblastic leukemia (ALL), myelodysplastic syndromes (MDS), myeloproliferative neoplasms (MPN), or combinations thereof.

46. A method of treating a disease or disorder associated with an autologous immune response in a subject in need thereof, comprising administering to the subject an effective amount of the composition of any one of claims 1 to 34 or the pharmaceutical formulation of any one of claims 35 to 39.

47. The method of claim 46, wherein the autologous immune response is caused by an autologous hematopoietic stem cell transplantation or an autologous organ transplantation.

48. The method of claim 46 or 47, wherein the disease or disorder associated with an autologous immune response comprises graft-versus-host-disease (GvHD), viral infection or reactivation, invasive infection, blood stream infection, inflammation, or combinations thereof.

49. The method of claim 48, wherein the GvHD comprises an acute graft versus host disease (aGvHD) or a chronic graft versus host disease (cGvHD).

50. The method of any one of claims 46 to 49, wherein the subject suffers from a cancer.

51. The method of claim 50, wherein the cancer comprises acute myeloid leukemia (AML), acute lymphoblastic leukemia (ALL), myelodysplastic syndromes (MDS), myeloproliferative neoplasms (MPN), or combinations thereof.

52. A method of treating, reducing, or alleviating a symptom associated with chemotherapy in a subject in need thereof, comprising administering to the subject an effective amount of the composition of any one of claims 1 to 34 or the pharmaceutical formulation of any one of claims 35 to 39.

53. The method of claim 52, wherein the symptom associated with chemotherapy comprises weight loss or an increase in the level of a proinflammatory mediator within a gastrointestinal tract of the subject.

54. The method of claim 53, wherein the weight loss is reduced by at least 10%, at least 20%, at least 30%, at least 40%, at least 50%, at least 60%, at least 70%, at least 80%, or at least 90% compared to a reference (e.g., corresponding value in a subject that did not receive the composition disclosed herein or corresponding value in the subject prior to the administration of the composition).

55. The method of claim 53, wherein the level of a proinflammatory mediator is reduced by at least 10%, at least 20%, at least 30%, at least 40%, at least 50%, at least 60%, at least 70%, at least 80%, or at least 90% compared to a reference (e.g., corresponding value in a subject that did not receive the composition disclosed herein or corresponding value in the subject prior to the administration of the composition).

56. The method of any one of claims 53 to 55, wherein the proinflammatory mediator comprises IFN-γ, IL-1b, IL-2, IL-6, IL-12, CXCL5, IL-17, CXCL1, VEGF, TNF-α, or combinations thereof.

57. The method of claim 56, wherein the level of a proinflammatory T cell is reduced by at least 10%, at least 20%, at least 30%, at least 40%, at least 50%, at least 60%, at least 70%, at least 80%, or at least 90% compared to a reference (e.g., corresponding value in a subject that did not receive the composition disclosed herein or corresponding value in the subject prior to the administration of the composition).

58. The method of claim 57, wherein the proinflammatory T cell comprises a CD8⁺ T cell.

59. The method of claim 56, wherein the level of an anti-inflammatory T cell is reduced by at least 10%, at least 20%, at least 30%, at least 40%, at least 50%, at least 60%, at least 70%, at least 80%, or at least 90% compared to a reference (e.g., corresponding value in a subject that did not receive the composition disclosed herein or corresponding value in the subject prior to the administration of the composition).

60. The method of claim 59, wherein the anti-inflammatory T cell comprises a FOXP3⁺ CD4⁺ T cell.

61. The method of claim 520, wherein the symptom associated with chemotherapy comprises inflammation.

62. The method of claim 52, wherein the treating, reducing, or alleviating a symptom associated with chemotherapy comprises treating, reducing, or alleviating inflammation.

63. The method of claim 62 wherein the treating, reducing, or alleviating inflammation is measured by analyzing one or more of G-CSF, IFNγ, IL-6, IP-10, KC, MCP-1, MIP-2, MIG, or TNFα.

64. The method of claim 62, wherein following the administration of an effective amount of the composition or pharmaceutical formulation the subject does not have an increased level of one or more of G-CSF, IFNγ, IL-6, IP-10, KC, MCP-1, MIP-2, MIG, or TNFα compared to a reference (e.g., corresponding value in a subject that did not receive the composition disclosed herein or corresponding value in the subject prior to the administration of the composition).

65. A method of preventing, reducing, or treating rejection in a subject undergoing transplantation (e.g., either HSCT or organ), comprising administering to the subject an effective amount of the composition of any one of claims 1 to 34 or the pharmaceutical formulation of any one of claims 35 to 39.

66. The method of claim 65, wherein the composition or the pharmaceutical formulation is administered to the subject prior to, during, and/or after the transplantation.

67. A method of modulating a biological activity in a subject in need thereof, comprising administering to the subject an effective amount of the composition of any one of claims 1 to 34 or the pharmaceutical formulation of any one of claims 35 to 39, wherein the biological activity comprises short-chain fatty acid production, medium-chain fatty acid production, tryptophan metabolite production, fucosidase activity, Wnt activation, anti-IL-8 activity, or combinations thereof.

68. The method of claim 67, wherein short-chain fatty acid production is increased by at least 10%, at least 20%, at least 30%, at least 40%, at least 50%, at least 60%, at least 70%, at least 80%, or at least 90% compared to a reference (e.g., corresponding activity in a subject that did not receive the composition disclosed herein or corresponding activity in the subject prior to the administration of the composition).

69. The method of claim 67, wherein medium-chain fatty acid production is increased by at least 10%, at least 20%, at least 30%, at least 40%, at least 50%, at least 60%, at least 70%, at least 80%, or at least 90% compared to a reference (e.g., corresponding activity in a subject that did not receive the composition disclosed herein or corresponding activity in the subject prior to the administration of the composition).

70. The method of claim 67, wherein tryptophan metabolite production is increased by at least 10%, at least 20%, at least 30%, at least 40%, at least 50%, at least 60%, at least 70%, at least 80%, or at least 90% compared to a reference (e.g., corresponding activity in a subject that did not receive the composition disclosed herein or corresponding activity in the subject prior to the administration of the composition).

71. The method of claim 67, wherein fucosidase activity is increased by at least 10%, at least 20%, at least 30%, at least 40%, at least 50%, at least 60%, at least 70%, at least 80%, or at least 90% compared to a reference (e.g., corresponding activity in a subject that did not receive the composition disclosed herein or corresponding activity in the subject prior to the administration of the composition).

72. The method of claim 67, wherein Wnt activation is increased by at least 10%, at least 20%, at least 30%, at least 40%, at least 50%, at least 60%, at least 70%, at least 80%, or at least 90% compared to a reference (e.g., corresponding activity in a subject that did not receive the composition disclosed herein or corresponding activity in the subject prior to the administration of the composition).

73. The method of claim 67, wherein anti-IL-8 activity is increased by at least 10%, at least 20%, at least 30%, at least 40%, at least 50%, at least 60%, at least 70%, at least 80%, or at least 90% compared to a reference (e.g., corresponding activity in a subject that did not receive the composition disclosed herein or corresponding activity in the subject prior to the administration of the composition).

74. A method of decreasing the number and/or relative abundance of antibiotic resistant bacteria in a gastrointestinal tract of a subject in need thereof, comprising administering to the subject an effective amount of the composition of any one of claims 1 to 34 or the pharmaceutical formulation of any one of claims 35 to 39.

75. The method of claim 74, wherein the number and/or relative abundance of antibiotic resistant bacteria is reduced by at least 10%, at least 20%, at least 30%, at least 40%, at least 50%, at least 60%, at least 70%, at least 80%, or at least 90%, or 1 log, 2 log, 3 log, 4 log, 5 log compared to a reference (e.g., corresponding number in a subject that did not receive the composition disclosed herein or corresponding number in the subject prior to the administration of the composition).

76. The method of claim 74 or 75, wherein the antibiotic resistant bacteria comprise vancomycin-resistant Enterococci or carbapenem-resistant Enterobacteriaceae.

77. The method of claim 74 or 75, wherein the antibiotic resistant bacteria comprise an ESKAPE pathogen (including Enterococcus faecium, Staphylococcus aureus, Klebsiella pneumoniae, Acinetobacter baumannii, Pseudomonas aeruginosa, and Enterobacter spp.).

78. The method of claim 74 or 75, wherein the antibiotic resistant bacteria comprise an Enterococcus species including an Enterococcus species selected from Enterococcus faecalis and Enterococcus faecium.

79. The method of claim 74 or 75, wherein the antibiotic resistant bacteria comprise an Enterobacteriaceae species including Klebsiella pneumonia.

80. The method of claim 74 or 75, wherein the antibiotic resistant bacteria comprise a drug resistant or multi-drug resistant (MDROs) bacteria including a vancomycin-resistant Enterococcus spp. selected from Enterococcus faecalis and Enterococcus faecium, a carbapenem-resistant Enterobacteriaceae spp. selected from Klebsiella pneumonia, Klebsiella oxytoca, Klebsiella aerogenes, and Enterobacter spp., an extended spectrum beta-lactamase (ESBL) selected from E. coli and Klebsiella species, a methicillin-resistant Staphylococcus aureus (MRSA), or combinations thereof.

81. A method of improving epithelial barrier status, reducing inflammation, and/or reducing mucositis in a gastrointestinal tract of a subject in need thereof, comprising administering to the subject an effective amount of the composition of any one of claims 1 to 34 or the pharmaceutical formulation of any one of claims 35 to 39.

82. The method of claim 81, wherein the epithelial barrier status in the gastrointestinal tract of the subject is improved by at least 5%, at least 10%, at least 20%, at least 30%, at least 40%, at least 50%, at least 60%, at least 70%, at least 80%, or at least 90% compared to a reference (e.g., corresponding value in a subject that did not receive the composition disclosed herein or corresponding activity in the subject prior to the administration of the composition).

83. The method of claim 81, wherein the inflammation in the gastrointestinal tract of the subject is reduced by at least 5%, at least 10%, at least 20%, at least 30%, at least 40%, at least 50%, at least 60%, at least 70%, at least 80%, or at least 90% compared to a reference (e.g., corresponding value in a subject that did not receive the composition disclosed herein or corresponding activity in the subject prior to the administration of the composition).

84. The method of claim 81, wherein the mucositis in the gastrointestinal tract of the subject is reduced by at least 5%, at least 10%, at least 20%, at least 30%, at least 40%, at least 50%, at least 60%, at least 70%, at least 80%, or at least 90% compared to a reference (e.g., corresponding value in a subject that did not receive the composition disclosed herein or corresponding activity in the subject prior to the administration of the composition).

85. A method of decreasing mortality due to an invasive infection in a subject in need thereof, comprising administering to the subject an effective amount of the composition of any one of claims 1 to 34 or the pharmaceutical formulation of any one of claims 35 to 39, wherein the subject is undergoing transplantation.

86. The method of claim 85, wherein the invasive infection in the subject is an antibiotic resistant infection.

87. A method of reducing transplantation-related complications in a subject in need thereof, comprising administering to the subject an effective amount of the composition of any one of claims 1 to 34 or the pharmaceutical formulation of any one of claims 35 to 39, wherein the subject is undergoing transplantation.

88. A method of increasing the overall survival and/or progression-free survival of a subject in need thereof, comprising administering to the subject an effective amount of the composition of any one of claims 1 to 34 or the pharmaceutical formulation of any one of claims to 39, wherein the subject is undergoing transplantation.

89. The method of any one of claims 40 to 88, wherein the subject is undergoing or has undergone transplantation.

90. The method of claim 89, wherein the transplantation is an allogeneic hematopoietic stem cell transplantation (allo-HSCT) or an allogeneic organ transplantation.

91. The method of claim 89, wherein the transplantation is an autologous hematopoietic stem cell transplantation or an autologous organ transplantation.

92. The method of any one of claims 52 to 91, wherein the subject suffers from a cancer.

93. The method of claim 92, wherein the cancer comprises acute myeloid leukemia (AML), acute lymphoblastic leukemia (ALL), myelodysplastic syndromes (MDS), myeloproliferative neoplasms (MPN), or combinations thereof.

94. A method of treating and/or reducing the risk of an infection in a subject in need thereof, comprising administering to the subject an effective amount of the composition of any one of claims 1 to 34 or the pharmaceutical formulation of any one of claims 35 to 39.

95. The method of claim 94, wherein the infection includes a blood stream infection, sepsis, tissue infection, invasive infection, a gastrointestinal infection, a viral infection or reactivation, or a combination thereof.

96. The method of claim 95, wherein the infection is a viral infection or reactivation.

97. The method of claim 94 or 95, wherein the infection is caused by an ESKAPE pathogen (including Enterococcus faecium, Staphylococcus aureus, Klebsiella pneumoniae, Acinetobacter baumannii, Pseudomonas aeruginosa, and Enterobacter spp.).

98. The method of claim 97, wherein the infection is caused by an Enterococcus species including an Enterococcus species selected from Enterococcus faecalis and Enterococcus faecium.

99. The method of claim 97, wherein the infection is caused by an Enterobacteriaceae species including Klebsiella pneumonia.

100. The method of claim 97, wherein the infection is caused by a bacterial species resistant to vancomycin or carbapenems.

101. The method of claim 97, wherein the infection is caused by a drug resistant or multi-drug resistant (MDROs) bacteria including a vancomycin-resistant Enterococcus spp. selected from Enterococcus faecalis and Enterococcus faecium, a carbapenem-resistant Enterobacteriaceae spp. selected from Klebsiella pneumonia, Klebsiella oxytoca, Klebsiella aerogenes, and Enterobacter spp., an extended spectrum beta-lactamase (ESBL) selected from E. coli and Klebsiella species, a methicillin-resistant Staphylococcus aureus (MRSA), or combinations thereof.

102. A method of treating and/or reducing the risk of graft-versus-host-disease (GvHD) in a subject in need thereof, comprising administering to the subject an effective amount of the composition of any one of claims 1 to 34 or the pharmaceutical formulation of any one of claims to 39.

103. The method of claim 102, wherein the GvHD comprises an acute graft versus host disease (aGvHD) or a chronic graft versus host disease (cGvHD).

104. A method of treating and/or reducing the risk of mucositis in a subject in need thereof, comprising administering to the subject an effective amount of the composition of any one of claims 1 to 34 or the pharmaceutical formulation of any one of claims 35 to 39.

105. The method of any one of claims 94 to 104, wherein the subject suffers from a cancer.

106. The method of claim 105, wherein the cancer comprises acute myeloid leukemia (AML), acute lymphoblastic leukemia (ALL), myelodysplastic syndromes (MDS), myeloproliferative neoplasms (MPN), or combinations thereof.

107. The method of any one of claims 40-106, wherein following administration of the composition or the pharmaceutical formulation, the composition or the pharmaceutical formulation increases frequency of Tregs and does not increase frequency of Th1 or Th17 effector T cells in the colon of the subject compared to a reference (e.g., a corresponding reference in a subject that did not receive the composition disclosed herein or corresponding reference in the subject prior to the administration of the composition).

108. The method of any one of claims 40-106, wherein following administration of the composition or the pharmaceutical formulation, the composition or the pharmaceutical formulation increases the ratio of Tregs to Th1 effector T cells or Tregs to Th17 effector T cells in the colon of the subject compared to a reference (e.g., a corresponding reference in a subject that did not receive the composition disclosed herein or corresponding reference in the subject prior to the administration of the composition).

109. The method of any one of claims 40-108, wherein the composition or the pharmaceutical formulation reduces the risk of one or more of: death, an immediate risk of death, a hospitalization, a prolongation of existing hospitalization, a significant disruption in the subject's ability to conduct normal life functions, an event associated with a pre-existing congenital anomaly or birth defect, or a need for medical or surgical intervention, in the subject compared to a reference (e.g., a corresponding reference in a subject that did not receive the composition or pharmaceutical formulation disclosed herein or corresponding reference in the subject prior to the administration of the composition or pharmaceutical formulation).

110. The method of claim 109, wherein the composition or pharmaceutical formulation reduces the risk of an invasive infection or a bloodstream infection in the subject compared to a subject that did not receive the composition or pharmaceutical formulation.

111. The method of claim 110, wherein the invasive infection or the bloodstream infection is one or more of bacterial meningitis, fungal meningitis, pleural empyema, pericardial empyema, hepatic abscess, splenic abscess, pulmonary abscess, urinary tract infection, or sterile site infection

112. The method of any one of claims 40-111, wherein following administration of the composition or the pharmaceutical formulation, one or more species of the composition or pharmaceutical formulation engrafts in the subject's gastrointestinal tract.

113. The method of claim 40-112, wherein the composition or the pharmaceutical formulation reduces the risk of one or more of:
a bloodstream infection,
a gastrointestinal infection,
acute GvHD, or
febrile neutropenia characterized by a body temperature of 38.0° C. and absolute neutrophil count less than 500 cells/mm 3 in the absence of an identified infectious agent,
in the subject compared to a subject that did not receive the composition or pharmaceutical formulation.

114. The method of claim 113, wherein the bloodstream infection is a bacterial infection or a fungal infection.

115. The method of claim 114, wherein the bacterial infection is VRE or CRE.

116. The method of claim 113, wherein the gastrointestinal infection is a bacterial infection, a viral infection, or a parasitic infection.

117. The method of claim 116, wherein the bacterial infection is of Clostridium difficile.

118. The method of claim 116, wherein the viral infection is of one or more of a norovirus, a rotavirus, or an adenovirus.

119. The method of claim 116, wherein the parasitic infection is of a Cryptosporidia species.

120. The method of any one of claims 113-119, wherein the composition or the pharmaceutical formulation reduces the risk of acute GvHD and gastrointestinal domination by one or more Enterococcus or Enterobacteriaceae species, or a combination thereof.

121. The method of any one of claims 113-119, wherein the composition or the pharmaceutical formulation reduces the risk of acute GvHD of severity Grade II, Grade III, or Grade IV.

122. The method of any one of claims 40-121, wherein following administration of the composition or the pharmaceutical formulation, the amount of a biomarker associated with one or more administered species is increased in the subject, or
a biomarker associated with acute GvHD is not detectable in the subject.

123. The method of claim 122, wherein the biomarker associated with one or more administered species is a urinary concentration of 3-indoxyl sulfate.

124. The method of claim 122 or claim 123, wherein the biomarker associated with acute GvHD is absent from the blood plasma of the subject, and is one or more of suppression of tumorigenicity 2 (ST2) or regenerating islet-derived 3α (REG3α).

125. The method of any one of claims 112-124, comprising a step of detecting that the one or more administered species have engrafted in the subject after administration of the composition or pharmaceutical composition.

126. The method of any one of claims 40-125, comprising a step of detecting the abundance of one or more of an Enterococcus species or an Enterobacteriaceae species after administration of the composition or pharmaceutical composition.

127. The method of claim 126, wherein the abundance of the one or more of an Enterococcus species or an Enterobacteriaceae species is decreased after administration of the composition or pharmaceutical composition.

128. The method of claim 127, wherein the one or more of an Enterococcus species or an Enterobacteriaceae species is a VRE species, a CRE species, or an ESBL species.

129. The method of any one of claims 125-128, wherein the one or more administered species or the one or more of an Enterococcus species or an Enterobacteriaceae species is detected in a stool of the subject prior to, after, or prior to and after administration of the composition or pharmaceutical composition.

130. The method of claim 129, wherein the one or more administered species or the one or more of an Enterococcus species or an Enterobacteriaceae species is detected in a stool of the subject by one or more steps comprising detecting the presence of a gene or genome of the one or more administered species or the one or more of an Enterococcus species or an Enterobacteriaceae species in a stool of the subject.

131. The method of claim 129 or 130, wherein the one or more administered species or the one or more of an Enterococcus species or an Enterobacteriaceae species is detected in a stool of the subject by one or more steps comprising cultivating the one or more administered species or the one or more of an Enterococcus species or an Enterobacteriaceae species from a stool of the subject.

132. The method of any one of claims 128-131, wherein administration of the composition or pharmaceutical composition reduces the risk of gastrointestinal domination in the subject by the one or more of an Enterococcus species or an Enterobacteriaceae species.

133. The method of any one of claims 40-132, wherein administration of the composition or pharmaceutical composition reduces one or more of:
the need for administration of one or more anti-infective agents to the subject,
the frequency of hospitalization of the subject,
the length of hospitalization of the subject, or
transplant-related mortality of the subject.

134. The method of any one of claims 113-133, wherein administration of the composition or pharmaceutical composition increases one or more of relapse-free survival of the subject and GvHD-free survival of the subject.

135. The method of any one of claims 113-134, wherein the gastrointestinal infection is determined with an enzyme immunoassay or a cell culture cytotoxicity neutralization assay.
